(19) United States
(12) Patent Application Publication
(10) Pub. No.: US 2014/0228363 A1
(43) Pub. Date: Aug. 14, 2014

(54) TETRASUBSTITUTED CYCLOHEXYL COMPOUNDS AS KINASE INHIBITORS

(71) Applicants: Matthew Burger, Albany, CA (US); Yu Ding, Union City, CA (US); Wooseok Han, San Ramon, CA (US); Gisele Nishiguchi, Albany, CA (US); Alice Rico, Castro Valley, CA (US); Robert Lowell Simons, San Francisco, CA (US); Aaron R. Smith, Fremont, CA (US); Victoriano Tamez, JR., Emeryville, CA (US); Huw Tanner, Alameda, CA (US); Lifeng Wan, Union City, CA (US)

(72) Inventors: Matthew Burger, Albany, CA (US); Yu Ding, Union City, CA (US); Wooseok Han, San Ramon, CA (US); Gisele Nishiguchi, Albany, CA (US); Alice Rico, Castro Valley, CA (US); Robert Lowell Simons, San Francisco, CA (US); Aaron R. Smith, Fremont, CA (US); Victoriano Tamez, JR., Emeryville, CA (US); Huw Tanner, Alameda, CA (US); Lifeng Wan, Union City, CA (US)

(73) Assignee: NOVARTIS AG, Basel (CH)

(21) Appl. No.: 14/258,764

(22) Filed: Apr. 22, 2014

Related U.S. Application Data
(63) Continuation of application No. 13/410,203, filed on Mar. 1, 2012, now abandoned.
(60) Provisional application No. 61/449,222, filed on Mar. 4, 2011.

(51) Int. Cl. C07D 401/12; A61K 45/06; A61K 31/506; C07D 417/12; A61K 31/4439; C07D 409/14; C07D 417/14; C07D 405/14; C07D 401/14; A61K 31/5377; C07D 413/14; A61K 31/444; A61K 31/497

(52) U.S. Cl. CPC C07D 401/12; A61K 31/444; A61K 45/06; A61K 31/506; C07D 417/12; A61K 31/4439; A61K 31/497; C07D 417/14; C07D 405/14; C07D 401/14; A61K 31/5377; C07D 413/14; C07D 409/14
USPC 514/235.5; 546/262; 514/332; 544/322; 514/256; 546/270.7; 514/342; 544/405; 514/255.05; 546/256; 514/333; 544/131

(57) ABSTRACT

The present invention provides a compound of formula (I):

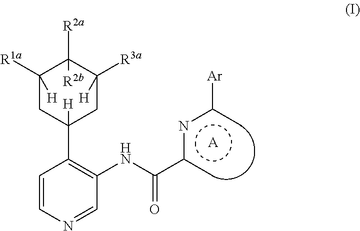

as further described herein, and pharmaceutically acceptable salts, enantiomers, rotamers, tautomers, or racemates thereof. Also provided are methods of treating a disease or condition mediated by PIM kinase using the compounds of Formula I, and pharmaceutical compositions comprising such compounds.

### CROSS-REFERENCE TO RELATED APPLICATIONS

This application is a continuation application of U.S. Ser. No. 13/410,203, filed Mar. 1, 2012, which claims the benefit of priority under 35 U.S.C. §119(e) to U.S. provisional application Ser. No. 61/449,222 filed on Mar. 4, 2011, and U.S. provisional application Ser. No. 61/479,996 filed on Apr. 28, 2011, which are incorporated herein by reference in their entirety.

### FIELD OF THE INVENTION

The present invention relates to new compounds and their tautomers and pharmaceutically acceptable salts, esters, metabolites or prodrugs thereof, compositions of the new compounds together with pharmaceutically acceptable carriers, and uses of the new compounds, either alone or in combination with at least one additional therapeutic agent, in the prophylaxis or treatment of cancer and other cellular proliferation disorders.

### BACKGROUND

Infection with the Maloney retrovirus and genome integration in the host cell genome results in development of lymphomas in mice. Provirus Integration of Maloney Kinase (PIM-Kinase) was identified as one of the frequent proto-oncogenes capable of being transcriptionally activated by this retrovirus integration event (Cuypers H T et al., “Murine leukemia virus-induced T-cell lymphomagenesis: integration of proviruses in a distinct chromosomal region,” Cell 37(1): 141-50 (1984); Selten G, et al., “Proviral activation of the putative oncogene Pim-1 in MuLV induced T-cell lymphomas” EMBO J 4(7): 1793-8 (1985)), thus establishing a correlation between over-expression of this kinase and its oncogenic potential. Sequence homology analysis demonstrated that there are three highly homologous Pim-Kinases (Pim1, 2 & 3), Pim1 being the proto-oncogene originally identified by retrovirus integration. Furthermore, transgenic mice over-expressing Pim1 or Pim2 show increased incidence of T-cell lymphomas (Breuer M et al., “Very high frequency of lymphoma induction by a chemical carcinogen in pim-1 transgenic mice” Nature 340(6228):61-3 (1989)), while over-expression in conjunction with c-myc is associated with incidence of B-cell lymphomas (Verbeek S et al., “Mice bearing the E mu-myc and E mu-pim-1 transgenes develop pre-B-cell leukemia prenatally” Mol Cell Biol 11(2):1176-9 (1991)). Thus, these animal models establish a strong correlation between Pim over-expression and oncogenesis in hematopoietic malignancies.

In addition to these animal models, Pim over-expression has been reported in many human malignancies. Pim1, 2 & 3 over-expression is frequently observed in hematopoietic malignancies (Amson R et al., “The human protooncogene product p33pim is expressed during fetal hematopoiesis and in diverse leukemias,” PNAS USA 86(22):8857-61 (1989); Cohen A M et al., “Increased expression of the hPim-2 gene in human chronic lymphocytic leukemia and non-Hodgkin lymphoma,” Leuk Lymph 45(5):951-5 (2004), Huttmann A et al., “Gene expression signatures separate B-cell chronic lymphocytic leukaemia prognostic subgroups defined by ZAP-70 and CD38 expression status,” Leukemia 20:1774-1782 (2006)) and in prostate cancer (Dhanasekaran S M, et al., “Delineation of prognostic biomarkers in prostate cancer,” Nature 412(6849):822-6 (2001); Cibull T L, et al., “Overexpression of Pim-1 during progression of prostatic adenocarcinoma,” J Clin Pathol 59(3):285-8 (2006)), while over-expression of Pim3 is frequently observed in hepatocellular carcinoma (Fujii C, et al., “Aberrant expression of serine/threonine kinase Pim-3 in hepatocellular carcinoma development and its role in the proliferation of human hepatoma cell lines,” Int J Cancer 114:209-218 (2005)) and pancreatic cancer (Li Y Y et al., “Pim-3, a proto-oncogene with serine/threonine kinase activity, is aberrantly expressed in human pancreatic cancer and phosphorylates bad to block bad-mediated apoptosis in human pancreatic cancer cell lines,” Cancer Res 66(13):6741-7 (2006)).

Pim1, 2 & 3 are Serine/Threonine kinases that normally function in survival and proliferation of hematopoietic cells in response to growth factors and cytokines. Cytokines signaling through the Jak/Stat pathway leads to activation of transcription of the Pim genes and synthesis of the proteins. No further post-translational modifications are required for the Kinase Pim activity. Thus, signaling downstream is primarily controlled at the transcriptional/translational and protein turnover level. Substrates for Pim kinases include regulators of apoptosis such as the Bcl-2 family member BAD (Aho T et al., “Pim-1 kinase promotes inactivation of the pro-apoptotic Bad protein by phosphorylating it on the Ser112 gatekeeper site: FEBS Letters 571: 43-49 (2004)), cell cycle regulators such as p21^{WFA1/CIP1} (Wang Z, et al., “Phosphorylation of the cell cycle inhibitor p21Cip1/WAF1 by Pim-1 kinase,” Biochem Biophys Acta 1593:45-55 (2002)), CDC25A (1999), C-TAK (Bachmann M et al., “The Oncogenic Serine/Threonine Kinase Pim-1 Phosphorylates and Inhibits the Activity of Cdc25C-associated Kinase 1 (C-TAK1). A novel role for Pim-1 at the G2/M cell cycle checkpoint,” J Biol Chem 179:48319-48328 (2004)) and NuMA (Bhattacharya N, et al., “Pim-1 associates with protein complexes necessary for mitosis,” Chromosoma 111(2):80-95 (2002)) and the protein synthesis regulator 4EBP1 (Hammerman P S et al., “Pim and Akt oncogenes are independent regulators of hematopoietic cell growth and survival,” Blood 105(11):4477-83 (2005)). The effects of Pim(s) in these regulators are consistent with a role in protection from apoptosis and promotion of cell proliferation and growth. Thus, over-expression of Pim(s) in cancer is thought to play a role in promoting survival and proliferation of cancer cells and, therefore, their inhibitions should be an effective way of treating cancers in which they are over-expressed. In fact several reports indicate that knocking down expression of Pim(s) with siRNA results in inhibition of proliferation and cell death (Dai J M, et al., “Antisense oligodeoxynucleotides targeting the serine/threonine kinase Pim-2 inhibited proliferation of DU-145 cells,” Acta Pharmacol Sin 26(3):364-8 (2005); Fujii et al. 2005; Li et al. 2006).

Furthermore, mutational activation of several well known oncogenes in hematopoietic malignancies is thought to exert its effects at least in part through Pim(s). For example, targeted down-regulation of Pim expression impairs survival of hematopoietic cells transformed by Flt3 and BCR/ABL (Adam et al. 2006). Thus, inhibitors to Pim1, 2 and 3 would be useful in the treatment of these malignancies.

In addition to a potential role in cancer treatment and myeloproliferative diseases, such inhibitor could be useful to control expansion of immune cells in other pathologic condition such as autoimmune diseases, allergic reactions and in organ transplantation rejection syndromes. This notion is supported by the findings that differentiation of Th1 Helper T-cells by IL-12 and IFN-α results in induction of expression of both Pim1 and Pim2 (Aho T et al., “Expression of human Pim family genes is selectively up-regulated by cytokines promoting T helper type 1, but not T helper type 2, cell differentiation,” Immunology 116: 82-88 (2005)). Moreover, Pim(s) expression is inhibited in both cell types by the immunosuppressive TGF-β (Aho et al. 2005). These results suggest that Pim kinases are involved in the early differentiation process of Helper T-cells, which coordinate the immunological responses in autoimmune diseases, allergic reaction and tissue transplant rejection. Recent reports demonstrate that Pim kinase inhibitors show activity in animal models of inflammation and autoimmune diseases. See J E Robinson “Targeting the Pim Kinase Pathway for Treatment of Autoimmune and Inflammatory Diseases,” for the Second Annual Conference on Anti-Inflammatories: Small Molecule Approaches,” San Diego, Calif. (Conf. April 2011; Abstract published earlier on-line).

A continuing need exists for compounds that inhibit the proliferation of capillaries, inhibit the growth of tumors, treat cancer, modulate cell cycle arrest, and/or inhibit molecules such as Pim1, Pim2 and Pim3, and pharmaceutical formulations and medicaments that contain such compounds. A need also exists for methods of administering such compounds, pharmaceutical formulations, and medicaments to patients or subjects in need thereof. The present invention addresses such needs.

Earlier patent applications have described compounds that inhibit Pims and function as anticancer therapeutics, see, e.g., WO 2008/106692 and PCT/EP2009/057606, and as treatment for inflammatory conditions such as Crohn's disease, inflammatory bowel disease, rheumatoid arthritis, and chronic inflammatory diseases, see e.g., WO 2008/022164. The present invention provides compounds that inhibit activity of one or more Pims and exhibit distinctive characteristics that may provide improved therapeutic effects. Compounds of the invention contain novel substitution patterns on one or more rings that appear to provide these distinctive properties.

### SUMMARY OF THE INVENTION

The invention provides compounds of Formula I, having four or more substituents on a cyclohexyl ring that is attached to a picolinamide moiety:

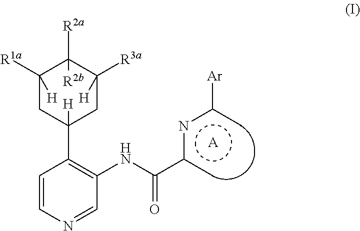

wherein:

groups attached to the cyclohexyl ring that are depicted inside the ring are all syn to each other, and all groups attached to the cyclohexyl ring that are depicted outside the cyclohexyl ring are syn to one another;

R^{1a} and R^{3a} are selected from hydroxyl, C1-C4 alkyl, —(CH₂)₁₋₃Z, C1-C4 haloalkyl, C1-C4 alkoxy, C1-C4 haloalkoxy, C1-C4 hydroxyalkyl, and amino,

R^{2a} is selected from C1-C4 alkyl, —(CH₂)₁₋₃Z, C1-C4 haloalkyl, and C1-C4 hydroxyalkyl,

wherein Z is —OH, NH₂, —NHC(O)Q, or —OC(O)Q, where Q is H or C1-C4 alkyl optionally substituted with one or more halo, OH, NH₂, OMe, or CN;

R^{2b} is OH;

ring A is a 5 or 6 membered aromatic ring selected from pyridinyl, pyrimidinyl, pyrazinyl, and thiazolyl and having N positioned as shown in Formula (I);

Ring A is optionally substituted with 1 or 2 groups selected from halo, CN, NH₂, hydroxy, C1-C4 alkyl, C1-C4 haloalkyl, C1-C4 alkoxy, and C1-C4 haloalkoxy;

Ar is an aromatic ring selected from phenyl, pyridinyl, pyrimidinyl, pyrazinyl, pyridazinyl, thiazolyl, and pyrazolyl, or a 3-6 membered cycloalkyl or cycloalkenyl; and

Ar is optionally substituted with up to three groups independently selected from halo, CN, NH₂, hydroxy, C1-C4 haloalkyl, —S(O)ₚ-Q², C1-C4 haloalkoxy, —(CH₂)₀₋₃—OQ², —O—(CH₂)₁₋₃—OQ², COOQ², C(O)Q², —(CR′₂)₁₋₃—OR′ or —(CR′₂)₁₋₃—OR′ where each R′ is independently H or Me, and an optionally substituted member selected from the group consisting of C₁₋₆ alkyl, C₁₋₆ alkoxy, C₁₋₆ alkylthio, C₁₋₆ alkylsulfonyl, C₃₋₇ cycloalkyl, C₃₋₇ heterocycloalkyl, C₅₋₁₀ heteroaryl, and C₆₋₁₀ aryl, each of which is optionally substituted with up to two groups selected from halo, CN, NH₂, hydroxy, C₁₋₄ haloalkyl, C₁₋₄alkoxy, and Q²;

where Q² is H or a 4-7 membered cyclic ether or C₁₋₆ alkyl, each of which is optionally substituted with one or more halo, oxo, OH, NH₂, COOH, COOMe, COOEt, OMe, OEt, or CN,

and p is 0-2;

or a pharmaceutically acceptable salt thereof. Additional embodiments of these compounds are described below.

These compounds are inhibitors of Pim kinases as further discussed herein. These compounds and their pharmaceutically acceptable salts, and pharmaceutical compositions containing these compounds and salts are useful for therapeutic methods such as treatment of cancers and autoimmune disorders that are caused by or exacerbated by excessive levels of Pim kinase activity.

### DETAILED DESCRIPTION OF EMBODIMENTS OF THE INVENTION

“PIM inhibitor” is used herein to refer to a compound that exhibits an IC₅₀ with respect to PIM Kinase activity of no more than about 100 μM and more typically not more than about 50 μM, as measured in the PIM depletion assays described herein below for at least one of Pim1, Pim2 and Pim3. Preferred compounds have on IC₅₀ below about 1 micromolar on at least one Pim, and generally have an IC₅₀ below 100 nM on each of Pim1, Pim2 and Pim3.

The phrase “alkyl” refers to hydrocarbon groups that do not contain heteroatoms, i.e., they consist of carbon atoms and hydrogen atoms. Thus the phrase includes straight chain alkyl groups such as methyl, ethyl, propyl, butyl, pentyl, hexyl, heptyl, octyl, nonyl, decyl, undecyl, dodecyl and the like. The phrase also includes branched chain isomers of straight chain alkyl groups, including but not limited to, the following which are provided by way of example: —CH(CH₃)₂, —CH(CH₃)(CH₂CH₃), —CH(CH₂CH₃)₂, —C(CH₃)₃, —C(CH₂CH₃)₃, —CH₂CH(CH₃)₂, —CH₂CH(CH₃)(CH₂CH₃), —CH₂CH(CH₂CH₃)₂, —CH₂C(CH₃)₃, —CH₂C(CH₂CH₃)₃, —CH(CH₃)CH(CH₃)(CH₂CH₃), —CH₂CH₂CH(CH₃)₂, —CH₂CH₂CH(CH₃)(CH₂CH₃), —CH₂CH₂CH(CH₂CH₃)₂, —CH₂CH₂C(CH₃)₃, —CH₂CH₂C(CH₂CH₃)₃, —CH(CH₃)CH₂CH(CH₃)₂, —CH(CH₃)CH(CH₃)CH(CH₃)₂, —CH(CH₂CH₃)CH(CH₃)CH(CH₃)(CH₂CH₃), and others.

Thus the term ‘alkyl’ includes primary alkyl groups, secondary alkyl groups, and tertiary alkyl groups. Typical alkyl groups include straight and branched chain alkyl groups having 1 to 12 carbon atoms, preferably 1-6 carbon atoms. The term ‘lower alkyl’ or “loweralkyl” and similar terms refer to alkyl groups containing up to 6 carbon atoms.

The term “alkenyl” refers to alkyl groups as defined above, wherein there is at least one carbon-carbon double bond, i.e., wherein two adjacent carbon atoms are attached by a double bond. The term “alkynyl” refers to alkyl groups wherein two adjacent carbon atoms are attached by a triple bond. Typical alkenyl and alkynyl groups contain 2-12 carbon atoms, preferably 2-6 carbon atoms. Lower alkenyl or lower alkynyl refers to groups having up to 6 carbon atoms. An alkenyl or alkynyl group may contain more than one unsaturated bond, and may include both double and triple bonds, but of course their bonding is consistent with well-known valence limitations.

The term ‘alkoxy” refers to —OR, wherein R is alkyl.

As used herein, the term “halogen” or “halo” refers to chloro, bromo, fluoro and iodo groups. Typical halo substituents are F and/or Cl. “Haloalkyl” refers to an alkyl radical substituted with one or more halogen atoms. The term “haloalkyl” thus includes monohalo alkyl, dihalo alkyl, trihalo alkyl, perhaloalkyl, and the like.

“Amino” refers herein to the group —NH₂. The term “alkylamino” refers herein to the group —NRR′ where R and R′ are each independently selected from hydrogen or a lower alkyl, provided —NRR′ is not —NH₂. The term “arylamino” refers herein to the group —NRR′ where R is aryl and R′ is hydrogen, a lower alkyl, or an aryl. The term “aralkylamino” refers herein to the group —NRR′ where R is a lower aralkyl and R′ is hydrogen, a loweralkyl, an aryl, or a loweraralkyl. The term cyano refers to the group —CN. The term nitro refers to the group —NO₂.

The term “alkoxyalkyl” refers to the group -alk₁-O-alk₂ where alk₁ is an alkyl or alkenyl linking group, and alk₂ is alkyl or alkenyl. The term “loweralkoxyalkyl” refers to an alkoxyalkyl where alk₁ is loweralkyl or loweralkenyl, and alk₂ is loweralkyl or loweralkenyl. The term “aryloxyalkyl” refers to the group -alkyl-O-aryl, where -alkyl- is a C₁₋₁₂ straight or branched chain alkyl linking group, preferably C₁₋₆. The term “aralkoxyalkyl” refers to the group -alkylenyl-O-aralkyl, where aralkyl is preferably a loweraralkyl.

The term “aminocarbonyl” refers herein to the group —C(O)—NH₂. “Substituted aminocarbonyl” refers herein to the group —C(O)—NRR′ where R is loweralkyl and R′ is hydrogen or a loweralkyl. In some embodiments, R and R′, together with the N atom attached to them may be taken together to form a “heterocycloalkylcarbonyl” group. The term “arylaminocarbonyl” refers herein to the group —C(O)—NRR′ where R is an aryl and R′ is hydrogen, loweralkyl or aryl. “aralkylaminocarbonyl” refers herein to the group —C(O)—NRR′ where R is loweraralkyl and R′ is hydrogen, loweralkyl, aryl, or loweraralkyl.

“Aminosulfonyl” refers herein to the group —S(O)₂—NH₂. “Substituted aminosulfonyl” refers herein to the group —S(O)₂—NRR′ where R is loweralkyl and R′ is hydrogen or a loweralkyl. The term “aralkylaminosulfonlyaryl” refers herein to the group -aryl-S(O)₂—NH-aralkyl, where the aralkyl is loweraralkyl.

“Carbonyl” refers to the divalent group —C(O)—. “Carboxy” refers to —C(═O)—OH. “Alkoxycarbonyl” refers to ester —C(═O)—OR wherein R is optionally substituted lower alkyl. “Loweralkoxycarbonyl” refers to ester —C(═O)—OR wherein R is optionally substituted lower loweralkyl. “Cycloalkyloxycarbonyl” refers to —C(═O)—OR wherein R is optionally substituted C3-C8 cycloalkyl.

“Cycloalkyl” refers to a mono- or polycyclic, carbocyclic alkyl substituent. Carbocycloalkyl groups are cycloalkyl groups in which all ring atoms are carbon. Typical cycloalkyl substituents have from 3 to 8 backbone (i.e., ring) atoms. When used in connection with cycloalkyl substituents, the term “polycyclic” refers herein to fused and non-fused alkyl cyclic structures. The term “partially unsaturated cycloalkyl”, “partially saturated cycloalkyl”, and “cycloalkenyl” all refer to a cycloalkyl group wherein there is at least one point of unsaturation, i.e., wherein to adjacent ring atoms are connected by a double bond or a triple bond. Such rings typically contain 1-2 double bonds for 5-6 membered rings, and 1-2 double bonds or one triple bond for 7-8 membered rings. Illustrative examples include cyclohexenyl, cyclooctynyl, cyclopropenyl, cyclobutenyl, cyclohexadienyl, and the like.

The term “heterocycloalkyl” refers herein to cycloalkyl substituents that have from 1 to 5, and more typically from 1 to 4 heteroatoms as ring members in place of carbon atoms. Preferably, heterocycloalkyl or “heterocyclyl” groups contain one or two heteroatoms as ring members, typically only one heteroatom for 3-5 membered rings and 1-2 heteroatoms for 6-8 membered rings. Suitable heteroatoms employed in heterocyclic groups of the present invention are nitrogen, oxygen, and sulfur. Representative heterocycloalkyl moieties include, for example, pyrrolidinyl, tetrahydrofuranyl, oxirane, oxetane, oxepane, thiirane, thietane, azetidine, morpholino, piperazinyl, piperidinyl and the like.

The terms “substituted heterocycle”, “heterocyclic group” or “heterocycle” as used herein refers to any 3- or 4-membered ring containing a heteroatom selected from nitrogen, oxygen, and sulfur or a 5- or 6-membered ring containing from one to three heteroatoms, preferably 1-2 heteroatoms, selected from the group consisting of nitrogen, oxygen, or sulfur; wherein the 5-membered ring has 0-2 double bonds and the 6-membered ring has 0-3 double bonds; wherein the nitrogen and sulfur atom maybe optionally oxidized; wherein the nitrogen and sulfur heteroatoms may be optionally quarternized; and including any bicyclic group in which any of the above heterocyclic rings is fused to a benzene ring or another 5- or 6-membered heterocyclic ring as described herein. Preferred heterocycles include, for example: diazapinyl, pyrrolinyl, pyrrolidinyl, pyrazolinyl, pyrazolidinyl, imidazolinyl, imidazolidinyl, piperidinyl, piperazinyl, N-methyl piperazinyl, azetidinyl, N-methylazetidinyl, oxazolidinyl, isoazolidinyl, morpholinyl, thiazolidinyl, isothiazolidinyl, and oxiranyl. The heterocyclic groups may be attached at various positions as will be apparent to those having skill in the organic and medicinal chemistry arts in conjunction with the disclosure herein.

Heterocyclic moieties can be unsubstituted or they can be substituted with one or more substituents independently selected from hydroxy, halo, oxo (C═O), alkylimino (RN═, wherein R is a loweralkyl or loweralkoxy group), amino, alkylamino, dialkylamino, acylaminoalkyl, alkoxy, thioalkoxy, lower alkoxyalkoxy, loweralkyl, cycloalkyl or haloalkyl. Typically, substituted heterocyclic groups will have up to four substituent groups. The term “cyclic ether” as used herein refers to a 3-7 membered ring containing one oxygen atom (O) as a ring member. Where the cyclic ether is “optionally substituted” it can be substituted at any carbon atom with a group suitable as a substituent for a heterocyclic group, typically up to three substituents selected from lower alkyl, lower alkoxy, halo, hydroxy, —C(O)-lower alkyl, and —C(O)-lower alkoxy. In preferred embodiments, halo, hydroxy and lower alkoxy are not attached to the carbon atoms of the ring that are bonded directly to the oxygen atom in the cyclic ether ring. Specific examples include oxirane, oxetane (e.g., 3-oxetane), tetrahydrofuran (including 2-tetrahydrofuranyl and 3-tetrahydrofuranyl), tetrahydropyran (e.g., 4-tetrahydropyranyl), and oxepane.

“Aryl” refers to monocyclic and polycyclic aromatic groups having from 5 to 14 backbone carbon or hetero atoms, and includes both carbocyclic aryl groups and heteroaromatic aryl groups. Carbocyclic aryl groups are aryl groups in which all ring atoms in the aromatic ring are carbon, typically including phenyl and naphthyl. Exemplary aryl moieties employed as substituents in compounds of the present invention include phenyl, pyridyl, pyrimidinyl, thiazolyl, indolyl, imidazolyl, oxadiazolyl, tetrazolyl, pyrazinyl, triazolyl, thiophenyl, furanyl, quinolinyl, purinyl, naphthyl, benzothiazolyl, benzopyridyl, and benzimidazolyl, and the like. When used in connection with aryl substituents, the term “polycyclic aryl” refers herein to fused and non-fused cyclic structures in which at least one cyclic structure is aromatic, such as, for example, benzodioxozolo (which has a heterocyclic structure fused to a phenyl group, naphthyl, and the like. Where “aryl” is used, the group is preferably a carbocyclic group; the term “heteroaryl” is used for aryl groups when ones containing one or more heteroatoms are preferred.

The term “heteroaryl” refers herein to aryl groups having from 1 to 4 heteroatoms as ring atoms in an aromatic ring with the remainder of the ring atoms being carbon atoms, in a 5-14 atom aromatic ring system that can be monocyclic or polycyclic. Monocyclic heteroaryl rings are typically 5-6 atoms in size. Exemplary heteroaryl moieties employed as substituents in compounds of the present invention include pyridyl, pyrimidinyl, thiazolyl, indolyl, imidazolyl, oxadiazolyl, tetrazolyl, pyrazinyl, triazolyl, thiophenyl, furanyl, quinolinyl, purinyl, benzothiazolyl, benzopyridyl, and benzimidazolyl, and the like.

“Aralkyl” or “arylalkyl” refers to an aryl group connected to a structure through an alkylene linking group, e.g., a structure such as —(CH₂)₁₋₄—Ar, where Ar represents an aryl group. “Lower aralkyl” or similar terms indicate that the alkyl linking group has up to 6 carbon atoms.

“Optionally substituted” or “substituted” refers to the replacement of one or more hydrogen atoms with a monovalent or divalent radical. Alkyl, alkenyl, alkynyl, cycloalkyl, heterocycloalkyl, aryl, and heteroaryl groups described herein may be substituted or unsubstituted. Suitable substitution groups include, for example, hydroxy, nitro, amino, imino, cyano, halo, thio, sulfonyl, thioamido, amidino, imidino, oxo, oxamidino, methoxamidino, imidino, guanidino, sulfonamido, carboxyl, formyl, loweralkyl, haloloweralkyl, loweralkylamino, haloloweralkylamino, loweralkoxy, haloloweralkoxy, loweralkoxyalkyl, alkylcarbonyl, aminocarbonyl, arylcarbonyl, aralkylcarbonyl, heteroarylcarbonyl, heteroaralkylcarbonyl, alkylthio, aminoalkyl, cyanoalkyl, aryl and the like, provided that oxo, imidino or other divalent substitution groups are not placed on aryl or heteroaryl rings due to the well known valence limitations of such rings.

The substitution group can itself be substituted where valence permits, i.e., where the substitution group contains at least one CH, NH or OH having a hydrogen atom that can be replaced. The group substituted onto the substitution group can be carboxyl, halo (on carbon only); nitro, amino, cyano, hydroxy, loweralkyl, loweralkoxy, C(O)R, —OC(O)R, —OC(O)OR, —NRCOR, —CONR₂, —NRCOOR, —C(S)NR₂, —NRC(S)R, —OC(O)NR₂, —SR, —SO₃H, —SO₂R or C3-8 cycloalkyl or 3-8 membered heterocycloalkyl, where each R is independently selected from hydrogen, lower haloalkyl, lower alkoxyalkyl, and loweralkyl, and where two R on the same atom or on directly connected atoms can be linked together to form a 5-6 membered heterocyclic ring.

When a substituted substituent includes a straight chain group, the substitution can occur either within the chain (e.g., 2-hydroxypropyl, 2-aminobutyl, and the like) or at the chain terminus (e.g., 2-hydroxyethyl, 3-cyanopropyl, and the like). Substituted substituents can be straight chain, branched or cyclic arrangements of covalently bonded carbon or heteroatoms.

It is understood that the above definitions are not intended to include impermissible substitution patterns (e.g., methyl substituted with five fluoro groups or a halogen atom substituted with another halogen atom). Such impermissible substitution patterns are well known to the skilled artisan.

“Syn” as used herein has its ordinary meaning, and is used in connection with Formula I to indicate that the specified groups are attached to sp³ hybridized (tetrahedral) carbon centers and extend out from one face of the cyclohexyl ring, i.e., those groups all project toward the ‘alpha’ face of the cyclohexyl ring, or they all project toward the ‘beta’ face of the ring. This is thus used as a convenient way to define the relative orientations of two or more groups, without limiting the compounds to a specific chiral configuration. This reflects the fact that the compounds of the invention have such groups in a specific relative orientation, but are not limited to either enantiomer of that specific relative orientation. Accordingly, unless described as optically active, such compounds may be racemic, but also include each of the two enantiomers having the specified relative stereochemistry. In some embodiments, the compounds of the invention are optically active form as further described herein, and in preferred embodiments of the invention, the compounds are obtained and used in optically active form. Preferably, the enantiomer having greater potency as an inhibitor of at least two of Pim1, Pim2 and Pim3 is selected.

It will also be apparent to those skilled in the art that the compounds of the invention, as well as the pharmaceutically acceptable salts, esters, metabolites and prodrugs of any of them, may be subject to tautomerization and may therefore exist in various tautomeric forms wherein a proton of one atom of a molecule shifts to another atom and the chemical bonds between the atoms of the molecules are consequently rearranged. See, e.g., March, Advanced Organic Chemistry: Reactions, Mechanisms and Structures, Fourth Edition, John Wiley & Sons, pages 69-74 (1992). As used herein, the term “tautomer” refers to the compounds produced by the proton shift, and it should be understood that all tautomeric forms, insofar as they may exist, are included within the invention.

The compounds of the invention comprise one or more asymmetrically substituted carbon atoms. Such asymmetrically substituted carbon atoms can result in the compounds of the invention existing in enantiomers, diastereomers, and other stereoisomeric forms that may be defined, in terms of absolute stereochemistry, such as in (R)- or (S)-forms. The compounds of the invention are sometimes depicted herein as single enantiomers, and are intended to encompass the specific configuration depicted and the enantiomer of that specific configuration (the mirror image isomer of the depicted configuration), unless otherwise specified. The depicted structures herein describe the relative stereochemistry of the compounds where two or more chiral centers, but the invention is not limited to the depicted enantiomer's absolute stereochemistry unless otherwise stated. The invention includes both enantiomers, each of which will exhibit Pim inhibition, even though one enantiomer will be more potent than the other. In some instances, compounds of the invention have been synthesized in racemic form and separated into individual isomers by chiral chromatography or similar conventional methods, and the analytical data about the two enantiomers do not provide definitive information about absolute stereochemical configuration. In such cases, the absolute stereochemistry of the most active enantiomer has been identified based on correlation with similar compounds of known absolute stereochemistry, rather than by a definitive physical method such as X-ray crystallography. Therefore, in certain embodiments, the preferred enantiomer of a compound described herein is the specific isomer depicted or its opposite enantiomer, whichever has the lower IC-50 for Pim kinase inhibition using the assay methods described herein, i.e., the enantiomer that is more potent as a Pim inhibitor for at least two of Pim1, Pim2, and Pim3.

The terms “S” and “R” configuration, as used herein, are as defined by the IUPAC 1974 RECOMMENDATIONS FOR SECTION E, FUNDAMENTAL STEREOCHEMISTRY, Pure Appl. Chem. 45:13-30 (1976). The terms α and β are employed for ring positions of cyclic compounds. The α-side of the reference plane is that side on which the preferred substituent lies at the lower numbered position. Those substituents lying on the opposite side of the reference plane are assigned β descriptor. It should be noted that this usage differs from that for cyclic stereoparents, in which “α” means “below the plane” and denotes absolute configuration. The terms α and β configuration, as used herein, are as defined by the CHEMICAL ABSTRACTS INDEX GUIDE-APPENDIX IV (1987) paragraph 203.

As used herein, the term “pharmaceutically acceptable salts” refers to the nontoxic acid or base addition salts of the compounds of Formula I or II, wherein the compound acquires a positive or negative charge as a result of adding or removing a proton; the salt then includes a counterion of opposite charge from the compound itself, and the counterion is preferably one suitable for pharmaceutical administration under the conditions where the compound would be used. These salts can be prepared in situ during the final isolation and purification of the compounds of Formula I or II, or by separately reacting the base or acid functions with a suitable organic or inorganic acid or base, respectively. Representative salts include but are not limited to the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxyethanesulfonate, lactate, maleate, methanesulfonate, nicotinate, 2-naphthalenesulfonate, oxalate, pamoate, pectinate, persulfate, 3-phenylproionate, picrate, pivalate, propionate, succinate, sulfate, tartrate, thiocyanate, p-toluenesulfonate and undecanoate.

Also, a basic nitrogen-containing group in compounds of the invention can be quaternized with such agents as loweralkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others. Water or oil-soluble or dispersible products are thereby obtained. These quaternized ammonium salts when paired with a pharmaceutically acceptable anion can also serve as pharmaceutically acceptable salts.

Examples of acids which may be employed to form pharmaceutically acceptable acid addition salts include such inorganic acids as hydrochloric acid, sulfuric acid and phosphoric acid and such organic acids as oxalic acid, maleic acid, methanesulfonic acid, succinic acid and citric acid. Basic addition salts can be prepared in situ during the final isolation and purification of the compounds of formula (I), or separately by reacting carboxylic acid moieties with a suitable base such as the hydroxide, carbonate or bicarbonate of a pharmaceutically acceptable metal cation or with ammonia, or an organic primary, secondary or tertiary amine. Counterions for pharmaceutically acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine and the like.

As used herein, the term “pharmaceutically acceptable ester” refers to esters, which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular pharmaceutically acceptable esters include formates, acetates, propionates, maleates, lactates, hydroxyacetates, butyrates, acrylates and ethylsuccinates.

The term “pharmaceutically acceptable prodrugs” as used herein refers to those prodrugs of the compounds of the present invention which are, within the scope of sound medical judgment, suitable for use in contact with the tissues of humans and lower animals without undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of the above formula, for example by hydrolysis in blood. A thorough discussion is provided in T. Higuchi and V. Stella, PRO-DRUGS AS NOVEL DELIVERY SYSTEMS, Vol. 14 of the A.C.S. Symposium Series, and in Edward B. Roche, ed., BIOREVERSIBLE CARRIERS IN DRUG DESIGN, American Pharmaceutical Association and Pergamon Press, 1987, both of which are incorporated herein by reference.

It will be apparent to those skilled in the art that the compounds of the invention, or their tautomers, prodrugs and stereoisomers, as well as the pharmaceutically acceptable salts, esters and prodrugs of any of them, may be processed in vivo through metabolism in a human or animal body or cell to produce metabolites. The term “metabolite” as used herein refers to the formula of any derivative produced in a subject after administration of a parent compound. The derivatives may be produced from the parent compound by various biochemical transformations in the subject such as, for example, oxidation, reduction, hydrolysis, or conjugation and include, for example, oxides and demethylated derivatives. The metabolites of a compound of the invention may be identified using routine techniques known in the art. See, e.g., Bertolini, G. et al., J. Med. Chem. 40:2011-2016 (1997); Shan, D. et al., J. Pharm. Sci. 86(7):765-767; Bagshawe K., Drug Dev. Res. 34:220-230 (1995); Bodor, N., Advances in Drug Res. 13:224-331 (1984); Bundgaard, H., Design of Prodrugs (Elsevier Press 1985); and Larsen, I. K., Design and Application of Prodrugs, Drug Design and Development (Krogsgaard-Larsen et al., eds., Harwood Academic Publishers, 1991). It should be understood that individual chemical compounds that are metabolites of the compounds of formula (I) or their tautomers, prodrugs and stereoisomers, as well as the pharmaceutically acceptable salts, esters and prodrugs of any of them, are included within the invention.

The following enumerated aspects and embodiments of the invention illustrate its scope.

1. In one aspect, the invention provides compounds of Formula I:

A compound of Formula (I) or (Ia):

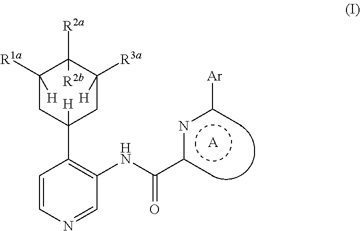

wherein:

groups attached to the cyclohexyl ring that are depicted inside the ring are all syn to each other, and all groups attached to the cyclohexyl ring that are depicted outside the cyclohexyl ring are syn to one another;

R^{1a} and R^{3a} are selected from hydroxyl, C1-C4 alkyl, —(CH₂)₁₋₃Z, C1-C4 haloalkyl, C1-C4 alkoxy, C1-C4 haloalkoxy, C1-C4 hydroxyalkyl, and amino,

R^{2a} is selected from C1-C4 alkyl, —(CH₂)₁₋₃Z, C1-C4 haloalkyl, and C1-C4 hydroxyalkyl,

wherein Z is —OH, NH₂, —NHC(O)Q, or —OC(O)Q, where Q is H or C1-C4 alkyl optionally substituted with one or more halo, OH, NH₂, OMe, or CN;

R^{2b} is OH;

ring A is a 5 or 6 membered aromatic ring selected from pyridinyl, pyrimidinyl, pyrazinyl, and thiazolyl and having N positioned as shown in Formula (I);

Ring A is optionally substituted with 1 or 2 groups selected from halo, CN, NH₂, hydroxy, C1-C4 alkyl, C1-C4 haloalkyl, C1-C4 alkoxy, and C1-C4 haloalkoxy;

Ar is an aromatic ring selected from phenyl, pyridinyl, pyrimidinyl, pyrazinyl, pyridazinyl, thiazolyl, and pyrazolyl, or a 3-6 membered cycloalkyl or cycloalkenyl, each of which is optionally fused to an additional C₅₋₆ cycloalkyl, C₅₋₆ heterocyclyl, C₅₋₆ heteroaryl or phenyl; and

Ar is optionally substituted with up to three groups independently selected from halo, CN, NH₂, hydroxy, C1-C4 haloalkyl, —S(O)ₚ-Q², C1-C4 haloalkoxy, —(CH₂)₀₋₃—OQ², —O—(CH₂)₁₋₃—OQ², —(CH₂)₁₋₃-Q², COOQ², C(O)Q², —(CR′₂)₁₋₃—OR′ or —(CR′₂)₁₋₃—OR′ where each R′ is independently H or Me or C₂₋₄ alkyl or C₃₋₆ cycloalkyl or C₅₋₆ heterocyclyl, and an optionally substituted member selected from the group consisting of C₁₋₆ alkyl, C₁₋₆ alkoxy, C₁₋₆ alkylthio, C₁₋₆ alkylsulfonyl, C₃₋₇ cycloalkyl, C₅₋₇ cycloalkenyl, C₃₋₇ heterocycloalkyl, C₄₋₆ cyclic ether, C₅₋₁₀ heteroaryl, and C₆₋₁₀ aryl, each of which is optionally substituted with up to two groups selected from halo, CN, NH₂, hydroxy, oxo, C₁₋₄haloalkyl, C₁₋₄alkoxy, and Q²;

where Q² is H or a 4-7 membered cyclic ether, phenyl, C₅₋₆ heteroaryl, or C₁₋₆ alkyl, each of which is optionally substituted with one or more halo, oxo, OH, NH₂, COOH, COOMe, COOEt, COONH₂, COONHMe, COONMe₂, OMe, OEt, or CN,

and p is 0-2;

or a pharmaceutically acceptable salt thereof.

This embodiment includes compounds of Formula (Ia), which form a subgenus of the compounds of Formula (I):

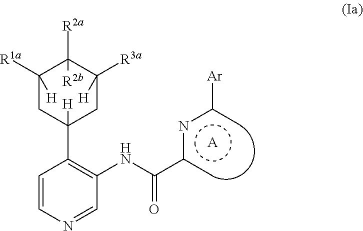

wherein:

groups attached to the cyclohexyl ring that are depicted inside the ring are all syn to each other, and all groups attached to the cyclohexyl ring that are depicted outside the cyclohexyl ring are syn to one another;

R^{1a} and R^{3a} are selected from hydroxyl, C1-C4 alkyl, —(CH₂)₁₋₃Z, C1-C4 haloalkyl, C1-C4 alkoxy, C1-C4 haloalkoxy, C1-C4 hydroxyalkyl, and amino,

R^{2a} is selected from C1-C4 alkyl, —(CH₂)₁₋₃Z, C1-C4 haloalkyl, and C1-C4 hydroxyalkyl,

wherein Z is —OH, NH₂, —NHC(O)Q, or —OC(O)Q, where Q is H or C1-C4 alkyl optionally substituted with one or more halo, OH, NH₂, OMe, or CN;

R^{2b} is OH;

ring A is a 5 or 6 membered aromatic ring selected from pyridinyl, pyrimidinyl, pyrazinyl, and thiazolyl and having N positioned as shown in Formula (Ia);

Ring A is optionally substituted with 1 or 2 groups selected from halo, CN, NH₂, hydroxy, C1-C4 alkyl, C1-C4 haloalkyl, C1-C4 alkoxy, and C1-C4 haloalkoxy;

Ar is an aromatic ring selected from phenyl, pyridinyl, pyrimidinyl, pyrazinyl, pyridazinyl, thiazolyl, and pyrazolyl, or a 3-6 membered cycloalkyl or cycloalkenyl;

Ar is optionally substituted with up to three groups independently selected from halo, CN, NH₂, hydroxy, C1-C4 haloalkyl, —S(O)ₚ-Q², C1-C4 haloalkoxy, —(CH₂)₀₋₃—OQ², —O—(CH₂)₁₋₃—OQ², COOQ², C(O)Q², —(CR′₂)₁₋₃—OR′ or —(CR′₂)₁₋₃—OR′ where each R′ is independently H or Me, and an optionally substituted member selected from the group consisting of C₁₋₆ alkyl, C₁₋₆ alkoxy, C₁₋₆ alkylthio, C₁₋₆ alkylsulfonyl, C₃₋₇ cycloalkyl, C₃₋₇ heterocycloalkyl, C₅₋₁₀ heteroaryl, and C₆₋₁₀ aryl, each of which is optionally substituted with up to two groups selected from halo, CN, NH₂, hydroxy, C₁₋₄ haloalkyl, C₁₋₄ alkoxy, and Q²;

where Q² is H or a 4-7 membered cyclic ether or C₁₋₆ alkyl, each of which is optionally substituted with one or more halo, oxo, OH, NH₂, COOH, COOMe, COOEt, OMe, OEt, or CN,

and p is 0-2;

or a pharmaceutically acceptable salt thereof.

In some embodiments, at least one substituent for Ar is selected from F, Cl, NH2, Me, Et, OMe, OEt, OCF3, OCHF2, OCH2CF3, CN, CF3, SMe, SOMe, SO2Me, —COOMe, —C(O)Me, —C(Me)2-OH, MeOCH2-, HOCH2-, hydroxyethyl, hydroxyethoxy, methoxyethyl, methoxyethoxy, oxetanyl (e.g., 3-oxetanyl), isopropoxy, tetrahydropyranyloxy (e.g., 4-tetrahydropyranyloxy), cyclopropyl, and CN. At least one substituent for Ar is preferably selected from Me, F, NH2, OMe, MeOCH2-, HOCH2-, hydroxyethyl, hydroxyethoxy, methoxyethyl, methoxyethoxy, and CN.

These compounds may be used in racemic form, or the individual enantiomers may be used, or mixtures of the enantiomers may be used. Each enantiomer can be used, and preferably the compound to be used is the enantiomer that has greater activity as a Pim inhibitor.

The cyclohexyl ring in these compounds has four substituents, not counting its attachment to the pyridinyl ring in Formula I. The invention provides novel combinations of substituents and their relative stereochemical orientation on the cyclohexyl ring, to provide advantageous biological activities.

2. In one embodiment, the invention provides a compound according to embodiment 1, wherein R^{1a} and R^{3a} are different. In some embodiments, one of these two groups is Me. In some of these embodiments, one of these two groups is NH₂.

3. In one embodiment, the invention provides a compound according to embodiment 1 or 2, wherein R^{1a} is OH.

In some of the foregoing embodiments, the groups represented by R^{1a} and R^{3a} are different from each other. In many embodiments, one of these represents NH₂ or OH, and the other often represents Me. In some embodiments, R^{1a} is Me; in some embodiments, R^{1a} is NH₂. In some embodiments, the cyclohexyl ring in the compound of Formula I is of this formula:

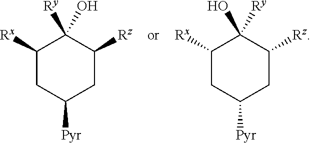

where Pyr represents the pyridine ring that is directly attached to the cyclohexyl ring in Formula I or Ia. In these embodiments, R^{y} is selected from Me, Et, CH₂F, CH₂OH, and CH₂OAc; one of R^{x} and R^{z} is Me or C₂₋₄ alkyl, and the other is selected from OH and NH₂. In a preferred embodiment, R^{x} is OH or NH₂ and R^{z} is Me. In another preferred embodiment, R^{x} is Me and R^{z} is OH or NH₂.

4. In one embodiment, the invention provides a compound according to any of embodiments 1-3, wherein R^{1a} is OH and R^{3a} is Me.

5. In one embodiment, the invention provides a compound according to either of embodiments 1 or 2, wherein R^{1a} is NH₂ and R^{3a} is Me.

6. In another embodiment, the invention provides a compound according to any of embodiments 1-5, wherein Ar is substituted with one to three groups selected from F, Cl, NH₂, Me, Et, OMe, OEt, OCF₃, OCHF₂, OCH₂CF₃, CN, CF₃, SMe, SOMe, SO₂Me, —COOMe, —C(O)Me, —C(Me)₂-OH, MeOCH₂—, HOCH₂—, hydroxyethyl, hydroxyethoxy, methoxyethyl, methoxyethoxy, oxetanyl (e.g., 3-oxetanyl), isopropoxy, tetrahydropyranyloxy (e.g., 4-tetrahydropyranyloxy), cyclopropyl, and CN. Preferably, substituents for Ar are selected from F, Cl, NH₂, Me, Et, OMe, OEt, OCF₃, OCHF₂, OCH₂CF₃, CN, CF₃, SMe, SOMe, SO₂Me, —COOMe, —C(O)Me, —C(Me)₂-OH, MeOCH₂—, HOCH₂—, hydroxyethyl, hydroxyethoxy, methoxyethyl, methoxyethoxy, and CN. In some embodiments, Ar is substituted by one such group; in other embodiments, Ar is substituted by at least two such groups. In some embodiments, Ar is substituted by three of these substituents, which may be the same or different. In some such embodiments, Ar is phenyl or pyridyl or pyrazolyl.

7. In one embodiment, the invention provides a compound according to any of the preceding embodiments, wherein Ar is substituted on at least one position adjacent to the ring atom of Ar that is attached to ring A.

8. In some embodiments, the invention provides a compound according to any of the preceding embodiments, wherein Ar is phenyl or 2-pyridinyl, and is substituted with up to three groups selected from F, Cl, Me, OMe, MeOCH₂—, HOCH₂—, hydroxyethyl, hydroxyethoxy, methoxyethyl, methoxyethoxy, oxetanyl (e.g., 3-oxetanyl), isopropoxy, tetrahydropyranyloxy (e.g., 4-tetrahydropyranyloxy), cyclopropyl, and CN. In some such embodiments, Ar is substituted with at least two groups, typically including one or two F.

9. In some embodiments, the invention provides a compound according to any of the preceding embodiments, wherein ring A is substituted with at least one halo or NH₂. Halo is often F.

In some of the foregoing embodiments, the compound is of one of the following formulas:

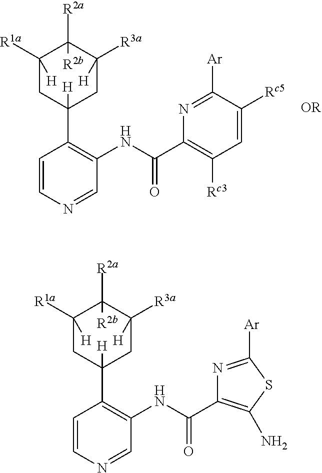

Wherein R^{1a}, R^{2a}, R^{2b}, R^{3a} and Ar are as described above, R^{c3} is H or NH₂, and R^{c5} is F or H.

10. In some embodiments, the invention provides a compound according to any of the preceding embodiments, wherein Ring A is pyridinyl. In specific embodiments, ring A is substituted with either F or NH₂. In other embodiments, ring A is unsubstituted. In such compounds, Ring A is often substituted with F at position 5 when the pyridinyl ring N is considered to be position 1 and Ar is at position 6. In other such compounds, Ring A is substituted with NH₂ at position 4 using this same method of counting ring positions. In yet other embodiments, Ring A has no substituents other than those depicted in Formula I (not counting the implicit H's present on the ring). Preferably, ring A is pyridinyl.

11. In some of the embodiments of embodiment 10, exactly one of R^{1a} and R^{3a} is the same as R^{2a}. In such embodiments, the two identical substituents are both Me, and in other such embodiments the two identical substituents are both —OH.

12. In some of examples of embodiment 11, the invention provides a compound wherein one of R^{1a} and R^{3a} is Me, and the other one is OH or NH₂.

13. In some embodiments, the invention provides a compound according to any of the preceding embodiments wherein R^{2a} is selected from CH₂F, —CH₂OH, —CH₂OAc, Et and Me.

14. In some embodiments, the invention provides a compound according to any of the preceding embodiments, wherein at least one of R^{1a} and R^{3a} is Me. The other one is typically —OH or NH₂.

15. In some embodiments, the invention provides a compound according to any of the preceding embodiments which is optically active. Preferably, the compound has a lower IC-50 than its opposite enantiomer on Pim kinase. Typically, the compound is substantially free of its opposite enantiomer, or is present in excess over its opposite enantiomer, having an enantiomeric excess of at least 80%, preferably at least 95%. The preferred enantiomer is the one having a lower IC-50 than its opposite enantiomer on Pim kinases, i.e., greater Pim inhibition on at least two of three Pim kinases, Pim1, Pim2 and Pim3.

16. In some embodiments, the invention provides a compound according to any of the preceding embodiments, which is an optically active compound of Formula IIa or IIb:

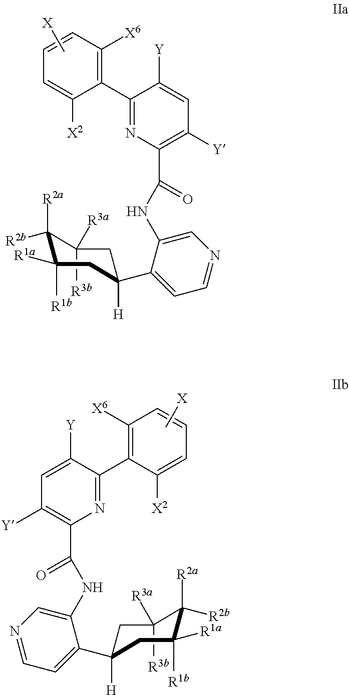

wherein, X, X² and X⁶ are independently selected from H, halo, CN, Me, OMe, OEt, OCHF₂, OCH₂CF₃, MeOCH₂—, HOCH₂—, hydroxyethyl, hydroxyethoxy, methoxyethyl, methoxyethoxy, oxetanyl (e.g., 3-oxetanyl), isopropoxy, tetrahydropyranyloxy (e.g., 4-tetrahydropyranyloxy), cyclopropyl, and NH₂;

R^{1b} and R^{3b} are both H;

Y and Y′ are independently selected from H, halo, and NH₂;

or a pharmaceutically acceptable salt thereof.

The optically active compound is a non-racemic compound, and may be a single enantiomer of Formula IIa or IIb, or it may be a mixture of enantiomers IIa and IIb, where either enantiomer IIa or enantiomer IIb is present in excess, preferably with an enantiomeric excess (ee) of at least 80%, and more preferably at least 95%.

In these embodiments, X² and X⁶ are often both halo, preferably F. In these embodiments, X can be H, halo, CN, Me, OMe, OEt, OCHF₂, OCH₂CF₃, MeOCH₂—, HOCH₂—, hydroxyethyl, hydroxyethoxy, methoxyethyl, methoxyethoxy, oxetanyl (e.g., 3-oxetanyl), isopropoxy, tetrahydropyranyloxy (e.g., 4-tetrahydropyranyloxy), cyclopropyl, or NH₂.

17. In some embodiments of the compounds of embodiment 16, X² and X⁶ are each F.

18. In certain embodiments, the invention provides a compound of embodiment 16 or 17, wherein Y is F and Y′ is H or NH₂. In other such embodiments, Y is H, and Y′ is H or NH₂.

19. In certain embodiments, the invention provides a compound of embodiment 16-18, wherein X is H, Me, F, NH₂, OMe, MeOCH₂—, HOCH₂—, hydroxyethyl, hydroxyethoxy, methoxyethyl, methoxyethoxy, or CN. In some embodiments, X is H, Me, F, NH₂, OMe, MeOCH₂—, HOCH₂—, hydroxyethyl, hydroxyethoxy, methoxyethyl, methoxyethoxy, CN, oxetanyl (e.g., 3-oxetanyl), isopropoxy, tetrahydropyranyloxy (e.g., 4-tetrahydropyranyloxy), cyclopropyl, and CN. In certain of these embodiments, X is H; in other embodiments, X is not H. In some embodiments, the isopropyl, oxetanyl or tetrahydropyranyl ring can be substituted with H, OH, CN, or COOH; suitable examples include:

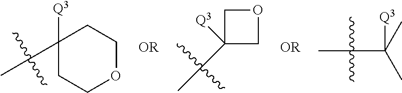

wherein Q³ can be H, CN, OH, COOH, or F.

20. In certain embodiments, the invention provides a compound of one of embodiments 16-19, wherein one of R^{1a} and R^{3a} is NH₂ or OH, and the other one is Me.

21. In certain embodiments, the invention provides a compound of one of embodiments 16-20, wherein R^{2b} is OH.

22. In certain embodiments, the invention provides a compound of one of embodiments 16-21, wherein R^{2a} is Me, —CH₂OH, —CH₂F, or Et.

23. In certain embodiments, the invention provides a compound of one of embodiments 16-22, which is a compound of Formula IIa.

24. In other embodiments, the invention provides a compound of one of embodiments 16-22, which is a compound of Formula IIb.

25. Specific embodiments of the invention include any one compound, or any subset of two or more compounds, selected from the group consisting of the compounds in Tables 1 and 2, and the pharmaceutically acceptable salts of these.

26. In another aspect, the invention provides a pharmaceutical composition comprising a compound of any of embodiments 1-25, admixed with at least one pharmaceutically acceptable excipient. Typically, the composition contains at least two such excipients. Suitable excipients are generally sterile.

27. In certain embodiments, the pharmaceutical composition of embodiment 26 comprises at least two pharmaceutically acceptable excipients.

28. In certain embodiments, the invention provides a composition of one of embodiments 26 or 27, which further comprises an additional agent for treatment of cancer.

29. In certain embodiments of the invention, the pharmaceutical composition of embodiment 24 contains an additional therapeutic agent selected from irinotecan, topotecan, gemcitabine, 5-fluorouracil, cytarabine, daunorubicin, PI3 Kinase inhibitors, mTOR inhibitors, DNA synthesis inhibitors, leucovorin, carboplatin, cisplatin, taxanes, tezacitabine, cyclophosphamide, vinca alkaloids, imatinib, anthracyclines, rituximab, and trastuzumab.

30. In another aspect, the invention provides a compound of any of embodiments 1-25 for use in the treatment of a condition that responds to inhibitors of Provirus Integration of Maloney Kinase (PIM Kinase) activity. Suitable conditions are known in the art.

31. In one embodiment of the embodiment 30, the condition is a cancer.

32. In selected embodiments of embodiment 31, the cancer is selected from carcinoma of the lungs, pancreas, thyroid, ovaries, bladder, breast, prostate or colon, melanoma, myeloid leukemia, multiple myeloma, erythro leukemia, villous colon adenoma, and osteosarcoma.

33. In other embodiments, the condition that responds to an inhibitor of Pim kinase is an autoimmune disorder.

34. In some embodiments, the invention provides a method of treating a disease or condition mediated by PIM kinase, comprising administering to a subject in need thereof a therapeutically effective amount of a compound according to any one of embodiments 1-25, or a pharmaceutically acceptable salt thereof. The method can include diagnosing the subject, e.g. a human, as one having such a disease or condition, and administering or directing administration of the compound or a pharmaceutical composition comprising the compound, optionally along with or in addition to administration of an additional therapeutic agent as described herein.

35. In the method of embodiment 34, the disease can be selected from carcinoma of the lungs, pancreas, thyroid, ovaries, bladder, breast, prostate or colon, melanoma, myeloid leukemia, multiple myeloma, erythro leukemia, villous colon adenoma, and osteosarcoma. In other embodiments, the disease is an autoimmune disorder.

36. In some examples of embodiment 35, the autoimmune disorder is selected from Crohn's disease, inflammatory bowel disease, rheumatoid arthritis, and chronic inflammatory diseases.

### Synthetic Methods

The compounds of the invention can be obtained through procedures known to those skilled in the art in view of the following Schemes and examples. For example, as shown in Scheme 1, cyclohexanediones can be converted via monotriflates to the corresponding cyclohexenoneboronate esters which can undergo palladium mediated carbon bond formation with 4-chloro, 3-nitro pyridine to yield nitropyridine substituted cyclohexenones I. Conversion of the ketone to the corresponding silyl enol ether, reaction with Eschenmoser's salt followed by methylation and elimination yields cyclohexadienone II. Reduction of the ketone yields the allylic alcohol III. Subsequent reaction with N-bromosuccinimide yields the bromohydrin, which upon silyl protection of the secondary hydroxyl and hydrogenation yields the tetrasubstituted cyclohexyl pyridyl aniline IV. Upon amide coupling and deprotection compounds V of the invention are obtained. In the amide product V, if R₂ is halo or triflate, the amide V can be further modified by standard modifications to introduce substituted aryls, alkyls and heteroaryls on place of R₂. For example, if R₂ is Br, by reaction with boronic acids or organometallic reagents, or conversion to the corresponding boronate ester and reaction with aryl/heteroaryl halides or triflates, a variety of R₂ replacements are possible.

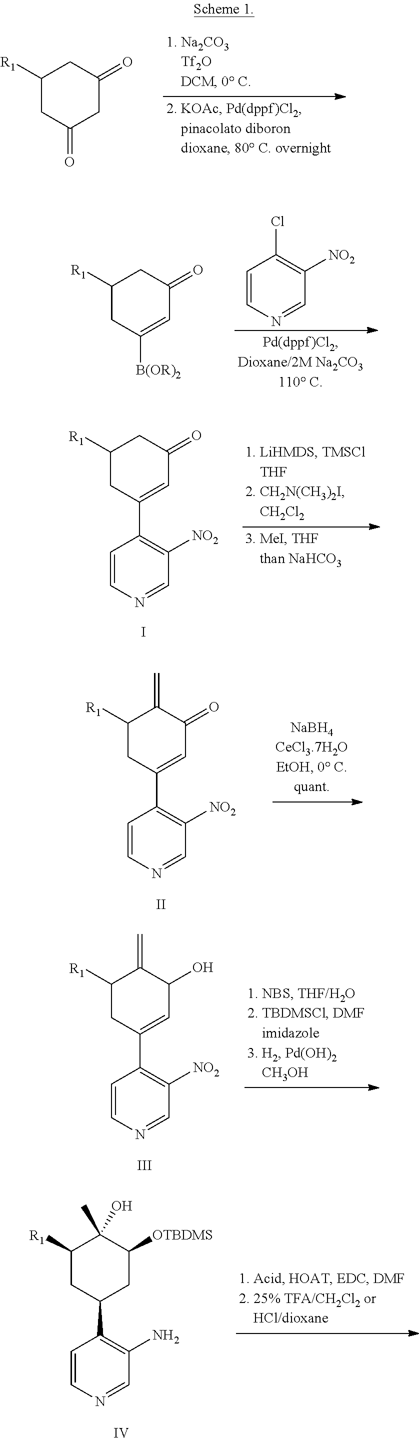

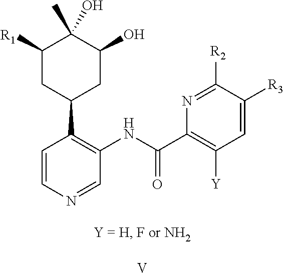

As shown in Scheme 2, cyclohexenol III can be manipulated to introduce a range of functionality in the cyclohexyl ring. Conversion to bromohydrin, secondary hydroxyl silylation, epoxide formation by base treatment, subsequent fluoride opening of the epoxide and hydrogenation yields the fluoromethyl substituted cyclohexyl pyridyl aniline VI. Alternatively, cyclohexenol III can be silyl protected, dihydroxylated, acetylated and hydrogenated to yield the acetoxy cyclohexyl pyridyl aniline VII. Additionally, the dihydroxylation product can be oxidized and converted to the corresponding alkyne which upon hydrogenation yields the ethyl substituted cyclohexyl pyridyl aniline VIII. The resulting cyclohexyl pyridyl anilines VI, VII and VIII can be converted to the corresponding pyridine amides IX by amide coupling, followed by acetate or silyl ether deprotection. If R₂ is halo or triflate, the amide IX can be further modified by standard modifications to introduce substituted aryls, alkyls and heteroaryls at R₂ after amide bond formation and prior to full deprotection. For example, if R₂ is Br, by reaction with boronic acids or organometallic reagents, or conversion to the corresponding boronate ester and reaction with aryl/heteroaryl halides or triflates, a variety of R₂ modifications are possible.

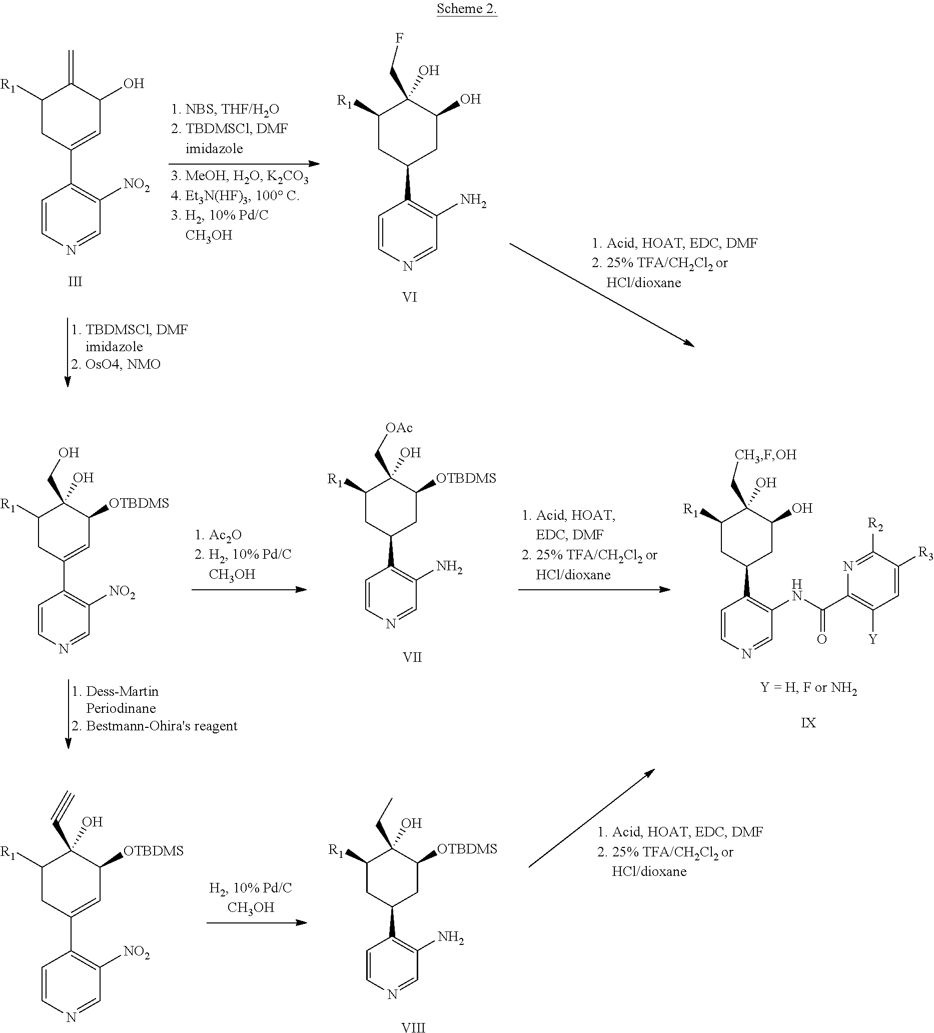

Allylic alcohol III can be converted to tetrasubstituted aminocyclohexyl compounds of the invention as depicted in Scheme 3. After bromohydrin formation, reaction with mesyl chloride in the presence of triethyl amine yields an endocyclic epoxide which can be opened up by treatment with sodium azide to form, after intramolecular bromide displacement, a cyclohexyl azido exocyclic epoxide. Upon hydrogenation, which cleaves the epoxide, reduces the nitro, cyclohexenyl alkene and azide, and protection of the resulting aliphatic amine by treatment with Boc₂O, the tetratsubstituted Bocamino pyridyl aniline X is obtained. The aniline X can be converted to the corresponding pyridine amides XI by amide coupling, followed by Boc deprotection. If R₂ is halo or triflate, the amides XI can be further modified by standard modifications to introduce substituted aryls, alkyls and heteroaryls at R₂ after amide bond formation and prior to full deprotection. For example, if R₂ is Br, by reaction with boronic acids or organometallic reagents, or conversion to the corresponding boronate ester and reaction with aryl/heteroaryl halides or triflates, a variety of R₂ modifications are possible.

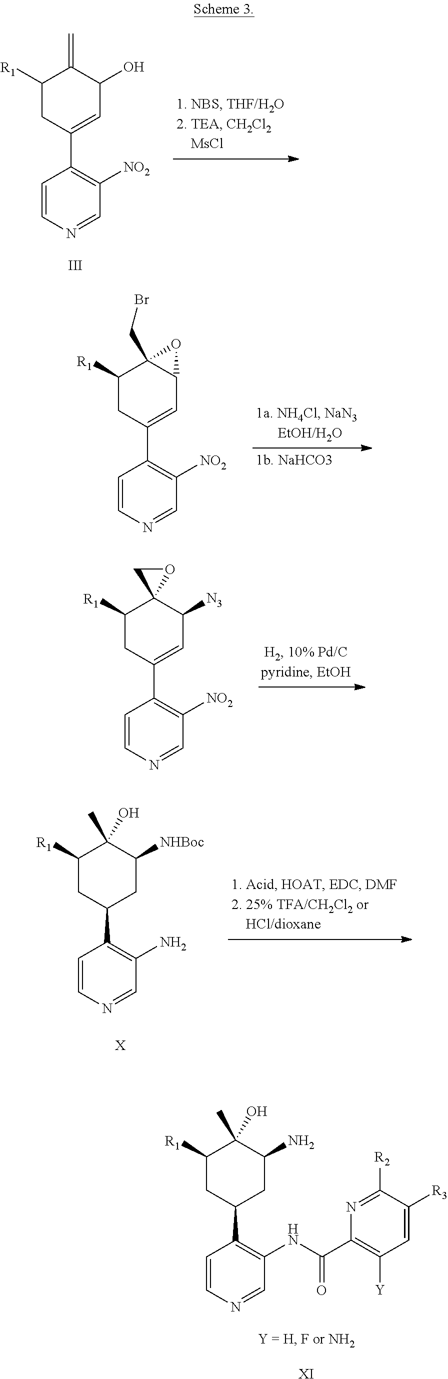

For purposes of the present invention, a therapeutically effective dose will generally be a total daily dose administered to a host in single or divided doses may be in amounts, for example, of from 0.001 to 1000 mg/kg body weight daily, typically 0.01 to 10 mg/kg per day, and more preferred from 0.1 to 30 mg/kg body weight daily. Generally, daily dosage amounts of 2 to 2000 mg, or from 10 to 1000 mg are anticipated for human subjects. Dosage unit compositions may contain such amounts of submultiples thereof to make up the daily dose.

The compounds of the present invention may be administered orally, parenterally, sublingually, by aerosolization or inhalation spray, rectally, or topically in dosage unit formulations containing conventional nontoxic pharmaceutically acceptable carriers, adjuvants, and vehicles as desired. Topical administration may also involve the use of transdermal administration such as transdermal patches or ionophoresis devices. The term parenteral as used herein includes subcutaneous injections, intravenous, intramuscular, intrasternal injection, or infusion techniques.

Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-propanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables.

Suppositories for rectal administration of the drug can be prepared by mixing the drug with a suitable nonirritating excipient such as cocoa butter and polyethylene glycols, which are solid at ordinary temperatures but liquid at the rectal temperature and will therefore melt in the rectum and release the drug.

Solid dosage forms for oral administration may include capsules, tablets, pills, powders, and granules. In such solid dosage forms, the active compound may be admixed with at least one inert diluent such as sucrose lactose or starch. Such dosage forms may also comprise, as is normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, cyclodextrins, and sweetening, flavoring, and perfuming agents.

The compounds of the present invention can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients, and the like. The preferred lipids are the phospholipids and phosphatidyl cholines (lecithins), both natural and synthetic. Methods to form liposomes are known in the art. See, for example, Prescott, Ed., Methods in Cell Biology, Volume XIV, Academic Press, New York, N.W., p. 33 et seq. (1976).

While the compounds of the invention can be administered as the sole active pharmaceutical agent, they can also be used in combination with one or more other agents used in the treatment of cancer. The compounds of the present invention are also useful in combination with known therapeutic agents and anti-cancer agents, and combinations of the presently disclosed compounds with other anti-cancer or chemotherapeutic agents are within the scope of the invention. Examples of such agents can be found in Cancer Principles and Practice of Oncology, V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Such anti-cancer agents include, but are not limited to, the following: estrogen receptor modulators, androgen receptor modulators, retinoid receptor modulators, cytotoxic/cytostatic agents, antiproliferative agents, prenyl-protein transferase inhibitors, HMG-CoA reductase inhibitors and other angiogenesis inhibitors, inhibitors of cell proliferation and survival signaling, apoptosis inducing agents and agents that interfere with cell cycle checkpoints. The compounds of the invention are also useful when co-administered with radiation therapy.

Therefore, in one embodiment of the invention, the compounds of the invention are also used in combination with known therapeutic or anticancer agents including, for example, estrogen receptor modulators, androgen receptor modulators, retinoid receptor modulators, cytotoxic agents, antiproliferative agents, prenyl-protein transferase inhibitors, HMG-CoA reductase inhibitors, HIV protease inhibitors, reverse transcriptase inhibitors, and other angiogenesis inhibitors.

In certain presently preferred embodiments of the invention, representative therapeutic agents useful in combination with the compounds of the invention for the treatment of cancer include, for example, irinotecan, topotecan, gemcitabine, 5-fluorouracil, cytarabine, daunorubicin, PI3 Kinase inhibitors, mTOR inhibitors, DNA synthesis inhibitors, leucovorin carboplatin, cisplatin, taxanes, tezacitabine, cyclophosphamide, vinca alkaloids, imatinib (Gleevec), anthracyclines, rituximab, trastuzumab, Revlimid, Velcade, dexamethasone, daunorubicin, cytaribine, clofarabine, Mylotarg, as well as other cancer chemotherapeutic agents including targeted therapeutics.

The above compounds to be employed in combination with the compounds of the invention will be used in therapeutic amounts as indicated in the Physicians' Desk Reference (PDR) 47th Edition (1993), which is incorporated herein by reference, or such therapeutically useful amounts as would be known to one of ordinary skill in the art, or provided in prescribing materials such as a drug label for the additional therapeutic agent.

The compounds of the invention and the other anticancer agents can be administered at the recommended maximum clinical dosage or at lower doses. Dosage levels of the active compounds in the compositions of the invention may be varied so as to obtain a desired therapeutic response depending on the route of administration, severity of the disease and the response of the patient. The combination can be administered as separate compositions or as a single dosage form containing both agents. When administered as a combination, the therapeutic agents can be formulated as separate compositions, which are given at the same time or different times, or the therapeutic agents, can be given as a single composition.

In one embodiment, the invention provides a method of inhibiting Pim1, Pim2 or Pim3 in a human or animal subject. The method includes administering an effective amount of a compound, or a pharmaceutically acceptable salt thereof, of any of the embodiments of compounds of Formula I or II to a subject in need thereof.

The present invention will be understood more readily by reference to the following examples, which are provided by way of illustration and are not intended to be limiting of the present invention.

### Examples

Referring to the examples that follow, compounds of the preferred embodiments were synthesized using the methods described herein, or other methods, which are known in the art.

The compounds and/or intermediates were characterized by high performance liquid chromatography (HPLC) using a Waters Millenium chromatography system with a 2695 Separation Module (Milford, Mass.). The analytical columns were reversed phase Phenomenex Luna C18-5μ, 4.6×50 mm, from Alltech (Deerfield, Ill.). A gradient elution was used (flow 2.5 mL/min), typically starting with 5% acetonitrile/95% water and progressing to 100% acetonitrile over a period of 10 minutes. All solvents contained 0.1% trifluoroacetic acid (TFA). Compounds were detected by ultraviolet light (UV) absorption at either 220 or 254 nm. HPLC solvents were from Burdick and Jackson (Muskegan, Mich.), or Fisher Scientific (Pittsburgh, Pa.).

In some instances, purity was assessed by thin layer chromatography (TLC) using glass or plastic backed silica gel plates, such as, for example, Baker-Flex Silica Gel 1B2-F flexible sheets. TLC results were readily detected visually under ultraviolet light, or by employing well-known iodine vapor and other various staining techniques.

Mass spectrometric analysis was performed on one of three LCMS instruments: a Waters System (Alliance HT HPLC and a Micromass ZQ mass spectrometer; Column: Eclipse XDB-C18, 2.1×50 mm; gradient: 5-95% (or 35-95%, or 65-95% or 95-95%) acetonitrile in water with 0.05% TFA over a 4 min period; flow rate 0.8 mL/min; molecular weight range 200-1500; cone Voltage 20 V; column temperature 40° C.), another Waters System (ACQUITY UPLC system and a ZQ 2000 system; Column: ACQUITY UPLC HSS-C18, 1.8 um, 2.1×50 mm; gradient: 5-95% (or 35-95%, or 65-95% or 95-95%) acetonitrile in water with 0.05% TFA over a 1.3 min period; flow rate 1.2 mL/min; molecular weight range 150-850; cone Voltage 20 V; column temperature 50° C.) or a Hewlett Packard System (Series 1100 HPLC; Column: Eclipse XDB-C18, 2.1×50 mm; gradient: 5-95% acetonitrile in water with 0.05% TFA over a 4 min period; flow rate 0.8 mL/min; molecular weight range 150-850; cone Voltage 50 V; column temperature 30° C.). All masses were reported as those of the protonated parent ions.

Nuclear magnetic resonance (NMR) analysis was performed on some of the compounds with a Varian 400 MHz NMR (Palo Alto, Calif.). The spectral reference was either TMS or the known chemical shift of the solvent.

Preparative separations are carried out using a Flash 40 chromatography system and KP-Sil, 60A (Biotage, Charlottesville, Va.), or by flash column chromatography using silica gel (230-400 mesh) packing material, or by HPLC using a Waters 2767 Sample Manager, C-18 reversed phase column, 30×50 mm, flow 75 mL/min. Typical solvents employed for the Flash 40 Biotage system and flash column chromatography are dichloromethane, methanol, ethyl acetate, hexane, acetone, aqueous ammonia (or ammonium hydroxide), and triethyl amine. Typical solvents employed for the reverse phase HPLC are varying concentrations of acetonitrile and water with 0.1% trifluoroacetic acid.

It should be understood that the organic compounds according to the preferred embodiments may exhibit the phenomenon of tautomerism. As the chemical structures within this specification can only represent one of the possible tautomeric forms, it should be understood that the preferred embodiments encompasses any tautomeric form of the drawn structure.

It is understood that the invention is not limited to the embodiments set forth herein for illustration, but embraces all such forms thereof as come within the scope of the above disclosure.

The examples below as well as throughout the application, the following abbreviations have the following meanings. If not defined, the terms have their generally accepted meanings.

ABBREVIATIONS

| | |
|---|---|
| Bestmann-Ohira reagent | dimethyl (1-diazo-2-oxopropyl)phosphonate |
| DAST | (diethylamino)sulfurtrifluoride |
| DCM | Dichloromethane |
| DIAD | diisopropylazodicarboxylate |
| DIEA | diisopropylethylamine |
| DMA | Dimethylacetamide |
| DMAP | 4-dimethylaminopyridine |
| DME | 1,2-dimethoxyethane |
| DMF | N,N-dimethylformamide |
| DPPF | 1,1′-bis(diphenylphosphino)ferrocene |
| EDC | 1-(3-Dimethylaminopropyl)-3-ethylcarbodiimide |
| | hydrochloride |
| EtOAc | ethyl acetate |
| EtOH | Ethanol |
| HOAT | Hydroxyazabenzotriazole |
| K₂CO₃ | Potassium carbonate |
| MeCN | Acetonitrile |
| MgSO₄ | Magnesium sulfate |
| MeOH | Methanol |
| Na₂CO₃ | sodium carbonate |
| NaCl | Sodium chloride |
| NaHCO₃ | sodium bicarbonate |
| NBS | N-bromosuccinimide |
| NMP | N-methyl-2-pyrrolidone |
| Pd₂(dba)₃ | Tris(dibenzylideneacetone)dipalladium(0) |
| Pd(PPh₃)₄ | Tetrakis(triphenylphospine)palladium(0) |
| Pd(dppf)Cl₂₋DCM | Dichloro-(1,2-bis(diphenylphosphino)ethan)- |
| | Palladium(II)dichloromothethane adduct |
| RT or rt | room temperature |
| TBDMS | tert-butyldimethylsilyl |
| TBDMSCl | tert-butyldimethylsilylchloride |
| TEA | Triethylamine |
| TMS | Trimethylsilyl |
| THF | tetrahydrofuran |

### Examples

### Synthesis of 5-methyl-3-oxocyclohex-1-enyltrifluoromethanesulfonate

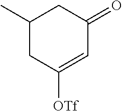 [SMILES: CC1CC(=O)C=C(OS(=O)(=O)C(F)(F)F)C1]

To a solution of 5-methylcyclohexane-1,3-dione (1.0 equiv.) in DCM (0.5M) was added Na₂CO₃ (1.1 equiv.) and cooled to 0° C. Added Tf₂O (1.0 equiv.) in DCM (5.0 M) dropwise over 1 hr at 0° C. under a nitrogen atmosphere. Upon addition, the reaction was stirred for 1 hr at room temperature (dark red solution). The solution was filtered and the filtrate was quenched by careful addition of saturated NaHCO₃ with vigorous stirring until pH=7. The solution was transferred to a separatory funnel and the layers were separated. The organic layer was washed with brine, dried with Na₂SO₄, filtered, concentrated under vacuo and dried under high vacuum for 15 min to yield 5-methyl-3-oxocyclohex-1-enyl trifluoromethanesulfonate as light yellow oil in 78% yield. The triflate decomposes upon storage and should be used immediately for the next reaction. LC/MS=259.1/300.1 (M+H and M+CH₃CN); Rt=0.86 min, LC=3.84 min. ¹H-NMR (400 MHz, CDCl₃) δ ppm: 6.05 (s, 1H), 2.70 (dd, J=17.2, 4.3, 1H), 2.53 (dd, J=16.6, 3.7, 1H), 2.48-2.31 (m, 2H), 2.16 (dd, J=16.4, 11.7, 1H), 1.16 (d, J=5.9, 3H).

### Synthesis of 5-methyl-3-(4,4,5,5-tetramethyl-1,3,2-dioxaborolan-2-yl)cyclohex-2-enone

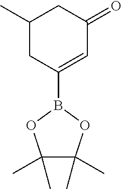 [SMILES: CC1CC(=O)C=C(B2OC(C)(C)C(C)(C)O2)C1]

To a solution of 5-methyl-3-oxocyclohex-1-enyl trifluoromethanesulfonate (1.0 equiv.) in degassed dioxane (0.7 M) was added bis(pinacolato)diboron (2.0 equiv.), KOAc (3.0 equiv.), and Pd(dppf)Cl₂-DCM (0.03 equiv.). The reaction was heated to 80° C. for 10 h (initial heating at large scale results in exothermic formation of an orange foam on top of the solution, the heating bath should be removed until the foam retracts, reheating to 80° C. at this point appears to be fine), then cooled to room temperature and filtered through a coarse frit glass funnel. The cake was rinsed with more dioxane and the filtrate solution was used for the next step without further purification. LC/MS=155.1 (M+H of boronic acid); Rt=0.41 min, LC=1.37 min.

### Synthesis of 5-methyl-3-(3-nitropyridin-4-yl)cyclohex-2-enone

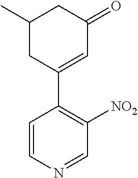 [SMILES: CC1CC(=O)C=C(c2ccncc2[N+](=O)[O-])C1]

To a solution of 5-methyl-3-(4,4,5,5-tetramethyl-1,3,2-dioxaborolan-2-yl)cyclohex-2-enone (1.0 equiv.) in degassed dioxane (0.5 M) and 2M Na₂CO₃ (2 equiv.) was added 4-chloro-3-nitropyridine (1.3 equiv.) and Pd(dppf)Cl₂-DCM (0.05 equiv.). The reaction was placed under a reflux condenser and heated in an oil bath to 110° C. for 1 h. Cooled to room temperature, filtered through a pad of Celite, washed the pad with ethyl acetate and concentrated the filtrate under vacuo. The residue was further pumped at 80° C. on a rotary evaporator for one hour to remove boronate by-products (M+H=101) via sublimation. The residue was partitioned between brine and ethyl acetate, and the layers were separated, the aqueous phase was further extracted with ethyl acetate (4×), the organics were combined, dried over sodium sulfate, filtered, and concentrated. The crude was purified via silica gel chromatography loading in DCM and eluting with 2-50% ethyl acetate and hexanes. The pure fractions were concentrated in vacuo to yield an orange oil. The oil was placed under high vacuum (˜500 mtorr) with seed crystals overnight to yield an orange solid. The solid was further purified via trituration in hexanes to yield 5-methyl-3-(3-nitropyridin-4-yl)cyclohex-2-enone (48% 2 steps). LC/MS=233.2 (M+H); Rt=0.69 min, LC=2.70 min. ¹H-NMR (400 MHz, CdCl₃) δ ppm: 9.31 (s, 1H), 8.88 (d, J=5.1, 1H), 7.30 (d, J=5.1, 1H), 6.00 (d, J=2.4, 1H), 2.62 (dd, J=16.4, 3.5, 1H), 2.53-2.34 (m, 3H), 2.23 (dd, J=16.1, 11.7, 1H), 1.16 (d, J=6.3, 3H).

### Synthesis of (+/−)-4-(5-methyl-3-(trimethylsilyloxy)cyclohexa-1,3-dienyl)-3-nitropyridine

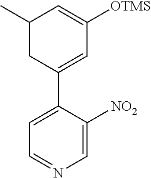

A solution of (+/−)-5-methyl-3-(3-nitropyridin-4-yl)cyclohex-2-enone (1.0 equiv.) and TMSCl (1.1 equiv.) in THF was added LiHMDS (1.0M in THF, 1.05 equiv.) at 0° C. slowly over 1 hour. The reaction mixture was warmed up to room temperature and stirred for 2 h. The reaction mixture was quenched with NaHCO₃ aqueous solution and removed THF in vacuo. The residue was extracted with EtOAc 3 times. The organic layer was washed with water and brine, dried over anhydrous K₂CO₃ and filtered, concentrated in vacuo to yield crude (+/−)-4-(5-methyl-3-(trimethylsilyloxy)cyclohexa-1,3-dienyl)-3-nitropyridine in 99% yield. ¹H NMR (400 MHz, CHLOROFORM-d) δ ppm 9.14-9.00 (m, 1H), 8.80-8.64 (m, 1H), 7.42-7.25 (m, 1H), 6.00-5.88 (m, 1H), 4.98 (br. s., 1H), 2.86-2.53 (m, 1H), 2.51-2.29 (m, 1H), 2.27-2.03 (m, 1H), 1.21-1.03 (m, 3H), 0.36-0.15 (m, 9H).

### Synthesis of (+/−)-6-((dimethylamino)methyl)-5-methyl-3-(3-nitropyridin-4-yl)cyclohex-2-enone

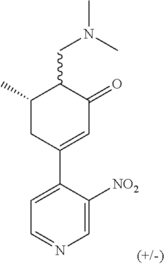 [SMILES: C[C@H]1CC(c2ccncc2[N+](=O)[O-])=CC(=O)C1CN(C)C]

To a solution of Eschenmoser's salt (1.1 equiv.) in DCM (0.3 M) was added (+/−)-4-(5-methyl-3-(trimethylsilyloxy)cyclohexa-1,3-dienyl)-3-nitropyridine in DCM (0.2 M) at 0° C. slowly over 60 min. The reaction mixture was allowed to warm up to room temperature and stirred for 18 h. After the reaction mixture was transferred to larger vessel and diluted with DCM (100 mL), 1 M HCl (60 mL) was added to the reaction mixture, which was stirred for 20 min in 0° C. 2 N NaOH (80 mL) was slowly added to aqueous phase at 0° C. The reaction mixture was stirred for 1 h, and then adjusted pH to 12 by 3N NaOH. After the organic layer was separated, aqueous phase was extracted with CH₂Cl₂ 3 times. The combined organic layer was dried over anhydrous Na₂SO₄ and concentrated in vacuo to yield crude (+/−)-6-((dimethylamino)methyl)-5-methyl-3-(3-nitropyridin-4-yl)cyclohex-2-enone in 99% yield. LCMS (m/z): 290.0 (MH⁺), Rₜ=0.40 min.

### Synthesis of (+/−)-5-methyl-6-methylene-3-(3-nitropyridin-4-yl)cyclohex-2-enone

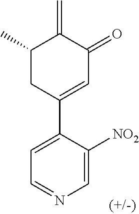 [SMILES: C=C1C(=O)C=C(c2ccncc2[N+](=O)[O-])C[C@@H]1C]

To a solution of (+/−)-6-((dimethylamino)methyl)-5-methyl-3-(3-nitropyridin-4-yl)cyclohex-2-enone (1.0 equiv.) in THF (0.3 M) was added iodomethane (1.3 equiv.) slowly at 0° C. The reaction mixture was allowed to warm up to room temperature and stirred at room temperature for 18 h. After saturated NaHCO₃ solution was added, the reaction mixture was stirred at room temperature for 5 h, diluted with EtOAc and stirred at room temperature for another 6 hr. After the organic layer was separated, the aqueous phase was extracted with EtOAc 3 times, the combined organic layer was washed with water and brine, dried over anhydrous Na₂SO₄, concentrated in vacuo to give crude (+/−)-5-methyl-6-methylene-3-(3-nitropyridin-4-yl)cyclohex-2-enone in 99% yield. LCMS (m/z): 245 (MH⁺), Rₜ=0.40 min. ¹H NMR (400 M Hz, CHLOROFORM-d) δ ppm 9.33 (s, 1H), 8.88 (d, J=5.1 Hz, 1H), 7.32-7.26 (m, 1H), 6.22-6.09 (m, 2H), 5.42 (s, 1H), 3.15 (dt, J=4.6, 2.2 Hz, 1H), 2.59 (dd, J=17.4, 5.3 Hz, 1H), 2.43 (ddd, J=7.3, 9.5, 2.2 Hz, 1H), 1.31 (d, J=6.7 Hz, 3H).

### Synthesis of (+/−)-(1R,5S)-5-methyl-6-methylene-3-(3-nitropyridin-4-yl)cyclohex-2-enol

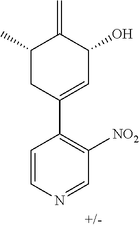 [SMILES: C=C1[C@H](O)C=C(c2ccncc2[N+](=O)[O-])C[C@@H]1C]

To a solution of (+/−)-5-methyl-6-methylene-3-(3-nitropyridin-4-yl)cyclohex-2-enone (1.0 equiv.) in methanol (0.3 M) was added CERIUM(III) CHLORIDE HEPTAHYDRATE (1.1 equiv.). The reaction mixture was stirred at room temperature for 1 h. After cooled down to at 0° C., NaBH₄ (1.0 equiv) was added slowly and stirred for 30 min. After quenched with water, the volatile materials were removed in vacuo and sat. NaHCO₃ was added into mixture with vigorous stirring. The reaction mixture was extracted with EtOAc and the organic layer was washed with brine, and dried over anhydrous sodium sulfate, filtered and concentrated in vacuo. The crude product was purified by silica chromatography (Heptane:EtOAc, 80:20 to 20:80) to give (+/−)-(1R,5S)-5-methyl-6-methylene-3-(3-nitropyridin-4-yl)cyclohex-2-enol as yellow solid in 50% yield. LCMS (m/z): 247 (MH⁺), Rₜ=0.70 min. ¹H NMR (400 M Hz, CHLOROFORM-d) δ ppm 9.13 (s, 1H), 8.75 (d, J=4.7 Hz, 1H), 7.26 (s, 1H), 5.73 (br. s., 1H), 5.25 (s, 1H), 5.03 (br. s., 1H), 4.86 (br. s., 1H), 2.67 (d, J=4.7 Hz, 1H), 2.39 (dd, J=16.6, 4.9 Hz, 1H), 2.11 (br. s., 1H), 1.79 (d, J=8.6 Hz, 1H), 1.23 (d, J=6.7 Hz, 3H).

### Synthesis of (+/−)-(1R,2R,6S)-1-(bromomethyl)-6-methyl-4-(3-nitropyridin-4-yl)cyclohex-3-ene-1,2-diol

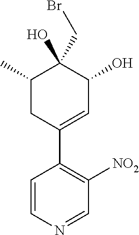 [SMILES: C[C@H]1CC(c2ccncc2[N+](=O)[O-])=C[C@@H](O)[C@@]1(O)CBr]

To a solution of (+/−)-(1R,5S)-5-methyl-6-methylene-3-(3-nitropyridin-4-yl)cyclohex-2-enol (1.0 equiv.) in THF:H₂O (1:1, 0.3 M) was added NBS (1.5 equiv.) at room temperature. The reaction mixture was stirred at room temperature for 5 min. After quenched with sodium thiosulfite, the reaction mixture was then extracted by EtOAc and washed with NaHCO₃ solution, water and brine, dried over anhydrous sodium sulfate, filtered and concentrated in vacuo. The crude product was used in next step reaction. LCMS (m/z): 342.9/344.9 (MH⁺), Rₜ=0.62 min. ¹H NMR (400 M Hz, CDCl₃) δ ppm 9.13 (s, 1H), 8.77 (d, J=5.1 Hz, 1H), 7.29 (d, J=5.1 Hz, 1H), 5.75-5.71 (m, 1H), 4.27 (br. s., 1H), 4.06 (d, J=10.6 Hz, 1H), 3.77 (d, J=11.0 Hz, 1H), 2.76-2.69 (m, 1H), 2.34 (br. s., 1H), 2.31-2.23 (m, 1H), 2.14 (dd, J=17.8, 5.7 Hz, 1H), 1.20 (d, J=7.4 Hz, 3H).

### Synthesis of (+/−)-(1R,2R,6S)-1-(bromomethyl)-2-(tert-butyldimethylsilyloxy)-6-methyl-4-(3-nitropyridin-4-yl)cyclohex-3-enol

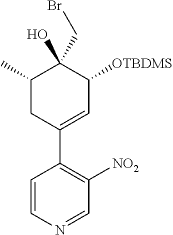 [SMILES: C[C@H]1CC(c2ccncc2[N+](=O)[O-])=C[C@@H](O[Si](C)(C)C(C)(C)C)[C@@]1(O)CBr]

To a solution of (+/−)-(1R,2R,6S)-1-(bromomethyl)-6-methyl-4-(3-nitropyridin-4-yl)cyclohex-3-ene-1,2-diol (1.0 equiv.) in DMF (0.5 M) was added TBDMSCl (1.5 equiv), IMIDAZOLE (2.0 equiv.) at room temperature. The reaction mixture was stirred at room temperature for 24 h. After quenched with NaHCO₃, the reaction mixture was extracted with EtOAc 3 times. The organic layer was washed with water and brine, dried over anhydrous sodium sulfate, filtered and concentrated in vacuo. (+/−)-(1R,2R,6S)-1-(bromomethyl)-2-(tert-butyldimethylsilyloxy)-6-methyl-4-(3-nitropyridin-4-yl)cyclohex-3-enol was isolated as a light yellow solid by flash column chromatography (EtOAc:Heptane, 10:90 to 90:10). LCMS (m/z): 459.0 (MH⁺), Rₜ=0.23 min. ¹H NMR (400 M Hz, CHLOROFORM-d) δ ppm 9.11 (s, 1H), 8.75 (d, J=5.1 Hz, 1H), 7.31-7.25 (m, 1H), 5.61 (br. s., 1H), 4.15-4.08 (m, J=3.5 Hz, 1H), 3.95 (d, J=10.6 Hz, 1H), 3.76 (d, J=10.2 Hz, 1H), 2.81 (dd, J=17.6, 5.9 Hz, 1H), 2.35 (s, 1H), 2.32-2.23 (m, 1H), 2.06 (dd, J=17.6, 3.5 Hz, 1H), 1.20 (d, J=7.4 Hz, 3H), 0.83-0.97 (m, 9H), 0.13 (s, 3H), 0.08 (s, 3H).

### Synthesis of (1R,2R,4R,6S)-4-(3-aminopyridin-4-yl)-2-(tert-butyldimethylsilyloxy)-1,6-dimethylcyclohexanol and (1S,2S,4S,6R)-4-(3-aminopyridin-4-yl)-2-((tert-butyldimethylsilyl)oxy)-1,6-dimethylcyclohexanol

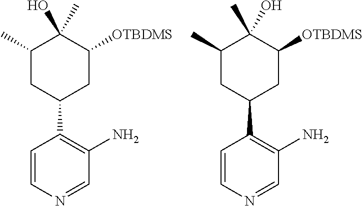 [SMILES: C[C@H]1C[C@@H](c2ccncc2N)C[C@@H](O[Si](C)(C)C(C)(C)C)[C@]1(C)O ; C[C@@H]1C[C@H](c2ccncc2N)C[C@H](O[Si](C)(C)C(C)(C)C)[C@@]1(C)O]

A solution of (+/−)-(1R,2R,6S)-1-(bromomethyl)-2-(tert-butyldimethylsilyloxy)-6-methyl-4-(3-nitropyridin-4-yl)cyclohex-3-enol (1.0 equiv.) in methanol (0.3 M) was degassed by nitrogen for 10 min followed by addition of 10% Pd(OH)₂/C (0.1 equiv), the reaction mixture in a steel bomb reactor was charged with hydrogen to 200 psi and stirred at room temperature for 4 days. The reaction mixture was filtered through Celite pad and the filtrate was concentrated to give crude (+/−)-(1R,2R,4R,6S)-4-(3-aminopyridin-4-yl)-2-((tert-butyldimethylsilyl)oxy)-1,6-dimethylcyclohexanol. LCMS (m/z): 351.1 (MH⁺), Rₜ=0.85 min). ¹H-NMR (400 MHz, CDCl₃) d ppm 8.6 (s, 1H), 8.03-8.01 (m, 2H), 6.99 (m, 1H), 3.62 (m, 1H), 2.69 (m, 1H), 1.85 (m, 2H), 1.61 (m, 1H), 1.39 (m, 1H), 1.26 (m, 1H), 1.21 (d, J=8 Hz, 3H), 0.89 (s, 9H), 0.87 (s, 3H), 0.04 (s, 3H), 0.02 (s, 3H). The racemic compound was resolved by chiral HPLC (AD column, 1 ml/min, heptane: IPA=95:05) to afford (1R,2R,4R,6S)-4-(3-aminopyridin-4-yl)-2-((tert-butyldimethylsilyl)oxy)-1,6-dimethylcyclohexanol (>99% ee, Rₜ=2.74 min) and (1S,2S,4S,6R)-4-(3-aminopyridin-4-yl)-2-((tert-butyldimethylsilyl)oxy)-1,6-dimethylcyclohexanol (99% ee, Rₜ=4.25 min)

### Synthesis of (+/−)-4-((3R,5S)-3-(tert-butyldimethylsilyloxy)-5-methyl-4-methylenecyclohex-1-enyl)-3-nitropyridine

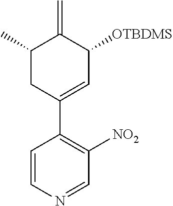 [SMILES: C=C1[C@@H](C)CC(c2ccncc2[N+](=O)[O-])=C[C@H]1O[Si](C)(C)C(C)(C)C]

To solution of (+/−)-(1R,5S)-5-methyl-6-methylene-3-(3-nitropyridin-4-yl)cyclohex-2-enol (1.0 equiv.) in DCM (0.5 M) was added IMIDAZOLE (1.5 equiv.) and TBDMSCl (1.1 equiv.). The reaction mixture was stirred for 18 hr at room temperature. DCM was removed in vacuo and the residue was partitioned between EtOAc and water. The combined organic layer was washed with water and brine, and dried over anhydrous sodium sulfate, filtered and concentrated in vacuo, the crude material was purified by flash column chromatography EtOAc:Heptane (10:90 to 90:10) to yield (+/−)-4-((3R,5S)-3-(tert-butyldimethylsilyloxy)-5-methyl-4-methylenecyclohex-1-enyl)-3-nitropyridine in 80% yield. LCMS (m/z): 361.0 (MH⁺), Rₜ=1.38 min. ¹H NMR (400 MHz, CHLOROFORM-d) δ ppm 9.12 (s, 1H), 8.73 (d, J=5.1 Hz, 1H), 7.27 (d, J=5.1 Hz, 1H) 5.57 (t, J=2.5 Hz, 1H), 5.24-5.20 (m, 1H), 4.98-4.94 (m, 1H), 4.84-4.92 (m, 1H), 2.57-2.72 (m, 1H), 2.37 (dd, J=16.6, 5.3 Hz, 1H), 2.11-2.01 (m, 1H), 1.20 (d, J=6.7 Hz, 3H), 0.92-0.99 (m, 9H), 0.15-0.12 (m, 6H).

### Synthesis of (+/−)-(1S,2R,6S)-2-(tert-butyldimethylsilyloxy)-1-(hydroxymethyl)-6-methyl-4-(3-nitropyridin-4-yl)cyclohex-3-enol

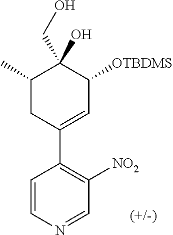 [SMILES: C[C@H]1CC(c2ccncc2[N+](=O)[O-])=C[C@@H](O[Si](C)(C)C(C)(C)C)[C@@]1(O)CO]

To a solution of (+/−)-4-((3R,5S)-3-(tert-butyldimethylsilyloxy)-5-methyl-4-methylenecyclohex-1-enyl)-3-nitropyridine (1.0 equiv.) in acetone/water (4:1, 0.1 M) was added OSMIUM TETROXIDE (4% in water, 0.05 equiv.) and NMO (6.0 equiv.). The reaction mixture was stirred at room temperature for 1 h at which time the reaction was quenched with sat. Na₂S₂O₃, acetone was removed in vacuo, and the reaction mixture was extracted with EtOAc, which was washed with water and brine, and dried over anhydrous sodium sulfate, filtered and concentrated in vacuo. The crude material was purified by flash column chromatography EtOAc:Heptane (0:100 to 90:10) to give (+/−)-(1S,2R,6S)-2-(tert-butyldimethylsilyloxy)-1-(hydroxymethyl)-6-methyl-4-(3-nitropyridin-4-yl)cyclohex-3-enol in 95% yield. LCMS (m/z): 395.0 (MH⁺), Rₜ=1.04 min.

### Synthesis of (+/−)-((1S,2R,6S)-2-(tert-butyldimethylsilyloxy)-1-hydroxy-6-methyl-4-(3-nitropyridin-4-yl)cyclohex-3-enyl)methyl acetate

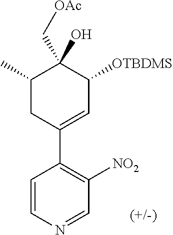 [SMILES: CC(=O)OC[C@@]1(O)[C@@H](C)CC(c2ccncc2[N+](=O)[O-])=C[C@H]1O[Si](C)(C)C(C)(C)C]

To a solution of (+/−)-(1S,2R,6S)-2-(tert-butyldimethylsilyloxy)-1-(hydroxymethyl)-6-methyl-4-(3-nitropyridin-4-yl)cyclohex-3-enol (1.0 equiv.) in DCM (0.1 M), was added PYRIDINE (3.0 equiv.). After the reaction mixture was cooled to 0° C., ACETYL CHLORIDE (1.1 equiv.) in DCM (0.3 M) was added into reaction at 0° C. over 5 min while stirring. The reaction was stirred further for 10 min at 0° C. and quenched by sat. NaHCO₃. After DCM was removed in vacuo, the aqueous phase was extracted with EtOAc 3 times. The combined organic layer was washed with water and brine, and dried over anhydrous sodium sulfate, filtered and concentrated in vacuo. The crude material was purified by flash column chromatography EtOAc:Heptane (0:100 to 90:10) to yield (+/−)-((1S,2R,6S)-2-(tert-butyldimethylsilyloxy)-1-hydroxy-6-methyl-4-(3-nitropyridin-4-yl)cyclohex-3-enyl)methyl acetate in 90% yield. LCMS (m/z): 437.1 (MH⁺), Rₜ=1.14 min.

### Synthesis of ((1R,2S,4S,6R)-4-(3-aminopyridin-4-yl)-2-(tert-butyldimethylsilyloxy)-1-hydroxy-6-methylcyclohexyl)methyl acetate and ((1S,2R,4R,6S)-4-(3-aminopyridin-4-yl)-2-(tert-butyldimethylsilyloxy)-1-hydroxy-6-methylcyclohexyl)methyl acetate

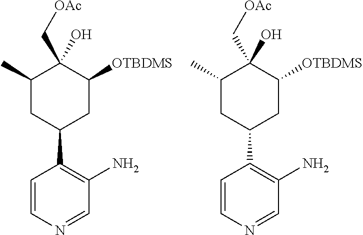 [SMILES: CC(=O)OC[C@]1(O)[C@H](C)C[C@H](c2ccncc2N)C[C@@H]1O[Si](C)(C)C(C)(C)C ; CC(=O)OC[C@@]1(O)[C@@H](C)C[C@@H](c2ccncc2N)C[C@H]1O[Si](C)(C)C(C)(C)C]

A solution of (+/−)-((1S,2R,6S)-2-(tert-butyldimethylsilyloxy)-1-hydroxy-6-methyl-4-(3-nitropyridin-4-yl)cyclohex-3-enyl)methyl acetate (1.0 equiv.) in methanol:EtOAc (3:1, 0.3 M) was degassed by nitrogen for 10 min, followed by addition of 10% Pd/C (0.1 equiv.). The reaction mixture was charged with hydrogen balloon and stirred at room temperature for 18 h. The reaction mixture was filtered through Celite pad and the volatile materials were concentrated to afford the crude (+/−)-((1S,2R,4R,6S)-4-(3-aminopyridin-4-yl)-2-(tert-butyldimethylsilyloxy)-1-hydroxy-6-methylcyclohexyl)methyl acetate. The crude (+/−)-((1S,2R,4R,6S)-4-(3-aminopyridin-4-yl)-2-(tert-butyldimethylsilyloxy)-1-hydroxy-6-methylcyclohexyl)methyl acetate was resolved by chiral SFC (OJ column, methanol/0.5% DEA) to afford ((1R,2S,4S,6R)-4-(3-aminopyridin-4-yl)-2-(tert-butyldimethylsilyloxy)-1-hydroxy-6-methylcyclohexyl)methyl acetate (99% ee, Rₜ=0.51; LCMS (m/z): 409.2 (MH⁺), Rₜ=0.82 min) and ((1S,2R,4R,6S)-4-(3-aminopyridin-4-yl)-2-(tert-butyldimethylsilyloxy)-1-hydroxy-6-methylcyclohexyl)methyl acetate (99% ee, Rₜ=0.82 min; LCMS (m/z): 409.2 (MH⁺), Rₜ=0.82 min).

### Synthesis of (+/−)-(1R,2R,6S)-2-(tert-butyldimethylsilyloxy)-1-hydroxy-6-methyl-4-(3-nitropyridin-4-yl)cyclohex-3-enecarbaldehyde

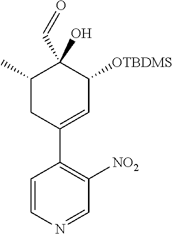 [SMILES: C[C@H]1CC(c2ccncc2[N+](=O)[O-])=C[C@@H](O[Si](C)(C)C(C)(C)C)[C@@]1(O)C=O]

To a solution of (+/−)-(1S,2R,6S)-2-(tert-butyldimethylsilyloxy)-1-(hydroxymethyl)-6-methyl-4-(3-nitropyridin-4-yl)cyclohex-3-enol (1.0 equiv.) in DCM (0.3 M) was added Dess-MartinPeriodinane (1.1 equiv). The reaction mixture was stirred at room temperature for 72 h. After quenched with Na₂S₂O₃ and NaHCO₃ solution (1:8) and stirred for 1 h, the reaction mixture was extracted with EtOAc, the organic layer was washed with water and brine, and dried by anhydrous sodium sulfate, filtered and concentrated in vacuo, the crude product was purified by automatic flash chromatography (0-40% EtOAC/hexanes) to give (+/−)-(1R,2R,6S)-2-(tert-butyldimethylsilyloxy)-1-hydroxy-6-methyl-4-(3-nitropyridin-4-yl)cyclohex-3-enecarbaldehyde as yellow solid in 83% yield. LCMS (m/z): 393.1 (MH⁺), Rₜ=1.20 min. ¹H NMR (400 MHz, CDCl₃) δ ppm 9.94-9.89 (m, 1H), 9.18 (s, 1H), 8.81 (d, J=4.7 Hz, 1H), 7.32 (d, J=5.1 Hz, 1H), 5.67 (s, 1H), 4.46-4.55 (m, 1H), 3.86-3.80 (s, 1H), 2.54 (d, J=3.1 Hz, 1H), 2.49-2.32 (m, 2H), 0.97 (d, J=6.7 Hz, 3H), 0.83 (s, 9H), 0.12-0.05 (m, 6H).

### Synthesis of (+/−)-(1R,2R,6S)-2-(tert-butyldimethylsilyloxy)-6-methyl-4-(3-nitropyridin-4-yl)-1-vinylcyclohex-3-enol

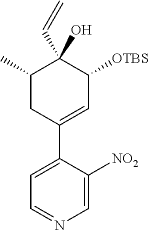 [SMILES: C=C[C@@]1(O)[C@@H](C)CC(c2ccncc2[N+](=O)[O-])=C[C@H]1O[Si](C)(C)C(C)(C)C]

A solution of METHYLTRIPHENYLPHOSPHONIUM BROMIDE (2.0 equiv.) and POTASSIUM TERT-BUTOXIDE (1.9 equiv.) in THF (0.15M) was hearted at 50° C. for 20 mins under Nitrogen, cooling down to room temperature. Then (+/−)-(1R,2R,6S)-2-(tert-butyldimethylsilyloxy)-1-hydroxy-6-methyl-4-(3-nitropyridin-4-yl)cyclohex-3-enecarbaldehyde (1.0 equiv.) in THF (2.0 M) was added slowly at room temperature, the reaction mixture was stirred at room temperature for 3 h. Quenched by NH₄Cl(sat.), the reaction mixture was then extracted by EtOAc; the organic layer was washed by water and brine, dried over anhydrous sodium sulfate, filtered and concentrated in vacuo. The crude material was purified by silica gel column chromatography eluting with ethyl acetate and hexanes (1:2) to give (+/−)-(1R,2R,6S)-2-(tert-butyldimethylsilyloxy)-6-methyl-4-(3-nitropyridin-4-yl)-1-vinylcyclohex-3-enol in 22% yield. LCMS (m/z): 393.1 (MH⁺), Rₜ=1.20 min

### Synthesis of (+/−)-(1R,2R,4R,6S)-4-(3-aminopyridin-4-yl)-2-(tert-butyldimethylsilyloxy)-1-ethyl-6-methylcyclohexanol

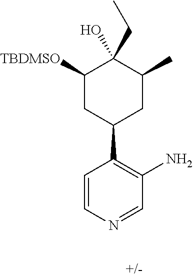 [SMILES: CC[C@@]1(O)[C@@H](C)C[C@@H](c2ccncc2N)C[C@H]1O[Si](C)(C)C(C)(C)C]

A solution of (+/−)-(1R,2R,6S)-2-(tert-butyldimethylsilyloxy)-6-methyl-4-(3-nitropyridin-4-yl)-1-vinylcyclohex-3-enol (1.0 equiv.) in methanol (0.3 M) was degassed by nitrogen for 10 minutes, 10% Pd/C (0.2 equiv.) was added. The reaction mixture was stirred at room temperature for 24 hours under hydrogen atmosphere. The reaction mixture was filtered through celite and washed by MeOH and EtOAc. The filtrate was concentrated in vacuo to give (+/−)-(1R,2R,4R,6S)-4-(3-aminopyridin-4-yl)-2-(tert-butyldimethylsilyloxy)-1-ethyl-6-methylcyclohexanol (>99% yield). LCMS (m/z): 365.1 (MH⁺), Rₜ=0.91 min.

### Synthesis of (+/−)-(1S,2R,6S)-2-(tert-butyldimethylsilyloxy)-1-ethynyl-6-methyl-4-(3-nitropyridin-4-yl)cyclohex-3-enol

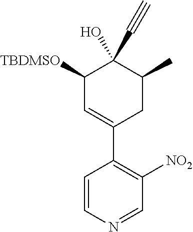 [SMILES: C#C[C@@]1(O)[C@@H](C)CC(c2ccncc2[N+](=O)[O-])=C[C@H]1O[Si](C)(C)C(C)(C)C]

To a solution of (+/−)-(1R,2R,6S)-2-(tert-butyldimethylsilyloxy)-1-hydroxy-6-methyl-4-(3-nitropyridin-4-yl)cyclohex-3-enecarbaldehyde (1.0 equiv.) in MeOH (0.02 M) was added Bestmann-Ohira's reagent (2.0 equiv.) in MeOH (2 mL) followed by addition of POTASSIUM CARBONATE (5.0 equiv.) at room temperature. The reaction mixture was stirred at room temperature for 1.5 h. After removing 90% of MeOH in vacuo and diluted with EtOAc, the organic layer was washed with saturated NH4Cl solution and brine. The organic phase was dried with sodium sulfate, filtered and concentrated. The crude material was purified via silica gel column chromatography eluting with ethyl acetate and heptanes (0-30% EtOAC/Heptane) to yield (+/−)-(1S,2R,6S)-2-(tert-butyldimethylsilyloxy)-1-ethynyl-6-methyl-4-(3-nitropyridin-4-yl)cyclohex-3-enol in 36% yield. LCMS (m/z): 389.2 (MH+), Rₜ=1.15 min. 1H NMR (400 MHz, CHLOROFORM-d) ppm, 9.12 (s, 1H) 8.74 (d, J=5.09 Hz, 1H) 7.29 (d, J=5.09 Hz, 1H) 5.44 (s, 1H) 4.33 (dt, J=3.33, 1.86 Hz, 1H) 2.66 (s, 1H) 2.45 (s, 1H) 2.38-2.30 (m, 2H) 2.28-2.19 (m, 1H) 1.17 (d, J=6.26 Hz, 3H) 0.93 (s, 9H) 0.17-0.09 (m, 6H).

### Synthesis of (+/−)-(1R,2R,4R,6S)-4-(3-aminopyridin-4-yl)-2-(tert-butyldimethylsilyloxy)-1-ethyl-6-methylcyclohexanol

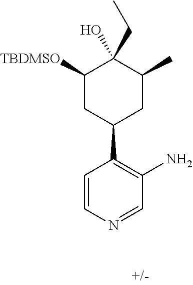 [SMILES: CC[C@@]1(O)[C@@H](C)C[C@@H](c2ccncc2N)C[C@H]1O[Si](C)(C)C(C)(C)C]

To a solution of (+/−)-(1S,2R,6S)-2-(tert-butyldimethylsilyloxy)-1-ethynyl-6-methyl-4-(3-nitropyridin-4-yl)cyclohex-3-enol (1.0 equiv.) in MeOH (0.04 M) was degassed by nitrogen for 10 min, then added 10% Pd/C (0.1 equiv), The reaction mixture was stirred at room temperature for 12 under hydrogen balloon The reaction mixture was filtered through celite and washed by MeOH and EtOAc, the filtrate was concentrated in vacuo to give the crude (+/−)-(1R,2R,4R,6S)-4-(3-aminopyridin-4-yl)-2-(tert-butyldimethylsilyloxy)-1-ethyl-6-methylcyclohexanol in >99% yield. LCMS (m/z): 365.1 (MH⁺), Rₜ=0.93 min.

### Synthesis of (1R,2R,4R,6S)-4-(3-aminopyridin-4-yl)-2-(tert-butyldimethylsilyloxy)-1-ethyl-6-methylcyclohexanol and (1S,2S,4S,6R)-4-(3-aminopyridin-4-yl)-2-(tert-butyldimethylsilyloxy)-1-ethyl-6-methylcyclohexanol

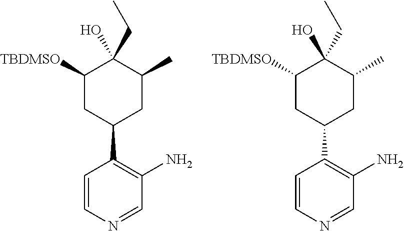 [SMILES: CC[C@@]1(O)[C@@H](C)C[C@@H](c2ccncc2N)C[C@H]1O[Si](C)(C)C(C)(C)C ; CC[C@]1(O)[C@H](C)C[C@H](c2ccncc2N)C[C@@H]1O[Si](C)(C)C(C)(C)C]

(+/−)-(1R,2R,4R,6S)-4-(3-aminopyridin-4-yl)-2-(tert-butyldimethylsilyloxy)-1-ethyl-6-methylcyclohexanol was resolved by chiral SFC (Chiralpak, 10×250, 15 mL/min, CO₂/EtOH+0.1% DEA, 85/15. 40° C.) to yield (1R,2R,4R,6S)-4-(3-aminopyridin-4-yl)-2-(tert-butyldimethylsilyloxy)-1-ethyl-6-methylcyclohexanol (99% ee, Rt=1.49 min) and (1S,2S,4S,6R)-4-(3-aminopyridin-4-yl)-2-(tert-butyldimethylsilyloxy)-1-ethyl-6-methylcyclohexanol (99% ee, Rt=1.91 min).

### Synthesis of (+/−)-4-((3S,4R,8S)-4-(tert-butyldimethylsilyloxy)-8-methyl-1-oxaspiro[2.5]oct-5-en-6-yl)-3-nitropyridine

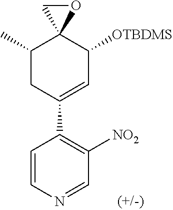

To a solution of (+/−)-(1R,2R,6S)-1-(bromomethyl)-2-(tert-butyldimethylsilyloxy)-6-methyl-4-(3-nitropyridin-4-yl)cyclohex-3-enol (1.0 equiv.) in MeOH:H₂O (10:1, 0.3 M) was added POTASSIUM CARBONATE (1.5 equiv.). The reaction mixture was vigorously stirred for 1 h at room temperature. MeOH was evaporated, then the reaction mixture was partitioned between EtOAc and water. The combined organic layer was washed with water and brine, dried over anhydrous sodium sulfate, filtered and concentrated in vacuo to yield (+/−)-4-((3S,4R,8S)-4-(tert-butyldimethylsilyloxy)-8-methyl-1-oxaspiro[2.5]oct-5-en-6-yl)-3-nitropyridine in 99% yield. LCMS (m/z): 377.1 (MH⁺), Rₜ=1.31 min: ¹H NMR (400 MHz, CHLOROFORM-d) δ ppm 9.14 (s, 1H), 8.76 (d, J=5.1 Hz, 1H), 7.31 (d, J=5.1 Hz, 1H), 5.59 (s, 1H), 4.49 (br. s., 1H), 2.98 (d, J=5.1 Hz, 1H), 2.72 (d, J=5.1 Hz, 1H), 2.54-2.37 (m, 2H), 2.27-2.21 (m, 1H), 0.98-0.91 (m, 3H), 0.91-0.85 (m, 9H), 0.13-0.05 (m, 6H).

### Synthesis of (+/−)-(1R,2R,6S)-1-(fluoromethyl)-6-methyl-4-(3-nitropyridin-4-yl)cyclohex-3-ene-1,2-diol

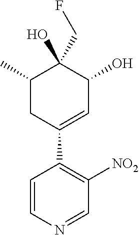

A solution of (+/−)-4-((3S,4R,8S)-4-(tert-butyldimethylsilyloxy)-8-methyl-1-oxaspiro[2.5]oct-5-en-6-yl)-3-nitropyridine (1.0 equiv.) in triethylamine trihydrofluoride (0.15 M) in a stainless steel reactor was heated at 100° C. for 8 h. Cooling down and quenched by Sat.NaHCO₃ solution. The reaction mixture was then partitioned between EtOAc and water. The combined organic layer was washed with water and brine, dried over anhydrous sodium sulfate. Filtered and concentrated in vacuo to yield (+/−)-(1R,2R,6S)-1-(fluoromethyl)-6-methyl-4-(3-nitropyridin-4-yl)cyclohex-3-ene-1,2-diol in 99% yield. LCMS (m/z): 283.0 (MH⁺), Rₜ=0.51 min.

### Synthesis of (+/−)-(1R,2R,4R,6S)-4-(3-aminopyridin-4-yl)-1-(fluoromethyl)-6-methylcyclohexane-1,2-diol

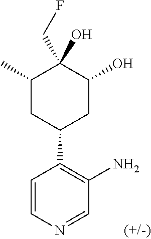 [SMILES: C[C@H]1C[C@@H](c2ccncc2N)C[C@@H](O)[C@@]1(O)CF]

A solution of (+/−)-(1R,2R,6S)-1-(fluoromethyl)-6-methyl-4-(3-nitropyridin-4-yl)cyclohex-3-ene-1,2-diol (1.0 equiv.) in MeOH (0.04 M) was degassed by nitrogen for 10 min, then added 10% Pd/C (0.1 equiv.), The reaction mixture was stirred at room temperature for 12 h under hydrogen balloon. The reaction mixture was filtered through celite and washed by MeOH and EtOAc, the filtrate was concentrated in vacuo to give (+/−)-(1R,2R,4R,6S)-4-(3-aminopyridin-4-yl)-1-(fluoromethyl)-6-methylcyclohexane-1,2-diol in 50% yield. LCMS (m/z): 255.0 (MH⁺), Rₜ=0.32 min.

### Synthesis of 4-((+/−)-6-(bromomethyl)-5-methyl-7-oxabicyclo[4.1.0]hept-2-en-3-yl)-3-nitropyridine

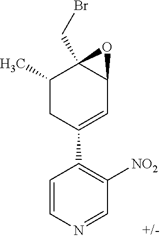

To a 0.15 M solution of (+/−)-1-(bromomethyl)-6-methyl-4-(3-nitropyridin-4-yl)cyclohex-3-ene-1,2-diol (1.0 equiv) in DCM was added TEA (2.0 equiv) at 0° C. MsCl (1.4 equiv) was added dropwise over 10 minutes. The reaction mixture was stirred at 0° C. for 1 hour. The reaction mixture was quenched with saturated aqueous sodium bicarbonate and stirred for 20 minutes. The reaction mixture was extracted with DCM. The combined organic layers were washed sequentially with water and brine, dried over anhydrous sodium sulfate, filtered and concentrated under reduced pressure to give crude 4-((+/−)-6-(bromomethyl)-5-methyl-7-oxabicyclo[4.1.0]hept-2-en-3-yl)-3-nitropyridine in quantitative yield. LC/MS (m/z): 325/327 (MH⁺), Rₜ=0.84 min.

### Synthesis of 4-((+/−)-4-azido-8-methyl-1-oxaspiro[2.5]oct-5-en-6-yl)-3-nitropyridine

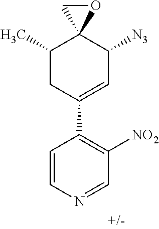

To a 0.25 M solution of 4-((+/−)-6-(bromomethyl)-5-methyl-7-oxabicyclo[4.1.0]hept-2-en-3-yl)-3-nitropyridine (1.0 equiv.) in 3:1 ethanol:water was added ammonium chloride (1.5 equiv.) and sodium azide (1.5 equiv.). The reaction mixture was stirred at room temperature for 18 hours. The reaction mixture was treated with an equal volume of saturated aqueous sodium bicarbonate and acetonitrile and stirred for 2 hours. Volatiles were removed under reduced pressure. The mixture was extracted with ethyl acetate. The combined organic layers were washed with brine, dried over anhydrous sodium sulfate, filtered, and concentrated. The crude material was purified by flash chromatography over silica gel (heptanes with 20% to 80% ethyl acetate gradient) to give 4-((+/−)-4-azido-8-methyl-1-oxaspiro[2.5]oct-5-en-6-yl)-3-nitropyridine in 57% yield as a yellow oil. LC/MS (m/z): 288.0 (MH⁺), Rₜ=0.80 min.

### Synthesis of tert-butyl (+/−)-5-(3-aminopyridin-4-yl)-2-hydroxy-2,3-dimethylcyclohexylcarbamate

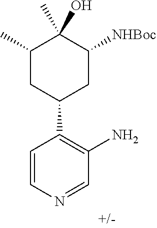 [SMILES: C[C@H]1C[C@@H](c2ccncc2N)C[C@@H](NC(=O)OC(C)(C)C)[C@]1(C)O]

A 0.05 M solution of 4-((+/−)-4-azido-8-methyl-1-oxaspiro[2.5]oct-5-en-6-yl)-3-nitropyridine (1.0 equiv.) in ethanol was degassed for 10 minutes. Pyridine (10 equiv.) and 10% palladium on carbon (0.3 equiv) were added. The reaction vessel was purged and flushed three times with hydrogen. The reaction was stirred under a hydrogen atmosphere for 4 days. The reaction mixture was purged of hydrogen, diluted with DCM/MeOH, and filtered. The filter cake was rinsed with additional DCM/MeOH. The filtrate was concentrated. The residue was dissolved in ethanol to make a 0.1 M solution and treated with di-tert-butyl dicarbonate (1.2 equiv.). The mixture was stirred for 1 hr at ambient temperature and then concentrated under reduced pressure. The residue was purified by flash chromatography over silica gel (95:5 DCM:MeOH+0.5% NH₄OH to 90:10 DCM:MeOH+1% NH₄OH) to give racemic tert-butyl (+/−)-5-(3-aminopyridin-4-yl)-2-hydroxy-2,3-dimethylcyclohexylcarbamate in 42% yield. The enantiomers could be separated using an AD column eluting with heptanes/IPA. LC/MS (m/z): 336.1 (MH⁺), Rₜ=0.50 min. ¹H-NMR (400 MHz, methanol-d4): δ ppm 7.94 (s, 1H) 7.78 (d, J=5.09 Hz, 1H) 7.08 (d, J=5.09 Hz, 1H) 3.67 (m, 1H) 2.84-3.04 (m, 1H) 1.69-1.95 (m, 2H) 1.69-1.79 (m, 1H) 1.41-1.57 (m, 10H) 1.29-1.41 (m, 1H) 1.08 (s, 3H) 1.03 (d, J=6.65 Hz, 3H)

### Synthesis of 6-bromo-5-fluoropicolinic acid

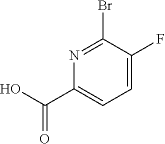 [SMILES: O=C(O)c1ccc(F)c(Br)n1]

To 2-bromo-3-fluoro-6-methylpyridine (1.0 equiv.) in H₂O (30 mL) was added potassium permanganate (1.0 equiv.). The solution was heated at 100° C. for 5 hours at which time more potassium permanganate (1.0 equiv.) was added. After heating for an additional 48 hours the material was filtered through celite (4 cm×2 inches) and rinsed with H₂O (150 mL). The combined aqueous was acidified with 1N HCl to pH=4, extracted with ethyl acetate (200 mL), washed with NaCl(sat.), dried over MgSO₄, filtered and concentrated to yield 6-bromo-5-fluoropicolinic acid (17%) as a white solid. LCMS (m/z): 221.9 (MH+); LC Rt=2.05 min.

### Synthesis of methyl 6-bromo-5-fluoropicolinate

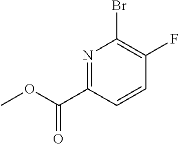

To a solution of 6-bromo-5-fluoropicolinic acid (1.0 equiv.) in methanol (0.2 M) was added H₂SO₄ (4.2 equiv.) and the reaction was stirred at room temperature for two hours. Upon completion of the reaction as monitored by LC/MS, the reaction was diluted with ethyl acetate and quenched slowly with saturated aqueous NaHCO₃. The reaction was poured into a separatory funnel and extracted with ethyl acetate. The organic phase was dried with magnesium sulfate, filtered, and concentrated in vacuo to provide methyl 6-bromo-5-fluoropicolinate as a white solid (>99%). LC/MS=233.9/235.9 (M+H), Rt=0.69 min.

### Synthesis of 2-(2,6-difluorophenyl)-3-fluoro-6-methylpyridine

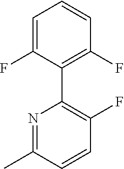 [SMILES: Cc1ccc(F)c(-c2c(F)cccc2F)n1]

To a solution of 2-bromo-3-fluoro-6-methylpyridine (1.0 equiv.) in THF and Water (10:1, 0.2 M) was added 2,6-difluorophenylboronic acid (2.0 equiv.) and potassium fluoride (3.3 equiv.). The reaction was degassed for 10 minutes, then Pd₂(dba)₃ (0.05 equiv.) was added, followed by tri-t-butylphosphine (0.1 equiv.). The reaction was stirred to 60° C. for 1 hour at which point, all starting material was consumed as indicated by LC/MS. The reaction was allowed to cool to room temperature, partitioned with ethyl acetate and water, the organic phase was dried with sodium sulfate, filtered, and concentrated. The crude material was diluted in EtOH to 0.1 M, and 0.5 equiv. of NaBH₄ was added to reduce the dba. The reaction was stirred for one hour at room temperature, then quenched with water and concentrated under vacuo to remove the ethanol. The product was extracted in ether, washed with brine, the organics were dried over sodium sulfate, filtered, and concentrated. The crude material was loaded on silica gel and purified via column chromatography (ISCO) eluting with hexanes and ethyl acetate (0%-10% ethyl acetate). The pure fractions were combined, and concentrated to yield 2-(2,6-difluorophenyl)-3-fluoro-6-methylpyridine as a light yellow oil in 86% yield. LC/MS=224.0 (M+H), Rₜ=0.84 min.

### Synthesis of 6-(2,6-difluorophenyl)-5-fluoropicolinic acid

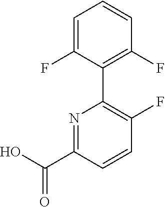 [SMILES: O=C(O)c1ccc(F)c(-c2c(F)cccc2F)n1]

To a solution of 2-(2,6-difluorophenyl)-3-fluoro-6-methylpyridine (1.0 equiv.) in water (0.05 M) was added KMnO₄ (2.0 equiv.) and the reaction was heated to reflux overnight. Another 2.0 equiv. of KMnO₄ were added and stirred at reflux for another 8 hours. The solution was cooled to room temperature, filtered through Celite and washed with water. The filtrate was acidified with 6N HCl to pH=3, the white precipitate was filtered. The filtrate was further acidified to pH=1 and filtered again. The filtrate was extracted with ethyl acetate until no more product in the aqueous layer. The organic phase was washed with brine and dried over magnesium sulfate, filtered, and concentrated. The residue was dissolved in ethyl acetate, washed with 1N NaOH, the aqueous layer was acidified to pH=1 and the white crystals were filtered. The combined products yielded 6-(2,6-difluorophenyl)-5-fluoropicolinic acid in 32% yield as a white solid. LC/MS=254.0 (M+H), Rₜ=0.71 min.

### Synthesis of 6-(2,6-difluoro-3-nitrophenyl)-5-fluoropicolinic acid

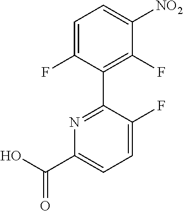 [SMILES: O=C(O)c1ccc(F)c(-c2c(F)ccc([N+](=O)[O-])c2F)n1]

To a solution of 6-(2,6-difluorophenyl)-5-fluoropicolinic acid (1.0 equiv.) in H₂SO₄ (1.7 M)) was added fuming nitric acid: H2SO4 (1:1 V %) mixture slowly at room temperature. The reaction mixture was stirred at room temperature for 2 h. The reaction mixture was added into ice water, solid was percipitated. The solid was filtered and washed with water, air dry followed by high vacuum dry to yield 6-(2,6-difluoro-3-nitrophenyl)-5-fluoropicolinic acid in 85% yield. LCMS (m/z): 299.1 (MH⁺), Rₜ=0.70 min. ¹H NMR (400 MHz, Acetone-d6) δ ppm 8.74 (br. s., 1H), 8.50 (dt, J=5.9, 8.8 Hz, 1H), 8.43 (dd, J=3.9, 8.6 Hz, 1H), 8.13 (t, J=8.8 Hz, 1H), 7.54 (t, J=8.8 Hz, 1H)

### Synthesis of ethyl 2-(2,6-difluorophenyl)thiazole-4-carboxylate

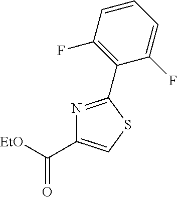 [SMILES: CCOC(=O)c1csc(-c2c(F)cccc2F)n1]

A solution of 2,6-difluorobenzothioamide (1.0 eq) and ethylbromopyruvate (1.0 eq.) in ethanol (1.0 M) was heated in the microwave at 130° C. for 30 minutes. Upon removal of volatiles in vacuo, ethyl acetate was added and the solution was washed with Na₂CO_{3(sat.)}, with NaCl_{(sat.)}, was dried over MgSO₄, filtered and concentrated yielding ethyl 2-(2,6-difluorophenyl)thiazole-4-carboxylate (84%). LCMS (m/z): 270.1 (MH⁺); LC Rₜ=3.79 min.

### Synthesis of 2-(2,6-difluorophenyl)thiazole-4-carboxylic acid

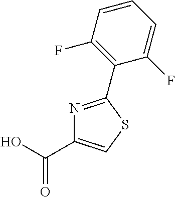 [SMILES: O=C(O)c1csc(-c2c(F)cccc2F)n1]

To a solution of ethyl 2-(2,6-difluorophenyl)thiazole-4-carboxylate (1.0 eq.) in 2:1 THF/MeOH (0.17 M) was added 1.0 M LiOH (2.0 eq.). After standing for 16 hours, 1.0 M HCl (2.0 eq.) was added and the THF/MeOH was removed in vacuo. The resulting solid was filtered, rinsed with H₂O and dried, yielding 2-(2,6-difluorophenyl)thiazole-4-carboxylic acid (88%) as a crusty solid. LCMS (m/z): 251.1 (MH⁺); LC Rₜ=2.68 min.

### Synthesis of Methyl 3-amino-5-fluoropicolinate

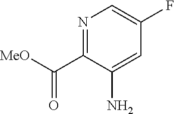 [SMILES: COC(=O)c1ncc(F)cc1N]

To a steel bomb reactor, 2-bromo-5-fluoropyridin-3-amine (1.0 equiv.), triethylamine (1.6 equiv.), Pd(BINAP)Cl₂ (0.0015 equiv.) and anhydrous methanol (0.4 M solution) were added. After degassed by nitrogen stream for 15 min, the steel bomb reactor was closed and filled with CO gas up to 60 psi. The reactor was then heated to 100° C. After 3 h, more Pd catalyst (0.0015 equiv.) was added and the reaction mixture was re-heated to the same temperature for 3 h. After cooling down to room temperature, a brown precipitate was filtered off and the filtrate was extracted with EtOAc, which was washed with water and brine, dried over anhydrous sodium sulfate, and filtered. After removing volatile materials, the crude yellow product was obtained and used for the next step without further purification (40%). LCMS (m/z): 271.2 (MH⁺); LC Rₜ=3.56 min.

### Synthesis of Methyl 3-amino-6-bromo-5-fluoropicolinate

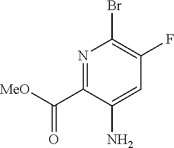 [SMILES: COC(=O)c1nc(Br)c(F)cc1N]

To a solution of methyl 3-amino-5-fluoropicolinate (1.0 equiv.) in acetonitrile (0.3 M solution) was added NBS (1.1 equiv.) for 2 minutes at room temperature. After quenched with water, the reaction mixture was extracted with EtOAc. The crude product was purified by silica column chromatography (20% to 50% EtOAc in hexanes) to give methyl 3-amino-6-bromo-5-fluoropicolinate (41%). LCMS (m/z): 249.1 (MH⁺); LC Rₜ=2.80 min.

### Synthesis of 2-chloro-6-phenylpyrazine

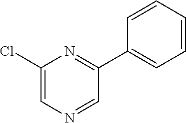 [SMILES: Clc1cncc(-c2ccccc2)n1]

To a solution of 2,6-dichloropyrazine (2.0 equiv.) in 3:1 DME:2M aqueous sodium carbonate (0.125 M) was added phenylboronic acid (1.0 equiv.) then PdCl₂(dppf)•DCM adduct (0.1 equiv.). The reaction was heated in the microwave at 120° C. for 15 minutes. The crude reaction mixture was diluted with ethyl acetate and washed with sat. aq. sodium bicarbonate then sat. NaCl. The organic phase was dried with magnesium sulfate, filtered, and concentrated. The crude material was purified by silica gel column chromatography with heptanes to 30% ethyl acetate in heptanes to give 2-chloro-6-phenylpyrazine in 75% yield. LC/MS (m/z): 191.0 (MH⁺), Rₜ=1.00 min.

### Synthesis of methyl 6-phenylpyrazine-2-carboxylate

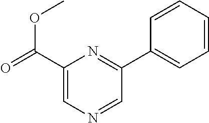 [SMILES: COC(=O)c1cncc(-c2ccccc2)n1]

To a steel pressure vessel with a stir bar was added a solution of 2-chloro-6-phenylpyrazine (1 equiv.) in MeOH (0.2 M) followed by triethylamine (1.5 equiv.) which was degassed with nitrogen for 5 min. DIEA (2.5 equiv.) was added. Pd (II) R-Binap (0.012 equiv.) was added then the reaction vessel was sealed and then carbon monoxide atmosphere was added to 70 psi. The mixture was then heated to 100° C. for 18 hours. The reaction mixture was diluted with ethyl acetate and washed with water then sat. NaCl. The organic phase was dried with sodium sulfate, filtered, and concentrated. The crude material was purified by silica gel column chromatography with heptanes to 20% ethyl acetate in heptanes to give 6-phenylpyrazine-2-carboxylate in 99% yield. LC/MS (m/z): 215.0 (MH⁺), Rₜ=0.73 min.

### Synthesis of 6-phenylpyrazine-2-carboxylic acid

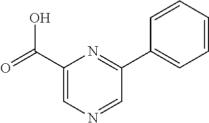 [SMILES: O=C(O)c1cncc(-c2ccccc2)n1]

To a solution of 6-phenylpyrazine-2-carboxylate (1.0 equiv.) in THF (0.2 M) was added a 2 M solution of LiOH (10 equiv.) and allowed to stir over two days at rt. The reaction mixture was acidified with 1N HCl until a white solid precipitated and then filtered. The solid was dried overnight on the high-vac to remove all water to yield 6-phenylpyrazine-2-carboxylic acid in 67% yield. LC/MS (m/z): 201.0 (MH⁺), Rₜ=0.62 min.

### Synthesis of methyl 3-amino-6-(thiazol-2-yl)picolinate

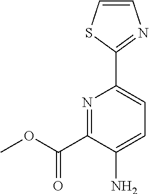 [SMILES: COC(=O)c1nc(-c2nccs2)ccc1N]

A solution of methyl 3-amino-6-bromopicolinate (1.0 equiv.), 2-thiazolylzinc bromide 0.5 M solution in THF (3.0 equiv.), and Pd(dppf)Cl₂-DCM (0.05 equiv.) was stirred at 80° C. for 1.5 hours. The reaction was filtered and washed with EtOAc. The organic was washed with H₂O (100 mL), and further washed with NaCl_{(sat.)} (50 mL), dried over MgSO₄, and the volatiles were removed in vacuo. The product was crystallized with hexane/EtOAc (1:1) to yield methyl 3-amino-6-(thiazol-2-yl)picolinate (51%). LCMS (m/z): 236.1 (MH⁺); LC Rₜ=2.3 min.

### Synthesis of 3-amino-6-(thiazol-2-yl)picolinic acid

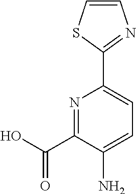 [SMILES: Nc1ccc(-c2nccs2)nc1C(=O)O]

To a solution of methyl 3-amino-6-(thiazol-2-yl)picolinate (1.0 equiv) in THF (0.5M), was added 1M LiOH (4.0 equiv). After stirring for 4 hours at 60° C., 1 N HCl (4.0 equiv.) was added and the THF was removed in vacuo. The resulting solid was filtered and rinsed with cold H₂O (3×20 mL) to yield 3-amino-6-(thiazol-2-yl)picolinic acid (61%). LCMS (m/z): 222.1 (MH+); LC Rₜ=1.9 min.

### Method 1

### Synthesis of methyl 6-(3-(benzyloxy)-2,6-difluorophenyl)-5-fluoropicolinate

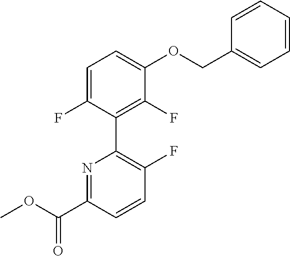 [SMILES: COC(=O)c1ccc(F)c(-c2c(F)ccc(OCc3ccccc3)c2F)n1]

To a solution of methyl 6-bromo-5-fluoropicolinate (1.0 equiv.) in THF and water (10:1, 0.1 M) was added 3-(benzyloxy)-2,6-difluorophenylboronic acid (2.5 equiv.) and potassium fluoride (3.3 equiv.). The reaction was degassed with nitrogen, then Pd₂(dba)₃ (0.25 equiv.) and tri-tert-butylphosphine (0.5 equiv.) were added and the reaction was heated to 80° C. for one hour. LC/MS analysis indicated complete conversion of the starting material to product. The reaction was cooled to room temperature, then concentrated in vacuo and fused to silica gel. The crude product was purified by ISCO flash chromatography eluting with ethyl acetate and hexanes (0% to 30% ethyl acetate) to provide methyl 6-(3-(benzyloxy)-2,6-difluorophenyl)-5-fluoropicolinate as the desired product as a light yellow oil in 96% yield. LC/MS=374.0 (M+H), Rt=1.07 min.

### Synthesis of methyl 6-(2,6-difluoro-4-methoxyphenyl)-5-fluoropicolinate

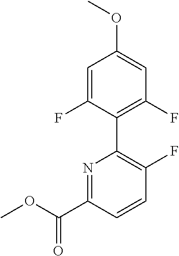 [SMILES: COC(=O)c1ccc(F)c(-c2c(F)cc(OC)cc2F)n1]

Method 1 was followed using 6-bromo-5-fluoropicolinate (1.0 equiv.) and 2,6-difluoro-4-methoxyphenylboronic acid (2.5 equiv.) to give methyl 6-(2,6-difluoro-4-methoxyphenyl)-5-fluoropicolinate as a white solid in 85% yield. LC/MS=298.0 (M+H), Rt=0.89 min.

### Synthesis of methyl 3-amino-6-(2,6-difluorophenyl)picolinate

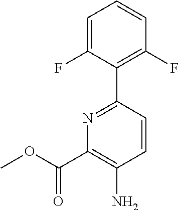 [SMILES: COC(=O)c1nc(-c2c(F)cccc2F)ccc1N]

A solution of methyl 3-amino-6-bromopicolinate (1.0 equiv.), 2,6-difluorophenylboronic acid (3.0 equiv), and Pd(dppf)Cl₂-DCM (0.1 equiv.) in 3:1 DME/2M Na₂CO₃ (0.5 M) was subjected to microwave irradiation at 120° C. for 15 min intervals. The reaction was filtered and washed with EtOAc. The organic was partitioned with H₂O (25 mL), was further washed with NaCl_{(sat.)} (25 mL), was dried over MgSO₄, and the volatiles were removed in vacuo. The residue was diluted in EtOAc and passed through a silica gel plug and the volatiles were removed in vacuo yielding methyl 3-amino-6-(2,6-difluorophenyl)picolinate (47%). LCMS (m/z): 265.1 (MH⁺); LC Rₜ=2.70 min.

### Method 2

### Synthesis of 6-(2,6-difluoro-4-methoxyphenyl)-5-fluoropicolinic acid

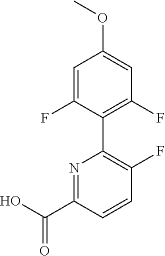 [SMILES: COc1cc(F)c(-c2nc(C(=O)O)ccc2F)c(F)c1]

To a solution of methyl 6-(2,6-difluoro-4-methoxyphenyl)-5-fluoropicolinate (1.0 equiv.) in THF/MeOH (2:1, 0.09 M) was added LiOH (1.5 equiv.) and the reaction was stirred at room temperature for 1 hour. The solution was quenched with 1N HCl, extracted with ethyl acetate, washed with brine, dried with sodium sulfate, filtered and concentrated to give 6-(2,6-difluoro-4-methoxyphenyl)-5-fluoropicolinic acid in 84% yield. LC/MS=284.1 (M+H), Rt=0.76 min.

### Synthesis of 3-amino-6-(2,6-difluorophenyl)picolinic acid

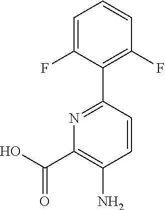 [SMILES: Nc1ccc(-c2c(F)cccc2F)nc1C(=O)O]

To a solution of methyl 3-amino-6-(2,6-difluorophenyl)picolinate (1.0 equiv) in THF (0.5 M), was added 1M LiOH (4.0 equiv). After stirring for 4 hours at 60° C., 1 N HCl (4.0 equiv.) was added and the THF was removed in vacuo. The resulting solid was filtered and rinsed with cold H₂O (3×20 mL) to yield 3-amino-6-(2,6-difluorophenyl)picolinic acid (90%). LCMS (m/z): 251.1 (MH⁺); LC Rₜ=2.1 min.

### Synthesis of 2-(2,6-difluoro-4-methylphenyl)-4,4,5,5-tetramethyl-1,3,2-dioxaboroane

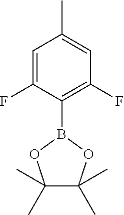 [SMILES: Cc1cc(F)c(B2OC(C)(C)C(C)(C)O2)c(F)c1]

To a solution of 1,3-difluoro-5-methylbenzene (1.0 eq) in dry THF (0.2M) under an atmosphere of N₂ at −78° C. was added n-butyllithium (1 eq, 1.6M in hexanes) slowly keeping the internal temperature below −65° C. The reaction was stirred for 2 hrs at −78° C., followed by the addition of 2-isopropoxy-4,4,5,5-tetramethyl-1,3,2-dioxaborolane (1.15 eq). The reaction was allowed to warm to room temperature. Upon completion, the reaction was quenched with NaHCO_{3 (sat.)} and extracted with EtOAc. The organics were washed with brine, dried over Na₂SO₄, filtered and concentrated to yield a 2-(2,6-difluoro-4-methylphenyl)-4,4,5,5-tetramethyl-1,3,2-dioxaboroane as a white solid in 92%. 1H NMR (400 MHz, <cdcl3>) δ ppm 6.67 (dd, J=9.39, 0.78 Hz, 2H), 2.34 (s, 3H), 1.38 (s, 12H).

### Synthesis of 6-(2,6-difluoro-4-methylphenyl)-5-fluoropicolinate

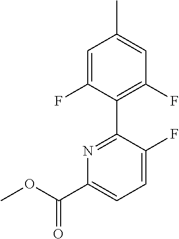 [SMILES: COC(=O)c1ccc(F)c(-c2c(F)cc(C)cc2F)n1]

Method 1 was followed using 6-bromo-5-fluoropicolinate (1.0 equiv.) and 2-(2,6-difluoro-4-methylphenyl)-4,4,5,5-tetramethyl-1,3,2-dioxaboroane (1.75 equiv.) to give methyl 6-(2,6-difluoro-4-methylphenyl)-5-fluoropicolinate as a solid in 85% yield. LC/MS=282.0 (M+H), Rt=0.87 min.

### Synthesis of 6-(2,6-difluoro-4-methylphenyl)-5-fluoropicolinic acid

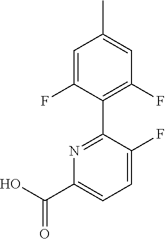 [SMILES: Cc1cc(F)c(-c2nc(C(=O)O)ccc2F)c(F)c1]

To a solution of 6-(2,6-difluoro-4-methylphenyl)-5-fluoropicolinate (1.0 eq) in THF (0.1M) was added LiOH (5.5 eq, 2M) and allowed to stir at room temperature for 4 hrs. The volatiles were removed in vacuo, and the residual aqueous was acidified with 2M HCl to pH 4. The precipitate was filtered and dried to yield 6-(2,6-difluoro-4-methylphenyl)-5-fluoropicolinic acid as al light yellow solid in 73.5%. LCMS (m/z): 268.0 (MH⁺), Rₜ=0.76 min.

### Synthesis of methyl 6-(2,6-difluoro-4-formylphenyl)-5-fluoropicolinate

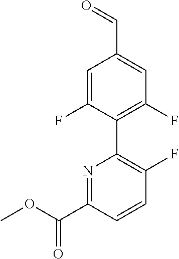 [SMILES: COC(=O)c1ccc(F)c(-c2c(F)cc(C=O)cc2F)n1]

Method 1 was followed using 6-bromo-5-fluoropicolinate (1.0 equiv.) and 3,5-difluoro-4-(4,4,5,5-tetramethyl-1,3,2-dioxaborolan-2-yl)benzaldehyde (1.8 equiv.) to give methyl 6-(2,6-difluoro-4-formylphenyl)-5-fluoropicolinate as an off-white solid in 66% yield. LC/MS=295.9 (M+H), Rt=0.73 min.

### Synthesis of methyl 6-(2,6-difluoro-4-vinylphenyl)-5-fluoropicolinate

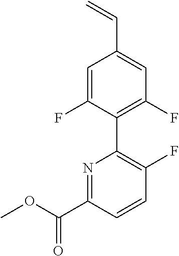 [SMILES: C=Cc1cc(F)c(-c2nc(C(=O)OC)ccc2F)c(F)c1]

To a solution of Methyltriphenylphosphonium bromide (1.5 equiv) in THF (0.1 M) was added potassium tert-butoxide (1.45 eq.) After stirring at rt for 2 hours the solution was cooled to −78° C. and a solution of methyl 6-(2,6-difluoro-4-formylphenyl)-5-fluoropicolinate (1.0 eq.) in THF was added dropwise. The solution was stirred for 16 hours as the temperature gradually warmed to rt. The solution was partitioned between EtOAc and water, washed with NaHCO_{3(sat.)}, NaCl_{(sat.)}, dried over MgSO₄, filtered, concentrated and purified by ISCO SiO₂ chromatography to yield methyl 6-(2,6-difluoro-4-vinylphenyl)-5-fluoropicolinate as a white solid in 63% yield. LC/MS=293.9 (M+H), Rₜ=0.90 min.

### Synthesis of 6-(2,6-difluoro-4-vinylphenyl)-5-fluoropicolinic acid

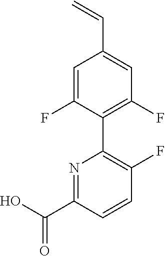 [SMILES: C=Cc1cc(F)c(-c2nc(C(=O)O)ccc2F)c(F)c1]

Method 2 was followed using methyl 6-(2,6-difluoro-4-vinylphenyl)-5-fluoropicolinate to give 6-(2,6-difluoro-4-vinylphenyl)-5-fluoropicolinic acid in 94% yield. LC/MS=279.9 (M+H), Rₜ=0.78 min.

### Synthesis of methyl 6-(2,6-difluoro-4-(hydroxymethyl)phenyl)-5-fluoropicolinate

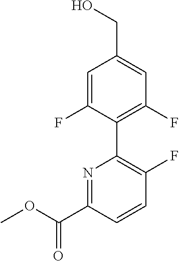 [SMILES: COC(=O)c1ccc(F)c(-c2c(F)cc(CO)cc2F)n1]

To a solution of methyl 6-(2,6-difluoro-4-formylphenyl)-5-fluoropicolinate (1.0 eq.) in THF (0.24 M) at 0° C. was added sodium borohydride. After stirring for 10 minutes, water was added and the solution was extracted with EtOAc, washed with NaCl(sat.), dried over Na₂SO₄, filtered and concentrated to yield methyl 6-(2,6-difluoro-4-(hydroxymethyl)phenyl)-5-fluoropicolinate. LC/MS=297.9 (M+H), Rₜ=0.66 min.

### Synthesis of methyl 6-(2,6-difluoro-4-(methoxymethyl)phenyl)-5-fluoropicolinate

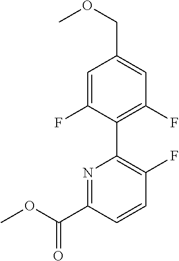 [SMILES: COCc1cc(F)c(-c2nc(C(=O)OC)ccc2F)c(F)c1]

To a solution of methyl 6-(2,6-difluoro-4-(hydroxymethyl)phenyl)-5-fluoropicolinate (1.0 eq.) in DMF (0.03 M) at 0° C. was added sodium hydride (1.5 eq). After stirring for 2 minutes, methyl iodide (1.5 eq.) was added. After stirring for 1 hour, water was added and the solution was extracted with EtOAc (3×), the combined organics were dried over Na₂SO₄, filtered, concentrated and purified by ISCO SiO₂ chromatography to yield methyl 6-(2,6-difluoro-4-(methoxymethyl)phenyl)-5-fluoropicolinate. LC/MS=311.9 (M+H), Rₜ=0.86 min.

### Synthesis of 6-(2,6-difluoro-4-(methoxymethyl)phenyl)-5-fluoropicolinic acid

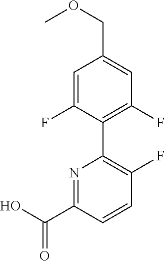 [SMILES: COCc1cc(F)c(-c2nc(C(=O)O)ccc2F)c(F)c1]

Method 2 was followed using methyl methyl 6-(2,6-difluoro-4-(methoxymethyl)phenyl)-5-fluoropicolinate to give 6-(2,6-difluoro-4-(methoxymethyl)phenyl)-5-fluoropicolinic acid in 84% yield. LC/MS=297.9 (M+H), Rₜ=0.78 min.

### Synthesis of 2-(2,6-difluoro-4-(methylthio)phenyl)-4,4,5,5-tetramethyl-1,3,2-dioxaborolane

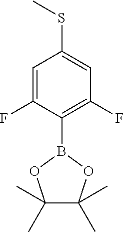 [SMILES: CSc1cc(F)c(B2OC(C)(C)C(C)(C)O2)c(F)c1]

To a solution of (3,5-difluorophenyl)(methyl)sulfane (1.0 eq) in dry THF (0.2M) under an atmosphere of N₂ at −78° C. was added n-butyllithium (1 eq, 1.6M in hexanes) slowly keeping the internal temperature below −65° C. The reaction was stirred for 2 hrs at −78° C., followed by the addition of 2-isopropoxy-4,4,5,5-tetramethyl-1,3,2-dioxaborolane (1.15 eq). The reaction was allowed to warm to room temperature. Upon completion, the reaction was quenched with NaHCO_{3 (sat.)} and extracted with EtOAc. The organics were washed with brine, dried over Na₂SO₄, filtered and concentrated to yield a 2-(2,6-difluoro-4-(methylthio)phenyl)-4,4,5,5-tetramethyl-1,3,2-dioxaborolane in 91%. 1H NMR (400 MHz, <cdcl3>) δ ppm 6.71 (2H), 2.48 (s, 3H), 1.37 (s, 12H).

### Synthesis of methyl 6-(2,6-difluoro-4-(methylthio)phenyl)-5-fluoropicolinate

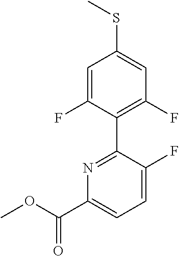 [SMILES: COC(=O)c1ccc(F)c(-c2c(F)cc(SC)cc2F)n1]

Method 1 was followed using 6-bromo-5-fluoropicolinate (1.0 equiv.) and 2-(2,6-difluoro-4-(methylthio)phenyl)-4,4,5,5-tetramethyl-1,3,2-dioxaborolane (1.75 equiv.) to give methyl 6-(2,6-difluoro-4-(methylthio)phenyl)-5-fluoropicolinate in 73% yield. LC/MS=313.9 (M+H), Rt=0.90 min.

### Synthesis of 6-(2,6-difluoro-4-(methylthio)phenyl)-5-fluoropicolinic acid

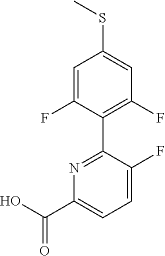 [SMILES: CSc1cc(F)c(-c2nc(C(=O)O)ccc2F)c(F)c1]

To a solution of 6-(2,6-difluoro-4-(methylthio)phenyl)-5-fluoropicolinate (1.0 eq) in THF (0.2 M) was added LiOH (5.5 eq, 2M) and allowed to stir at rt for 3 hrs. The volatiles were removed in vacuo, and the residual aqueous was acidified with 2M HCl to pH 4. The precipitate was filtered and dried to yield 6-(2,6-difluoro-4-(methylthio)phenyl)-5-fluoropicolinic acid as a solid in 92% yield. LCMS (m/z): 299.9 (MH⁺), Rₜ=0.78 min.

### Synthesis of methyl 6-(2,6-difluoro-4-(methylsulfonyl)phenyl)-5-fluoropicolinate

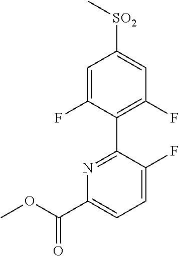 [SMILES: COC(=O)c1ccc(F)c(-c2c(F)cc(S(C)(=O)=O)cc2F)n1]

To a solution of methyl 6-(2,6-difluoro-4-(methylthio)phenyl)-5-fluoropicolinate (1.0 equiv) in CH₂Cl₂ (0.2 M) at 0° C. was added MCPBA (3.2 equiv.). After stirring for 40 minutes, the reaction was quenched with Na₂S₂O_{3(aq.)}, diluted with EtOAc, washed with NaHCO_{3(sat.)}, brine, dried over MgSO₄, filtered, concentrate, purified by SiO₂ chromatography to yield methyl 6-(2,6-difluoro-4-(methylsulfonyl)phenyl)-5-fluoropicolinate in 56% yield. LC/MS=345.9 (M+H), Rt=0.69 min.

### Synthesis of 6-(2,6-difluoro-4-(methylsulfonyl)phenyl)-5-fluoropicolinic acid

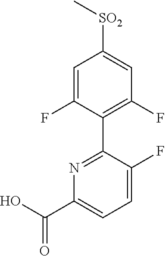 [SMILES: CS(=O)(=O)c1cc(F)c(-c2nc(C(=O)O)ccc2F)c(F)c1]

To a solution of 6-(2,6-difluoro-4-(methylsulfonyl)phenyl)-5-fluoropicolinate (1.0 eq) in THF (0.1M) was added LiOH (5.5 eq, 2M) and allowed to stir at 37° C. for 2 hrs. The volatiles were removed in vacuo, and the residual aqueous was acidified with 2M HCl to pH 4. The precipitate was filtered and dried to yield 6-(2,6-difluoro-4-(methylsulfonyl)phenyl)-5-fluoropicolinic acid as a solid in 91% yield. LCMS (m/z): 331.8 (MH⁺), Rₜ=0.59 min.

### Synthesis of tert-butyl(3,5-difluorophenoxy)dimethylsilane

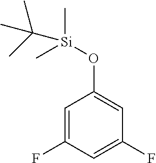 [SMILES: CC(C)(C)[Si](C)(C)Oc1cc(F)cc(F)c1]

To a solution of 3,5-difluorophenol (1.0 equiv.) and imidazole (2.2 equiv.) in DMF (0.8 M) at 0° C. was added TBDMSC1 (1.1 equiv.). The ice bath was removed and after stirring for 3 hours the solution was diluted with EtOAc, washed with water, brine, dried over MgSO₄, filtered, concentrated and purified by SiO₂ chromatography to yield tert-butyl(3,5-difluorophenoxy)dimethylsilane in 73%. ¹H NMR (400 MHz, <cdcl3>) δ ppm 0.23 (s, 6H) 0.99 (s, 9H) 6.33-6.40 (m, 2H) 6.44 (tt 1H).

### Synthesis of tert-butyl(3,5-difluoro-4-(4,4,5,5-tetramethyl-1,3,2-dioxaborolan-2-yl)phenoxy)dimethylsilane

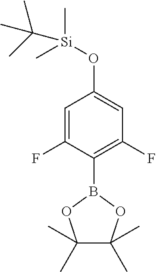 [SMILES: CC1(C)OB(c2c(F)cc(O[Si](C)(C)C(C)(C)C)cc2F)OC1(C)C]

To a solution of tert-butyl(3,5-difluorophenoxy)dimethylsilane (1.0 eq) in dry THF (0.2M) under an atmosphere of N₂ at −78° C. was added n-butyllithium (1 eq, 1.6M in hexanes) slowly keeping the internal temperature below −65° C. The reaction was stirred for 1 hr at −78° C., followed by the addition of 2-isopropoxy-4,4,5,5-tetramethyl-1,3,2-dioxaborolane (2.1 eq). The reaction was allowed to warm to room temperature. Upon completion, the reaction was quenched with NaHCO_{3 (sat.)} and extracted with EtOAc. The organics were washed with brine, dried over Na₂SO₄, filtered and concentrated to yield tert-butyl(3,5-difluoro-4-(4,4,5,5-tetramethyl-1,3,2-dioxaborolan-2-yl)phenoxy)dimethylsilane in 91%. ¹H NMR (400 MHz, <cdcl3>) δ ppm 0.21 (s, 6H) 0.97 (s, 9H) 1.37 (s, 12H) 6.33 (d, J=9.39 Hz, 2H).

### Synthesis of methyl 6-(2,6-difluoro-4-hydroxyphenyl)-5-fluoropicolinate

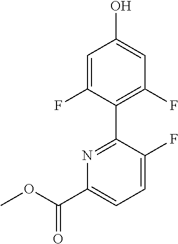 [SMILES: COC(=O)c1ccc(F)c(-c2c(F)cc(O)cc2F)n1]

Method 1 was followed using 6-bromo-5-fluoropicolinate (1.0 equiv.) and tert-butyl(3,5-difluoro-4-(4,4,5,5-tetramethyl-1,3,2-dioxaborolan-2-yl)phenoxy)dimethylsilane (1.75 equiv.) to give methyl 6-(2,6-difluoro-4-hydroxyphenyl)-5-fluoropicolinate in 65% yield. The reaction was heated for an additional 30 minutes at 100° C. in the microwave to drive to completion the deprotection of the TBDMS group. LC/MS=283.9 (M+H), Rt=0.69 min.

### Synthesis of methyl 6-(4-(2-(tert-butyldimethylsilyloxy)ethoxy)-2,6-difluorophenyl)-5-fluoropicolinate

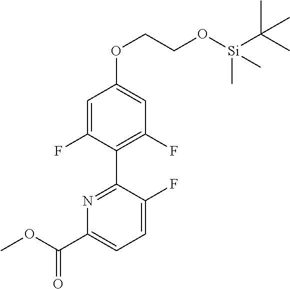 [SMILES: COC(=O)c1ccc(F)c(-c2c(F)cc(OCCO[Si](C)(C)C(C)(C)C)cc2F)n1]

To a solution of methyl 6-(2,6-difluoro-4-hydroxyphenyl)-5-fluoropicolinate (1.0 equiv.) and potassium carbonate (4.0 equiv.) in DMF (0.4 M) was (2-bromoethoxy)(tert-butyl)dimethylsilane (2 equiv.). After stirring for 72 hours at rt the heterogeneous solution was diluted with water, extracted with EtOAc, dried over MgSO₄, filtered, concentrated and purified by ISCO SiO₂ chromatography to yield methyl 6-(4-(2-(tert-butyldimethylsilyloxy)ethoxy)-2,6-difluorophenyl)-5-fluoropicolinate in 74%. LC/MS=442.1 (M+H), Rₜ=1.22 min.

### Synthesis of 6-(4-(2-(tert-butyldimethylsilyloxy)ethoxy)-2,6-difluorophenyl)-5-fluoropicolinic acid

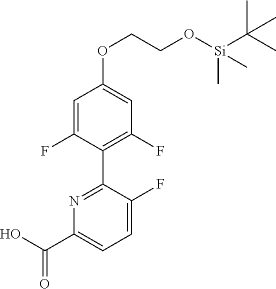 [SMILES: CC(C)(C)[Si](C)(C)OCCOc1cc(F)c(-c2nc(C(=O)O)ccc2F)c(F)c1]

Method 2 was followed using methyl 6-(4-(2-(tert-butyldimethylsilyloxy)ethoxy)-2,6-difluorophenyl)-5-fluoropicolinate to give 6-(4-(2-(tert-butyldimethylsilyloxy)ethoxy)-2,6-difluorophenyl)-5-fluoropicolinic acid in 94% yield. LC/MS=428.1 (M+H), Rₜ=1.13 min.

### Synthesis of methyl 6-(4-ethoxy-2,6-difluorophenyl)-5-fluoropicolinate

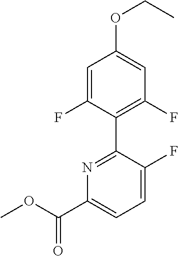 [SMILES: CCOc1cc(F)c(-c2nc(C(=O)OC)ccc2F)c(F)c1]

To a solution of methyl 6-(2,6-difluoro-4-hydroxyphenyl)-5-fluoropicolinate (1.0 equiv.), ethanol (3.0 eq.) and triphenylphosphine (3.0 eq.) in THF (0.18 M) at 0° C. was added diisopropyl azaodicarboxylate (3.0 eq.) After stirring for 16 hours at rt as the solution slowly warmed to rt, the volatiles were removed in vacuo and the residue was purified by ISCO SiO₂ chromatography to yield methyl 6-(4-ethoxy-2,6-difluorophenyl)-5-fluoropicolinate in 99% yield. LC/MS=311.9 (M+H), Rₜ=0.91 min.

### Synthesis of 6-(4-ethoxy-2,6-difluorophenyl)-5-fluoropicolinic acid

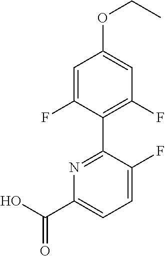 [SMILES: CCOc1cc(F)c(-c2nc(C(=O)O)ccc2F)c(F)c1]

Method 2 was followed using methyl 6-(4-ethoxy-2,6-difluorophenyl)-5-fluoropicolinate to give 6-(4-ethoxy-2,6-difluorophenyl)-5-fluoropicolinic acid in 38% yield. LC/MS=297.9 (M+H), Rₜ=0.80 min.

### Synthesis of 1,3-difluoro-5-(2-methoxyethoxy)benzene

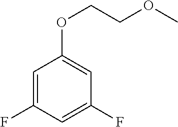

To a solution of 3,5-difluorophenol (1.0 equiv.), 2-methoxyethanol (3.0 equiv.) and triphenylphosphine (3.0 equiv) in THF (0.1 M) was added DIAD (3.0 equiv.). After stirring at rt for 18 hours, the volatiles were removed in vacuo and the residue was purified by SiO_{2 chromatography to yield} 1,3-difluoro-5-(2-methoxyethoxy)benzene in 95%. ¹H NMR (400 MHz, <cdcl3>) δ ppm 6.41-6.47 m (3H), 4.08 (t, 2H), 3.74 (t, 2H), 3.45 (s, 3H).

### Method 3

### Synthesis of 2-(2,6-difluoro-4-(2-methoxyethoxy)phenyl)-4,4,5,5-tetramethyl-1,3,2-dioxaborolane

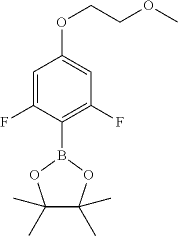 [SMILES: COCCOc1cc(F)c(B2OC(C)(C)C(C)(C)O2)c(F)c1]

To a solution of 1,3-difluoro-5-(2-methoxyethoxy)benzene (1.0 eq) in dry THF (0.2M) under an atmosphere of N₂ at −78° C. was added n-butyllithium (1 eq, 1.6M in hexanes) slowly keeping the internal temperature below −65° C. The reaction was stirred for 1 hr at −78° C., followed by the addition of 2-isopropoxy-4,4,5,5-tetramethyl-1,3,2-dioxaborolane (2.1 eq). The reaction was allowed to warm to room temperature. Upon completion, the reaction was quenched with NaHCO_{3 (sat.)} and extracted with EtOAc. The organics were washed with brine, dried over Na₂SO₄, filtered and concentrated to yield 2-(2,6-difluoro-4-(2-methoxyethoxy)phenyl)-4,4,5,5-tetramethyl-1,3,2-dioxaborolane. ¹H NMR (400 MHz, <cdcl3>) δ ppm 6.42 (d, 2H), 4.10 (m, 2H), 3.74 (m, 2H), 3.44 (s, 3H), 1.37 (s, 12H).

### Synthesis of methyl 6-(2,6-difluoro-4-(2-methoxyethoxy)phenyl)-5-fluoropicolinate

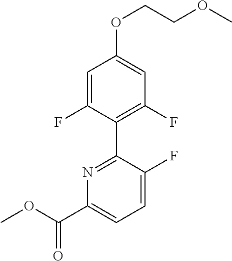

Method 1 was followed using methyl 6-bromo-5-fluoropicolinate (1.0 equiv.) and 2-(2,6-difluoro-4-(2-methoxyethoxy)phenyl)-4,4,5,5-tetramethyl-1,3,2-dioxaborolane (1.75 equiv.) at 80° C. for 1 hour to give methyl 6-(2,6-difluoro-4-(2-methoxyethoxy)phenyl)-5-fluoropicolinate in 95% yield. LC/MS=341.9 (M+H), Rₜ=0.89 min.

### Synthesis of 6-(2,6-difluoro-4-(2-methoxyethoxy)phenyl)-5-fluoropicolinic acid

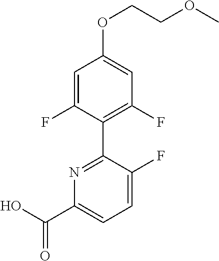 [SMILES: COCCOc1cc(F)c(-c2nc(C(=O)O)ccc2F)c(F)c1]

Method 2 was followed using methyl 6-(2,6-difluoro-4-(2-methoxyethoxy)phenyl)-5-fluoropicolinate to give 6-(2,6-difluoro-4-(2-methoxyethoxy)phenyl)-5-fluoropicolinic acid in 98% yield. LC/MS=327.9 (M+H), Rₜ=0.71 min.

### Synthesis of 3-amino-6-phenylpyrazine-2-carboxylic acid

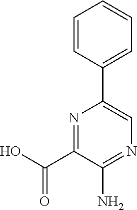 [SMILES: Nc1ncc(-c2ccccc2)nc1C(=O)O]

Method 1 and 2 were followed using methyl 3-amino-6-bromopyrazine-2-carboxylate (1.0 equiv.) and phenylboronic acid (2.0 equiv.) and Pd(dppf)Cl₂-DCM (0.05 equiv.) to give 3-amino-6-phenylpyrazine-2-carboxylic acid in 70% yield over the two steps. LCMS (m/z): 216.0 (MH⁺), Rₜ=0.67 min.

### Synthesis of methyl 3-amino-6-(2,6-difluorophenyl)-5-fluoropicolinate

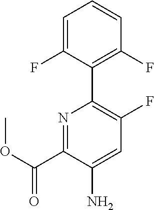 [SMILES: COC(=O)c1nc(-c2c(F)cccc2F)c(F)cc1N]

Method 1 was followed using methyl 3-amino-6-bromo-5-fluoropicolinate (1.0 equiv.) and 2,6-difluorophenylboronic acid (1.3 equiv.) and Pd(dppf)Cl₂-DCM (0.05 equiv.) to give 3-amino-6-(2,6-difluorophenyl)-5-fluoropicolinate in 94% yield. LCMS (m/z): 283.0 (MH⁺), Rₜ=0.76 min.

### Synthesis of 3-amino-6-(2,6-difluorophenyl)-5-fluoropicolinic acid

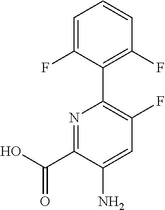 [SMILES: Nc1cc(F)c(-c2c(F)cccc2F)nc1C(=O)O]

Method 2 was followed using 3-amino-6-(2,6-difluorophenyl)-5-fluoropicolinate (1.0 equiv.) and LiOH (1.0 equiv.) to give 3-amino-6-(2,6-difluorophenyl)-5-fluoropicolinic acid in 79% yield. LCMS (m/z): 269.0 (MH⁺), Rₜ=0.79 min.

### Synthesis of 2-(2,6-difluorophenyl)pyrimidine-4-carboxylic acid

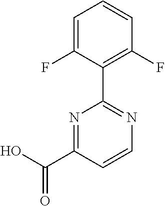 [SMILES: O=C(O)c1ccnc(-c2c(F)cccc2F)n1]

To a solution of 2-chloropyrimidine-4-carboxylic acid (1.0 equiv.) in DME and 2M Na₂CO₃ (3:1, 0.25 M) was added 2,6-difluorophenylboronic acid (1.3 equiv.) and Pd(dppf)Cl₂-DCM (0.05 equiv.) in a microwave vial. The vial was heated in the microwave at 120° C. for 30 minutes. The mixture was diluted with ethyl acetate and 1N NaOH was added. The organic phase was separated and extracted three more times with 1N NaOH and once with 6N NaOH. The combined aqueous phases were filtered and acidified to pH 1 by the addition of concentrated HCl and extracted with ethyl acetate. The organic layer was dried over magnesium sulfate, filtered, and concentrated to give 2-(2,6-difluorophenyl)pyrimidine-4-carboxylic acid in 81%. LCMS (m/z): 237.0 (MH⁺), Rₜ=0.54 min.

### Synthesis of 6-(2,6-difluorophenyl)picolinic acid

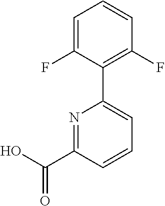 [SMILES: O=C(O)c1cccc(-c2c(F)cccc2F)n1]

Method 3 was followed using 6-bromopicolinic acid (1.0 equiv.) and 2,6-difluorophenylboronic acid (1.5 equiv.) and Pd(dppf)Cl₂-DCM (0.05 equiv.) to give 6-(2,6-difluorophenyl)picolinic acid in 38% yield. LCMS (m/z): 236.0 (MH⁺), Rₜ=0.87 min.

### Synthesis of methyl 6-(2,6-difluoro-3-hydroxyphenyl)-5-fluoropicolinate

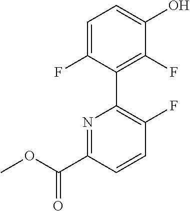 [SMILES: COC(=O)c1ccc(F)c(-c2c(F)ccc(O)c2F)n1]

To a solution of methyl 6-(3-(benzyloxy)-2,6-difluorophenyl)-5-fluoropicolinate (1.0 equiv.) in methanol (0.1 M) was added 10% Pd/C (0.1 equiv.) in ethyl acetate. The reaction was placed under an atmosphere of hydrogen and stirred for 2 hours. Upon completion, the solution was filtered over a pad of Celite, the pad was washed with methanol, the filtrate was concentrated in vacuo to give methyl 6-(2,6-difluoro-3-hydroxyphenyl)-5-fluoropicolinate as a grey oil in 86% yield. LC/MS=284.0 (M+H), Rt=0.90 min.

### Synthesis of methyl 6-(2,6-difluoro-3-(2-methoxyethoxy)phenyl)-5-fluoropicolinate

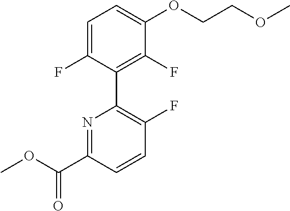

To a solution of methyl 6-(2,6-difluoro-3-hydroxyphenyl)-5-fluoropicolinate (1.0 equiv.) and cesium carbonate (2.0 equiv.) in DMF (0.4 M) was 2-methoxy-1-bromoethane (2 equiv.). After stirring for 16 hours the heterogeneous solution was diluted with water, extracted with EtOAc, dried over MgSO₄, filtered and concentrated to yield methyl 6-(2,6-difluoro-3-(2-methoxyethoxy)phenyl)-5-fluoropicolinate in 99%. LC/MS=342.0 (M+H), Rₜ=0.79 min.

### Synthesis of 6-(2,6-difluoro-3-(2-methoxyethoxy)phenyl)-5-fluoropicolinic acid

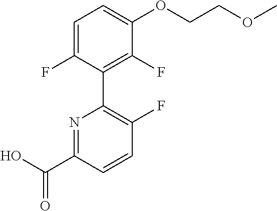 [SMILES: COCCOc1ccc(F)c(-c2nc(C(=O)O)ccc2F)c1F]

Method 2 was followed using methyl 6-(2,6-difluoro-3-(2-methoxyethoxy)phenyl)-5-fluoropicolinate to give 6-(2,6-difluoro-3-(2-methoxyethoxy)phenyl)-5-fluoropicolinic acid in 95% yield. LC/MS=328.1 (M+H), Rₜ=0.68 min.

### Synthesis of methyl 6-(3-(2-(tert-butyldimethylsilyloxy)ethoxy)-2,6-difluorophenyl)-5-fluoropicolinate

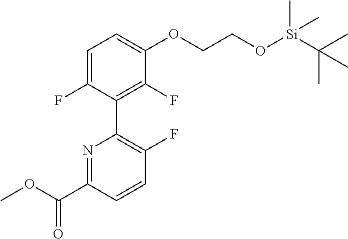 [SMILES: COC(=O)c1ccc(F)c(-c2c(F)ccc(OCCO[Si](C)(C)C(C)(C)C)c2F)n1]

To a solution of methyl 6-(2,6-difluoro-3-hydroxyphenyl)-5-fluoropicolinate (1.0 equiv.) and cesium carbonate (4.0 equiv.) in DMF (0.4 M) was (2-bromoethoxy)(tert-butyl)dimethylsilane (2 equiv.). After stirring for 16 hours at rt and 2 hours at 60° C. the heterogeneous solution was diluted with water, extracted with EtOAc, dried over MgSO₄, filtered, concentrated and purified by ISCO SiO₂ chromatography to yield methyl 6-(3-(2-(tert-butyldimethylsilyloxy)ethoxy)-2,6-difluorophenyl)-5-fluoropicolinate in 90%. LC/MS=442.1 (M+H), Rₜ=1.18 min.

### Synthesis of 6-(3-(2-(tert-butyldimethylsilyloxy)ethoxy)-2,6-difluorophenyl)-5-fluoropicolinic acid

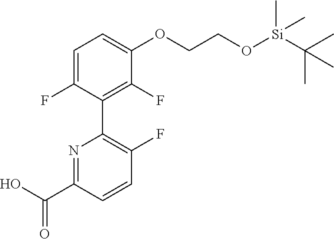 [SMILES: CC(C)(C)[Si](C)(C)OCCOc1ccc(F)c(-c2nc(C(=O)O)ccc2F)c1F]

Method 2 was followed using methyl 6-(3-(2-(tert-butyldimethylsilyloxy)ethoxy)-2,6-difluorophenyl)-5-fluoropicolinate to give 6-(3-(2-(tert-butyldimethylsilyloxy)ethoxy)-2,6-difluorophenyl)-5-fluoropicolinic acid in 87% yield. LC/MS=428.1 (M+H), Rₜ=1.08 min.

### Method 4

### Synthesis of 2-(2,6-difluorophenyl)pyrimidine-4-carboxylic acid

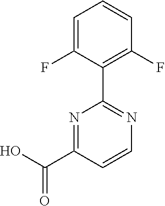 [SMILES: O=C(O)c1ccnc(-c2c(F)cccc2F)n1]

To a solution of 2-chloropyrimidine-4-carboxylic acid (1.0 equiv.) in DME and 2M Na₂CO₃ (3:1, 0.25 M) was added 2,6-difluorophenylboronic acid (1.3 equiv.) and Pd(dppf)Cl₂-DCM (0.05 equiv.) in a microwave vial. The vial was heated in the microwave at 120° C. for 30 minutes. The mixture was diluted with ethyl acetate and 1N NaOH was added. The organic phase was separated and extracted three more times with 1N NaOH and once with 6N NaOH. The combined aqueous phases were filtered and acidified to pH 1 by the addition of concentrated HCl and extracted with ethyl acetate. The organic layer was dried over magnesium sulfate, filtered, and concentrated to give 2-(2,6-difluorophenyl)pyrimidine-4-carboxylic acid in 81%. LCMS (m/z): 237.0 (MH⁺), Rₜ=0.54 min.

### Synthesis of 6-(2,6-difluorophenyl)picolinic acid

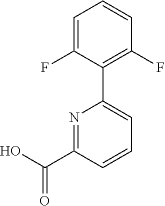 [SMILES: O=C(O)c1cccc(-c2c(F)cccc2F)n1]

Method 4 was followed using 6-bromopicolinic acid (1.0 equiv.) and 2,6-difluorophenylboronic acid (1.5 equiv.) and Pd(dppf)Cl₂-DCM (0.05 equiv.) to give 6-(2,6-difluorophenyl)picolinic acid in 38% yield. LCMS (m/z): 236.0 (MH⁺), Rₜ=0.87 min.

### Synthesis of ethyl 2-amino-2-cyanoacetate

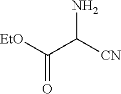

To a solution of ethyl 2-cyano-2-(hydroxyimino)acetate (1 eq) in 70 mL of water and 56 mL of aq. sat. sodium bicarbonate was added portionwise throughout 10 minutes Na₂S₂O₄ (2.8 eq) The reaction mixture was stirred at room temperature for 1 hour. The solution was saturated with sodium chloride, extracted with methylene chloride (300 mL×3) and then the combined organic layers were dried over anhydrous Na₂SO₄, filtered, and concentrated in vacuo to give ethyl 2-amino-2-cyanoacetate, which was used to next step without further (55%). LC/MS (m/z): 129.0 (MH⁺), Rₜ: 0.25 min.

### Synthesis of ethyl 2-cyano-2-(2,6-difluorobenzamido)acetate

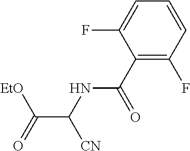 [SMILES: CCOC(=O)C(C#N)NC(=O)c1c(F)cccc1F]

To a solution of ethyl 2-amino-2-cyanoacetate (1 eq) in 6 mL of dichloromethane was added pyridine (1.5 eq) and 2,6-difluorobenzoyl chloride (1 eq) at 0° C. The reaction mixture was stirred at room temperature for 3 hours. The mixture was diluted with ethyl acetate, washed with brine, then dried over anhydrous MgSO₄, filtered, and concentrated in vacuo. The crude residue was purified by flash chromatography (EtOAc:hexanes=1:1) to give ethyl 2-cyano-2-(2,6-difluorobenzamido)acetate (84%). LC/MS (m/z): 269.1 (MH⁺), Rₜ: 0.69 min.

### Synthesis of 5-amino-2-(2,6-difluorophenyl)thiazole-4-carboxylic acid

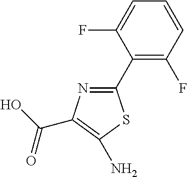 [SMILES: Nc1sc(-c2c(F)cccc2F)nc1C(=O)O]

To a solution of the ethyl 2-cyano-2-(2,6-difluorobenzamido)acetate (1 eq) in 10 mL of toluene was added Lawesson reagent. The mixture was stirred at 95° C. for 2 days. Solvents were removed under reduced pressure. The crude residue was purified by flash chromatography (EtOAc:hexanes=1:1) to give the ethyl 5-amino-2-(2,6-difluorophenyl)thiazole-4-carboxylate, which was dissolved in 5 mL of methanol and 5 mL of THF. Then the mixture was added 1M sodium hydroxide (2 eq). The reaction mixture was stirred at room temperature overnight. The reaction was concentrated to remove most of solvents. The residue was extracted with ethyl acetate. The aqueous layer was acidified to pH=4-5 by 1N HCl. The resulting mixture was extracted by ethyl acetate. The organic layer was separated, washed with brine, then dried over anhydrous MgSO₄, filtered, and concentrated in vacuo to give 5-amino-2-(2,6-difluorophenyl)thiazole-4-carboxylic acid (34%). LC/MS (m/z): 257.1 (MH⁺), Rₜ: 0.61 min.

### Method 5

### Synthesis of 5-amino-2-(2,6-difluorophenyl)pyrimidine-4-carboxylic acid

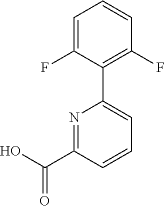 [SMILES: O=C(O)c1cccc(-c2c(F)cccc2F)n1]

A 2.68 M NaOEt in EtOH solution (3 eq) was added to an ice-bath cooled mixture of 2,6-difluorobenzimidamide hydrochloride (2 eq) in EtOH (0.1 M). The resulting mixture was allowed to warm to rt and stirred under N₂ for 30 min. To the reaction mixture was added drop wise a solution of mucobromic acid (1 eq) in EtOH and the reaction was heated in a 50° C. oil bath for 2.5 hr. After cooling to rt the reaction mixture was concentrated in vacuo. H₂O and 1.0 N NaOH were added and the aqueous mixture was washed with EtOAc. The aqueous phase was acidified to pH=4 with 1.0 N HCl then extracted with EtOAc. Combined organic extracts were washed once with brine, then dried over anhydrous Na₂SO₄, filtered, and concentrated in vacuo to give 5-bromo-2-(2,6-difluorophenyl)pyrimidine-4-carboxylic acid. The crude product was used for the next step without further purification. LC/MS (m/z): 316.9 (MH⁺). LC: Rₜ: 2.426 min.

CuSO₄ (0.1 eq) was added to a mixture of 5-bromo-2-(2,6-difluorophenyl)pyrimidine-4-carboxylic acid (1 eq) and 28% aqueous ammonium hydroxide solution in a microwave reaction vessel. The reaction mixture was heated in a microwave reactor at 110° C. for 25 min. The reaction vessel was cooled in dry ice for 30 min then unsealed and concentrated in vacuo. To the resulting solids was added 1.0 N HCl and the mixture was extracted with EtOAc. Combined organic extracts were washed once with brine, then dried over anhydrous Na₂SO₄, filtered, and concentrated in vacuo to give 5-amino-2-(2,6-difluorophenyl)pyrimidine-4-carboxylic acid. The crude product was used for the next step without further purification. LCMS (m/z): 252.0 (MH⁺), Rₜ=2.0 min.

### Synthesis of 5-amino-2-(2-fluorophenyl)pyrimidine-4-carboxylic acid

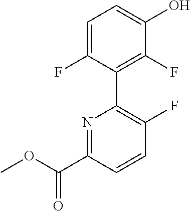 [SMILES: COC(=O)c1ccc(F)c(-c2c(F)ccc(O)c2F)n1]

Following METHOD 5, 5-amino-2-(2-fluorophenyl)pyrimidine-4-carboxylic acid was prepared starting from 2-fluorobenzimidamide hydrochloride. LC/MS (m/z): 234.0 (MH⁺), Rₜ: 0.70 min.

### Synthesis of 5-amino-2-phenylpyrimidine-4-carboxylic acid

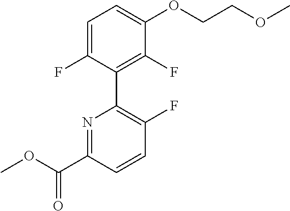

Following METHOD 5, 5-amino-2-phenylpyrimidine-4-carboxylic acid was prepared starting from benzimidamide hydrochloride. LC/MS (m/z): 216.1 (MH⁺).

### Synthesis of methyl 6-(2,6-difluoro-4-(2-hydroxypropan-2-yl)phenyl)-5-fluoropicolinate

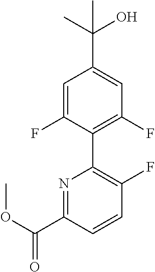 [SMILES: COC(=O)c1ccc(F)c(-c2c(F)cc(C(C)(C)O)cc2F)n1]

Method 1 was followed using 6-bromo-5-fluoropicolinate (1.0 equiv.) and (2-(3,5-difluoro-4-(4,4,5,5-tetramethyl-1,3,2-dioxaborolan-2-yl)phenyl)propan-2-yloxy)triisopropylsilane (1.6 equiv.) at 100° C. for 30 min in the microwave to give methyl 6-(2,6-difluoro-4-(2-hydroxypropan-2-yl)phenyl)-5-fluoropicolinate in 90% yield. LC/MS=325.9 (MH⁺), Rₜ=0.81 min. ¹H NMR (400 MHz, <cdcl3>) δ ppm 1.59 (s, 6H), 4.00 (s, 3H), 7.15 (d, J=9.00 Hz, 2H), 7.62-7.68 (m, 1H), 8.23-8.29 (m, 1H).

### Synthesis of 6-(2,6-difluoro-4-(2-hydroxypropan-2-yl)phenyl)-5-fluoropicolinic acid

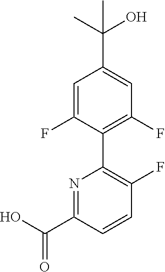 [SMILES: CC(C)(O)c1cc(F)c(-c2nc(C(=O)O)ccc2F)c(F)c1]

Method 2 was followed using methyl 6-(2,6-difluoro-4-(2-hydroxypropan-2-yl)phenyl)-5-fluoropicolinate to give 6-(2,6-difluoro-4-(2-hydroxypropan-2-yl)phenyl)-5-fluoropicolinic acid in 94% yield. LC/MS=312.0 (MH⁺), Rₜ=0.69 min.

### Synthesis of 4-(3,5-difluorophenyl)tetrahydro-2H-pyran-4-ol

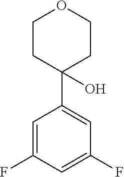 [SMILES: OC1(c2cc(F)cc(F)c2)CCOCC1]

To a solution of 1-bromo-3,5-difluorobenzene in THF (0.16 M) under N₂ was added Mg turnings (1.6 equiv.). A reflux condenser was attached and the solution was submerged in a 90° C. oil bath and refluxed for 2 hours at which time the heat was removed and the solution cooled to 0° C. Dihydro-2H-pyran-4(3H)-one (1.0 equiv.) in THF was added and the solution was stirred under N₂ allowing to warm to rt for 16 hrs. The reaction was quenched by addition of sat. NH₄Cl and the solution was extracted with EtOAc, washed with brine, dried over sodium sulfate, filtered, concentrated. The crude material was purified by ISCO SiO₂ chromatography eluting with 0-100% EtOAc/n-heptanes to yield 4-(3,5-difluorophenyl)tetrahydro-2H-pyran-4-ol in 37% yield. ¹H NMR (400 MHz, <cdcl3>) δ ppm 1.63 (d, J=12.13 Hz, 2H), 2.11 (ddd, J=13.50, 11.15, 6.65 Hz, 2H), 3.84-3.90 (m, 4H), 6.72 (tt, J=8.75, 2.20 Hz, 1H), 6.97-7.05 (m, 2H).

### Synthesis of 4-(3,5-difluorophenyl)-3,6-dihydro-2H-pyran

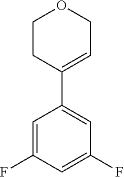 [SMILES: Fc1cc(F)cc(C2=CCOCC2)c1]

4-(3,5-difluorophenyl)tetrahydro-2H-pyran-4-ol (1.0 equiv.) was dissolved in DCM (0.2 M) and cooled to 0° C. TEA (2.8 equiv.) was added to the solution, followed by MsCl (1.3 equiv.). The reaction was stirred at rt for 2 hrs. The solution was cooled to 0° C. and DBU (3.0 equiv.) was added. The reaction was stirred at rt for 18 hrs. The solution was concentrated and the residue was purified by SiO₂ chromatography (0-100% EtOAc in Heptanes) to afford 4-(3,5-difluorophenyl)-3,6-dihydro-2H-pyran in 38% yield. ¹H NMR (400 MHz, <cdcl3>) δ ppm 2.42-2.49 (m, 2H), 3.93 (t, J=5.48 Hz, 2H), 4.32 (q, J=2.74 Hz, 2H), 6.16-6.22 (m, 1H), 6.70 (tt, J=8.80, 2.35 Hz, 1H), 6.85-6.94 (m, 2H).

### Synthesis of 4-(3,5-difluorophenyl)tetrahydro-2H-pyran

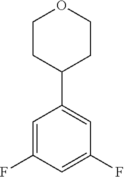 [SMILES: Fc1cc(F)cc(C2CCOCC2)c1]

To a solution of 4-(3,5-difluorophenyl)-3,6-dihydro-2H-pyran (1.0 equiv.) in methanol (0.2 M) was added 10% Pd/C (0.05 equiv.). The reaction was placed under an atmosphere of hydrogen and stirred for 18 hours. Upon completion, the solution was filtered over a pad of Celite, the pad was washed with DCM, the filtrate was concentrated in vacuo to give 4-(3,5-difluorophenyl)tetrahydro-2H-pyran in 71% yield. ¹H NMR (400 MHz, <cdcl3>) δ ppm 1.76 (br. s., 4H), 2.75 (br. s., 1H), 3.50 (br. s., 2H), 4.08 (d, J=9.78 Hz, 2H), 6.56-6.94 (m, 3H).

### Synthesis of 2-(2,6-difluoro-4-(tetrahydro-2H-pyran-4-yl)phenyl)-4,4,5,5-tetramethyl-1,3,2-dioxaborolane

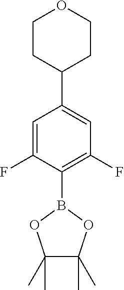 [SMILES: CC1(C)OB(c2c(F)cc(C3CCOCC3)cc2F)OC1(C)C]

Method 3 was followed using 2-isopropoxy-4,4,5,5-tetramethyl-1,3,2-dioxaborolane (2.2 equiv.), butyllithium (1.1 equiv.) and 4-(3,5-difluorophenyl)tetrahydro-2H-pyran (1.0 equiv.) to give 2-(2,6-difluoro-4-(tetrahydro-2H-pyran-4-yl)phenyl)-4,4,5,5-tetramethyl-1,3,2-dioxaborolane in 100% yield. ¹H NMR (400 MHz, <cdcl3>) δ ppm 1.16-1.19 (m, 12H), 1.65-1.74 (m, 4H), 2.60-2.75 (m, 1H), 3.37-3.51 (m, 2H), 4.01 (dt, J=11.54, 3.42 Hz, 2H), 6.67 (d, J=8.22 Hz, 2H).

### Synthesis of methyl 6-(2,6-difluoro-4-(tetrahydro-2H-pyran-4-yl)phenyl)-5-fluoropicolinate

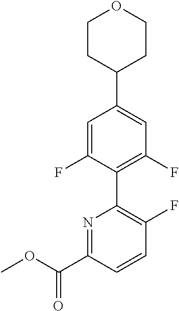 [SMILES: COC(=O)c1ccc(F)c(-c2c(F)cc(C3CCOCC3)cc2F)n1]

Method 1 was followed using methyl 6-bromo-5-fluoropicolinate (1.0 equiv.) and 2-(2,6-difluoro-4-(tetrahydro-2H-pyran-4-yl)phenyl)-4,4,5,5-tetramethyl-1,3,2-dioxaborolane (3.0 equiv.) at 100° C. for 20 min in microwave to give methyl 6-(2,6-difluoro-4-(tetrahydro-2H-pyran-4-yl)phenyl)-5-fluoropicolinate in 59% yield. LC/MS=352.2 (MH⁺), Rₜ=0.92 min.

### Synthesis of 6-(2,6-difluoro-4-(tetrahydro-2H-pyran-4-yl)phenyl)-5-fluoropicolinic acid

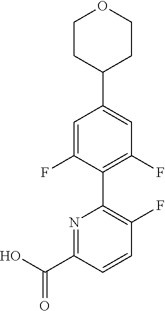 [SMILES: O=C(O)c1ccc(F)c(-c2c(F)cc(C3CCOCC3)cc2F)n1]

Method 2 was followed using methyl 6-(2,6-difluoro-4-(pyridazin-4-yl)phenyl)-5-fluoropicolinate to give 6-(2,6-difluoro-4-(tetrahydro-2H-pyran-4-yl)phenyl)-5-fluoropicolinic acid in 71% yield. LC/MS=338.1 (MH⁺), Rₜ=0.80 min.

### Synthesis of 3-(3,5-difluorophenyl)oxetan-3-ol

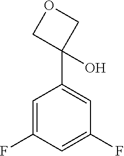 [SMILES: OC1(c2cc(F)cc(F)c2)COC1]

To a solution of 1-bromo-3,5-difluorobenzene in THF (0.27 M) under Ar was added Mg turnings (1.6 M). A reflux condenser was attached and the solution was submerged in a 90° C. oil bath and refluxed for two hours. The oxetan-3-one (1.0 equiv.) was added in THF via syringe. The solution was left stirring at rt under Ar overnight. The reaction solution was quenched by addition of NH₄Cl₍ₛₐₜ₎ and the solution was extracted with EtOAc, washed with NaCl_{(sat.)}, dried over MgSO₄, filtered, concentrated and purified by ISCO SiO₂ chromatography (0-100% EtOAc/n-heptanes gradient) to yield 3-(3,5-difluorophenyl)oxetan-3-ol in 56% yield. ¹H NMR (400 MHz, CHLOROFORM-d) δ ppm 4.82 (d, J=7.63 Hz, 2H), 4.91 (d, J=7.63 Hz, 2H), 7.16-7.23 (m, 2H).

### Synthesis of 3-(3,5-difluoro-4-(4,4,5,5-tetramethyl-1,3,2-dioxaborolan-2-yl)phenyl)oxetan-3-ol

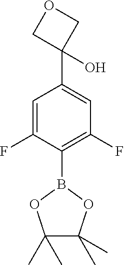 [SMILES: CC1(C)OB(c2c(F)cc(C3(O)COC3)cc2F)OC1(C)C]

Method 3 was followed using 2-isopropoxy-4,4,5,5-tetramethyl-1,3,2-dioxaborolane (2.5 equiv.), butyllithium (2.4 equiv.) and 3-(3,5-difluorophenyl)oxetan-3-ol (1.0 equiv.) to give 3-(3,5-difluoro-4-(4,4,5,5-tetramethyl-1,3,2-dioxaborolan-2-yl)phenyl)oxetan-3-ol in 79% yield. ¹H NMR (400 MHz, <cdcl3>) δ ppm 1.34-1.42 (m, 12H), 4.79 (d, J=7.63 Hz, 2H), 4.90 (d, J=7.34 Hz, 2H), 7.17 (d, J=8.22 Hz, 2H).

### Synthesis of methyl 6-(2,6-difluoro-4-(3-hydroxyoxetan-3-yl)phenyl)-5-fluoropicolinate

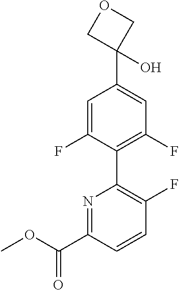 [SMILES: COC(=O)c1ccc(F)c(-c2c(F)cc(C3(O)COC3)cc2F)n1]

Method 1 was followed using methyl 6-bromo-5-fluoropicolinate (1.0 equiv.) and 3-(3,5-difluoro-4-(4,4,5,5-tetramethyl-1,3,2-dioxaborolan-2-yl)phenyl)oxetan-3-ol (1.4 equiv.) at 100° C. for 20 min in microwave to give methyl 6-(2,6-difluoro-4-(3-hydroxyoxetan-3-yl)phenyl)-5-fluoropicolinate in 43% yield. LC/MS=340.1 (MH⁺), Rₜ=0.69 min.

### Synthesis of 6-(2,6-difluoro-4-(3-hydroxyoxetan-3-yl)phenyl)-5-fluoropicolinic acid

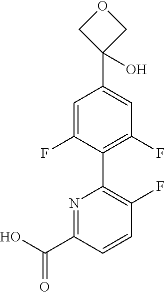 [SMILES: O=C(O)c1ccc(F)c(-c2c(F)cc(C3(O)COC3)cc2F)n1]

Method 2 was followed using methyl 6-(2,6-difluoro-4-(3-hydroxyoxetan-3-yl)phenyl)-5-fluoropicolinate to give 6-(2,6-difluoro-4-(3-hydroxyoxetan-3-yl)phenyl)-5-fluoropicolinic acid in 99% yield. LC/MS=325.9 (MH⁺) Rₜ=0.60 min.

### Synthesis of methyl 6-(2,6-difluoro-4-(3-methoxyoxetan-3-yl)phenyl)-5-fluoropicolinate

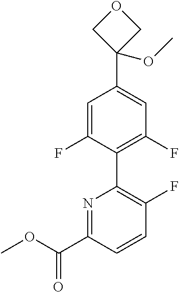 [SMILES: COC(=O)c1ccc(F)c(-c2c(F)cc(C3(OC)COC3)cc2F)n1]

To a solution of methyl 6-(2,6-difluoro-4-(3-hydroxyoxetan-3-yl)phenyl)-5-fluoropicolinate (1.0 equiv.) in DMF (0.34 M) at 0° C. was added NaH dispersion (1.4 equiv.). The solution was stirred in the ice bath for 1 hour, at which time MeI (1.5 equiv) was added. The solution was left stirring under Ar as the bath was allowed to warm up to rt and stirred at rt overnight. The solution was diluted with H₂O, and extracted with EtOAc. The organic was washed with H₂O, NaCl_{(sat.)}, dried over MgSO₄, filtered, concentrated and purified by ISCO SiO₂ chromatography (0-100% EtOAc/n-heptanes) to yield methyl 6-(2,6-difluoro-4-(3-methoxyoxetan-3-yl)phenyl)-5-fluoropicolinate in 46% yield. LC/MS=354.0 (MH⁺) Rt=0.82 min.

### Synthesis of 6-(2,6-difluoro-4-(3-methoxyoxetan-3-yl)phenyl)-5-fluoropicolinic acid

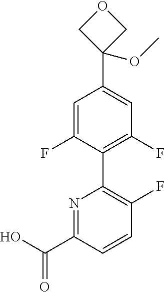 [SMILES: COC1(c2cc(F)c(-c3nc(C(=O)O)ccc3F)c(F)c2)COC1]

Method 2 was followed using methyl 6-(2,6-difluoro-4-(3-methoxyoxetan-3-yl)phenyl)-5-fluoropicolinate to give 6-(2,6-difluoro-4-(3-methoxyoxetan-3-yl)phenyl)-5-fluoropicolinic acid in 86% yield. LC/MS=339.9 (MH⁺), Rt=0.71 min.

### Synthesis of methyl 6-(2,6-difluoro-4-(3-fluorooxetan-3-yl)phenyl)-5-fluoropicolinate

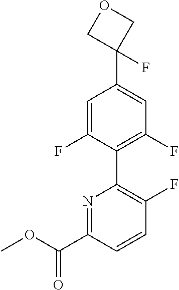 [SMILES: COC(=O)c1ccc(F)c(-c2c(F)cc(C3(F)COC3)cc2F)n1]

To a solution of methyl 6-(2,6-difluoro-4-(3-hydroxyoxetan-3-yl)phenyl)-5-fluoropicolinate (1.0 equiv.) in CH₂Cl₂ (0.04 M) at −78° C. under Ar was added methylDAST (1.7 equiv.). After addition, the solution was stirred under Ar at −78° C. for 10 minutes and then the bath was removed. The reaction was allowed to warm up to rt and quenched by addition of NaHCO_{3(sat.)}. The solution was diluted with EtOAc, washed with NaHCO3(sat.), NaCl(sat.), dried over MgSO4, filtered, concentrated, purified by ISCO SiO2 chromatography (24 gram column, 0-100 EtOAc/n-heptanes) to yield methyl 6-(2,6-difluoro-4-(3-fluorooxetan-3-yl)phenyl)-5-fluoropicolinate in 56% yield. LC/MS=342.0 (MH+), Rₜ=0.85 min.

### Synthesis of 6-(2,6-difluoro-4-(3-fluorooxetan-3-yl)phenyl)-5-fluoropicolinic acid

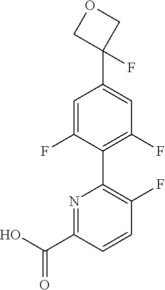 [SMILES: O=C(O)c1ccc(F)c(-c2c(F)cc(C3(F)COC3)cc2F)n1]

Method 2 was followed using methyl 6-(2,6-difluoro-4-(3-fluorooxetan-3-yl)phenyl)-5-fluoropicolinate to give 6-(2,6-difluoro-4-(3-fluorooxetan-3-yl)phenyl)-5-fluoropicolinic acid in 99% yield. LC/MS=327.9 (MH⁺), Rₜ=0.74 min.

### Synthesis of 4-(3,5-difluorophenyl)tetrahydro-2H-pyran-4-ol

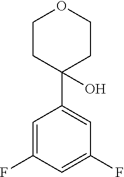 [SMILES: OC1(c2cc(F)cc(F)c2)CCOCC1]

To a solution of 1-bromo-3,5-difluorobenzene (1.6 equiv.) in THF (0.26 M) under Ar was added Mg turnings (1.6 equiv.). A reflux condenser was attached and the solution was submerged in a 90° C. oil bath and refluxed for two hours. The oxetan-3-one (1.0 equiv.) was added in THF via syringe. The solution was left stirring at rt under Ar for 5 hrs. The reaction solution was quenched by addition of NH₄Cl₍ₛₐₜ₎ and the solution was extracted with EtOAc, washed with NaCl_{(sat.)}, dried over MgSO₄, filtered, concentrated and purified by ISCO SiO₂ chromatography (0-100% EtOAc/n-heptanes gradient) to yield 4-(3,5-difluorophenyl)tetrahydro-2H-pyran-4-ol in 71% yield. ¹H NMR (400 MHz, CHLOROFORM-d) δ ppm 1.59-1.68 (m, 3H), 2.07-2.19 (m, 2H), 3.87-3.93 (m, 4H), 6.72 (tt, J=8.75, 2.20 Hz, 1H), 6.97-7.06 (m, 2H).

### Synthesis of 4-(3,5-difluoro-4-(4,4,5,5-tetramethyl-1,3,2-dioxaborolan-2-yl)phenyl)tetrahydro-2H-pyran-4-ol

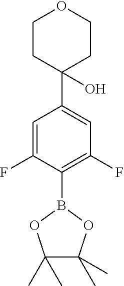 [SMILES: CC1(C)OB(c2c(F)cc(C3(O)CCOCC3)cc2F)OC1(C)C]

Method 3 was followed using 2-isopropoxy-4,4,5,5-tetramethyl-1,3,2-dioxaborolane (2.5 equiv.), butyllithium (2.4 equiv.) and 4-(3,5-difluorophenyl)tetrahydro-2H-pyran-4-ol (1.0 equiv.) to give 4-(3,5-difluoro-4-(4,4,5,5-tetramethyl-1,3,2-dioxaborolan-2-yl)phenyl)tetrahydro-2H-pyran-4-ol in 97% yield. ¹H NMR (400 MHz, <cdcl3>) δ ppm 1.32-1.42 (m, 12H), 1.56-1.65 (m, 2H), 2.11 (d, J=3.13 Hz, 2H), 3.86-3.92 (m, 4H), 6.99 (d, J=9.00 Hz, 2H).

### Synthesis of methyl 6-(2,6-difluoro-4-(4-hydroxytetrahydro-2H-pyran-4-yl)phenyl)-5-fluoropicolinate

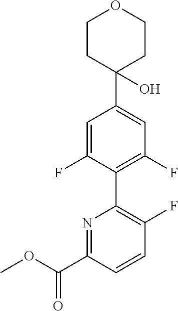 [SMILES: COC(=O)c1ccc(F)c(-c2c(F)cc(C3(O)CCOCC3)cc2F)n1]

Method 1 was followed using methyl 6-bromo-5-fluoropicolinate (1.0 equiv.) and 4-(3,5-difluoro-4-(4,4,5,5-tetramethyl-1,3,2-dioxaborolan-2-yl)phenyl)tetrahydro-2H-pyran-4-ol (1.8 equiv.) at 100° C. for 20 min in microwave to give methyl 6-(2,6-difluoro-4-(4-hydroxytetrahydro-2H-pyran-4-yl)phenyl)-5-fluoropicolinate in 28% yield. LC/MS=368.0 (MH⁺), Rₜ=0.75 min.

### Synthesis of 6-(2,6-difluoro-4-(4-hydroxytetrahydro-2H-pyran-4-yl)phenyl)-5-fluoropicolinic acid

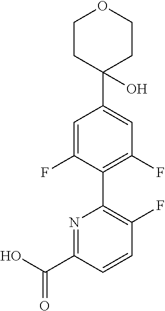 [SMILES: O=C(O)c1ccc(F)c(-c2c(F)cc(C3(O)CCOCC3)cc2F)n1]

Method 2 was followed using methyl 6-(2,6-difluoro-4-(4-hydroxytetrahydro-2H-pyran-4-yl)phenyl)-5-fluoropicolinate to give 6-(2,6-difluoro-4-(4-hydroxytetrahydro-2H-pyran-4-yl)phenyl)-5-fluoropicolinic acid in 69% yield. LC/MS=354.0 (MH⁺), Rₜ=0.64 min.

### Synthesis of methyl 6-(2,6-difluoro-4-(4-fluorotetrahydro-2H-pyran-4-yl)phenyl)-5-fluoropicolinate

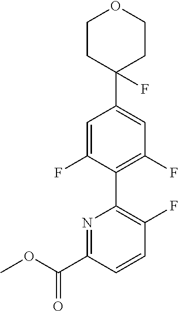 [SMILES: COC(=O)c1ccc(F)c(-c2c(F)cc(C3(F)CCOCC3)cc2F)n1]

To a solution of methyl 6-(2,6-difluoro-4-(4-hydroxytetrahydro-2H-pyran-4-yl)phenyl)-5-fluoropicolinate (1.0 equiv.) in CH₂Cl₂ (0.04 M) at −78° C. under Ar was added methylDAST (2.0 equiv.). After addition, the solution was stirred under Ar at −78° C. for 10 minutes and then the bath was removed. The reaction was allowed to warm up to rt and quenched by addition of NaHCO_{3(sat.)}. The solution was diluted with EtOAc, washed with NaHCO_{3(sat.)}, NaCl_{(sat.)}, dried over MgSO4, filtered, concentrated, purified by ISCO SiO₂ chromatography (0-100 EtOAc/n-heptanes) to yield methyl 6-(2,6-difluoro-4-(4-fluorotetrahydro-2H-pyran-4-yl)phenyl)-5-fluoropicolinate in 100% yield. LC/MS=370.0 (MH⁺), Rₜ=0.94 min.

### Synthesis of 6-(2,6-difluoro-4-(4-fluorotetrahydro-2H-pyran-4-yl)phenyl)-5-fluoropicolinic acid

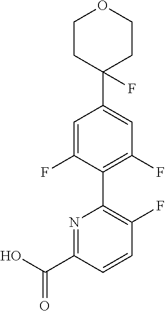 [SMILES: O=C(O)c1ccc(F)c(-c2c(F)cc(C3(F)CCOCC3)cc2F)n1]

Method 2 was followed using methyl 6-(2,6-difluoro-4-(4-fluorotetrahydro-2H-pyran-4-yl)phenyl)-5-fluoropicolinate to give 6-(2,6-difluoro-4-(4-fluorotetrahydro-2H-pyran-4-yl)phenyl)-5-fluoropicolinic acid in 95% yield. LC/MS=355.9 (MH⁺), Rₜ=0.81 min.

### Synthesis of 1-(3,5-difluorophenyl)cyclobutanol

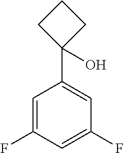 [SMILES: OC1(c2cc(F)cc(F)c2)CCC1]

To a solution of 1-bromo-3,5-difluorobenzene (1.0 equiv.) in THF (0.26 M) under Ar was added Mg turnings (1.6 equiv.). A reflux condenser was attached and the solution was submerged in a 90° C. oil bath and refluxed for two hours. The oxetan-3-one (1.0 equiv.) was added in THF via syringe. The solution was left stirring at rt under Ar for 5 hrs. The reaction solution was quenched by addition of NH₄Cl₍ₛₐₜ₎ and the solution was extracted with EtOAc, washed with NaCl(sat.), dried over MgSO4, filtered, concentrated and purified by ISCO SiO₂ chromatography (0-100% EtOAc/n-heptanes gradient) to yield 1-(3,5-difluorophenyl)cyclobutanol in 54% yield. ¹H NMR (400 MHz, CHLOROFORM-d) δ ppm 1.69-1.83 (m, 1H), 2.03-2.13 (m, 1H), 2.31-2.43 (m, 2H), 2.45-2.56 (m, 2H), 6.71 (tt, J=8.80, 2.35 Hz, 1H), 6.98-7.07 (m, 2H).

### Synthesis of 1-(3,5-difluoro-4-(4,4,5,5-tetramethyl-1,3,2-dioxaborolan-2-yl)phenyl)cyclobutanol

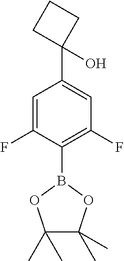 [SMILES: CC1(C)OB(c2c(F)cc(C3(O)CCC3)cc2F)OC1(C)C]

Method 3 was followed using 2-isopropoxy-4,4,5,5-tetramethyl-1,3,2-dioxaborolane (2.5 equiv.), butyllithium (2.4 equiv.) and 1-(3,5-difluorophenyl)cyclobutanol (1.0 equiv.) to give 1-(3,5-difluoro-4-(4,4,5,5-tetramethyl-1,3,2-dioxaborolan-2-yl)phenyl)cyclobutanol in 100% yield. ¹H NMR (400 MHz, <cdcl3>) δ ppm 1.23-1.25 (m, 12H), 1.69-1.82 (m, 1H), 2.05-2.12 (m, 1H), 2.37 (br. s., 2H), 2.47 (br. s., 2H), 7.00 (d, J=8.80 Hz, 2H).

### Synthesis of methyl 6-(2,6-difluoro-4-(1-hydroxycyclobutyl)phenyl)-5-fluoropicolinate

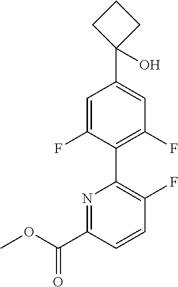 [SMILES: COC(=O)c1ccc(F)c(-c2c(F)cc(C3(O)CCC3)cc2F)n1]

Method 1 was followed using methyl 6-bromo-5-fluoropicolinate (1.0 equiv.) and 1-(3,5-difluoro-4-(4,4,5,5-tetramethyl-1,3,2-dioxaborolan-2-yl)phenyl)cyclobutanol (1.6 equiv.) at 100° C. for 30 min in microwave to give methyl 6-(2,6-difluoro-4-(1-hydroxycyclobutyl)phenyl)-5-fluoropicolinate in 71% yield. LC/MS=338.0 (MH⁺), Rₜ=0.85 min.

### Synthesis of 6-(2,6-difluoro-4-(1-hydroxycyclobutyl)phenyl)-5-fluoropicolinic acid

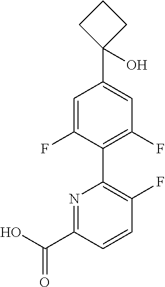 [SMILES: O=C(O)c1ccc(F)c(-c2c(F)cc(C3(O)CCC3)cc2F)n1]

Method 2 was followed using methyl 6-(2,6-difluoro-4-(1-hydroxycyclobutyl)phenyl)-5-fluoropicolinate to give 6-(2,6-difluoro-4-(1-hydroxycyclobutyl)phenyl)-5-fluoropicolinic acid in 90% yield. LC/MS=323.9 (MH+), Rₜ=0.74 min.

### Synthesis of methyl 6-(2,6-difluoro-4-((tetrahydro-2H-pyran-4-yl)oxy)phenyl)-5-fluoropicolinate

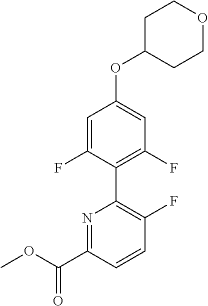 [SMILES: COC(=O)c1ccc(F)c(-c2c(F)cc(OC3CCOCC3)cc2F)n1]

To a solution of DIAD (3.0 equiv.) and triphenylphosphine (3.0 equiv.) in THF (0.24 M) was added tetrahydro-4-pyranol (1.2 equiv.). The mixture was stirred for 10 min. methyl 6-(2,6-difluoro-4-hydroxyphenyl)-5-fluoropicolinate (1.0 equiv.) was added. The mixture was stirred at ambient temperature overnight. Additional triphenylphosphine (3.0 equiv.) and DIAD (3.0 equiv.) were added, and the mixture was stirred overnight. After overnight, the reaction was essentially complete. The mixture was concentrated and purified by flash chromatography over silica gel (heptanes:ethyl acetate gradient) to give methyl 6-(2,6-difluoro-4-((tetrahydro-2H-pyran-4-yl)oxy)phenyl)-5-fluoropicolinate in 77% yield. LC/MS=368.0 (MH+), Rt=0.95 min.

### Synthesis of 6-(2,6-difluoro-4-((tetrahydro-2H-pyran-4-yl)oxy)phenyl)-5-fluoropicolinic acid

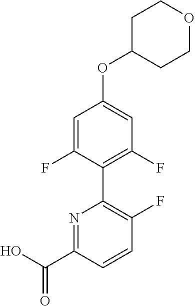 [SMILES: O=C(O)c1ccc(F)c(-c2c(F)cc(OC3CCOCC3)cc2F)n1]

Method 2 was followed using methyl 6-(2,6-difluoro-4-((tetrahydro-2H-pyran-4-yl)oxy)phenyl)-5-fluoropicolinate to give 6-(2,6-difluoro-4-((tetrahydro-2H-pyran-4-yl)oxy)phenyl)-5-fluoropicolinic acid in 100% yield. LC/MS=353.9 (MH+), Rₜ=0.82 min.

### Synthesis of 4-(3,5-difluorophenoxy)tetrahydro-2H-pyran

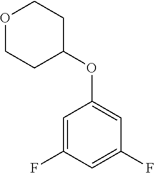 [SMILES: Fc1cc(F)cc(OC2CCOCC2)c1]

To a solution of 3,5-difluorophenol (1.0 equiv.), tetrahydro-2H-pyran-4-ol (1.2 equiv.), and triphenylphosphine (2.0 equiv.) in THF (0.33 M) at 0° C. was added DIAD (2.0 equiv.) dropwise. The reaction mixture was stirred at rt overnight. The mixture was concentrated and purified by flash chromatography over silica gel (heptanes:ethyl acetate gradient) to give 4-(3,5-difluorophenoxy)tetrahydro-2H-pyran in 90% yield. 1H NMR (400 MHz, <cdcl3>) δ ppm 1.72-1.84 (m, 2H), 1.96-2.09 (m, 2H), 3.59 (ddd, J=11.64, 8.31, 3.52 Hz, 2H), 3.90-4.04 (m, 2H), 4.44 (tt, J=7.78, 3.77 Hz, 1H), 6.32-6.53 (m, 3H).

### Synthesis of 2-(2,6-difluoro-4-((tetrahydro-2H-pyran-4-yl)oxy)phenyl)-4,4,5,5-tetramethyl-1,3,2-dioxaborolane

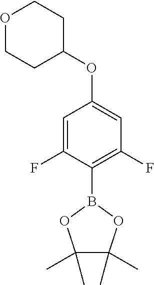 [SMILES: CC1(C)OB(c2c(F)cc(OC3CCOCC3)cc2F)OC1(C)C]

Method 3 was followed using 2-isopropoxy-4,4,5,5-tetramethyl-1,3,2-dioxaborolane (1.5 equiv.), butyllithium (1.3 equiv.) and 4-(3,5-difluorophenoxy)tetrahydro-2H-pyran (1.0 equiv.) to give 2-(2,6-difluoro-4-((tetrahydro-2H-pyran-4-yl)oxy)phenyl)-4,4,5,5-tetramethyl-1,3,2-dioxaborolane in 33% yield. ¹H NMR (400 MHz, <cdcl3>) δ ppm 1.21-1.34 (m, 12H), 1.78 (dtd, J=12.72, 8.31, 8.31, 3.91 Hz, 2H), 1.93-2.09 (m, 2H), 3.59 (ddd, J=11.64, 8.31, 3.13 Hz, 2H), 3.89-4.01 (m, 2H), 4.48 (tt, J=7.78, 3.77 Hz, 1H), 6.40 (d, J=9.39 Hz, 2H).

### Synthesis of (S)-methyl 6-(2,6-difluoro-4-((tetrahydro-2H-pyran-3-yl)oxy)phenyl)-5-fluoropicolinate and (R)-methyl 6-(2,6-difluoro-4-((tetrahydro-2H-pyran-3-yl)oxy)phenyl)-5-fluoropicolinate

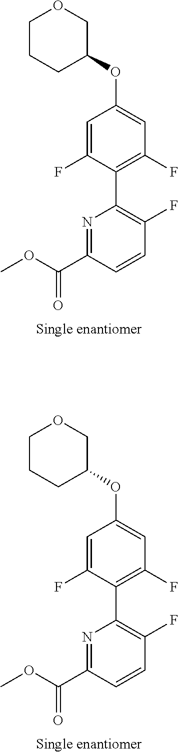 [SMILES: COC(=O)c1ccc(F)c(-c2c(F)cc(O[C@H]3CCCOC3)cc2F)n1 ; COC(=O)c1ccc(F)c(-c2c(F)cc(O[C@@H]3CCCOC3)cc2F)n1]

To a solution of DIAD (2.0 equiv.) and triphenylphosphine (2.0 equiv.) in THF (0.24 M) was added tetrahydro-2H-pyran-3-ol (1.2 equiv.). The mixture was stirred for 10 min. methyl 6-(2,6-difluoro-4-hydroxyphenyl)-5-fluoropicolinate (1.0 equiv.) was added. The mixture was stirred at ambient temperature overnight. Additional triphenylphosphine (2.0 equiv.) and DIAD (2.0 equiv.) were added, and the mixture was stirred overnight. The mixture was concentrated and purified by flash chromatography over silica gel (heptanes:ethyl acetate gradient) to give methyl 6-(2,6-difluoro-4-((tetrahydro-2H-pyran-3-yl)oxy)phenyl)-5-fluoropicolinate in 39% yield. Purification was completed via chiral HPLC (EtOH/heptane)=15/85, 20 mL/min, AD column) to yield (S)-methyl 6-(2,6-difluoro-4-((tetrahydro-2H-pyran-3-yl)oxy)phenyl)-5-fluoropicolinate (18% yield, 99% ee) and (R)-methyl 6-(2,6-difluoro-4-((tetrahydro-2H-pyran-3-yl)oxy)phenyl)-5-fluoropicolinate (18% yield, 99% ee). LC/MS=368.2 (MH+), Rt=0.92 min. ¹H NMR (400 MHz, CHLOROFORM-d) δ ppm 1.65 (ddd, J=12.81, 8.51, 4.11 Hz, 1H), 1.78-1.97 (m, 2H), 2.06-2.16 (m, 1H), 3.57-3.67 (m, 2H), 3.72-3.80 (m, 1H), 3.95 (dd, J=11.54, 2.15 Hz, 1H), 3.99-4.01 (m, 3H), 4.32 (dt, J=6.95, 3.37 Hz, 1H), 6.54-6.62 (m, 2H), 7.59-7.67 (m, 1H), 8.19-8.28 (m, 1H).

### Synthesis of (R)-6-(2,6-difluoro-4-((tetrahydro-2H-pyran-3-yl)oxy)phenyl)-5-fluoropicolinic acid

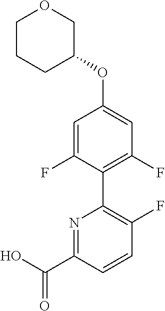 [SMILES: O=C(O)c1ccc(F)c(-c2c(F)cc(O[C@@H]3CCCOC3)cc2F)n1]

Method 2 was followed using (R)-methyl 6-(2,6-difluoro-4-((tetrahydro-2H-pyran-3-yl)oxy)phenyl)-5-fluoropicolinate to give (R)-6-(2,6-difluoro-4-((tetrahydro-2H-pyran-3-yl)oxy)phenyl)-5-fluoropicolinic acid in 93% yield. LC/MS=353.9 (MH+), Rt=0.81 min.

### Synthesis of (S)-6-(2,6-difluoro-4-((tetrahydro-2H-pyran-3-yl)oxy)phenyl)-5-fluoropicolinic acid

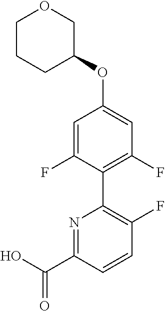 [SMILES: O=C(O)c1ccc(F)c(-c2c(F)cc(O[C@H]3CCCOC3)cc2F)n1]

Method 2 was followed using (S)-methyl 6-(2,6-difluoro-4-((tetrahydro-2H-pyran-3-yl)oxy)phenyl)-5-fluoropicolinate to give (S)-6-(2,6-difluoro-4-((tetrahydro-2H-pyran-3-yl)oxy)phenyl)-5-fluoropicolinic acid in 94% yield. LC/MS=353.9 (MH⁺), Rₜ=0.81 min.

### Synthesis of methyl 6-(4-(ethoxymethyl)-2,6-difluorophenyl)-5-fluoropicolinate

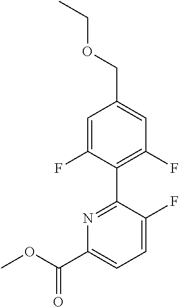 [SMILES: CCOCc1cc(F)c(-c2nc(C(=O)OC)ccc2F)c(F)c1]

To a solution of methyl 6-(2,6-difluoro-4-(hydroxymethyl)phenyl)-5-fluoropicolinate (1.0 equiv.) in DMF (0.20 M) (colorless) at 0° C. was added sodium hydride (1.2 equiv.) and the reaction was stirred at 0° C. for 2 min. Ethyl iodide (1.2 equiv.) was added and the reaction was allowed to warm to room temperature. After 1 h, additional 1.0 equiv. of NaH was added and stirred for 15 ml. Reaction was quenched by the addition of sat. Ammonium chloride. The aqueous was acidified with conc HCl to pH3 and extracted with ethyl acetate three times. The organics were combined, dried with MgSO4, filtered and concentrated. The crude mixture was used as is. LC/MS=326.0 (MH⁺), Rₜ=0.94 min.

### Synthesis of 6-(4-(ethoxymethyl)-2,6-difluorophenyl)-5-fluoropicolinic acid

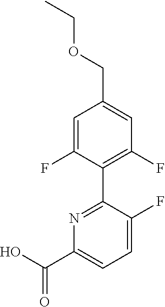 [SMILES: CCOCc1cc(F)c(-c2nc(C(=O)O)ccc2F)c(F)c1]

Method 2 was followed using methyl 6-(4-(ethoxymethyl)-2,6-difluorophenyl)-5-fluoropicolinate to give 6-(4-(ethoxymethyl)-2,6-difluorophenyl)-5-fluoropicolinic acid in 27% yield. LC/MS=311.9 (MH⁺), Rₜ=0.82 min.

### Synthesis of methyl 6-(4-(difluoromethyl)-2,6-difluorophenyl)-5-fluoropicolinate

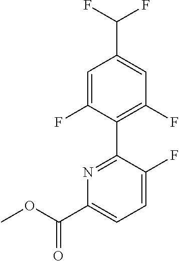 [SMILES: COC(=O)c1ccc(F)c(-c2c(F)cc(C(F)F)cc2F)n1]

To a solution of methyl 6-(2,6-difluoro-4-formylphenyl)-5-fluoropicolinate (1.0 equiv.) in DCM (0.14 M) at 0° C. was added DAST (1.4 equiv.) dropwise. The resulting mixture was then allowed to warm to RT over 3 h. The reaction mixture was quenched with water and diluted with EtOAc. The aqueous layer was separated then extracted with EtOAc. The combined organics were dried over MgSO4 and concentrated in vacuo. The crude was further purified by column chromatography eluting with 100% heptanes to 10% EtOAc: heptanes to yield methyl 6-(4-(difluoromethyl)-2,6-difluorophenyl)-5-fluoropicolinate as a colourless solid in 88% yield. LC/MS=317.9 (MH+), Rt=0.92 min.

### Synthesis of 6-(4-(difluoromethyl)-2,6-difluorophenyl)-5-fluoropicolinic acid

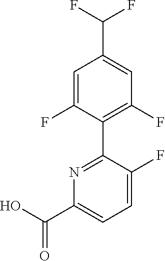 [SMILES: O=C(O)c1ccc(F)c(-c2c(F)cc(C(F)F)cc2F)n1]

Method 2 was followed using methyl 6-(4-(difluoromethyl)-2,6-difluorophenyl)-5-fluoropicolinate to give 6-(4-(difluoromethyl)-2,6-difluorophenyl)-5-fluoropicolinic acid in 92% yield. LC/MS=303.8 (MH+), Rt=0.80 min.

### Synthesis of 1,3-difluoro-5-isopropoxybenzene

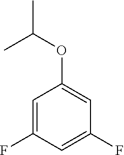

To a solution of 3,5-difluorophenol (1.0 equiv.) in DMF (0.26 M) was added potassium carbonate (2.2 equiv.) followed by 2-iodopropane (1.1 equiv.) and the reaction was stirred overnight at room temperature. The reaction was poured into a separatory funnel and diluted with a 3:1 (v/v) solution of EtOAc:heptanes. The organic phase was washed with water, then sat'd NaHCO3. The remaining organic phase was dried over MgSO4, filtered and concentrated in vacuo to provide 1,3-difluoro-5-isopropoxybenzene in 88% yield. 1H NMR (400 MHz, <cdcl3>) δ ppm 1.33 (d, J=6.26 Hz, 6H), 4.48 (dt, J=11.93, 6.16 Hz, 1H), 6.31-6.47 (m, 3H).

### Synthesis of 2-(2,6-difluoro-4-isopropoxyphenyl)-4,4,5,5-tetramethyl-1,3,2-dioxaborolane

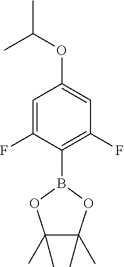 [SMILES: CC(C)Oc1cc(F)c(B2OC(C)(C)C(C)(C)O2)c(F)c1]

Method 3 was followed using 2-isopropoxy-4,4,5,5-tetramethyl-1,3,2-dioxaborolane (2.2 equiv.), butyllithium (1.2 equiv.) and 1,3-difluoro-5-isopropoxybenzene (1.0 equiv.) to give 2-(2,6-difluoro-4-isopropoxyphenyl)-4,4,5,5-tetramethyl-1,3,2-dioxaborolane in 99% yield. 1H NMR (400 MHz, <cdcl3>) δ ppm 1.24 (s, 12H), 1.31-1.33 (m, 6H), 4.43-4.56 (m, 1H), 6.31-6.44 (m, 2H).

### Synthesis of methyl 6-(2,6-difluoro-4-isopropoxyphenyl)-5-fluoropicolinate

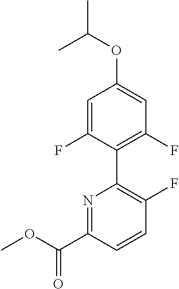 [SMILES: COC(=O)c1ccc(F)c(-c2c(F)cc(OC(C)C)cc2F)n1]

Method 1 was followed using methyl 6-bromo-5-fluoropicolinate (0.8 equiv.) and 2-(2,6-difluoro-4-isopropoxyphenyl)-4,4,5,5-tetramethyl-1,3,2-dioxaborolane (1.0 equiv.) at 70° C. for 1 hour to give methyl 6-(2,6-difluoro-4-isopropoxyphenyl)-5-fluoropicolinate in 27% yield. LC/MS=325.9 (MH+), Rt=1.04 min.

### Synthesis of 6-(2,6-difluoro-4-isopropoxyphenyl)-5-fluoropicolinic acid

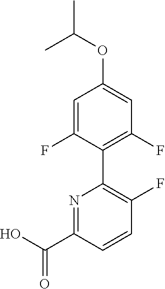 [SMILES: CC(C)Oc1cc(F)c(-c2nc(C(=O)O)ccc2F)c(F)c1]

Method 2 was followed using methyl 6-(2,6-difluoro-4-isopropoxyphenyl)-5-fluoropicolinate to give 6-(2,6-difluoro-4-isopropoxyphenyl)-5-fluoropicolinic acid in 35% yield. LC/MS=311.9 (MH+), Rt=0.92 min.

### Synthesis of 3-(3,5-difluorophenyl)oxetane

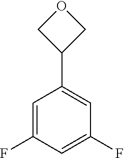 [SMILES: Fc1cc(F)cc(C2COC2)c1]

3,5-difluorophenylboronic acid (2.0 equiv.), (1R,2R)-2-aminocyclohexanol (0.06 equiv.), NaHMDS (2.0 equiv.), and nickel(II) iodide (0.06 equiv.) were dissolved in 2-propanol (0.35 M). The mixture was degassed with N2, stirred at rt for 10 min and then a solution of 3-iodooxetane (1.0 equiv.) in 2-Propanol (0.70 M) was added. The mixture was sealed and heated at 80° C. in the microwave for 20 min. The mixture was filtered through celite, eluting with EtOH and concentrated. The crude residue was purified by ISCO SiO2 chromatography eluting with 0-100% EtOAc in Heptanes to afford 3-(3,5-difluorophenyl)oxetane in 63% yield. 1H NMR (400 MHz, <cdcl3>) δ 6.88-6.96 (m, 2H), 6.72 (tt, J=2.20, 8.95 Hz, 1H), 5.08 (dd, J=6.26, 8.22 Hz, 2H), 4.71 (t, J=6.26 Hz, 2H), 4.14-4.24 (m, 1H).

### Synthesis of 2-(2,6-difluoro-4-(oxetan-3-yl)phenyl)-4,4,5,5-tetramethyl-1,3,2-dioxaborolane

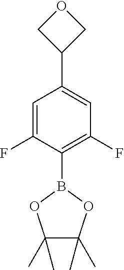 [SMILES: CC1(C)OB(c2c(F)cc(C3COC3)cc2F)OC1(C)C]

Method 3 was followed using 2-isopropoxy-4,4,5,5-tetramethyl-1,3,2-dioxaborolane (1.3 equiv.), butyllithium (1.1 equiv.) and 3-(3,5-difluorophenyl)oxetane (1.0 equiv.) to give 2-(2,6-difluoro-4-(oxetan-3-yl)phenyl)-4,4,5,5-tetramethyl-1,3,2-dioxaborolane in 8% yield. 1H NMR (400 MHz, <cdcl3>) δ ppm 6.90 (d, J=8.22 Hz, 2H), 5.07 (dd, J=6.06, 8.41 Hz, 2H), 4.70 (t, J=6.26 Hz, 2H), 4.13-4.23 (m, 1H), 1.39 (s, 12H).

### Synthesis of methyl 6-(2,6-difluoro-4-(oxetan-3-yl)phenyl)-5-fluoropicolinate

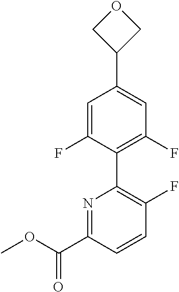 [SMILES: COC(=O)c1ccc(F)c(-c2c(F)cc(C3COC3)cc2F)n1]

Method 1 was followed using methyl 6-bromo-5-fluoropicolinate (1.2 equiv.) and 2-(2,6-difluoro-4-(oxetan-3-yl)phenyl)-4,4,5,5-tetramethyl-1,3,2-dioxaborolane (1.0 equiv.) at 80° C. for 15 min in microwave to give methyl 6-(2,6-difluoro-4-(oxetan-3-yl)phenyl)-5-fluoropicolinate in 47% yield. LC/MS=324.0 (MH+), Rt=0.75 min.

### Synthesis of 6-(2,6-difluoro-4-(oxetan-3-yl)phenyl)-5-fluoropicolinic acid

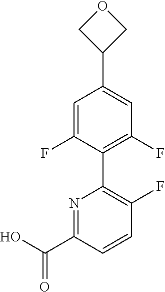 [SMILES: O=C(O)c1ccc(F)c(-c2c(F)cc(C3COC3)cc2F)n1]

Method 2 was followed using methyl 6-(2,6-difluoro-4-(oxetan-3-yl)phenyl)-5-fluoropicolinate to give 6-(2,6-difluoro-4-(oxetan-3-yl)phenyl)-5-fluoropicolinic acid in 71% yield. LC/MS=309.9 (MH+), Rt=0.69 min.

### Synthesis of methyl 3-amino-6-(2,6-difluoro-4-(2-methoxyethoxy)phenyl)-5-fluoropicolinate

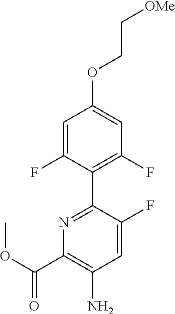 [SMILES: COCCOc1cc(F)c(-c2nc(C(=O)OC)c(N)cc2F)c(F)c1]

Method 1 was followed using methyl 3-amino-6-bromo-5-fluoropicolinate (1.0 equiv.) and 2-(2,6-difluoro-4-(2-methoxyethoxy)phenyl)-4,4,5,5-tetramethyl-1,3,2-dioxaborolane (1.5 equiv.) at 100° C. for 20 min in microwave to give methyl 3-amino-6-(2,6-difluoro-4-(2-methoxyethoxy)phenyl)-5-fluoropicolinate in 36% yield. LC/MS=357.2 (MH+), Rt=0.82 min. 1H NMR (400 MHz, <cdcl3>) δ ppm 3.46 (s, 3H), 3.76 (dd, J=5.28, 3.72 Hz, 2H), 3.95 (s, 3H), 4.12 (dd, J=5.48, 3.91 Hz, 2H), 6.01 (br. s., 2H), 6.49-6.63 (m, 2H), 6.82 (d, J=9.78 Hz, 1H).

### Synthesis of 3-amino-6-(2,6-difluoro-4-(2-methoxyethoxy)phenyl)-5-fluoropicolinic acid

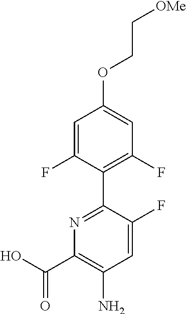 [SMILES: COCCOc1cc(F)c(-c2nc(C(=O)O)c(N)cc2F)c(F)c1]

Method 2 was followed using methyl 3-amino-6-(2,6-difluoro-4-(2-methoxyethoxy)phenyl)-5-fluoropicolinate to give 3-amino-6-(2,6-difluoro-4-(2-methoxyethoxy)phenyl)-5-fluoropicolinic acid in 98% yield. LC/MS=343.0 (MH+), Rt=0.82 min.

### Synthesis of methyl 3-amino-6-(2,6-difluoro-4-(2-hydroxypropan-2-yl)phenyl)-5-fluoropicolinate

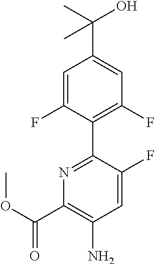 [SMILES: COC(=O)c1nc(-c2c(F)cc(C(C)(C)O)cc2F)c(F)cc1N]

Method 1 was followed using methyl 3-amino-6-bromo-5-fluoropicolinate (1.0 equiv.) and 2-(3,5-difluoro-4-(4,4,5,5-tetramethyl-1,3,2-dioxaborolan-2-yl)phenyl)propan-2-ol (2.0 equiv.) at 100° C. for 20 min in microwave to give methyl 3-amino-6-(2,6-difluoro-4-(2-hydroxypropan-2-yl)phenyl)-5-fluoropicolinate in 87% yield. LC/MS=340.9 (MH+), Rt=0.77 min.

### Synthesis of 3-amino-6-(2,6-difluoro-4-(2-hydroxypropan-2-yl)phenyl)-5-fluoropicolinic acid

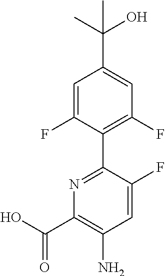 [SMILES: CC(C)(O)c1cc(F)c(-c2nc(C(=O)O)c(N)cc2F)c(F)c1]

Method 2 was followed using methyl 3-amino-6-(2,6-difluoro-4-(2-hydroxypropan-2-yl)phenyl)-5-fluoropicolinate to give 3-amino-6-(2,6-difluoro-4-(2-hydroxypropan-2-yl)phenyl)-5-fluoropicolinic acid in 98% yield. LC/MS=326.8 (MH+), Rt=0.68 min. 1H NMR (400 MHz, <cdcl3>) δ ppm 2.10 (s, 6H), 6.92 (d, J=9.78 Hz, 1H), 7.09-7.19 (m, 2H).

### Synthesis of 3-(3,5-difluorophenyl)-3-methoxyoxetane

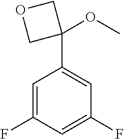

A solution of 3-(3,5-difluorophenyl)oxetan-3-ol (1.0 equiv.) in DMF (0.23 M) was cooled in an ice water bath. NaH, 60% dispersion in mineral oil (1.1 equiv.) was added. The mixture was stirred for 1 hr. iodomethane (1.1 equiv.) was added in a dropwise fashion. The ice bath was removed, and the mixture was stirred for 2 hr at ambient temperature. The reaction mixture was quenched by the addition of water. The mixture was extracted with ether. The combined extracts were washed sequentially with water and brine, dried over sodium sulfate, filtered, and concentrated. The crude material was purified by flash chromatography over silica gel (2:1 pentane:ether) to give 3-(3,5-difluorophenyl)-3-methoxyoxetane in 83% yield. ¹H NMR (400 MHz, CHLOROFORM-d) δ ppm 3.18 (s, 3H), 4.70 (d, J=7.04 Hz, 2H), 4.92 (d, J=7.43 Hz, 2H), 6.80 (tt, J=8.66, 2.30 Hz, 1H), 6.99-7.08 (m, 2H).

### Synthesis of 2-(2,6-difluoro-4-(3-methoxyoxetan-3-yl)phenyl)-4,4,5,5-tetramethyl-1,3,2-dioxaborolane

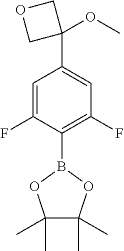 [SMILES: COC1(c2cc(F)c(B3OC(C)(C)C(C)(C)O3)c(F)c2)COC1]

Method 3 was followed using 2-isopropoxy-4,4,5,5-tetramethyl-1,3,2-dioxaborolane (1.3 equiv.), butyllithium (1.3 equiv.) and 3-(3,5-difluorophenyl)-3-methoxyoxetane (1.0 equiv.) to give 2-(2,6-difluoro-4-(3-methoxyoxetan-3-yl)phenyl)-4,4,5,5-tetramethyl-1,3,2-dioxaborolane in 100% yield. 1H NMR (400 MHz, CHLOROFORM-d) δ ppm 1.22-1.26 (m, 12H), 3.16 (s, 3H), 4.67-4.73 (m, 2H), 4.89-4.94 (m, 2H), 7.00 (d, J=8.22 Hz, 2H).

### Synthesis of methyl 3-amino-6-(2,6-difluoro-4-isopropoxyphenyl)-5-fluoropicolinate

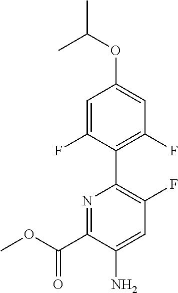 [SMILES: COC(=O)c1nc(-c2c(F)cc(OC(C)C)cc2F)c(F)cc1N]

Method 1 was followed using methyl 3-amino-6-bromo-5-fluoropicolinate (1.0 equiv.) and 2-(2,6-difluoro-4-isopropoxyphenyl)-4,4,5,5-tetramethyl-1,3,2-dioxaborolane (1.6 equiv.) at 70° C. for 1 hr to give methyl 3-amino-6-(2,6-difluoro-4-isopropoxyphenyl)-5-fluoropicolinate in 44% yield. LC/MS=340.9 (MH+), Rt=0.98 min.

### Synthesis of 3-amino-6-(2,6-difluoro-4-isopropoxyphenyl)-5-fluoropicolinic acid

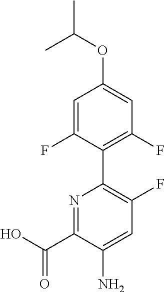 [SMILES: CC(C)Oc1cc(F)c(-c2nc(C(=O)O)c(N)cc2F)c(F)c1]

Method 2 was followed using methyl 3-amino-6-(2,6-difluoro-4-isopropoxyphenyl)-5-fluoropicolinate to give 3-amino-6-(2,6-difluoro-4-isopropoxyphenyl)-5-fluoropicolinic acid in 84% yield. LC/MS=327.0 (MH+), Rt=0.94 min.

### Synthesis of methyl 6-(2,6-difluoro-4-(2-(2-oxopyrrolidin-1-yl)ethoxy)phenyl)-5-fluoropicolinate

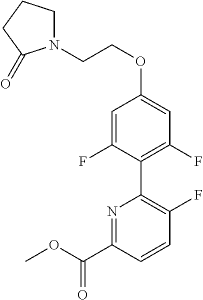 [SMILES: COC(=O)c1ccc(F)c(-c2c(F)cc(OCCN3CCCC3=O)cc2F)n1]

To a solution of triphenylphosphine (1.5 equiv.), methyl 6-(2,6-difluoro-4-hydroxyphenyl)-5-fluoropicolinate (1.0 equiv.) and 1-(2-hydroxyethyl)pyrrolidin-2-one (1.2 equiv.) in THF (0.14 M) at 0° C. was added DIAD (1.5 equiv.) dropwise. The reaction was allowed to warm to rt and stirred for 6 hrs. The reaction mixture was concentrated under vacuo and purified via ISCO (ethyl acetate and heptanes 0-100%) to give methyl 6-(2,6-difluoro-4-(2-(2-oxopyrrolidin-1-yl)ethoxy)phenyl)-5-fluoropicolinate in 96% yield. LC/MS=395.0 (MH+), Rt=0.80 min. ¹H NMR (400 MHz, <cdcl3>) δ ppm 1.97-2.14 (m, 2H), 2.31-2.50 (m, 2H), 3.57 (t, J=7.04 Hz, 2H), 3.71 (t, J=5.09 Hz, 2H), 4.00 (s, 3H), 4.08-4.20 (m, 3H), 6.56 (d, J=9.00 Hz, 2H), 7.63 (t, J=8.41 Hz, 1H), 8.24 (dd, J=8.61, 3.91 Hz, 1H).

### Synthesis of 6-(2,6-difluoro-4-(2-(2-oxopyrrolidin-1-yl)ethoxy)phenyl)-5-fluoropicolinic acid

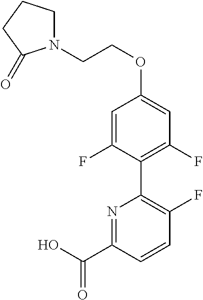 [SMILES: O=C(O)c1ccc(F)c(-c2c(F)cc(OCCN3CCCC3=O)cc2F)n1]

Method 2 was followed using methyl 6-(2,6-difluoro-4-(2-(2-oxopyrrolidin-1-yl)ethoxy)phenyl)-5-fluoropicolinate to give 6-(2,6-difluoro-4-(2-(2-oxopyrrolidin-1-yl)ethoxy)phenyl)-5-fluoropicolinic acid in 70% yield. LC/MS=381.0 (MH+), Rt=0.70 min.

### Synthesis of methyl 6-(4-(bromomethyl)-2,6-difluorophenyl)-5-fluoropicolinate

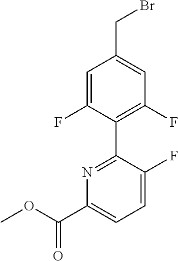 [SMILES: COC(=O)c1ccc(F)c(-c2c(F)cc(CBr)cc2F)n1]

A solution of bromine (1.0 equiv.) in DCM (0.20 M) was added to triphenylphosphine (1.0 equiv.). The mixture became homogeneous and colorless and was stirred for an additional 30 min. This heterogeneous mixture was added to methyl 6-(2,6-difluoro-4-(hydroxymethyl)phenyl)-5-fluoropicolinate (1.0 equiv.). The light yellow solution was stirred at 50° C. for 3 hrs. The reaction mixture was concentrated and purified by flash chromatography over silica gel to give methyl 6-(4-(bromomethyl)-2,6-difluorophenyl)-5-fluoropicolinate in 71% yield. LC/MS=362.1 (MH+), Rt=0.92 min.

### Synthesis of methyl 6-(4-(cyanomethyl)-2,6-difluorophenyl)-5-fluoropicolinate

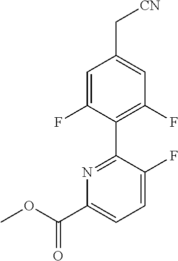 [SMILES: COC(=O)c1ccc(F)c(-c2c(F)cc(CC#N)cc2F)n1]

A solution of sodium cyanide (1.4 equiv.) in water (0.65 M) was stirred at 50° C. A solution of methyl 6-(4-(bromomethyl)-2,6-difluorophenyl)-5-fluoropicolinate (1.0 equiv.) in ACN (0.07 M) was added in a dropwise fashion over 15 min. The colorless solution was stirred at 50° C. for 2 hrs. The cooled reaction mixture was concentrated. Water was added, and the product was extracted with ethyl acetate. The combined extracts were dried over sodium sulfate, filtered, and concentrated to give methyl 6-(4-(cyanomethyl)-2,6-difluorophenyl)-5-fluoropicolinate in 89% yield. LC/MS=307.1 (MH+), Rt=0.77 min.

### Synthesis of methyl 6-(4-(2-cyanopropan-2-yl)-2,6-difluorophenyl)-5-fluoropicolinate

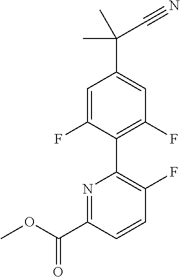 [SMILES: COC(=O)c1ccc(F)c(-c2c(F)cc(C(C)(C)C#N)cc2F)n1]

Sodium hydride (2.2 equiv.) was added to a solution of methyl 6-(4-(cyanomethyl)-2,6-difluorophenyl)-5-fluoropicolinate (1.0 equiv.) in DMSO (0.26 M). The red mixture was stirred for 15 min at ambient temperature. iodomethane (2.1 equiv.) was added in a dropwise fashion. The reaction mixture was stirred for 20 min at ambient temperature. The reaction mixture was diluted with water and extracted with ethyl acetate. The combined organics were washed sequentially with water and brine, dried over sodium sulfate, filtered, concentrated, and purified by flash chromatography (heptanes:ethyl acetate gradient) over silica gel to give methyl 6-(4-(2-cyanopropan-2-yl)-2,6-difluorophenyl)-5-fluoropicolinate in 35% yield. LC/MS=335.1 (MH+), Rt=0.90 min.

### Synthesis of 6-(4-(2-cyanopropan-2-yl)-2,6-difluorophenyl)-5-fluoropicolinic acid

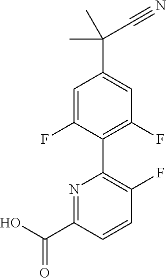 [SMILES: CC(C)(C#N)c1cc(F)c(-c2nc(C(=O)O)ccc2F)c(F)c1]

Method 2 was followed using methyl 6-(4-(2-cyanopropan-2-yl)-2,6-difluorophenyl)-5-fluoropicolinate to give 6-(4-(2-cyanopropan-2-yl)-2,6-difluorophenyl)-5-fluoropicolinic acid in 99% yield. LC/MS=321.2 (MH+), Rt=0.79 min.

### Synthesis of methyl 6-(4-(4-cyanotetrahydro-2H-pyran-4-yl)-2,6-difluorophenyl)-5-fluoropicolinate

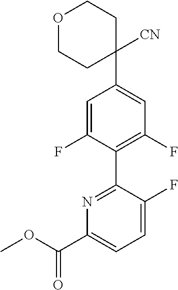 [SMILES: COC(=O)c1ccc(F)c(-c2c(F)cc(C3(C#N)CCOCC3)cc2F)n1]

Sodium hydride (2.2 equiv.) was added to a solution of methyl 6-(4-(cyanomethyl)-2,6-difluorophenyl)-5-fluoropicolinate (1.0 equiv.) in DMSO (0.51 M). The red mixture was stirred for 15 min at ambient temperature. bis(2-bromoethyl)ether (1.1 equiv.) was added in a dropwise fashion. After stirred at rt for 30 min, the mixture was diluted with water and extracted with ethyl acetate. The combined extracts were dried over sodium sulfate, filtered, concentrated and purified by flash chromatography (heptanes:ethyl acetate gradient) over silica gel to give methyl 6-(4-(4-cyanotetrahydro-2H-pyran-4-yl)-2,6-difluorophenyl)-5-fluoropicolinate in 15% yield. LC/MS=377.2 (MH+), Rt=0.85 min.

### Synthesis of 6-(4-(4-cyanotetrahydro-2H-pyran-4-yl)-2,6-difluorophenyl)-5-fluoropicolinic acid

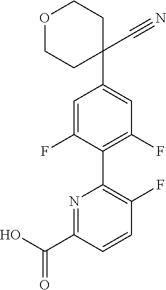 [SMILES: N#CC1(c2cc(F)c(-c3nc(C(=O)O)ccc3F)c(F)c2)CCOCC1]

Method 2 was followed using methyl 6-(4-(4-cyanotetrahydro-2H-pyran-4-yl)-2,6-difluorophenyl)-5-fluoropicolinate to give 6-(4-(4-cyanotetrahydro-2H-pyran-4-yl)-2,6-difluorophenyl)-5-fluoropicolinic acid in 96% yield. LC/MS=363.2 (MH+), Rt=0.74 min.

### Synthesis of 4-(3,5-difluorophenyl)morpholine

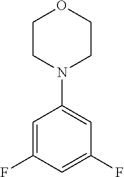 [SMILES: Fc1cc(F)cc(N2CCOCC2)c1]

Tert-amyl alcohol was degassed by bubbling N2 through it for 15 min. 1-bromo-3,5-difluorobenzene (1.0 equiv.), Pd₂(dba)₃ (0.03 equiv.), X-Phos (0.14 equiv.), potassium carbonate (1.0 equiv.) and morpholine (0.92 equiv.) were added and the mixture heated to 100° C. for 18 hrs under N2. The solution was diluted with water and ether. The aqueous was extracted with ether. The combined organics were dried over sodium sulfate, filtered and concentrated to afford a red heterogeneous mixture. The crude oil was purified by ISCO SiO2 chromatography, eluting with 0-30% Ether in Pentanes, then eluting with 0-100% DCM in Pentanes to afford 4-(3,5-difluorophenyl)morpholine in 30% yield. ¹H NMR (400 MHz, CHLOROFORM-d) δ ppm 3.14 (d, J=9.78 Hz, 3H), 3.83 (d, J=5.09 Hz, 4H), 6.28 (tt, J=8.90, 2.05 Hz, 1H), 6.32-6.40 (m, 2H).

### Synthesis of 4-(3,5-difluoro-4-(4,4,5,5-tetramethyl-1,3,2-dioxaborolan-2-yl)phenyl)morpholine

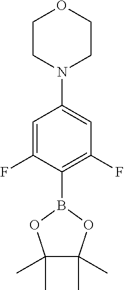 [SMILES: CC1(C)OB(c2c(F)cc(N3CCOCC3)cc2F)OC1(C)C]

Method 3 was followed using 2-isopropoxy-4,4,5,5-tetramethyl-1,3,2-dioxaborolane (2.1 equiv.), butyllithium (1.0 equiv.) and 4-(3,5-difluorophenyl)morpholine (1.0 equiv.) to give 4-(3,5-difluoro-4-(4,4,5,5-tetramethyl-1,3,2-dioxaborolan-2-yl)phenyl)morpholine in 100% yield. 1H NMR (400 MHz, CHLOROFORM-d) δ ppm 6.26-6.34 (m, 2H), 3.80-3.84 (m, 4H), 3.18-3.23 (m, 4H), 1.36 (s, 12H).

### Synthesis of methyl 6-(2,6-difluoro-4-morpholinophenyl)-5-fluoropicolinate

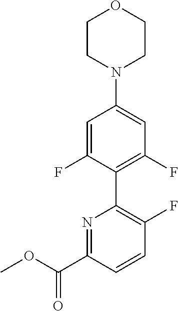 [SMILES: COC(=O)c1ccc(F)c(-c2c(F)cc(N3CCOCC3)cc2F)n1]

Method 1 was followed using methyl 6-bromo-5-fluoropicolinate (1.0 equiv.) and 4-(3,5-difluoro-4-(4,4,5,5-tetramethyl-1,3,2-dioxaborolan-2-yl)phenyl)morpholine (1.5 equiv.) at 100° C. for 30 min in microwave to give methyl 6-(2,6-difluoro-4-morpholinophenyl)-5-fluoropicolinate in 75% yield. LC/MS=353.3 (MH+), Rt=0.86 min. 1H NMR (400 MHz, <cdcl3>) δ 8.21 (dd, J=3.91, 8.61 Hz, 1H), 7.61 (t, J=8.41 Hz, 1H), 6.43-6.52 (m, 2H), 4.00 (s, 3H), 3.83-3.89 (m, 4H), 3.19-3.25 (m, 4H).

### Synthesis of 6-(2,6-difluoro-4-morpholinophenyl)-5-fluoropicolinic acid

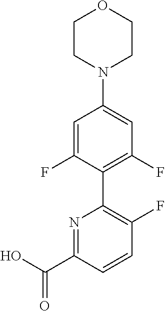 [SMILES: O=C(O)c1ccc(F)c(-c2c(F)cc(N3CCOCC3)cc2F)n1]

Method 2 was followed using methyl 6-(2,6-difluoro-4-morpholinophenyl)-5-fluoropicolinate to give 6-(2,6-difluoro-4-morpholinophenyl)-5-fluoropicolinic acid in 68% yield. LC/MS=339.1 (MH+), Rt=0.75 min. 1H NMR (400 MHz, <dmso>) δ 13.40 (br. s., 1H), 8.17 (dd, J=3.91, 8.61 Hz, 1H), 8.00 (t, J=8.80 Hz, 1H), 6.78-6.87 (m, 2H), 3.70-3.76 (m, 4H), 3.26-3.30 (m, 4H).

### Synthesis of 1,3-difluoro-5-(isopropoxymethyl)benzene

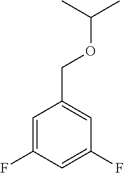

2-propanol (1.0 equiv.) was dissolved in DMF (0.20 M). Sodium hydride, 60% in mineral oil (1.1 equiv.) was added. The reaction mixture was stirred at ambient temperature for 1 hr. 3,5-difluorobenzyl bromide (1.1 equiv.) was added in a dropwise fashion. The mixture was stirred overnight at ambient temperature. The reaction mixture was quenched by the addition of water. The mixture was extracted with ether. The combined extracts were washed sequentially with water and brine, dried over sodium sulfate, filtered, and concentrated. The crude material was purified by flash chromatography over silica gel (4:1 pentane:ether) to give 1,3-difluoro-5-(isopropoxymethyl)benzene in 54% yield. 1H NMR (400 MHz, CHLOROFORM-d) δ ppm 1.22 (d, J=5.87 Hz, 6H), 3.68 (spt, J=6.13 Hz, 1H), 4.48 (s, 2H), 6.69 (tt, J=9.00, 2.35 Hz, 1H), 6.83-6.92 (m, 2H).

### Synthesis of 2-(2,6-difluoro-4-(isopropoxymethyl)phenyl)-4,4,5,5-tetramethyl-1,3,2-dioxaborolane

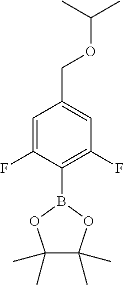 [SMILES: CC(C)OCc1cc(F)c(B2OC(C)(C)C(C)(C)O2)c(F)c1]

Method 3 was followed using 2-isopropoxy-4,4,5,5-tetramethyl-1,3,2-dioxaborolane (1.5 equiv.), butyllithium (1.5 equiv.) and 1,3-difluoro-5-(isopropoxymethyl)benzene (1.0 equiv.) to give 2-(2,6-difluoro-4-(isopropoxymethyl)phenyl)-4,4,5,5-tetramethyl-1,3,2-dioxaborolane in 95% yield.

### Synthesis of methyl 6-(2,6-difluoro-4-(isopropoxymethyl)phenyl)-5-fluoropicolinate

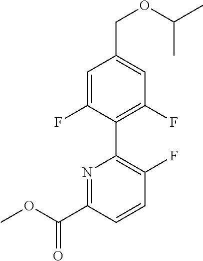 [SMILES: COC(=O)c1ccc(F)c(-c2c(F)cc(COC(C)C)cc2F)n1]

Method 1 was followed using methyl 6-bromo-5-fluoropicolinate (1.0 equiv.) and 2-(2,6-difluoro-4-(isopropoxymethyl)phenyl)-4,4,5,5-tetramethyl-1,3,2-dioxaborolane (2.5 equiv.) at 90° C. for 1 hr to give methyl 6-(2,6-difluoro-4-(isopropoxymethyl)phenyl)-5-fluoropicolinate in 61% yield. LC/MS=340.2 (MH+), Rt=0.99 min.

### Synthesis of 6-(2,6-difluoro-4-(isopropoxymethyl)phenyl)-5-fluoropicolinic acid

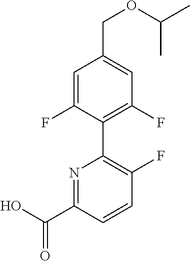 [SMILES: CC(C)OCc1cc(F)c(-c2nc(C(=O)O)ccc2F)c(F)c1]

Method 2 was followed using methyl 6-(2,6-difluoro-4-(isopropoxymethyl)phenyl)-5-fluoropicolinate to give 6-(2,6-difluoro-4-(isopropoxymethyl)phenyl)-5-fluoropicolinic acid in 96% yield. LC/MS=326.2 (MH+), Rt=0.87 min.

### Synthesis of 4-((3,5-difluorobenzyl)oxy)tetrahydro-2H-pyran

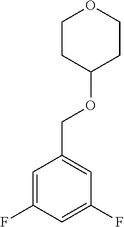 [SMILES: Fc1cc(F)cc(COC2CCOCC2)c1]

Tetrahydro-2H-pyran-4-ol (1.0 equiv.) was dissolved in DMF (0.20 M). Sodium hydride, 60% in mineral oil (1.1 equiv.) was added. The reaction mixture was stirred at ambient temperature for 1 hr. 3,5-difluorobenzyl bromide (1.1 equiv.) was added in a dropwise fashion. The mixture was stirred overnight at ambient temperature. The reaction mixture was quenched by the addition of water. The mixture was extracted with ether. The combined extracts were washed sequentially with water and brine, dried over sodium sulfate, filtered, and concentrated. The crude material was purified by flash chromatography over silica gel (5:2 pentane:ether) to give 4-((3,5-difluorobenzyl)oxy)tetrahydro-2H-pyran in 49% yield. ¹H NMR (400 MHz, CHLOROFORM-d) δ ppm 1.61-1.72 (m, 2H), 1.89-1.98 (m, 2H), 3.46 (ddd, J=11.64, 9.49, 2.74 Hz, 2H), 3.59 (tt, J=8.66, 4.26 Hz, 1H), 3.97 (dt, J=11.74, 4.50 Hz, 2H), 4.54 (s, 2H), 6.71 (tt, J=8.95, 2.20 Hz, 1H), 6.83-6.92 (m, 2H).

### Synthesis of 2-(2,6-difluoro-4-(((tetrahydro-2H-pyran-4-yl)oxy)methyl)phenyl)-4,4,5,5-tetramethyl-1,3,2-dioxaborolane

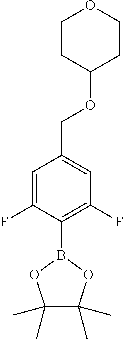 [SMILES: CC1(C)OB(c2c(F)cc(COC3CCOCC3)cc2F)OC1(C)C]

Method 3 was followed using 2-isopropoxy-4,4,5,5-tetramethyl-1,3,2-dioxaborolane (1.6 equiv.), butyllithium (1.6 equiv.) and 4-((3,5-difluorobenzyl)oxy)tetrahydro-2H-pyran (1.0 equiv.) to give 2-(2,6-difluoro-4-(((tetrahydro-2H-pyran-4-yl)oxy)methyl)phenyl)-4,4,5,5-tetramethyl-1,3,2-dioxaborolane in 97% yield.

### Synthesis of methyl 6-(2,6-difluoro-4-(((tetrahydro-2H-pyran-4-yl)oxy)methyl)phenyl)-5-fluoropicolinate

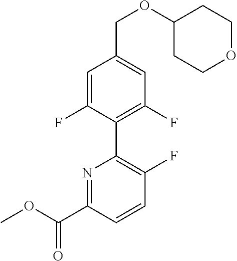 [SMILES: COC(=O)c1ccc(F)c(-c2c(F)cc(COC3CCOCC3)cc2F)n1]

Method 1 was followed using methyl 6-bromo-5-fluoropicolinate (1.0 equiv.) and 2-(2,6-difluoro-4-((tetrahydro-2H-pyran-4-yloxy)methyl)phenyl)-4,4,5,5-tetramethyl-1,3,2-dioxaborolane (2.5 equiv.) at 90° C. for 1 hr to give methyl 6-(2,6-difluoro-4-(((tetrahydro-2H-pyran-4-yl)oxy)methyl)phenyl)-5-fluoropicolinate in 98% yield. LC/MS=382.2 (MH+), Rt=0.88 min.

### Synthesis of 6-(2,6-difluoro-4-(((tetrahydro-2H-pyran-4-yl)oxy)methyl)phenyl)-5-fluoropicolinic acid

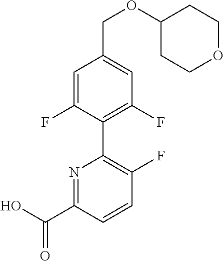 [SMILES: O=C(O)c1ccc(F)c(-c2c(F)cc(COC3CCOCC3)cc2F)n1]

Method 2 was followed using methyl 6-(2,6-difluoro-4-(((tetrahydro-2H-pyran-4-yl)oxy)methyl)phenyl)-5-fluoropicolinate to give 6-(2,6-difluoro-4-(((tetrahydro-2H-pyran-4-yl)oxy)methyl)phenyl)-5-fluoropicolinic acid in 97% yield. LC/MS=368.1 (MH+), Rt=0.77 min.

### Synthesis of methyl 6-(2,6-difluoro-4-((2-oxopyrrolidin-1-yl)methyl)phenyl)-5-fluoropicolinate

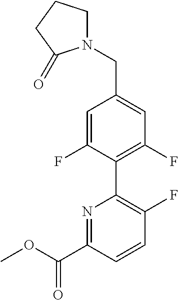 [SMILES: COC(=O)c1ccc(F)c(-c2c(F)cc(CN3CCCC3=O)cc2F)n1]

To a solution of methyl 6-(2,6-difluoro-4-formylphenyl)-5-fluoropicolinate (1.0 equiv.) in MeOH (0.10 M) was added methyl 4-aminobutanoate (1.2 equiv.), followed by TEA (1.4 equiv.). The homogeneous solution was stirred at rt for 30 min, then sodium borohydride (1.0 equiv.) was added. The reaction was heated to 45° C. for 2 days. Upon cooling to rt, the mixture was diluted with water, concentrated the volatiles in vacuo and partitioned between ethyl acetate and water. The organics were dried with sodium sulfate, filtered and concentrated to yield methyl 6-(2,6-difluoro-4-((2-oxopyrrolidin-1-yl)methyl)phenyl)-5-fluoropicolinate in 100% yield. The crude material was used for the next step without further purification. LC/MS=365.2 (MH+), Rt=0.75 min.

### Synthesis of 6-(2,6-difluoro-4-((2-oxopyrrolidin-1-yl)methyl)phenyl)-5-fluoropicolinic acid

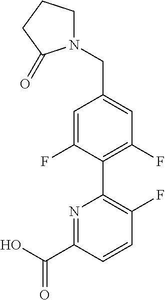 [SMILES: O=C(O)c1ccc(F)c(-c2c(F)cc(CN3CCCC3=O)cc2F)n1]

Method 2 was followed using methyl 6-(2,6-difluoro-4-((2-oxopyrrolidin-1-yl)methyl)phenyl)-5-fluoropicolinate to give 6-(2,6-difluoro-4-((2-oxopyrrolidin-1-yl)methyl)phenyl)-5-fluoropicolinic acid in 75% yield. LC/MS=351.1 (MH+), Rt=0.65 min.

### Synthesis of 1-(3,5-difluorophenyl)cyclopentanol

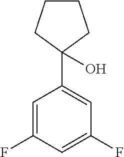 [SMILES: OC1(c2cc(F)cc(F)c2)CCCC1]

To a solution of Mg (6.7 equiv.) in THF (0.14 M) under nitrogen at 0° C. was added 1,4-dibromo butane (3.5 equiv.) dropwise. The reaction was allowed to warm to rt. After stirring for 1 hr at rt, the reaction was cooled to 0° C. and methyl 3,5-difluorobenzoate (1.0 equiv.) in THF (0.14 M) was added dropwise. The cloudy solution became clear and allowed to warm to rt. After 1 hr, the reaction was quenched by the addition of NH4Cl (sat.) and extracted with ethyl acetate. The organic phase was dried with sodium sulfate, filtered and concentrated. The crude material was purified via ISCO SiO2 chromatography (ethyl acetate and heptanes 0-20% ethyl acetate). The pure fractions were concentrated to give 1-(3,5-difluorophenyl)cyclopentanolin 100% yield. 1H NMR (400 MHz, <cdcl3>) δ ppm 1.77-2.11 (m, 8H), 6.67 (tt, J=8.80, 2.35 Hz, 1H), 6.92-7.08 (m, 2H).

### Synthesis of 1-(3,5-difluoro-4-(4,4,5,5-tetramethyl-1,3,2-dioxaborolan-2-yl)phenyl)cyclopentanol

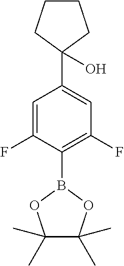 [SMILES: CC1(C)OB(c2c(F)cc(C3(O)CCCC3)cc2F)OC1(C)C]

Method 3 was followed using 2-isopropoxy-4,4,5,5-tetramethyl-1,3,2-dioxaborolane (2.5 equiv.), butyllithium (2.4 equiv.) and 1-(3,5-difluorophenyl)cyclopentanol (1.0 equiv.) to give 1-(3,5-difluoro-4-(4,4,5,5-tetramethyl-1,3,2-dioxaborolan-2-yl)phenyl)cyclopentanol in 100% yield. 1H NMR (400 MHz, <cdcl3>) δ ppm 1.24 (s, 12H), 1.80-2.04 (m, 8H), 6.97 (d, J=9.00 Hz, 2H).

### Synthesis of methyl 6-(2,6-difluoro-4-(1-hydroxycyclopentyl)phenyl)-5-fluoropicolinate

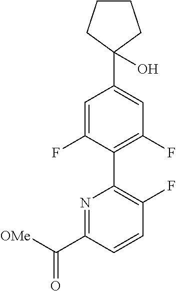 [SMILES: COC(=O)c1ccc(F)c(-c2c(F)cc(C3(O)CCCC3)cc2F)n1]

Method 1 was followed using methyl 6-bromo-5-fluoropicolinate (1.0 equiv.) and 1-(3,5-difluoro-4-(4,4,5,5-tetramethyl-1,3,2-dioxaborolan-2-yl)phenyl)cyclopentanol (1.3 equiv.) at 100° C. for 20 min in microwave to give methyl 6-(2,6-difluoro-4-(1-hydroxycyclopentyl)phenyl)-5-fluoropicolinate in 97% yield. LC/MS=352.2 (MH+), Rt=0.88 min. 1H NMR (400 MHz, <cdcl3>) δ ppm 1.80-2.12 (m, 8H), 4.00 (s, 3H), 7.16 (d, J=9.39 Hz, 2H), 7.65 (t, J=8.41 Hz, 1H), 8.26 (dd, J=8.61, 3.91 Hz, 1H).

### Synthesis of 6-(2,6-difluoro-4-(1-hydroxycyclopentyl)phenyl)-5-fluoropicolinic acid

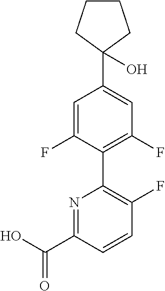 [SMILES: O=C(O)c1ccc(F)c(-c2c(F)cc(C3(O)CCCC3)cc2F)n1]

Method 2 was followed using methyl 6-(2,6-difluoro-4-(1-hydroxycyclopentyl)phenyl)-5-fluoropicolinate to give 6-(2,6-difluoro-4-(1-hydroxycyclopentyl)phenyl)-5-fluoropicolinic acid in 83% yield. LC/MS=338.2 (MH+), Rt=0.78 min.

### Synthesis of 4-(3,5-difluorophenyl)-3,5-dimethylisoxazole

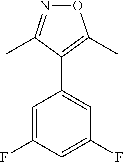

4-bromo-3,5-dimethylisoxazole (1.0 equiv.), 3,5-difluorophenylboronic acid (1.3 equiv.), and PdCl₂(dppf). CH₂Cl₂ adduct (0.1 equiv.) were combined in a microwave vial and 1,4-Dioxane (0.3 M) was added followed by 2M sodium carbonate (2.0 equiv.). The mixture was purged with N₂, sealed and heated at 120° C. for 40 min in the microwave. The mixture was partitioned between EtOAc and brine. The organic layer was dried over sodium sulfate, filtered and concentrated to afford a black solid. The crude black material was purified by ISCO SiO₂ chromatography eluting with 0-100% DCM in Heptanes to afford 4-(3,5-difluorophenyl)-3,5-dimethylisoxazole in 60% yield. LC/MS (m/z): 210.1 (MH+), Rt=0.88 min. 1H NMR (400 MHz, <cdcl3>) δ 6.73-6.87 (m, 3H), 2.43 (s, 3H), 2.29 (s, 3H).

### Synthesis of 4-(3,5-difluoro-4-(4,4,5,5-tetramethyl-1,3,2-dioxaborolan-2-yl)phenyl)-3,5-dimethylisoxazole

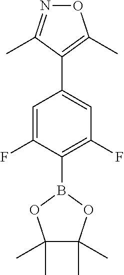 [SMILES: Cc1noc(C)c1-c1cc(F)c(B2OC(C)(C)C(C)(C)O2)c(F)c1]

Method 3 was followed using 2-isopropoxy-4,4,5,5-tetramethyl-1,3,2-dioxaborolane (2.0 equiv.), butyllithium (1.05 equiv.) and 4-(3,5-difluorophenyl)-3,5-dimethylisoxazole (1.0 equiv.) to give 4-(3,5-difluoro-4-(4,4,5,5-tetramethyl-1,3,2-dioxaborolan-2-yl)phenyl)-3,5-dimethylisoxazole in 97% yield. 1H NMR (400 MHz, CHLOROFORM-d) δ ppm 1.38-1.42 (s, 12H), 2.28 (s, 3H), 2.43 (s, 3H), 6.76 (d, J=8.22 Hz, 2H).

### Synthesis of methyl 6-(4-(3,5-dimethylisoxazol-4-yl)-2,6-difluorophenyl)-5-fluoropicolinate

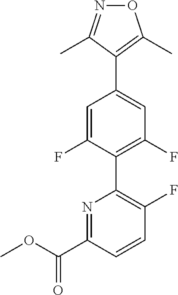 [SMILES: COC(=O)c1ccc(F)c(-c2c(F)cc(-c3c(C)noc3C)cc2F)n1]

Method 1 was followed using methyl 6-bromo-5-fluoropicolinate (1.0 equiv.) and 4-(3,5-difluoro-4-(4,4,5,5-tetramethyl-1,3,2-dioxaborolan-2-yl)phenyl)-3,5-dimethylisoxazole (2.5 equiv.) at 80° C. for 15 min in microwave to give methyl 6-(4-(3,5-dimethylisoxazol-4-yl)-2,6-difluorophenyl)-5-fluoropicolinate in 89% yield. LC/MS=363.1 (MH+), Rt=0.90 min.

### Synthesis of 6-(4-(3,5-dimethylisoxazol-4-yl)-2,6-difluorophenyl)-5-fluoropicolinic acid

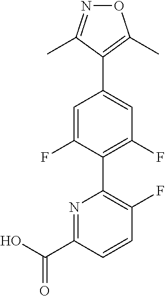 [SMILES: Cc1noc(C)c1-c1cc(F)c(-c2nc(C(=O)O)ccc2F)c(F)c1]

Method 2 was followed using methyl 6-(4-(3,5-dimethylisoxazol-4-yl)-2,6-difluorophenyl)-5-fluoropicolinate to give 6-(4-(3,5-dimethylisoxazol-4-yl)-2,6-difluorophenyl)-5-fluoropicolinic acid in 63% yield. LC/MS=349.2 (MH+), Rt=0.80 min.

### Synthesis of tert-butyl 2-(3,5-difluorophenyl)-2-methylpropanoate

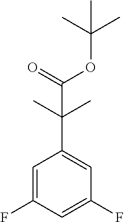 [SMILES: CC(C)(C)OC(=O)C(C)(C)c1cc(F)cc(F)c1]

To a solution of 2-(3,5-difluorophenyl)-2-methylpropanoic acid (1.0 equiv.) dissolved in DCM (0.20 M) was added oxalyl chloride (1.8 equiv.) followed by 5 drops of DMF. The mixture was stirred at rt for 30 min and then the solvents were removed in vacuo. The residue was taken up in THF (0.20 M) and cooled to 0° C. on an ice bath. Potassium tert-butoxide (1.2 equiv., 1M solution in THF) was added drop wise over 10 min. The reaction was stirred for 18 hrs. The reaction was diluted with ether and washed with water, brine, dried over sodium sulfate, filtered and concentrated to yield tert-butyl 2-(3,5-difluorophenyl)-2-methylpropanoate in 97% yield. 1H NMR (400 MHz, CHLOROFORM-d) δ ppm 1.39 (s, 9H), 1.50 (s, 6H), 6.67 (s, 1H), 6.86 (dd, J=9.00, 1.96 Hz, 2H).

### Synthesis of tert-butyl 2-(3,5-difluoro-4-(4,4,5,5-tetramethyl-1,3,2-dioxaborolan-2-yl)phenyl)-2-methylpropanoate

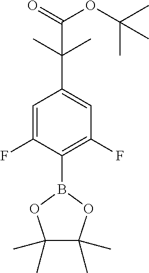 [SMILES: CC(C)(C)OC(=O)C(C)(C)c1cc(F)c(B2OC(C)(C)C(C)(C)O2)c(F)c1]

Method 3 was followed using 2-isopropoxy-4,4,5,5-tetramethyl-1,3,2-dioxaborolane (2.2 equiv.), butyllithium (1.1 equiv.) and tert-butyl 2-(3,5-difluorophenyl)-2-methylpropanoate (1.0 equiv.) to give tert-butyl 2-(3,5-difluoro-4-(4,4,5,5-tetramethyl-1,3,2-dioxaborolan-2-yl)phenyl)-2-methylpropanoate in 100% yield. 1H NMR (400 MHz, CHLOROFORM-d) δ ppm 1.27 (s, 9H), 1.36 (s, 12H), 1.48 (s, 6H), 6.83 (d, J=9.39 Hz, 2H).

### Synthesis of methyl 6-(4-(1-(tert-butoxy)-2-methyl-1-oxopropan-2-yl)-2,6-difluorophenyl)-5-fluoropicolinate

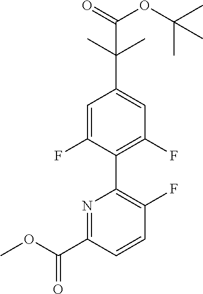 [SMILES: COC(=O)c1ccc(F)c(-c2c(F)cc(C(C)(C)C(=O)OC(C)(C)C)cc2F)n1]

Method 1 was followed using methyl 6-bromo-5-fluoropicolinate (1.0 equiv.) and tert-butyl 2-(3,5-difluoro-4-(4,4,5,5-tetramethyl-1,3,2-dioxaborolan-2-yl)phenyl)-2-methylpropanoate (2.0 equiv.) at 80° C. for 15 min in microwave to give methyl 6-(4-(1-(tert-butoxy)-2-methyl-1-oxopropan-2-yl)-2,6-difluorophenyl)-5-fluoropicolinate in 73% yield. LC/MS=410.1 (MH+), Rt=1.11 min.

### Synthesis of 6-(4-(1-(tert-butoxy)-2-methyl-1-oxopropan-2-yl)-2,6-difluorophenyl)-5-fluoropicolinic acid

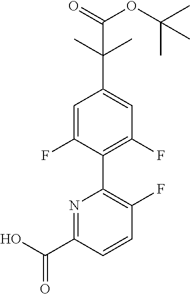 [SMILES: CC(C)(C)OC(=O)C(C)(C)c1cc(F)c(-c2nc(C(=O)O)ccc2F)c(F)c1]

Method 2 was followed using methyl 6-(4-(1-(tert-butoxy)-2-methyl-1-oxopropan-2-yl)-2,6-difluorophenyl)-5-fluoropicolinate to give 6-(4-(1-(tert-butoxy)-2-methyl-1-oxopropan-2-yl)-2,6-difluorophenyl)-5-fluoropicolinic acid in 82% yield. LC/MS=396.1 (MH+), Rt=1.00 min.

### Synthesis of methyl 6-(2,6-difluoro-4-(3-methoxypropoxy)phenyl)-5-fluoropicolinate

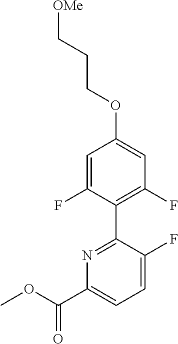 [SMILES: COCCCOc1cc(F)c(-c2nc(C(=O)OC)ccc2F)c(F)c1]

To a solution of triphenylphosphine (2.0 equiv.), methyl 6-(2,6-difluoro-4-hydroxyphenyl)-5-fluoropicolinate (1.0 equiv.) and 3-methoxypropan-1-ol (1.2 equiv.) in THF (0.14 M) was added DIAD (2.0 equiv.) dropwise. The mixture was allowed to stir overnight at rt. The reaction was concentrated to dryness and purified via silica gel column chromatography (ISCO, ethyl acetate and heptanes 0-50% ethyl acetate). The pure fractions were concentrated to yield methyl 6-(2,6-difluoro-4-(3-methoxypropoxy)phenyl)-5-fluoropicolinate in 100% yield. LC/MS=356.1 (MH+), Rt=0.93 min.

### Synthesis of 6-(2,6-difluoro-4-(3-methoxypropoxy)phenyl)-5-fluoropicolinic acid

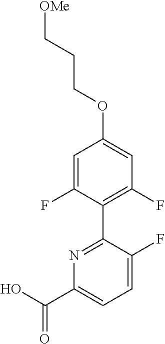 [SMILES: COCCCOc1cc(F)c(-c2nc(C(=O)O)ccc2F)c(F)c1]

Method 2 was followed using methyl 6-(2,6-difluoro-4-(3-methoxypropoxy)phenyl)-5-fluoropicolinate to give 6-(2,6-difluoro-4-(3-methoxypropoxy)phenyl)-5-fluoropicolinic acid 64% yield. LC/MS=342.1 (MH+), Rt=0.83 min.

### Synthesis of 2-(5,7-difluoro-2,3-dihydrobenzofuran-6-yl)-4,4,5,5-tetramethyl-1,3,2-dioxaborolane

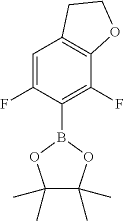 [SMILES: CC1(C)OB(c2c(F)cc3c(c2F)OCC3)OC1(C)C]

Method 3 was followed using 2-isopropoxy-4,4,5,5-tetramethyl-1,3,2-dioxaborolane (1.3 equiv.), butyllithium (1.3 equiv.) and 5,7-difluoro-2,3-dihydrobenzofuran (1.0 equiv.) to give 2-(5,7-difluoro-2,3-dihydrobenzofuran-6-yl)-4,4,5,5-tetramethyl-1,3,2-dioxaborolane in 30% yield. 1H NMR (400 MHz, CHLOROFORM-d) δ ppm 1.37 (s, 12H), 3.24 (td, J=8.71, 4.11 Hz, 2H), 4.51-4.78 (m, 2H) 6.70 (d, J=7.43 Hz, 1H).

### Synthesis of methyl 6-(5,7-difluoro-2,3-dihydrobenzofuran-6-yl)-5-fluoropicolinate

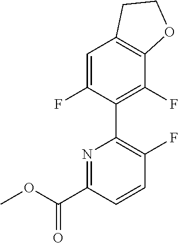 [SMILES: COC(=O)c1ccc(F)c(-c2c(F)cc3c(c2F)OCC3)n1]

Method 1 was followed using methyl 6-bromo-5-fluoropicolinate (1.0 equiv.) and 2-(5,7-difluoro-2,3-dihydrobenzofuran-6-yl)-4,4,5,5-tetramethyl-1,3,2-dioxaborolane (1.5 equiv.) at 90° C. for 90 min in oil bath to give methyl 6-(5,7-difluoro-2,3-dihydrobenzofuran-6-yl)-5-fluoropicolinate in 90% yield. LC/MS=310.1 (MH+), Rt=0.86 min.

### Synthesis of 6-(5,7-difluoro-2,3-dihydrobenzofuran-6-yl)-5-fluoropicolinic acid

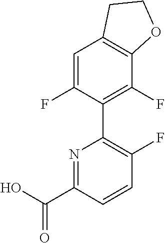 [SMILES: O=C(O)c1ccc(F)c(-c2c(F)cc3c(c2F)OCC3)n1]

Method 2 was followed using methyl 6-(5,7-difluoro-2,3-dihydrobenzofuran-6-yl)-5-fluoropicolinate to give 6-(5,7-difluoro-2,3-dihydrobenzofuran-6-yl)-5-fluoropicolinic acid 90% yield. LC/MS=296.1 (MH+), Rt=0.73 min.

### Synthesis of methyl 6-(2,6-difluoro-4-((tetrahydro-2H-pyran-4-yl)methoxy)phenyl)-5-fluoropicolinate

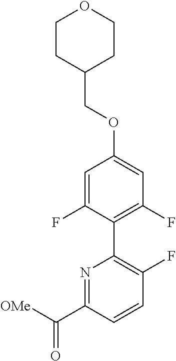 [SMILES: COC(=O)c1ccc(F)c(-c2c(F)cc(OCC3CCOCC3)cc2F)n1]

A mixture of methyl 6-(2,6-difluoro-4-hydroxyphenyl)-5-fluoropicolinate (1.0 equiv.), 4-(bromomethyl)tetrahydro-2H-pyran (2.0 equiv.) and K₂CO₃ (4.0 equiv.) in DMF (0.20 M) was heated at 100° C. for 20 min in microwave. The reaction mixture was cooled off to rt and partitioned between EtOAc and H₂O. The organic layer was washed with brine, dried over Na2SO4 and concentrated to give methyl 6-(2,6-difluoro-4-((tetrahydro-2H-pyran-4-yl)methoxy)phenyl)-5-fluoropicolinate in 100% yield. LC/MS=382.0 (MH+), Rt=0.97 min.

### Synthesis of 6-(2,6-difluoro-4-((tetrahydro-2H-pyran-4-yl)methoxy)phenyl)-5-fluoropicolinic acid

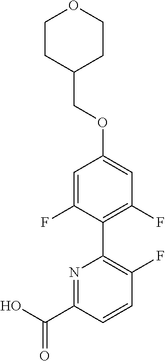 [SMILES: O=C(O)c1ccc(F)c(-c2c(F)cc(OCC3CCOCC3)cc2F)n1]

Method 2 was followed using methyl 6-(2,6-difluoro-4-((tetrahydro-2H-pyran-4-yl)methoxy)phenyl)-5-fluoropicolinate to give 6-(2,6-difluoro-4-((tetrahydro-2H-pyran-4-yl)methoxy)phenyl)-5-fluoropicolinic acid in 81% yield. LC/MS=368.0 (MH+), Rt=0.85 min.

### Synthesis of methyl 2′,6,6′-trifluoro-4′-(trifluoromethylsulfonyloxy)biphenyl-3-carboxylate

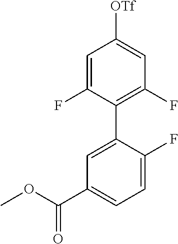 [SMILES: COC(=O)c1ccc(F)c(-c2c(F)cc(OS(=O)(=O)C(F)(F)F)cc2F)c1]

To a solution of methyl 2′,6,6′-trifluoro-4′-hydroxybiphenyl-3-carboxylate (1.0 equiv.) in DCM (0.35 M) at 0° C. was added pyridine (1.5 equiv.) and allowed to stir for 5 mins, followed by the addition of TriflicAnhydride (1.1 equiv.). The reaction was allowed to stir warming to RT. The reaction was quenched with NaHCO3(sat), extracted in DCM and the organics were washed with water and brine. The organics were dried over Na2SO4, filtered, and concentrated to yield methyl 2′,6,6′-trifluoro-4′-(trifluoromethylsulfonyloxy)biphenyl-3-carboxylate in 81% yield.

### Synthesis of methyl 6-(4-(3,6-dihydro-2H-thiopyran-4-yl)-2,6-difluorophenyl)-5-fluoropicolinate

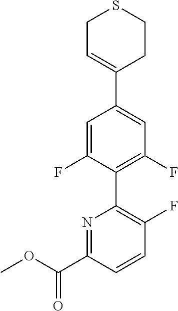 [SMILES: COC(=O)c1ccc(F)c(-c2c(F)cc(C3=CCSCC3)cc2F)n1]

To a degassed solution of methyl 6-(2,6-difluoro-4-(trifluoromethylsulfonyloxy)phenyl)-5-fluoropicolinate (1.0 equiv.) and 3,6-dihydro-2H-thiopyran-4-ylboronic acid (1.5 equiv.) in DME/2M Na2CO3 (3/1, 0.10 M) was added PdCl2(dppf). CH2Cl2 adduct (0.10 equiv.). The reaction was heated to 90° C. in an oil bath for 15 min. The reaction mixture was partitioned with water and EtOAc; the organics were dried over MgSO4, filtered, and concentrated. The crude was purified via ISCO. Pure fractions were combined and concentrated to yield methyl 6-(4-(3,6-dihydro-2H-thiopyran-4-yl)-2,6-difluorophenyl)-5-fluoropicolinate in 60% yield. LC/MS=366.1 (M+H), Rt=1.00 min.

### Synthesis of methyl 6-(4-(1,1-dioxido-3,6-dihydro-2H-thiopyran-4-yl)-2,6-difluorophenyl)-5-fluoropicolinate

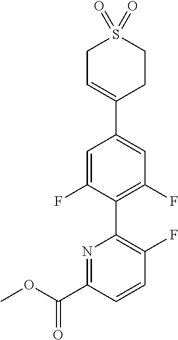 [SMILES: COC(=O)c1ccc(F)c(-c2c(F)cc(C3=CCS(=O)(=O)CC3)cc2F)n1]

To a solution of methyl 6-(4-(3,6-dihydro-2H-thiopyran-4-yl)-2,6-difluorophenyl)-5-fluoropicolinate (1.0 equiv.) in DCM (0.10 M) at rt was added oxone (6.0 equiv.) in one portion. The resulting mixture was stirred at RT overnight, and then refluxed at 40° C. for 4 hrs. 10.0 equiv. of oxone were added and the reaction was allowed to stir at 40° C. over the weekend. The reaction mixture was then diluted with DCM and washed with water the aqueous layer was then separated and extracted with DCM. The combined organic were then dried over MgSO4 and concentrated in vacuo to yield methyl 6-(4-(1,1-dioxido-3,6-dihydro-2H-thiopyran-4-yl)-2,6-difluorophenyl)-5-fluoropicolinate in 100% yield. LC/MS=398.0 (M+H), Rt=0.76 min.

### Synthesis of 6-(4-(1,1-dioxido-3,6-dihydro-2H-thiopyran-4-yl)-2,6-difluorophenyl)-5-fluoropicolinic acid

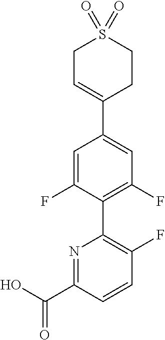 [SMILES: O=C(O)c1ccc(F)c(-c2c(F)cc(C3=CCS(=O)(=O)CC3)cc2F)n1]

Method 2 was followed using methyl 6-(4-(1,1-dioxido-3,6-dihydro-2H-thiopyran-4-yl)-2,6-difluorophenyl)-5-fluoropicolinate to give 6-(4-(1,1-dioxidotetrahydro-2H-thiopyran-4-yl)-2,6-difluorophenyl)-5-fluoropicolinic acid in 74% yield. LC/MS=384.0 (M+H), Rt=0.64 min.

### Synthesis of 6-(4-(1,1-dioxidotetrahydro-2H-thiopyran-4-yl)-2,6-difluorophenyl)-5-fluoropicolinic acid

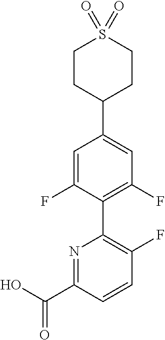 [SMILES: O=C(O)c1ccc(F)c(-c2c(F)cc(C3CCS(=O)(=O)CC3)cc2F)n1]

To a degassed solution of 6-(4-(1,1-dioxido-3,6-dihydro-2H-thiopyran-4-yl)-2,6-difluorophenyl)-5-fluoropicolinic acid (1.0 equiv.) in EtOH (0.10 M) was added Pd/C (0.1 equiv.). The mixture was stirred at rt under H2 for 16 hrs. Add Pd/C (0.1 equiv.) and the reaction was stirred for additional 16 hrs. The reaction was taken up and filtered through a syringe filter. The combined organics were concentrated to yield 6-(4-(1,1-dioxidotetrahydro-2H-thiopyran-4-yl)-2,6-difluorophenyl)-5-fluoropicolinic acid in 100% yield. LC/MS=386.0 (M+H), Rt=0.64 min.

### Synthesis of methyl 6-(2,6-difluoro-4-(2,2,2-trifluoroethoxy)phenyl)-5-fluoropicolinate

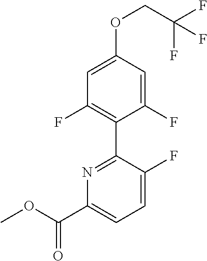 [SMILES: COC(=O)c1ccc(F)c(-c2c(F)cc(OCC(F)(F)F)cc2F)n1]

To a solution of methyl 6-(2,6-difluoro-4-hydroxyphenyl)-5-fluoropicolinate (1.0 equiv.) in DMF (0.35 M) was added potassium carbonate (3.0 equiv.) and 2,2,2-trifluoroethyl trifluoromethanesulfonate (1.2 equiv.). The mixture was stirred at ambient temperature for 3 hrs. The reaction mixture was diluted with ethyl acetate, and filtered. The filtrate was washed with water and brine, concentrated, and purified by flash chromatography to give methyl 6-(2,6-difluoro-4-(2,2,2-trifluoroethoxy)phenyl)-5-fluoropicolinate in 100% yield. LC/MS=366.0 (M+H), Rt=0.95 min.

### Synthesis of 6-(2,6-difluoro-4-(2,2,2-trifluoroethoxy)phenyl)-5-fluoropicolinic acid

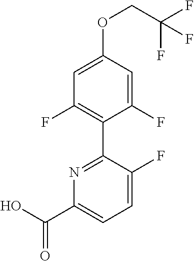 [SMILES: O=C(O)c1ccc(F)c(-c2c(F)cc(OCC(F)(F)F)cc2F)n1]

Method 2 was followed using methyl 6-(2,6-difluoro-4-(2,2,2-trifluoroethoxy)phenyl)-5-fluoropicolinate to give 6-(2,6-difluoro-4-(2,2,2-trifluoroethoxy)phenyl)-5-fluoropicolinic acid in 100% yield. LC/MS=352.1 (M+H), Rt=0.85 min.

### Synthesis of methyl 6-(2,6-difluoro-4-(prop-1-en-2-yl)phenyl)-5-fluoropicolinate

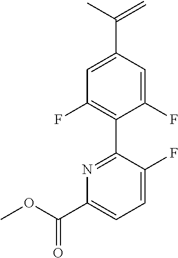 [SMILES: C=C(C)c1cc(F)c(-c2nc(C(=O)OC)ccc2F)c(F)c1]

To a degassed solution of methyl 6-(2,6-difluoro-4-(trifluoromethylsulfonyloxy)phenyl)-5-fluoropicolinate (1.0 equiv.) in DME/2M Na2CO3 (3/1, 0.09 M) was added 4,4,5,5-tetramethyl-2-(prop-1-en-2-yl)-1,3,2-dioxaborolane (1.5 equiv.) and PdCl2(dppf)-CH2Cl2Adduct (0.1 equiv.), followed by. The reaction was heated to 90° C. in an oil bath for 15 min. The mixture was cooled to rt and partitioned between water and ethyl acetate. The organic phase was dried with sodium sulfate, filtered and concentrated. The crude material was purified via silica gel column chromatography (Analogix, eluting with 0-100% ethyl acetate). The pure fractions were concentrated to yield methyl 6-(2,6-difluoro-4-(prop-1-en-2-yl)phenyl)-5-fluoropicolinate. LC/MS=308.2 (M+H), Rt=0.99 min. 1H NMR (400 MHz, <cdcl3>) δ ppm 2.15 (s, 3H), 4.01 (s, 3H), 5.23 (s, 1H), 5.47 (s, 1H), 7.11 (d, J=9.39 Hz, 2H), 7.65 (t, J=8.41 Hz, 1H), 8.26 (dd, J=8.61, 3.91 Hz, 1H).

### Synthesis of methyl 6-(2,6-difluoro-4-isopropylphenyl)-5-fluoropicolinate

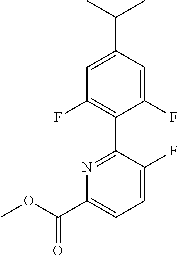 [SMILES: COC(=O)c1ccc(F)c(-c2c(F)cc(C(C)C)cc2F)n1]

To a degassed solution of methyl 6-(2,6-difluoro-4-(prop-1-en-2-yl)phenyl)-5-fluoropicolinate (1.0 equiv.) in MeOH (0.09 M) was added Pd/C (0.1 equiv.) and the reaction was stirred at rt under an atmosphere of hydrogen. After overnight stirring, filtered through a pad of Celite and washed with Methanol. The filtrate was concentrated and dried under vacuo to give methyl 6-(2,6-difluoro-4-isopropylphenyl)-5-fluoropicolinate. LC/MS=310.0 (M+H), Rt=1.00 min.

### Synthesis of 6-(2,6-difluoro-4-isopropylphenyl)-5-fluoropicolinic acid

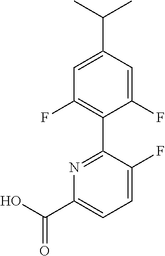 [SMILES: CC(C)c1cc(F)c(-c2nc(C(=O)O)ccc2F)c(F)c1]

Method 2 was followed using methyl 6-(2,6-difluoro-4-isopropylphenyl)-5-fluoropicolinate to give 6-(2,6-difluoro-4-isopropylphenyl)-5-fluoropicolinic acid in 100% yield. LC/MS=296.2 (M+H), Rt=0.89 min.

### Synthesis of methyl 6-(4-((1-(tert-butoxycarbonyl)piperidin-4-yl)oxy)-2,6-difluorophenyl)-5-fluoropicolinate

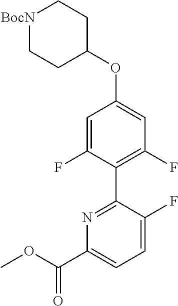 [SMILES: COC(=O)c1ccc(F)c(-c2c(F)cc(OC3CCN(C(=O)OC(C)(C)C)CC3)cc2F)n1]

To a solution of methyl 6-(2,6-difluoro-4-hydroxyphenyl)-5-fluoropicolinate (1.0 equiv.), tert-butyl 4-hydroxypiperidine-1-carboxylate (3.0 equiv.) and triphenylphosphine (2.0 equiv.) in THF (0.04 at 0° C. was added DIAD (3.0 equiv.) was added. The mixture was stirred at ambient temperature overnight. The mixture was concentrated and partitioned between EtOAc and Water. The organic layer was washed with sat. NaHCO₃, then brine, dried over Na₂SO4 and concentrated to give methyl 6-(4-((1-(tert-butoxycarbonyl)piperidin-4-yl)oxy)-2,6-difluorophenyl)-5-fluoropicolinate in 100% yield. LC/MS=411.0 (M-tBu+H⁺), Rt=1.12 min.

### Synthesis of 6-(4-((1-(tert-butoxycarbonyl)piperidin-4-yl)oxy)-2,6-difluorophenyl)-5-fluoropicolinic acid

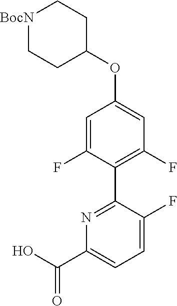 [SMILES: CC(C)(C)OC(=O)N1CCC(Oc2cc(F)c(-c3nc(C(=O)O)ccc3F)c(F)c2)CC1]

Method 2 was followed using methyl 6-(4-((1-(tert-butoxycarbonyl)piperidin-4-yl)oxy)-2,6-difluorophenyl)-5-fluoropicolinate to give 6-(4-((1-(tert-butoxycarbonyl)piperidin-4-yl)oxy)-2,6-difluorophenyl)-5-fluoropicolinic acid in 31% yield. LC/MS (-tBu)=397.0 (M-tBu+H⁺), Rt=1.01 min.

### Synthesis of methyl 6-(4-(1-((benzyloxy)carbonyl)-1,2,3,6-tetrahydropyridin-4-yl)-2,6-difluorophenyl)-5-fluoropicolinate

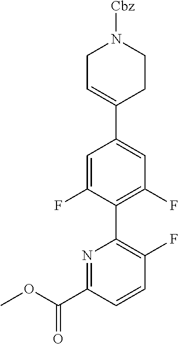 [SMILES: COC(=O)c1ccc(F)c(-c2c(F)cc(C3=CCN(C(=O)OCc4ccccc4)CC3)cc2F)n1]

To a degassed solution of methyl 6-(2,6-difluoro-4-(trifluoromethylsulfonyloxy)phenyl)-5-fluoropicolinate (1.0 equiv.) and benzyl 4-(4,4,5,5-tetramethyl-1,3,2-dioxaborolan-2-yl)-5,6-dihydropyridine-1(2H)-carboxylate (1.5 equiv.) in THF/H2O (3/1, 0.19 M) was added PdCl₂(dppf). CH₂Cl₂ adduct (0.10 equiv.). The reaction was heated at 100° C. in microwave for 15 min. The reaction mixture was partitioned with water and EtOAc; the organics were dried over MgSO₄, filtered, and concentrated. The crude was purified via ISCO. Pure fractions were combined and concentrated to yield methyl 6-(4-(1-((benzyloxy)carbonyl)-1,2,3,6-tetrahydropyridin-4-yl)-2,6-difluorophenyl)-5-fluoropicolinate in 100% yield. LC/MS=483.2 (MH⁺), Rt=1.11 min.

### Synthesis of 6-(4-(1-((benzyloxy)carbonyl)-1,2,3,6-tetrahydropyridin-4-yl)-2,6-difluorophenyl)-5-fluoropicolinic acid

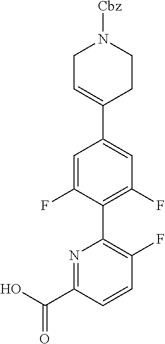 [SMILES: O=C(O)c1ccc(F)c(-c2c(F)cc(C3=CCN(C(=O)OCc4ccccc4)CC3)cc2F)n1]

Method 2 was followed using methyl 6-(4-(1-((benzyloxy)carbonyl)-1,2,3,6-tetrahydropyridin-4-yl)-2,6-difluorophenyl)-5-fluoropicolinate to give 6-(4-(1-((benzyloxy)carbonyl)-1,2,3,6-tetrahydropyridin-4-yl)-2,6-difluorophenyl)-5-fluoropicolinic acid in 98% yield. LC/MS=469.2 (MH⁺), Rt=1.00 min.

### Synthesis of benzyl 4-(4-(6-((4-((1R,3R,4R,5S)-3,4-dihydroxy-4,5-dimethylcyclohexyl)pyridin-3-yl)carbamoyl)-3-fluoropyridin-2-yl)-3,5-difluorophenyl)-5,6-dihydropyridine-1(2H)-carboxylate

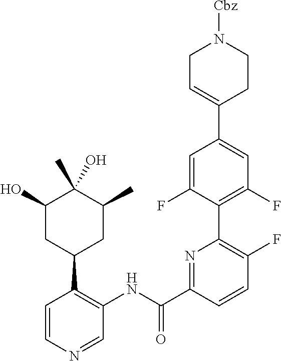 [SMILES: C[C@H]1C[C@@H](c2ccncc2NC(=O)c2ccc(F)c(-c3c(F)cc(C4=CCN(C(=O)OCc5ccccc5)CC4)cc3F)n2)C[C@@H](O)[C@]1(C)O]

Method 6 was followed using (1R,2R,4R,6S)-4-(3-aminopyridin-4-yl)-1,6-dimethylcyclohexane-1,2-diol (1.0 equiv.) and 6-(4-(1-(benzyloxycarbonyl)-1,2,3,6-tetrahydropyridin-4-yl)-2,6-difluorophenyl)-5-fluoropicolinic acid (1.0 equiv.) to give benzyl 4-(4-(6-((4-((1R,3R,4R,5S)-3,4-dihydroxy-4,5-dimethylcyclohexyl)pyridin-3-yl)carbamoyl)-3-fluoropyridin-2-yl)-3,5-difluorophenyl)-5,6-dihydropyridine-1(2H)-carboxylate in 39% yield. LC/MS=687.3 (MH⁺), Rt=0.94 min.

### Synthesis of N-(4-((1R,3R,4R,5S)-3,4-dihydroxy-4,5-dimethylcyclohexyl)pyridin-3-yl)-6-(4-(1-ethylpiperidin-4-yl)-2,6-difluorophenyl)-5-fluoropicolinamide

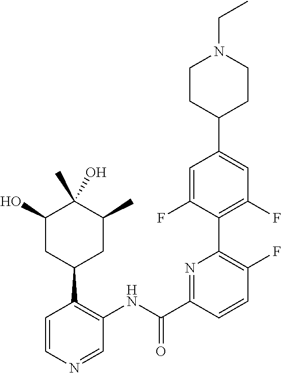 [SMILES: CCN1CCC(c2cc(F)c(-c3nc(C(=O)Nc4cnccc4[C@H]4C[C@@H](O)[C@](C)(O)[C@@H](C)C4)ccc3F)c(F)c2)CC1]

To a degassed solution of benzyl 4-(4-(6-(4-((1R,3R,4R,5S)-3,4-dihydroxy-4,5-dimethylcyclohexyl)pyridin-3-ylcarbamoyl)-3-fluoropyridin-2-yl)-3,5-difluorophenyl)-5,6-dihydropyridine-1(2H)-carboxylate (1.0 equiv.) in EtOH (0.03 M) was added Pd/C (0.5 equiv.). The mixture was allowed to stir under and atm. of H2 overnight. The reaction mixture was filtered and concentrated. The crude was taken up in DMSO and purified via reverse prep-HPLC. Pure factions were combined, flash-frozen, and placed on the lyophilizer to dry to yield N-(4-((1R,3R,4R,5S)-3,4-dihydroxy-4,5-dimethylcyclohexyl)pyridin-3-yl)-6-(4-(1-ethylpiperidin-4-yl)-2,6-difluorophenyl)-5-fluoropicolinamide in 91% yield. LC/MS=583.4 (MH⁺), Rt=0.64 min.

### Synthesis of methyl 6-(2,6-difluoro-4-(pyridin-4-yloxy)phenyl)-5-fluoropicolinate

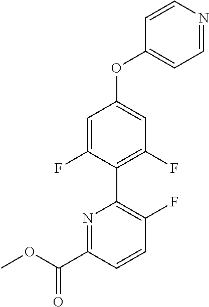 [SMILES: COC(=O)c1ccc(F)c(-c2c(F)cc(Oc3ccncc3)cc2F)n1]

To a solution of methyl 6-(2,6-difluoro-4-hydroxyphenyl)-5-fluoropicolinate (1.0 equiv.), pyridin-4-ylboronic acid (2.0 equiv.) and Cu(OAc)₂ (4.0 equiv.) in DCM (0.04 M) with dry-powdered molecular sieves was added Et₃N (5.0 equiv.). The reaction mixture was stirred at rt overnight and then filtered through a pad of celite and the cake was washed with EtOAc. The organics were concentrated. The crude was purified via reverse prep-HPLC. Pure fractions were combined and free-based with NaHSO₄₍ₛₐₜ₎ and washed with EtOAc. The combined organics were dried over MgSO₄, filtered, and concentrated. LC/MS=361.0 (MH⁺), Rt=0.63 min.

### Synthesis of 6-(2,6-difluoro-4-(pyridin-4-yloxy)phenyl)-5-fluoropicolinic acid

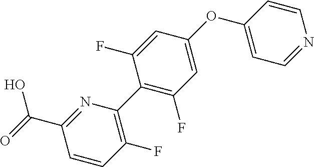 [SMILES: O=C(O)c1ccc(F)c(-c2c(F)cc(Oc3ccncc3)cc2F)n1]

Method 2 was followed using methyl 6-(2,6-difluoro-4-(pyridin-4-yloxy)phenyl)-5-fluoropicolinate to give 6-(2,6-difluoro-4-(pyridin-4-yloxy)phenyl)-5-fluoropicolinic acid in 69% yield. LC/MS=346.9 (MH⁺), Rt=0.54 min.

### Synthesis of ethyl 2-bromo-5-((tert-butoxycarbonyl)amino)thiazole-4-carboxylate

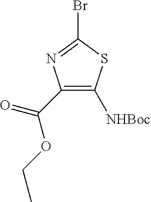 [SMILES: CCOC(=O)c1nc(Br)sc1NC(=O)OC(C)(C)C]

To a solution of ethyl 5-(tert-butoxycarbonylamino)thiazole-4-carboxylate (1.0 equiv.) in DCM (0.20 M) was added NBS (1.6 equiv) at RT. The resulting mixture was stirred at RT for 2 hrs. The reaction mixture was then concentrated in vacuo to give ethyl 2-bromo-5-((tert-butoxycarbonyl)amino)thiazole-4-carboxylate in 100% yield and utilised in the next reaction without further purification. LC/MS=352.9 (MH+), Rt=1.12 min.

### Synthesis of ethyl 5-((tert-butoxycarbonyl)amino)-2-(2,6-difluoro-4-(tetrahydro-2H-pyran-4-yl)phenyl)thiazole-4-carboxylate

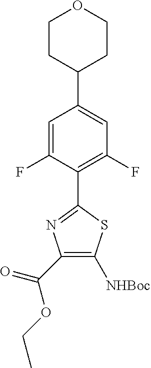 [SMILES: CCOC(=O)c1nc(-c2c(F)cc(C3CCOCC3)cc2F)sc1NC(=O)OC(C)(C)C]

Method 1 was followed using ethyl 2-bromo-5-(tert-butoxycarbonylamino)thiazole-4-carboxylate (1.0 equiv.) and 2-(2,6-difluoro-4-(tetrahydro-2H-pyran-4-yl)phenyl)-4,4,5,5-tetramethyl-1,3,2-dioxaborolane (2.0 equiv.) at 100° C. for 20 min in microwave to give ethyl 5-(tert-butoxycarbonylamino)-2-(2,6-difluoro-4-(tetrahydro-2H-pyran-4-yl)phenyl)thiazole-4-carboxylate in 84% yield. LC/MS=469.2 (MH+), Rt=1.21 min.

### Synthesis of 5-((tert-butoxycarbonyl)amino)-2-(2,6-difluoro-4-(tetrahydro-2H-pyran-4-yl)phenyl)thiazole-4-carboxylic acid

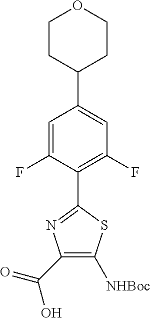 [SMILES: CC(C)(C)OC(=O)Nc1sc(-c2c(F)cc(C3CCOCC3)cc2F)nc1C(=O)O]

Method 2 was followed using ethyl 5-((tert-butoxycarbonyl)amino)-2-(2,6-difluoro-4-(tetrahydro-2H-pyran-4-yl)phenyl)thiazole-4-carboxylate to give 5-((tert-butoxycarbonyl)amino)-2-(2,6-difluoro-4-(tetrahydro-2H-pyran-4-yl)phenyl)thiazole-4-carboxylic acid in 72% yield. LC/MS=441.1 (MH+), Rt=1.02 min.

### Synthesis of ethyl 5-((tert-butoxycarbonyl)amino)-2-(2,6-difluoro-4-(4-hydroxytetrahydro-2H-pyran-4-yl)phenyl)thiazole-4-carboxylate

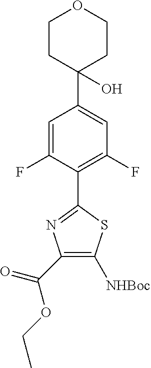 [SMILES: CCOC(=O)c1nc(-c2c(F)cc(C3(O)CCOCC3)cc2F)sc1NC(=O)OC(C)(C)C]

Method 1 was followed using ethyl 2-bromo-5-(tert-butoxycarbonylamino)thiazole-4-carboxylate (1.0 equiv.) and 4-(3,5-difluoro-4-(4,4,5,5-tetramethyl-1,3,2-dioxaborolan-2-yl)phenyl)tetrahydro-2H-pyran-4-ol (2.0 equiv.) at 100° C. for 20 min in microwave to give ethyl 5-(tert-butoxycarbonylamino)-2-(2,6-difluoro-4-(tetrahydro-2H-pyran-4-yl)phenyl)thiazole-4-carboxylate in 70% yield. LC/MS=485.1 (MH+), Rt=1.07 min.

### Synthesis of 5-((tert-butoxycarbonyl)amino)-2-(2,6-difluoro-4-(4-hydroxytetrahydro-2H-pyran-4-yl)phenyl)thiazole-4-carboxylic acid

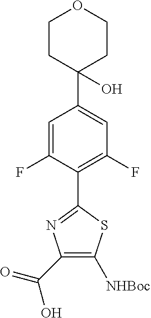 [SMILES: CC(C)(C)OC(=O)Nc1sc(-c2c(F)cc(C3(O)CCOCC3)cc2F)nc1C(=O)O]

Method 2 was followed using ethyl 5-((tert-butoxycarbonyl)amino)-2-(2,6-difluoro-4-(4-hydroxytetrahydro-2H-pyran-4-yl)phenyl)thiazole-4-carboxylate to give 5-((tert-butoxycarbonyl)amino)-2-(2,6-difluoro-4-(4-hydroxytetrahydro-2H-pyran-4-yl)phenyl)thiazole-4-carboxylic acid in 86% yield. LC/MS=457.0 (MH+), Rt=0.86 min.

### Synthesis of ethyl 5-((tert-butoxycarbonyl)amino)-2-(2,6-difluoro-4-(4-fluorotetrahydro-2H-pyran-4-yl)phenyl)thiazole-4-carboxylate

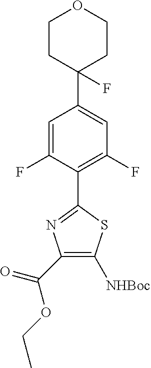 [SMILES: CCOC(=O)c1nc(-c2c(F)cc(C3(F)CCOCC3)cc2F)sc1NC(=O)OC(C)(C)C]

To a solution of ethyl 5-((tert-butoxycarbonyl)amino)-2-(2,6-difluoro-4-(4-hydroxytetrahydro-2H-pyran-4-yl)phenyl)thiazole-4-carboxylate (1.0 equiv.) in CH2Cl2 (0.01 M) at −78° C. was added DASTF (1.0 equiv.) dropwise. The resulting mixture was allowed to warm to RT and stirred at this temperature for a further 2 hrs. The reaction mixture was then quenched with NaHCO3 and diluted with EtOAc. The aqueous layer was separated then extracted with EtOAc. The combined organics were dried over MgSO4 and concentrated in vacuo to yield ethyl 5-((tert-butoxycarbonyl)amino)-2-(2,6-difluoro-4-(4-fluorotetrahydro-2H-pyran-4-yl)phenyl)thiazole-4-carboxylate in 100% yield. LC/MS=487.1 (MH+), Rt=1.21 min. The product was utilized in the subsequent reaction without further purification.

### Synthesis of 5-((tert-butoxycarbonyl)amino)-2-(2,6-difluoro-4-(4-fluorotetrahydro-2H-pyran-4-yl)phenyl)thiazole-4-carboxylic acid

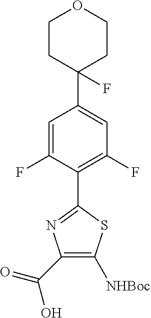 [SMILES: CC(C)(C)OC(=O)Nc1sc(-c2c(F)cc(C3(F)CCOCC3)cc2F)nc1C(=O)O]

Method 2 was followed using ethyl 5-((tert-butoxycarbonyl)amino)-2-(2,6-difluoro-4-(4-fluorotetrahydro-2H-pyran-4-yl)phenyl)thiazole-4-carboxylate to give 5-((tert-butoxycarbonyl)amino)-2-(2,6-difluoro-4-(4-fluorotetrahydro-2H-pyran-4-yl)phenyl)thiazole-4-carboxylic acid in 62% yield. LC/MS=459.0 (MH+), Rt=1.01 min.

### Method 6

A homogeneous solution of 1 eq each of amine, carboxylic acid, HOAT and EDC in DMF, at a concentration of 0.5 M, was left standing for 24 hours at which time water and ethyl acetate were added. The organic phase was dried with sodium sulfate and purified via silica gel column chromatography eluting with ethyl acetate and hexanes to give the desired protected amide product. Alternatively the crude reaction mixture was directly purified by HPLC. Upon lyophilization, the TFA salt of the protected amide product was obtained. Alternatively, the HPLC fractions could be added to EtOAc and solid Na₂CO₃, separated and washed with NaCl_{(sat.)}. Upon drying over MgSO₄, filtering and removing the volatiles in vacuo, the protected amide product was obtained as a free base. Alternatively, the crude reaction mixture was used for the deprotection step without further purification.

If an N-Boc protected amine was present, it was removed by treating with excess 4M HCl/dioxane for 14 hours or by treating with 25% TFA/CH₂Cl₂ for 2 hours. Upon removal of the volatiles in vacuo, the material was purified by RP HPLC yielding after lyophilization the amide product as the TFA salt. Alternatively, the HPLC fractions could be added to EtOAc and solid Na₂CO₃, separated and washed with NaCl_{(sat.)}. Upon drying over MgSO₄, filtering and removing the volatiles in vacuo the free base was obtained. Upon dissolving in MeCN/H₂O, adding 1 eq. of 1 N HCl and lyophilizing, the HCl salt of the amide product was obtained.

If an N-Boc, OAc group were present, prior to Boc deprotection, the acetate group could be cleaved by treating with K₂CO₃ (2.0 equiv.) in ethanol at a concentration of 0.1 M for 24 hours.

If a TBDMS ether was present, it was deprotected prior to Boc removal by treating with 6N HCl, THF, methanol (1:2:1) at room temperature for 12 h. After removal of volatiles in vacuo, the Boc amino group was deprotected as described above. Alternatively, the TBDMS ether and Boc group could be both deprotected with 6N HCl, THF, methanol (1:2:1) if left at rt for 24 hours, or heated at 60° C. for 3 hours.

If a OMe group was present, it was deprotected by treating with 1 M BBr₃ in DCM (2.0 equiv.) for 24 hours. Water was added dropwise and the volatiles were removed in vacuo. The material was purified via reverse phase HPLC as described above.

If a OBn group was present, it was deprotected by treatment with 10% Pd/C (0.2 equiv.) under an atmosphere of hydrogen in ethyl acetate and methanol (1:2). Upon completion, the reaction was filtered through Celite, washed with methanol, and the filtrate was concentrated in vacuo. If a nitro group was present, it could be reduced to the corresponding amino by treating with above described hydrogenation conditions. If an alkenyl group was present, it could be converted to alkyl by treating with the above described hydrogenation conditions.

If a CO₂Me group was present, it could be converted to the corresponding CO₂H following Method 2.

Following the procedures of Method 6, the following compounds were prepared:

TABLE 1

| | | | LC/MS | |
|---|---|---|---|---|
| | | LC/MS | (Rf | |
| Ex. | | (MH+ on | on | |
| No. | Structure | UPLC) | UPLC | Chemical Name |
| 1 | 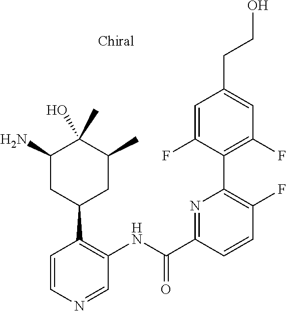 [SMILES: C[C@H]1C[C@@H](c2ccncc2NC(=O)c2ccc(F)c(-c3c(F)cc(CCO)cc3F)n2)C[C@@H](N)[C@]1(C)O] | 515.0 | 0.56 | N-(4-((1R,3R,4R,5S)-3- amino-4-hydroxy-4,5- dimethylcyclohexyl)pyridin- 3-yl)-6-(2,6-difluoro-4-(2- hydroxyethyl)phenyl)-5- fluoropicolinamide |
| 2 | 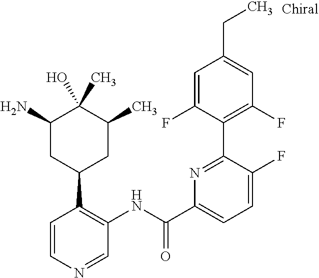 [SMILES: CCc1cc(F)c(-c2nc(C(=O)Nc3cnccc3[C@H]3C[C@@H](N)[C@](C)(O)[C@@H](C)C3)ccc2F)c(F)c1] | 499.1 | 0.67 | N-(4-((1R,3R,4R,5S)-3- amino-4-hydroxy-4,5- dimethylcyclohexyl)pyridin- 3-yl)-6-(4-ethyl-2,6- difluorophenyl)-5- fluoropicolinamide |
| 3 | 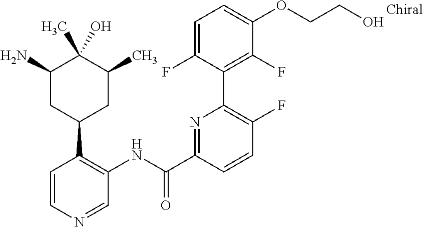 [SMILES: C[C@H]1C[C@@H](c2ccncc2NC(=O)c2ccc(F)c(-c3c(F)ccc(OCCO)c3F)n2)C[C@@H](N)[C@]1(C)O] | 531.1 | 0.51 | N-(4-((1R,3R,4R,5S)-3- amino-4-hydroxy-4,5- dimethylcyclohexyl)pyridin- 3-yl)-6-(2,6-difluoro-3-(2- hydroxyethoxy)phenyl)-5- fluoropicolinamide |
| 4 | 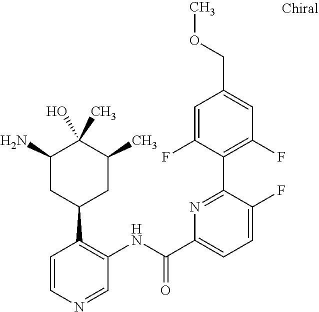 [SMILES: COCc1cc(F)c(-c2nc(C(=O)Nc3cnccc3[C@H]3C[C@@H](N)[C@](C)(O)[C@@H](C)C3)ccc2F)c(F)c1] | 515.1 | 0.60 | N-(4-((1R,3R,4R,5S)-3- amino-4-hydroxy-4,5- dimethylcyclohexyl)pyridin- 3-yl)-6-(2,6-difluoro-4- (methoxymethyl)phenyl)- 5-fluoropicolinamide |
| 5 | 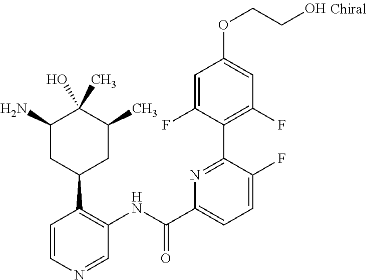 [SMILES: C[C@H]1C[C@@H](c2ccncc2NC(=O)c2ccc(F)c(-c3c(F)cc(OCCO)cc3F)n2)C[C@@H](N)[C@]1(C)O] | 531.1 | 0.62 | N-(4-((1R,3R,4R,5S)-3- amino-4-hydroxy-4,5- dimethylcyclohexyl)pyridin- 3-yl)-6-(2,6-difluoro-4-(2- hydroxyethoxy)phenyl)-5- fluoropicolinamide |
| 6 | 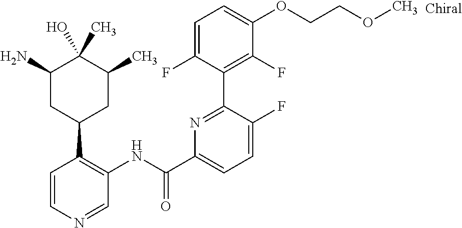 [SMILES: COCCOc1ccc(F)c(-c2nc(C(=O)Nc3cnccc3[C@H]3C[C@@H](N)[C@](C)(O)[C@@H](C)C3)ccc2F)c1F] | 545.1 | 0.65 | N-(4-((1R,3R,4R,5S)-3- amino-4-hydroxy-4,5- dimethylcyclohexyl)pyridin- 3-yl)-6-(2,6-difluoro-3-(2- methoxyethoxy)phenyl)-5- fluoropicolinamide |
| 7 | 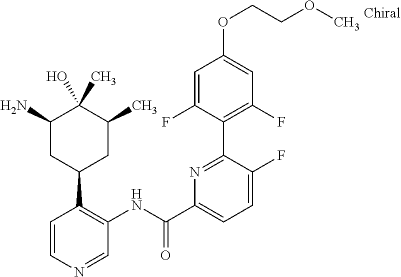 [SMILES: COCCOc1cc(F)c(-c2nc(C(=O)Nc3cnccc3[C@H]3C[C@@H](N)[C@](C)(O)[C@@H](C)C3)ccc2F)c(F)c1] | 545.1 | 0.67 | N-(4-((1R,3R,4R,5S)-3- amino-4-hydroxy-4,5- dimethylcyclohexyl)pyridin- 3-yl)-6-(2,6-difluoro-4-(2- methoxyethoxy)phenyl)-5- fluoropicolinamide |
| 8 | 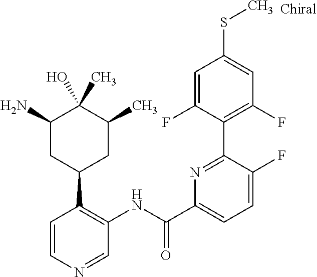 [SMILES: CSc1cc(F)c(-c2nc(C(=O)Nc3cnccc3[C@H]3C[C@@H](N)[C@](C)(O)[C@@H](C)C3)ccc2F)c(F)c1] | 517.1 | 0.69 | N-(4-((1R,3R,4R,5S)-3- amino-4-hydroxy-4,5- dimethylcyclohexyl)pyridin- 3-yl)-6-(2,6-difluoro-4- (methylthio)phenyl)-5- fluoropicolinamide |
| 9 | 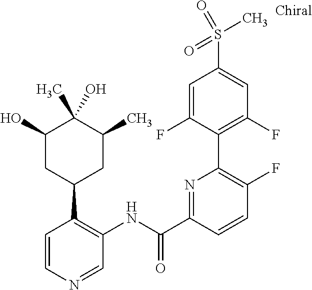 [SMILES: C[C@H]1C[C@@H](c2ccncc2NC(=O)c2ccc(F)c(-c3c(F)cc(S(C)(=O)=O)cc3F)n2)C[C@@H](O)[C@]1(C)O] | 550.0 | 0.59 | 6-(2,6-difluoro-4- (methylsulfonyl)phenyl)-N- (4-((1R,3R,4R,5S)-3,4- dihydroxy-4,5- dimethylcyclohexyl)pyridin- 3-yl)-5-fluoropicolinamide |
| 10 | 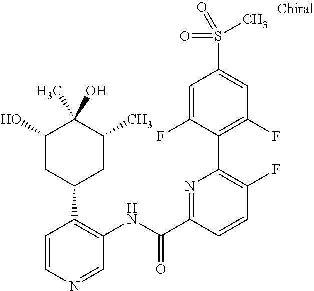 [SMILES: C[C@@H]1C[C@H](c2ccncc2NC(=O)c2ccc(F)c(-c3c(F)cc(S(C)(=O)=O)cc3F)n2)C[C@H](O)[C@@]1(C)O] | 550.0 | 0.59 | 6-(2,6-difluoro-4- (methylsulfonyl)phenyl)-N- (4-((1S,3S,4S,5R)-3,4- dihydroxy-4,5- dimethylcyclohexyl)pyridin- 3-yl)-5-fluoropicolinamide |
| 11 | 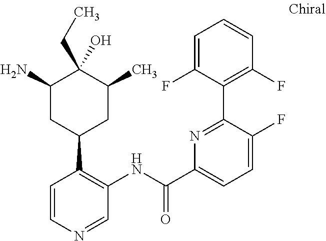 [SMILES: CC[C@]1(O)[C@H](N)C[C@H](c2ccncc2NC(=O)c2ccc(F)c(-c3c(F)cccc3F)n2)C[C@@H]1C] | 485.1 | 0.60 | N-(4-((1R,3R,4R,5S)-3- amino-4-ethyl-4-hydroxy- 5- methylcyclohexyl)pyridin- 3-yl)-6-(2,6- difluorophenyl)-5- fluoropicolinamide |
| 12 | 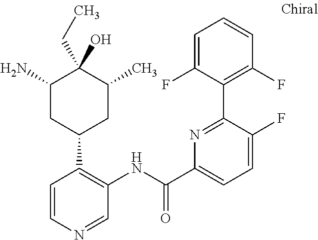 [SMILES: CC[C@]1(O)[C@H](C)C[C@H](c2ccncc2NC(=O)c2ccc(F)c(-c3c(F)cccc3F)n2)C[C@@H]1N] | 485.1 | 0.60 | N-(4-((1S,3S,4S,5R)-3- amino-4-ethyl-4-hydroxy- 5- methylcyclohexyl)pyridin- 3-yl)-6-(2,6- difluorophenyl)-5- fluoropicolinamide |
| 13 | 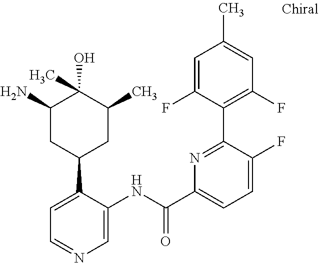 [SMILES: Cc1cc(F)c(-c2nc(C(=O)Nc3cnccc3[C@H]3C[C@@H](N)[C@](C)(O)[C@@H](C)C3)ccc2F)c(F)c1] | 485.1 | 0.62 | N-(4-((1R,3R,4R,5S)-3- amino-4-hydroxy-4,5- dimethylcyclohexyl)pyridin- 3-yl)-6-(2,6-difluoro-4- methylphenyl)-5- fluoropicolinamide |
| 14 | 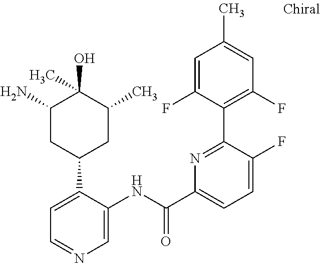 [SMILES: Cc1cc(F)c(-c2nc(C(=O)Nc3cnccc3[C@H]3C[C@@H](C)[C@](C)(O)[C@@H](N)C3)ccc2F)c(F)c1] | 485.1 | 0.62 | N-(4-((1S,3S,4S,5R)-3- amino-4-hydroxy-4,5- dimethylcyclohexyl)pyridin- 3-yl)-6-(2,6-difluoro-4- methylphenyl)-5- fluoropicolinamide |
| 15 | 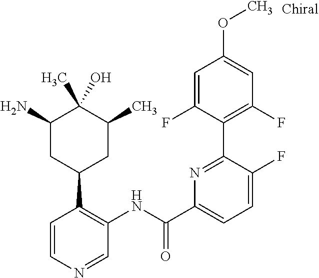 [SMILES: COc1cc(F)c(-c2nc(C(=O)Nc3cnccc3[C@H]3C[C@@H](N)[C@](C)(O)[C@@H](C)C3)ccc2F)c(F)c1] | 501.2 | 0.61 | N-(4-((1R,3R,4R,5S)-3- amino-4-hydroxy-4,5- dimethylcyclohexyl)pyridin- 3-yl)-6-(2,6-difluoro-4- methoxyphenyl)-5- fluoropicolinamide |
| 16 | 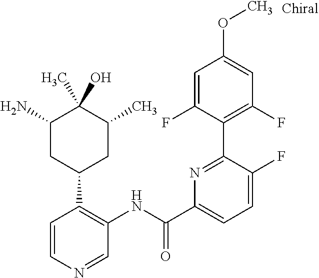 [SMILES: COc1cc(F)c(-c2nc(C(=O)Nc3cnccc3[C@H]3C[C@@H](C)[C@](C)(O)[C@@H](N)C3)ccc2F)c(F)c1] | 501.2 | 0.61 | N-(4-((1S,3S,4S,5R)-3- amino-4-hydroxy-4,5- dimethylcyclohexyl)pyridin- 3-yl)-6-(2,6-difluoro-4- methoxyphenyl)-5- fluoropicolinamide |
| 17 | 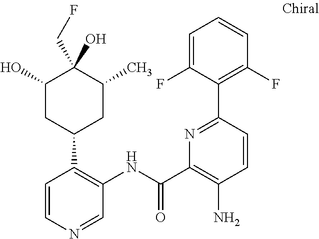 [SMILES: C[C@@H]1C[C@H](c2ccncc2NC(=O)c2nc(-c3c(F)cccc3F)ccc2N)C[C@H](O)[C@]1(O)CF] | 487.1 | 0.66 | 3-amino-6-(2,6- difluorophenyl)-N-(4- ((1S,3S,4S,5R)-4- (fluoromethyl)-3,4- dihydroxy-5- methylcyclohexyl)pyridin- 3-yl)picolinamide |
| 18 | 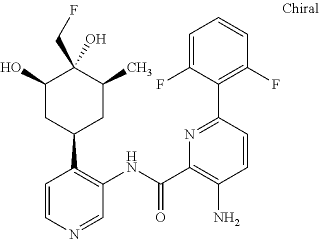 [SMILES: C[C@H]1C[C@@H](c2ccncc2NC(=O)c2nc(-c3c(F)cccc3F)ccc2N)C[C@@H](O)[C@@]1(O)CF] | 487.1 | 0.66 | 3-amino-6-(2,6- difluorophenyl)-N-(4- ((1R,3R,4R,5S)-4- (fluoromethyl)-3,4- dihydroxy-5- methylcyclohexyl)pyridin- 3-yl)picolinamide |
| 19 | 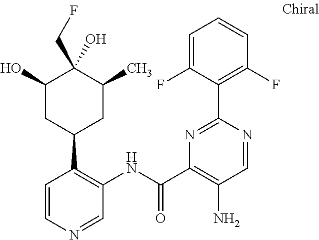 [SMILES: C[C@H]1C[C@@H](c2ccncc2NC(=O)c2nc(-c3c(F)cccc3F)ncc2N)C[C@@H](O)[C@@]1(O)CF] | 488.1 | 0.57 | 5-amino-2-(2,6- difluorophenyl)-N-(4- ((1R,3R,4R,5S)-4- (fluoromethyl)-3,4- dihydroxy-5- methylcyclohexyl)pyridin- 3-yl)pyrimidine-4- carboxamide |
| 20 | 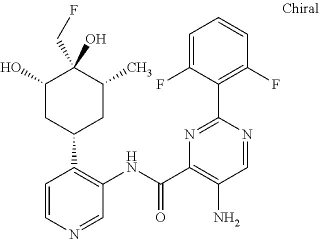 [SMILES: C[C@@H]1C[C@H](c2ccncc2NC(=O)c2nc(-c3c(F)cccc3F)ncc2N)C[C@H](O)[C@]1(O)CF] | 488.1 | 0.57 | 5-amino-2-(2,6- difluorophenyl)-N-(4- ((1S,3S,4S,5R)-4- (fluoromethyl)-3,4- dihydroxy-5- methylcyclohexyl)pyridin- 3-yl)pyrimidine-4- carboxamide |
| 21 | 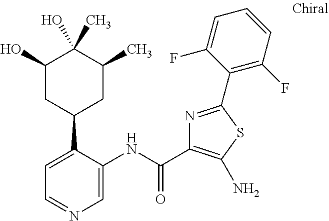 [SMILES: C[C@H]1C[C@@H](c2ccncc2NC(=O)c2nc(-c3c(F)cccc3F)sc2N)C[C@@H](O)[C@]1(C)O] | 475.1 | 0.63 | 5-amino-2-(2,6- difluorophenyl)-N-(4- ((1R,3R,4R,5S)-3,4- dihydroxy-4,5- dimethylcyclohexyl)pyridin- 3-yl)thiazole-4- carboxamide |
| 22 | 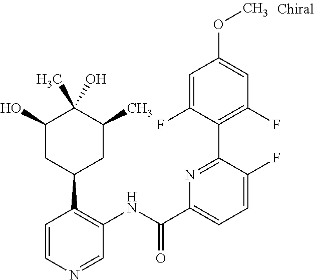 [SMILES: COc1cc(F)c(-c2nc(C(=O)Nc3cnccc3[C@H]3C[C@@H](O)[C@](C)(O)[C@@H](C)C3)ccc2F)c(F)c1] | 502.1 | 0.70 | 6-(2,6-difluoro-4- methoxyphenyl)-N-(4- ((1R,3R,4R,5S)-3,4- dihydroxy-4,5- dimethylcyclohexyl)pyridin- 3-yl)-5-fluoropicolinamide |
| 23 | 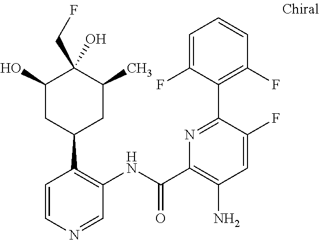 [SMILES: C[C@H]1C[C@@H](c2ccncc2NC(=O)c2nc(-c3c(F)cccc3F)c(F)cc2N)C[C@@H](O)[C@@]1(O)CF] | 505.1 | 0.69 | 3-amino-6-(2,6- difluorophenyl)-5-fluoro-N- (4-((1R,3R,4R,5S)-4- (fluoromethyl)-3,4- dihydroxy-5- methylcyclohexyl)pyridin- 3-yl)picolinamide |
| 24 | 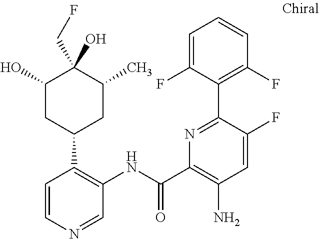 [SMILES: C[C@@H]1C[C@H](c2ccncc2NC(=O)c2nc(-c3c(F)cccc3F)c(F)cc2N)C[C@H](O)[C@]1(O)CF] | 505.1 | 0.69 | 3-amino-6-(2,6- difluorophenyl)-5-fluoro-N- (4-((1S,3S,4S,5R)-4- (fluoromethyl)-3,4- dihydroxy-5- methylcyclohexyl)pyridin- 3-yl)picolinamide |
| 25 | 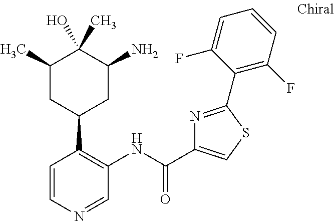 [SMILES: C[C@@H]1C[C@H](c2ccncc2NC(=O)c2csc(-c3c(F)cccc3F)n2)C[C@H](N)[C@@]1(C)O] | 459.1 | 0.56 | N-(4-((1S,3S,4S,5R)-3- amino-4-hydroxy-4,5- dimethylcyclohexyl)pyridin- 3-yl)-2-(2,6- difluorophenyl)thiazole-4- carboxamide |
| 26 | 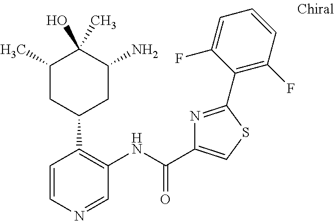 [SMILES: C[C@H]1C[C@@H](c2ccncc2NC(=O)c2csc(-c3c(F)cccc3F)n2)C[C@@H](N)[C@]1(C)O] | 459.1 | 0.56 | N-(4-((1R,3R,4R,5S)-3- amino-4-hydroxy-4,5- dimethylcyclohexyl)pyridin- 3-yl)-2-(2,6- difluorophenyl)thiazole-4- carboxamide |
| 27 | 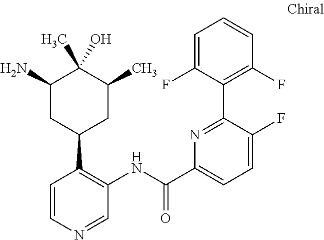 [SMILES: C[C@H]1C[C@@H](c2ccncc2NC(=O)c2ccc(F)c(-c3c(F)cccc3F)n2)C[C@@H](N)[C@]1(C)O] | 471.1 | 0.56 | N-(4-((1R,3R,4R,5S)-3- amino-4-hydroxy-4,5- dimethylcyclohexyl)pyridin- 3-yl)-6-(2,6- difluorophenyl)-5- fluoropicolinamide |
| 28 | 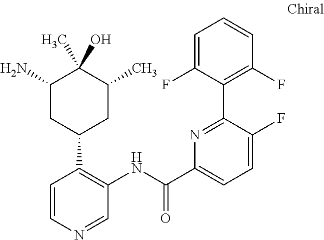 [SMILES: C[C@@H]1C[C@H](c2ccncc2NC(=O)c2ccc(F)c(-c3c(F)cccc3F)n2)C[C@H](N)[C@@]1(C)O] | 471.1 | 0.56 | N-(4-((1S,3S,4S,5R)-3- amino-4-hydroxy-4,5- dimethylcyclohexyl)pyridin- 3-yl)-6-(2,6- difluorophenyl)-5- fluoropicolinamide |
| 29 | 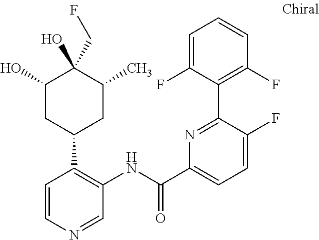 [SMILES: C[C@@H]1C[C@H](c2ccncc2NC(=O)c2ccc(F)c(-c3c(F)cccc3F)n2)C[C@H](O)[C@]1(O)CF] | 490.1 | 0.65 | 6-(2,6-difluorophenyl)-5- fluoro-N-(4- ((1S,3S,4S,5R)-4- (fluoromethyl)-3,4- dihydroxy-5- methylcyclohexyl)pyridin- 3-yl)picolinamide |
| 30 | 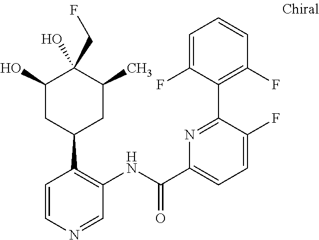 [SMILES: C[C@H]1C[C@@H](c2ccncc2NC(=O)c2ccc(F)c(-c3c(F)cccc3F)n2)C[C@@H](O)[C@@]1(O)CF] | 490.1 | 0.65 | 6-(2,6-difluorophenyl)-5- fluoro-N-(4- ((1R,3R,4R,5S)-4- (fluoromethyl)-3,4- dihydroxy-5- methylcyclohexyl)pyridin- 3-yl)picolinamide |
| 31 | 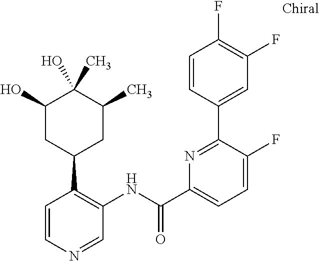 [SMILES: C[C@H]1C[C@@H](c2ccncc2NC(=O)c2ccc(F)c(-c3ccc(F)c(F)c3)n2)C[C@@H](O)[C@]1(C)O] | 472.1 | 0.66 | 6-(3,4-difluorophenyl)-N- (4-((1R,3R,4R,5S)-3,4- dihydroxy-4,5- dimethylcyclohexyl)pyridin- 3-yl)-5-fluoropicolinamide |
| 32 | 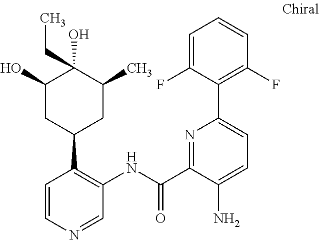 [SMILES: CC[C@]1(O)[C@H](O)C[C@H](c2ccncc2NC(=O)c2nc(-c3c(F)cccc3F)ccc2N)C[C@@H]1C] | 483.2 | 0.69 | 3-amino-6-(2,6- difluorophenyl)-N-(4- ((1R,3R,4R,5S)-4-ethyl- 3,4-dihydroxy-5- methylcyclohexyl)pyridin- 3-yl)picolinamide |
| 33 | 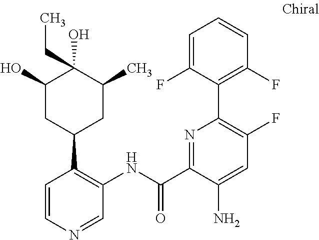 [SMILES: CC[C@]1(O)[C@H](O)C[C@H](c2ccncc2NC(=O)c2nc(-c3c(F)cccc3F)c(F)cc2N)C[C@@H]1C] | 501.2 | 0.71 | 3-amino-6-(2,6- difluorophenyl)-N-(4- ((1R,3R,4R,5S)-4-ethyl- 3,4-dihydroxy-5- methylcyclohexyl)pyridin- 3-yl)-5-fluoropicolinamide |
| 34 | 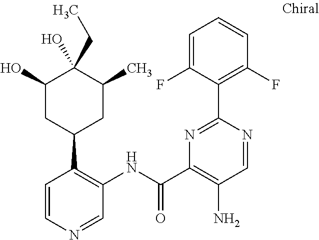 [SMILES: CC[C@]1(O)[C@H](O)C[C@H](c2ccncc2NC(=O)c2nc(-c3c(F)cccc3F)ncc2N)C[C@@H]1C] | 484.1 | 0.59 | 5-amino-2-(2,6- difluorophenyl)-N-(4- ((1R,3R,4R,5S)-4-ethyl- 3,4-dihydroxy-5- methylcyclohexyl)pyridin- 3-yl)pyrimidine-4- carboxamide |
| 35 | 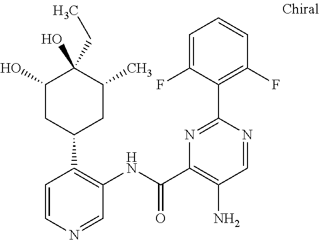 [SMILES: CC[C@]1(O)[C@H](C)C[C@H](c2ccncc2NC(=O)c2nc(-c3c(F)cccc3F)ncc2N)C[C@@H]1O] | 484.1 | 0.59 | 5-amino-2-(2,6- difluorophenyl)-N-(4- ((1S,3S,4S,5R)-4-ethyl- 3,4-dihydroxy-5- methylcyclohexyl)pyridin- 3-yl)pyrimidine-4- carboxamide |
| 36 | 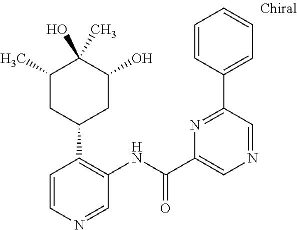 [SMILES: C[C@H]1C[C@@H](c2ccncc2NC(=O)c2cncc(-c3ccccc3)n2)C[C@@H](O)[C@]1(C)O] | 419.2 | 0.53 | N-(4-((1R,3R,4R,5S)-3,4- dihydroxy-4,5- dimethylcyclohexyl)pyridin- 3-yl)-6-phenylpyrazine-2- carboxamide |
| 37 | 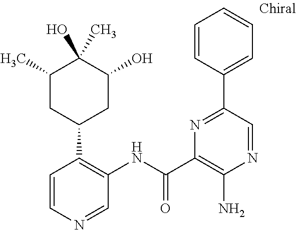 [SMILES: C[C@H]1C[C@@H](c2ccncc2NC(=O)c2nc(-c3ccccc3)cnc2N)C[C@@H](O)[C@]1(C)O] | 434.1 | 0.56 | 3-amino-N-(4- ((1R,3R,4R,5S)-3,4- dihydroxy-4,5- dimethylcyclohexyl)pyridin- 3-yl)-6-phenylpyrazine-2- carboxamide |
| 38 | 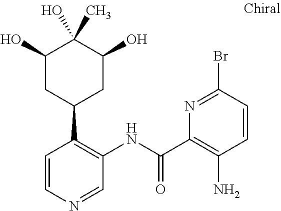 [SMILES: C[C@]1(O)[C@H](O)C[C@H](c2ccncc2NC(=O)c2nc(Br)ccc2N)C[C@@H]1O] | 435.0/ 437.0 | 0.49 | 3-amino-6-bromo-N-(4- ((1R,3R,4R,5S)-3,4- dihydroxy-4,5- dimethylcyclohexyl)pyridin- 3-yl)picolinamide |
| 39 | 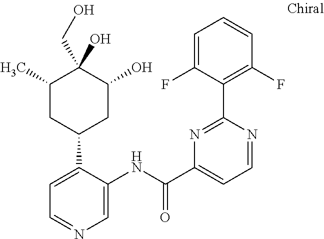 [SMILES: C[C@H]1C[C@@H](c2ccncc2NC(=O)c2ccnc(-c3c(F)cccc3F)n2)C[C@@H](O)[C@@]1(O)CO] | 471.1 | 0.50 | 2-(2,6-difluorophenyl)-N- (4-((1R,3R,4S,5S)-3,4- dihydroxy-4- (hydroxymethyl)-5- methylcyclohexyl)pyridin- 3-yl)pyrimidine-4- carboxamide |
| 40 | 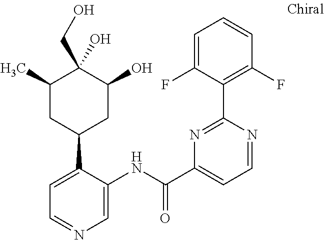 [SMILES: C[C@@H]1C[C@H](c2ccncc2NC(=O)c2ccnc(-c3c(F)cccc3F)n2)C[C@H](O)[C@]1(O)CO] | 471.1 | 0.50 | 2-(2,6-difluorophenyl)-N- (4-((1S,3S,4R,5R)-3,4- dihydroxy-4- (hydroxymethyl)-5- methylcyclohexyl)pyridin- 3-yl)pyrimidine-4- carboxamide |
| 41 | 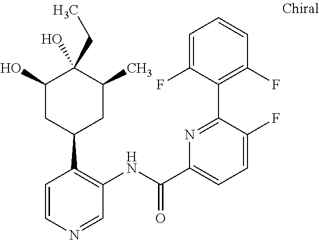 [SMILES: CC[C@]1(O)[C@H](O)C[C@H](c2ccncc2NC(=O)c2ccc(F)c(-c3c(F)cccc3F)n2)C[C@@H]1C] | 486.1 | 0.70 | 6-(2,6-difluorophenyl)-N- (4-((1R,3R,4R,5S)-4- ethyl-3,4-dihydroxy-5- methylcyclohexyl)pyridin- 3-yl)-5-fluoropicolinamide |
| 42 | 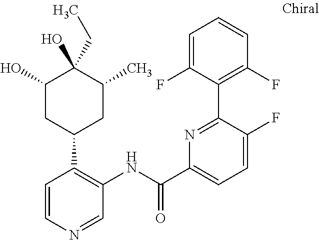 [SMILES: CC[C@]1(O)[C@H](C)C[C@H](c2ccncc2NC(=O)c2ccc(F)c(-c3c(F)cccc3F)n2)C[C@@H]1O] | 486.1 | 0.70 | 6-(2,6-difluorophenyl)-N- (4-((1S,3S,4S,5R)-4-ethyl- 3,4-dihydroxy-5- methylcyclohexyl)pyridin- 3-yl)-5-fluoropicolinamide |
| 43 | 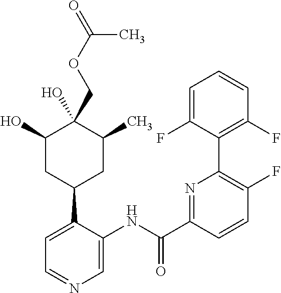 [SMILES: CC(=O)OC[C@]1(O)[C@H](O)C[C@H](c2ccncc2NC(=O)c2ccc(F)c(-c3c(F)cccc3F)n2)C[C@@H]1C] | 530.1 | 0.67 | ((1S,2R,4R,6S)-4-(3-(6- (2,6-difluorophenyl)-5- fluoropicolinamido)pyridin- 4-yl)-1,2-dihydroxy-6- methylcyclohexyl)methyl- acetate |
| 44 | 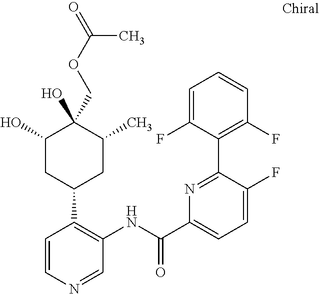 [SMILES: CC(=O)OC[C@]1(O)[C@H](C)C[C@H](c2ccncc2NC(=O)c2ccc(F)c(-c3c(F)cccc3F)n2)C[C@@H]1O] | 530.1 | 0.67 | ((1R,2S,4S,6R)-4-(3-(6- (2,6-difluorophenyl)-5- fluoropicolinamido)pyridin- 4-yl)-1,2-dihydroxy-6- methylcyclohexyl)methyl- acetate |
| 45 | 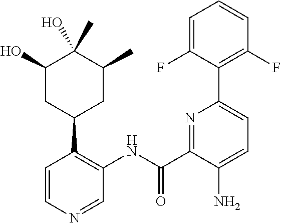 [SMILES: C[C@H]1C[C@@H](c2ccncc2NC(=O)c2nc(-c3c(F)cccc3F)ccc2N)C[C@@H](O)[C@]1(C)O] | 469.1 | 0.63 | 3-amino-6-(2,6- difluorophenyl)-N-(4- ((1R,3R,4R,5S)-3,4- dihydroxy-4,5- dimethylcyclohexyl)pyridin- 3-yl)picolinamide |
| 46 | 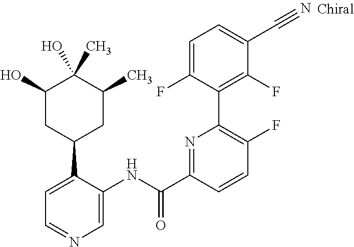 [SMILES: C[C@H]1C[C@@H](c2ccncc2NC(=O)c2ccc(F)c(-c3c(F)ccc(C#N)c3F)n2)C[C@@H](O)[C@]1(C)O] | 497.2 | 0.62 | 6-(3-cyano-2,6- difluorophenyl)-N-(4- ((1R,3R,4R,5S)-3,4- dihydroxy-4,5- dimethylcyclohexyl)pyridin- 3-yl)-5-fluoropicolinamide |
| 47 | 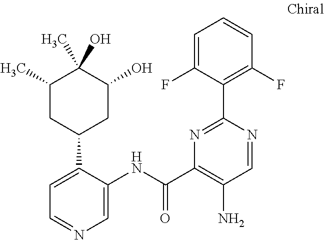 [SMILES: C[C@H]1C[C@@H](c2ccncc2NC(=O)c2nc(-c3c(F)cccc3F)ncc2N)C[C@@H](O)[C@]1(C)O] | 470.2 | 0.56 | 5-amino-2-(2,6- difluorophenyl)-N-(4- ((1R,3R,4R,5S)-3,4- dihydroxy-4,5- dimethylcyclohexyl)pyridin- 3-yl)pyrimidine-4- carboxamide |
| 48 | 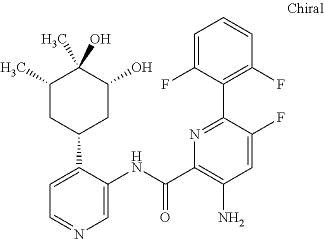 [SMILES: C[C@H]1C[C@@H](c2ccncc2NC(=O)c2nc(-c3c(F)cccc3F)c(F)cc2N)C[C@@H](O)[C@]1(C)O] | 487.1 | 0.66 | 3-amino-6-(2,6- difluorophenyl)-N-(4- ((1R,3R,4R,5S)-3,4- dihydroxy-4,5- dimethylcyclohexyl)pyridin- 3-yl)-5-fluoropicolinamide |
| 49 | 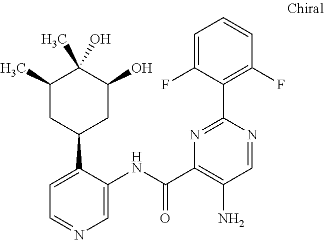 [SMILES: C[C@@H]1C[C@H](c2ccncc2NC(=O)c2nc(-c3c(F)cccc3F)ncc2N)C[C@H](O)[C@@]1(C)O] | 470.1 | 0.53 | 5-amino-2-(2,6- difluorophenyl)-N-(4- ((1S,3S,4S,5R)-3,4- dihydroxy-4,5- dimethylcyclohexyl)pyridin- 3-yl)pyrimidine-4- carboxamide |
| 50 | 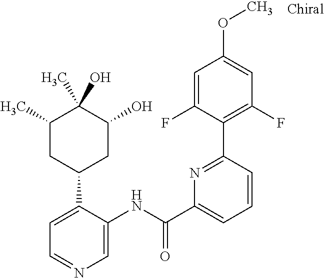 [SMILES: COc1cc(F)c(-c2cccc(C(=O)Nc3cnccc3[C@H]3C[C@@H](O)[C@](C)(O)[C@@H](C)C3)n2)c(F)c1] | 484.1 | 0.68 | 6-(2,6-difluoro-4- methoxyphenyl)-N-(4- ((1R,3R,4R,5S)-3,4- dihydroxy-4,5- dimethylcyclohexyl)pyridin- 3-yl)picolinamide |
| 51 | 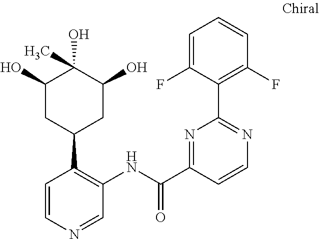 [SMILES: C[C@]1(O)[C@H](O)C[C@H](c2ccncc2NC(=O)c2ccnc(-c3c(F)cccc3F)n2)C[C@@H]1O] | 455.1 | 0.54 | 2-(2,6-difluorophenyl)-N- (4-((1R,3R,4R,5S)-3,4- dihydroxy-4,5- dimethylcyclohexyl)pyridin- 3-yl)pyrimidine-4- carboxamide |
| 52 | 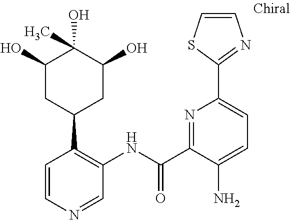 [SMILES: C[C@]1(O)[C@H](O)C[C@H](c2ccncc2NC(=O)c2nc(-c3nccs3)ccc2N)C[C@@H]1O] | 440.1 | 0.59 | 3-amino-N-(4- ((1R,3R,4R,5S)-3,4- dihydroxy-4,5- dimethylcyclohexyl)pyridin- 3-yl)-6-(thiazol-2- yl)picolinamide |
| 53 | 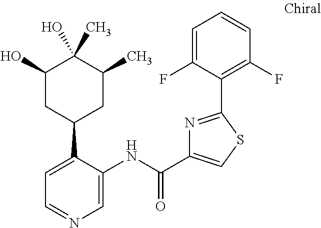 [SMILES: C[C@H]1C[C@@H](c2ccncc2NC(=O)c2csc(-c3c(F)cccc3F)n2)C[C@@H](O)[C@]1(C)O] | 459.9 | 0.60 | 2-(2,6-difluorophenyl)-N- (4-((1R,3R,4R,5S)-3,4- dihydroxy-4,5- dimethylcyclohexyl)pyridin- 3-yl)thiazole-4- carboxamide |
| 54 | 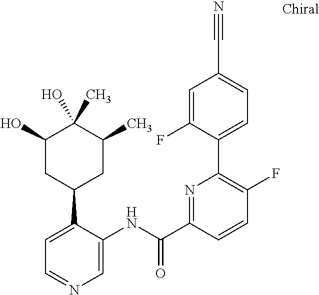 [SMILES: C[C@H]1C[C@@H](c2ccncc2NC(=O)c2ccc(F)c(-c3ccc(C#N)cc3F)n2)C[C@@H](O)[C@]1(C)O] | 479.0 | 0.62 | 6-(4-cyano-2- fluorophenyl)-N-(4- ((1R,3R,4R,5S)-3,4- dihydroxy-4,5- dimethylcyclohexyl)pyridin- 3-yl)-5-fluoropicolinamide |
| 55 | 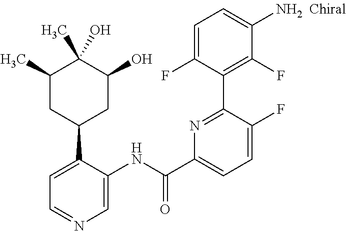 [SMILES: C[C@@H]1C[C@H](c2ccncc2NC(=O)c2ccc(F)c(-c3c(F)ccc(N)c3F)n2)C[C@H](O)[C@@]1(C)O] | 487.3 | 0.55 | 6-(3-amino-2,6- difluorophenyl)-N-(4- ((1S,3S,4S,5R)-3,4- dihydroxy-4,5- dimethylcyclohexyl)pyridin- 3-yl)-5-fluoropicolinamide |
| 56 | 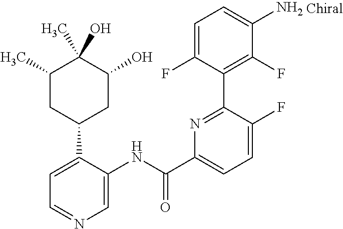 [SMILES: C[C@H]1C[C@@H](c2ccncc2NC(=O)c2ccc(F)c(-c3c(F)ccc(N)c3F)n2)C[C@@H](O)[C@]1(C)O] | 487.3 | 0.55 | 6-(3-amino-2,6- difluorophenyl)-N-(4- ((1R,3R,4R,5S)-3,4- dihydroxy-4,5- dimethylcyclohexyl)pyridin- 3-yl)-5-fluoropicolinamide |
| 57 | 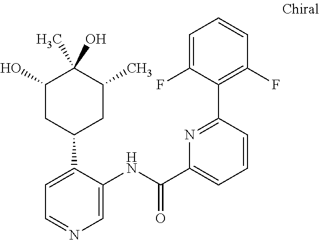 [SMILES: C[C@@H]1C[C@H](c2ccncc2NC(=O)c2cccc(-c3c(F)cccc3F)n2)C[C@H](O)[C@@]1(C)O] | 454.3 | 0.60 | 6-(2,6-difluorophenyl)-N- (4-((1S,3S,4S,5R)-3,4- dihydroxy-4,5- dimethylcyclohexyl)pyridin- 3-yl)picolinamide |
| 58 | 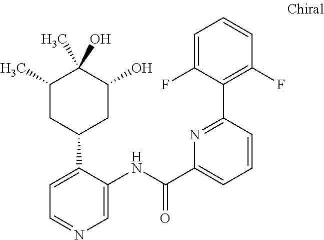 [SMILES: C[C@H]1C[C@@H](c2ccncc2NC(=O)c2cccc(-c3c(F)cccc3F)n2)C[C@@H](O)[C@]1(C)O] | 454.3 | 0.60 | 6-(2,6-difluorophenyl)-N- (4-((1R,3R,4R,5S)-3,4- dihydroxy-4,5- dimethylcyclohexyl)pyridin- 3-yl)picolinamide |
| 59 | 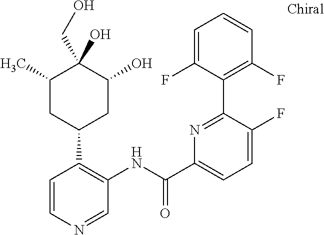 [SMILES: C[C@H]1C[C@@H](c2ccncc2NC(=O)c2ccc(F)c(-c3c(F)cccc3F)n2)C[C@@H](O)[C@@]1(O)CO] | 488 .2 | 0.60 | 6-(2,6-difluorophenyl)-N- (4-((1R,3R,4R,5S)-3,4- dihydroxy-4- (hydroxymethyl)-5- methylcyclohexyl)pyridin- 3-yl)-5-fluoropicolinamide |
| 60 | 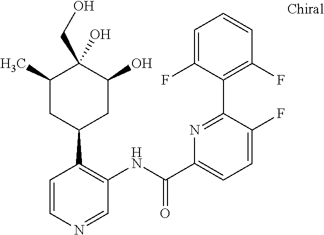 [SMILES: C[C@@H]1C[C@H](c2ccncc2NC(=O)c2ccc(F)c(-c3c(F)cccc3F)n2)C[C@H](O)[C@]1(O)CO] | 488.2 | 0.60 | 6-(2,6-difluorophenyl)-N- (4-((1S,3S,4R,5R)-3,4- dihydroxy-4- (hydroxymethyl)-5- methylcyclohexyl)pyridin- 3-yl)-5-fluoropicolinamide |
| 61 | 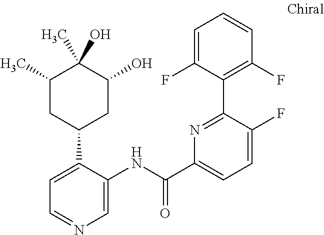 [SMILES: C[C@H]1C[C@@H](c2ccncc2NC(=O)c2ccc(F)c(-c3c(F)cccc3F)n2)C[C@@H](O)[C@]1(C)O] | 472.3 | 0.64 | 6-(2,6-difluorophenyl)-N- (4-((1R,3R,4R,5S)-3,4- dihydroxy-4,5- dimethylcyclohexyl)pyridin- 3-yl)-5-fluoropicolinamide |
| 62 | 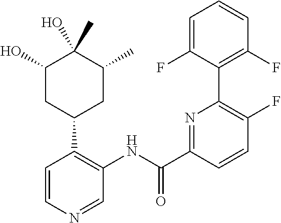 [SMILES: C[C@@H]1C[C@H](c2ccncc2NC(=O)c2ccc(F)c(-c3c(F)cccc3F)n2)C[C@H](O)[C@]1(C)O] | 472.3 | 0.64 | 6-(2,6-difluorophenyl)-N- (4-((1S,3S,4S,5R)-3,4- dihydroxy-4,5- dimethylcyclohexyl)pyridin- 3-yl)-5-fluoropicolinamide |
| 63 | 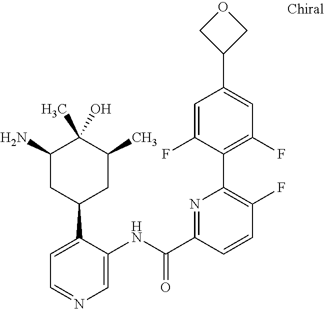 [SMILES: C[C@H]1C[C@@H](c2ccncc2NC(=O)c2ccc(F)c(-c3c(F)cc(C4COC4)cc3F)n2)C[C@@H](N)[C@]1(C)O] | 527.1 | 0.59 | N-(4-((1R,3R,4R,5S)-3- amino-4-hydroxy-4,5- dimethylcyclohexyl)pyridin- 3-yl)-6-(2,6-difluoro-4- (oxetan-3-yl)phenyl)-5- fluoropicolinamide |
| 64 | 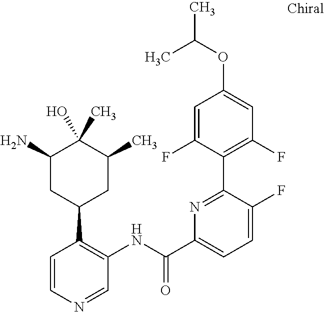 [SMILES: CC(C)Oc1cc(F)c(-c2nc(C(=O)Nc3cnccc3[C@H]3C[C@@H](N)[C@](C)(O)[C@@H](C)C3)ccc2F)c(F)c1] | 529.0 | 0.76 | N-(4-((1R,3R,4R,5S)-3- amino-4-hydroxy-4,5- dimethylcyclohexyl)pyridin- 6-difluoro-4- isopropoxyphenyl)-5- fluoropicolinamide |
| 65 | 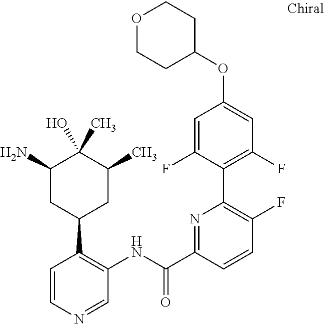 [SMILES: C[C@H]1C[C@@H](c2ccncc2NC(=O)c2ccc(F)c(-c3c(F)cc(OC4CCOCC4)cc3F)n2)C[C@@H](N)[C@]1(C)O] | 571.2 | 0.65 | N-(4-((1R,3R,4R,5S)-3- amino-4-hydroxy-4,5- dimethylcyclohexyl)pyridin- 3-yl)-6-(2,6-difluoro-4- (tetrahydro-2H-pyran-4- yloxy)phenyl)-5- fluoropicolinamide |
| 66 | 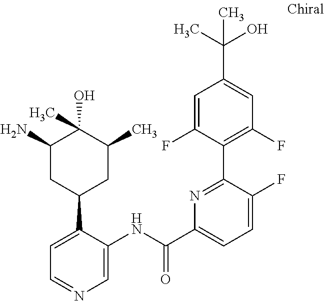 [SMILES: C[C@H]1C[C@@H](c2ccncc2NC(=O)c2ccc(F)c(-c3c(F)cc(C(C)(C)O)cc3F)n2)C[C@@H](N)[C@]1(C)O] | 529.1 | 0.59 | N-(4-((1R,3R,4R,5S)-3- amino-4-hydroxy-4,5- dimethylcyclohexyl)pyridin- 3-yl)-6-(2,6-difluoro-4-(2- hydroxypropan-2- yl)phenyl)-5- fluoropicolinamide |
| 67 | 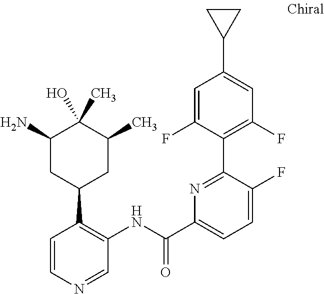 [SMILES: C[C@H]1C[C@@H](c2ccncc2NC(=O)c2ccc(F)c(-c3c(F)cc(C4CC4)cc3F)n2)C[C@@H](N)[C@]1(C)O] | 511.0 | 0.69 | N-(4-((1R,3R,4R,5S)-3- amino-4-hydroxy-4,5- dimethylcyclohexyl)pyridin- 3-yl)-6-(4-cyclopropyl- 2,6-difluorophenyl)-5- fluoropicolinamide |
| 68 | 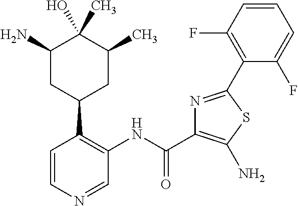 [SMILES: C[C@H]1C[C@@H](c2ccncc2NC(=O)c2nc(-c3c(F)cccc3F)sc2N)C[C@@H](N)[C@]1(C)O] | 474.2 | 0.59 | 5-amino-N-(4- ((1R,3R,4R,5S)-3-amino- 4-hydroxy-4,5- dimethylcyclohexyl)pyridin- 3-yl)-2-(2,6- difluorophenyl)thiazole-4- carboxamide |
| 69 | 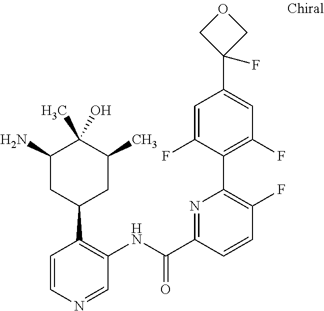 [SMILES: C[C@H]1C[C@@H](c2ccncc2NC(=O)c2ccc(F)c(-c3c(F)cc(C4(F)COC4)cc3F)n2)C[C@@H](N)[C@]1(C)O] | 545.30 | 0.62 | N-(4-((1R,3R,4R,5S)-3- amino-4-hydroxy-4,5- dimethylcyclohexyl)pyridin- 3-yl)-6-(2,6-difluoro-4-(3- fluorooxetan-3-yl)phenyl)- 5-fluoropicolinamide |
| 70 | 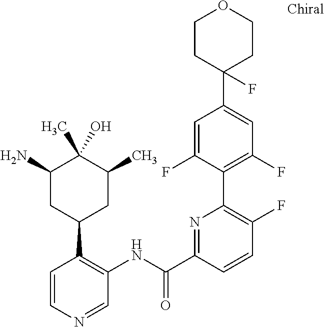 [SMILES: C[C@H]1C[C@@H](c2ccncc2NC(=O)c2ccc(F)c(-c3c(F)cc(C4(F)CCOCC4)cc3F)n2)C[C@@H](N)[C@]1(C)O] | 573.30 | 0.67 | N-(4-((1R,3R,4R,5S)-3- amino-4-hydroxy-4,5- dimethylcyclohexyl)pyridin- 3-yl)-6-(2,6-difluoro-4-(4- fluorotetrahydro-2H- pyran-4-yl)phenyl)-5- fluoropicolinamide |
| 71 | 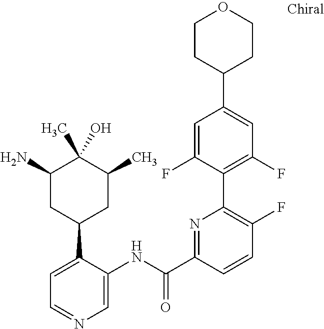 [SMILES: C[C@H]1C[C@@H](c2ccncc2NC(=O)c2ccc(F)c(-c3c(F)cc(C4CCOCC4)cc3F)n2)C[C@@H](N)[C@]1(C)O] | 555.2 | 0.66 | N-(4-((1R,3R,4R,5S)-3- amino-4-hydroxy-4,5- dimethylcyclohexyl)pyridin- 3-yl)-6-(2,6-difluoro-4- (tetrahydro-2H-pyran-4- yl)phenyl)-5- fluoropicolinamide |
| 72 | 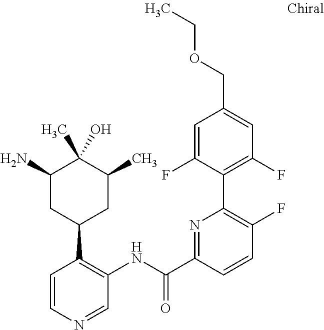 [SMILES: CCOCc1cc(F)c(-c2nc(C(=O)Nc3cnccc3[C@H]3C[C@@H](N)[C@](C)(O)[C@@H](C)C3)ccc2F)c(F)c1] | 529.3 | 0.66 | N-(4-((1R,3R,4R,5S)-3- amino-4-hydroxy-4,5- dimethylcyclohexyl)pyridin- 3-yl)-6-(4-(ethoxymethyl)- 2,6-difluorophenyl)-5- fluoropicolinamide |
| 73 | 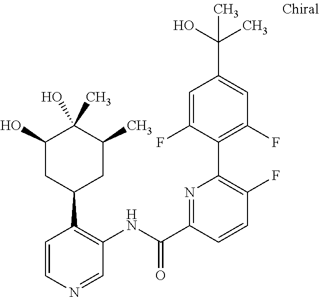 [SMILES: C[C@H]1C[C@@H](c2ccncc2NC(=O)c2ccc(F)c(-c3c(F)cc(C(C)(C)O)cc3F)n2)C[C@@H](O)[C@]1(C)O] | 530.3 | 0.67 | 6-(2,6-difluoro-4-(2- hydroxypropan-2- yl)phenyl)-N-(4- ((1R,3R,4R,5S)-3,4- dihydroxy-4,5- dimethylcyclohexyl)pyridin- 3-yl)-5-fluoropicolinamide |
| 74 | 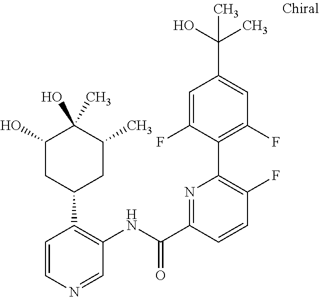 [SMILES: C[C@@H]1C[C@H](c2ccncc2NC(=O)c2ccc(F)c(-c3c(F)cc(C(C)(C)O)cc3F)n2)C[C@H](O)[C@@]1(C)O] | 530.3 | 0.67 | 6-(2,6-difluoro-4-(2- hydroxypropan-2- yl)phenyl)-N-(4- ((1S,3S,4S,5R)-3,4- dihydroxy-4,5- dimethylcyclohexyl)pyridin- 3-yl)-5-fluoropicolinamide |
| 75 | 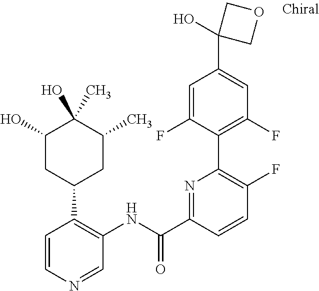 [SMILES: C[C@@H]1C[C@H](c2ccncc2NC(=O)c2ccc(F)c(-c3c(F)cc(C4(O)COC4)cc3F)n2)C[C@H](O)[C@@]1(C)O] | 544.2 | 0.62 | 6-(2,6-difluoro-4-(3- hydroxyoxetan-3- yl)phenyl)-N-(4- ((1S,3S,4S,5R)-3,4- dihydroxy-4,5- dimethylcyclohexyl)pyridin- 3 |
| 76 | 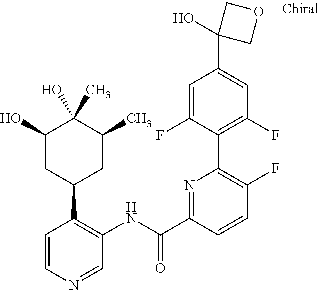 [SMILES: C[C@H]1C[C@@H](c2ccncc2NC(=O)c2ccc(F)c(-c3c(F)cc(C4(O)COC4)cc3F)n2)C[C@@H](O)[C@]1(C)O] | 544.3 | 0.62 | 6-(2,6-difluoro-4-(3- hydroxyoxetan-3- yl)phenyl)-N-(4- ((1R,3R,4R,5S)-3,4- dihydroxy-4,5- dimethylcyclohexyl)pyridin- 3-yl)-5-fluoropicolinamide- yl)-5-fluoropicolinamide |
| 77 | 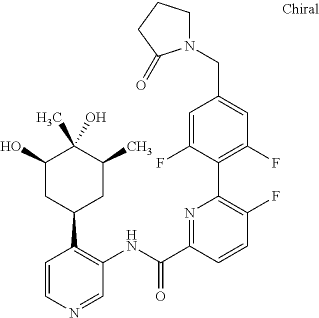 [SMILES: C[C@H]1C[C@@H](c2ccncc2NC(=O)c2ccc(F)c(-c3c(F)cc(CN4CCCC4=O)cc3F)n2)C[C@@H](O)[C@]1(C)O] | 569.2 | 0.66 | 6-(2,6-difluoro-4-((2- oxopyrrolidin-1- yl)methyl)phenyl)-N-(4- ((1R,3R,4R,5S)-3,4- dihydroxy-4,5- dimethylcyclohexyl)pyridin- 3-yl)-5-fluoropicolinamide |
| 78 | 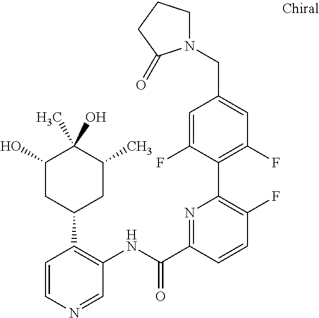 [SMILES: C[C@@H]1C[C@H](c2ccncc2NC(=O)c2ccc(F)c(-c3c(F)cc(CN4CCCC4=O)cc3F)n2)C[C@H](O)[C@@]1(C)O] | 569.2 | 0.66 | 6-(2,6-difluoro-4-((2- oxopyrrolidin-1- yl)methyl)phenyl)-N-(4- ((1S,3S,4S,5R)-3,4- dihydroxy-4,5- dimethylcyclohexyl)pyridin- 3-yl)-5-fluoropicolinamide |
| 79 | 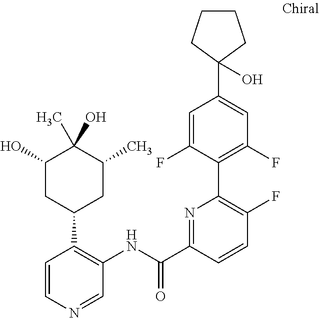 [SMILES: C[C@@H]1C[C@H](c2ccncc2NC(=O)c2ccc(F)c(-c3c(F)cc(C4(O)CCCC4)cc3F)n2)C[C@H](O)[C@@]1(C)O] | 556.3 | 0.73 | 6-(2,6-difluoro-4-(1- hydroxycyclopentyl)phenyl)- N-(4-((1S,3S,4S,5R)- 3,4-dihydroxy-4,5- dimethylcyclohexyl)pyridin- 3-yl)-5-fluoropicolinamide |
| 80 | 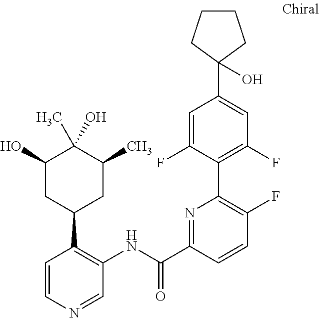 [SMILES: C[C@H]1C[C@@H](c2ccncc2NC(=O)c2ccc(F)c(-c3c(F)cc(C4(O)CCCC4)cc3F)n2)C[C@@H](O)[C@]1(C)O] | 556.3 | 0.73 | 6-(2,6-difluoro-4-(1- hydroxycyclopentyl)phenyl)- N-(4-((1R,3R,4R,5S)- 3,4-dihydroxy-4,5- dimethylcyclohexyl)pyridin- 3-yl)-5-fluoropicolinamide |
| 81 | 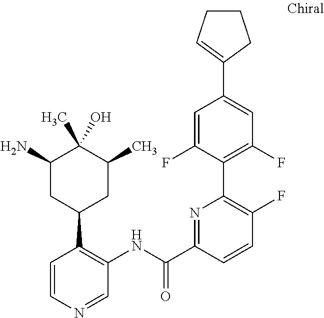 [SMILES: C[C@H]1C[C@@H](c2ccncc2NC(=O)c2ccc(F)c(-c3c(F)cc(C4=CCCC4)cc3F)n2)C[C@@H](N)[C@]1(C)O] | 537.3 | 0.74 | N-(4-((1R,3R,4R,5S)-3- amino-4-hydroxy-4,5- dimethylcyclohexyl)pyridin- 3-yl)-6-(4-cyclopentenyl- 2,6-difluorophenyl)-5- fluoropicolinamide |
| 82 | 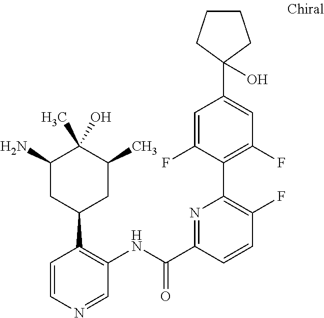 [SMILES: C[C@H]1C[C@@H](c2ccncc2NC(=O)c2ccc(F)c(-c3c(F)cc(C4(O)CCCC4)cc3F)n2)C[C@@H](N)[C@]1(C)O] | 555.3 | 0.63 | N-(4-((1R,3R,4R,5S)-3- amino-4-hydroxy-4,5- dimethylcyclohexyl)pyridin- 3-yl)-6-(2,6-difluoro-4-(1- hydroxycyclopentyl)phenyl)- 5-fluoropicolinamide |
| 83 | 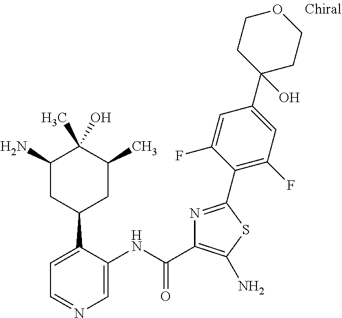 [SMILES: C[C@H]1C[C@@H](c2ccncc2NC(=O)c2nc(-c3c(F)cc(C4(O)CCOCC4)cc3F)sc2N)C[C@@H](N)[C@]1(C)O] | 574.3 | 0.56 | 5-amino-N-(4- ((1R,3R,4R,5S)-3-amino- 4-hydroxy-4,5- dimethylcyclohexyl)pyridin- 3-yl)-2-(2,6-difluoro-4-(4- hydroxytetrahydro-2H- pyran-4- yl)phenyl)thiazole-4- carboxamide |
| 84 | 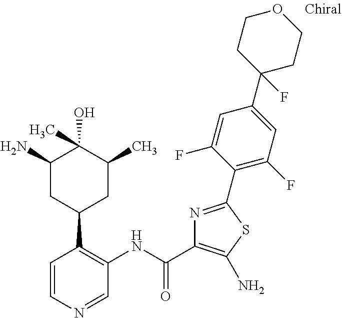 [SMILES: C[C@H]1C[C@@H](c2ccncc2NC(=O)c2nc(-c3c(F)cc(C4(F)CCOCC4)cc3F)sc2N)C[C@@H](N)[C@]1(C)O] | 576.3 | 0.66 | 5-amino-N-(4- ((1R,3R,4R,5S)-3-amino- 4-hydroxy-4,5- dimethylcyclohexyl)pyridin- 3-yl)-2-(2,6-difluoro-4-(4- fluorotetrahydro-2H- pyran-4- yl)phenyl)thiazole-4- carboxamide |
| 85 | 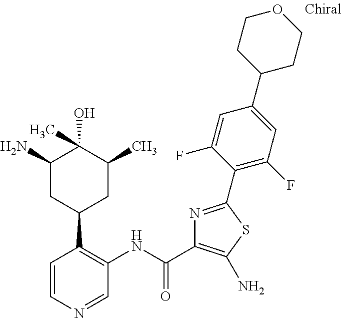 [SMILES: C[C@H]1C[C@@H](c2ccncc2NC(=O)c2nc(-c3c(F)cc(C4CCOCC4)cc3F)sc2N)C[C@@H](N)[C@]1(C)O] | 558.3 | 0.66 | 5-amino-N-(4- ((1R,3R,4R,5S)-3-amino- 4-hydroxy-4,5- dimethylcyclohexyl)pyridin- 3-yl)-2-(2,6-difluoro-4- (tetrahydro-2H-pyran-4- yl)phenyl)thiazole-4- carboxamide |
| 86 | 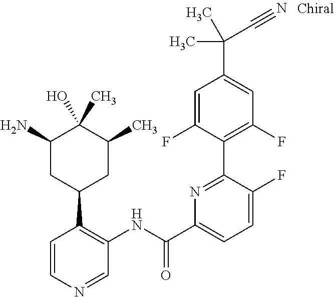 [SMILES: C[C@H]1C[C@@H](c2ccncc2NC(=O)c2ccc(F)c(-c3c(F)cc(C(C)(C)C#N)cc3F)n2)C[C@@H](N)[C@]1(C)O] | 538.2 | 0.68 | N-(4-((1R,3R,4R,5S)-3- amino-4-hydroxy-4,5- dimethylcyclohexyl)pyridin- 3-yl)-6-(4-(2- cyanopropan-2-yl)-2,6- difluorophenyl)-5- fluoropicolinamide |
| 87 | 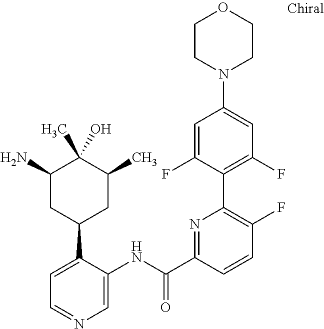 [SMILES: C[C@H]1C[C@@H](c2ccncc2NC(=O)c2ccc(F)c(-c3c(F)cc(N4CCOCC4)cc3F)n2)C[C@@H](N)[C@]1(C)O] | 556.3 | 0.66 min | N-(4-((1R,3R,4R,5S)-3- amino-4-hydroxy-4,5- dimethylcyclohexyl)pyridin- 3-yl)-6-(2,6-difluoro-4- morpholinophenyl)-5- fluoropicolinamide |
| 88 | 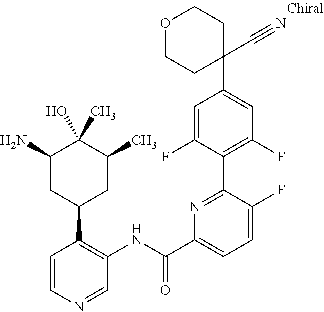 [SMILES: C[C@H]1C[C@@H](c2ccncc2NC(=O)c2ccc(F)c(-c3c(F)cc(C4(C#N)CCOCC4)cc3F)n2)C[C@@H](N)[C@]1(C)O] | 580.3 | 0.66 | N-(4-((1R,3R,4R,5S)-3- amino-4-hydroxy-4,5- dimethylcyclohexyl)pyridin- 3-yl)-6-(4-(4- cyanotetrahydro-2H- pyran-4-yl)-2,6- difluorophenyl)-5- fluoropicolinamide |
| 148 | 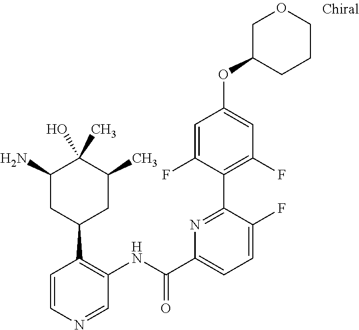 [SMILES: C[C@H]1C[C@@H](c2ccncc2NC(=O)c2ccc(F)c(-c3c(F)cc(O[C@@H]4CCCOC4)cc3F)n2)C[C@@H](N)[C@]1(C)O] | 571.3 | 0.75 | N-(4-((1R,3R,4R,5S)-3- amino-4-hydroxy-4,5- dimethylcyclohexyl)pyridin- 3-yl)-6-(2,6-difluoro-4- ((R)-tetrahydro-2H-pyran- 3-yloxy)phenyl)-5- fluoropicolinamide |
| 89 | 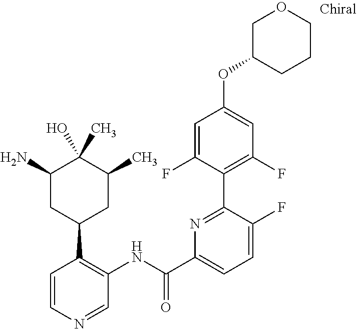 [SMILES: C[C@H]1C[C@@H](c2ccncc2NC(=O)c2ccc(F)c(-c3c(F)cc(O[C@H]4CCCOC4)cc3F)n2)C[C@@H](N)[C@]1(C)O] | 571.3 | 0.65 | N-(4-((1R,3R,4R,5S)-3- amino-4-hydroxy-4,5- dimethylcyclohexyl)pyridin- 3-yl)-6-(2,6-difluoro-4- ((S)-tetrahydro-2H-pyran- 3-yloxy)phenyl)-5- fluoropicolinamide |
| 90 | 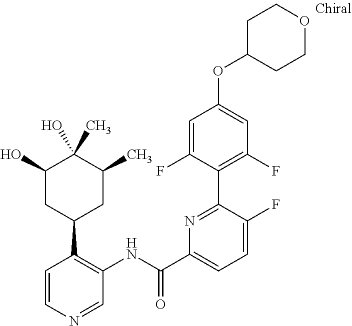 [SMILES: C[C@H]1C[C@@H](c2ccncc2NC(=O)c2ccc(F)c(-c3c(F)cc(OC4CCOCC4)cc3F)n2)C[C@@H](O)[C@]1(C)O] | 572.2 | 0.73 | 6-(2,6-difluoro-4- (tetrahydro-2H-pyran-4- yloxy)phenyl)-N-(4- ((1R,3R,4R,5S)-3,4- dihydroxy-4,5- dimethylcyclohexyl)pyridin- 3-yl)-5-fluoropicolinamide |
| 91 | 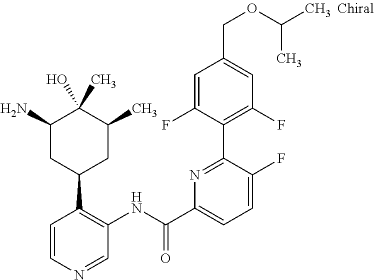 [SMILES: CC(C)OCc1cc(F)c(-c2nc(C(=O)Nc3cnccc3[C@H]3C[C@@H](N)[C@](C)(O)[C@@H](C)C3)ccc2F)c(F)c1] | 543.3 | 0.69 | N-(4-((1R,3R,4R,5S)-3- amino-4-hydroxy-4,5- dimethylcyclohexyl)pyridin- 3-yl)-6-(2,6-difluoro-4- (isopropoxymethyl)phenyl)- 5-fluoropicolinamide |
| 92 | 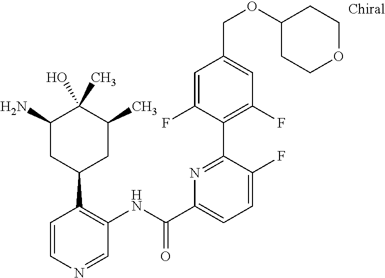 [SMILES: C[C@H]1C[C@@H](c2ccncc2NC(=O)c2ccc(F)c(-c3c(F)cc(COC4CCOCC4)cc3F)n2)C[C@@H](N)[C@]1(C)O] | 585.3 | 0.63 | N-(4-((1R,3R,4R,5S)-3- amino-4-hydroxy-4,5- dimethylcyclohexyl)pyridin- 3-yl)-6-(2,6-difluoro-4- ((tetrahydro-2H-pyran-4- yloxy)methyl)phenyl)-5- fluoropicolinamide |
| 93 | 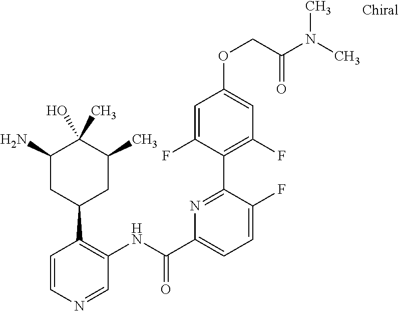 [SMILES: C[C@H]1C[C@@H](c2ccncc2NC(=O)c2ccc(F)c(-c3c(F)cc(OCC(=O)N(C)C)cc3F)n2)C[C@@H](N)[C@]1(C)O] | 572.2 | 0.57 | N-(4-((1R,3R,4R,5S)-3- amino-4-hydroxy-4,5- dimethylcyclohexyl)pyridin- 3-yl)-6-(4-(2- (dimethylamino)-2- oxoethoxy)-2,6- difluorophenyl)-5- fluoropicolinamide |
| 94 | 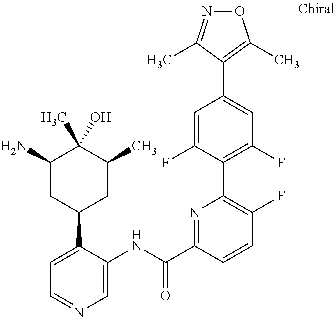 [SMILES: Cc1noc(C)c1-c1cc(F)c(-c2nc(C(=O)Nc3cnccc3[C@H]3C[C@@H](N)[C@](C)(O)[C@@H](C)C3)ccc2F)c(F)c1] | 566.3 | 0.68 min | N-(4-((1R,3R,4R,5S)-3- amino-4-hydroxy-4,5- dimethylcyclohexyl)pyridin- 3-yl)-6-(4-(3,5- dimethylisoxazol-4-yl)-2,6- difluorophenyl)-5- fluoropicolinamide |
| 95 | 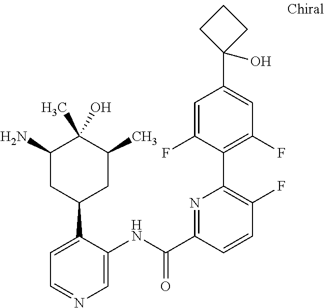 [SMILES: C[C@H]1C[C@@H](c2ccncc2NC(=O)c2ccc(F)c(-c3c(F)cc(C4(O)CCC4)cc3F)n2)C[C@@H](N)[C@]1(C)O] | 541.3 | 0.64 min | N-(4-((1R,3R,4R,5S)-3- amino-4-hydroxy-4,5- dimethylcyclohexyl)pyridin- 3-yl)-6-(2,6-difluoro-4-(1- hydroxycyclobutyl)phenyl)- 5-fluoropicolinamide |
| 96 | 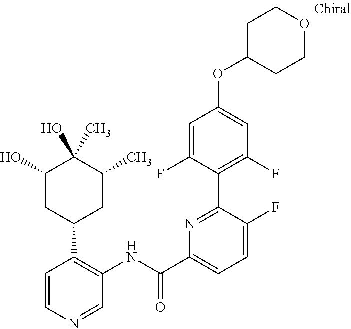 [SMILES: C[C@@H]1C[C@H](c2ccncc2NC(=O)c2ccc(F)c(-c3c(F)cc(OC4CCOCC4)cc3F)n2)C[C@H](O)[C@@]1(C)O] | 572.2 | 0.73 | 6-(2,6-difluoro-4- (tetrahydro-2H-pyran-4- yloxy)phenyl)-N-(4- ((1S,3S,4S,5R)-3,4- dihydroxy-4,5- dimethylcyclohexyl)pyridin- 3-yl)-5-fluoropicolinamide |
| 97 | 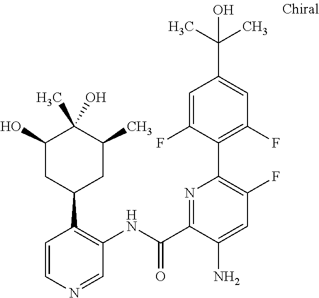 [SMILES: C[C@H]1C[C@@H](c2ccncc2NC(=O)c2nc(-c3c(F)cc(C(C)(C)O)cc3F)c(F)cc2N)C[C@@H](O)[C@]1(C)O] | 545.4 | 0.68 | 3-amino-6-(2,6-difluoro-4- (2-hydroxypropan-2- yl)phenyl)-N-(4- ((1R,3R,4R,5S)-3,4- dihydroxy-4,5- dimethylcyclohexyl)pyridin- 3-yl)-5-fluoropicolinamide |
| 98 | 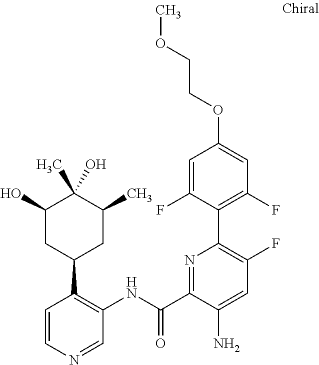 [SMILES: COCCOc1cc(F)c(-c2nc(C(=O)Nc3cnccc3[C@H]3C[C@@H](O)[C@](C)(O)[C@@H](C)C3)c(N)cc2F)c(F)c1] | 561.2 | 0.72 | 3-amino-6-(2,6-difluoro-4- (2- methoxyethoxy)phenyl)- N-(4-((1R,3R,4R,5S)-3,4- dihydroxy-4,5- dimethylcyclohexyl)pyridin- 3-yl)-5-fluoropicolinamide |
| 99 | 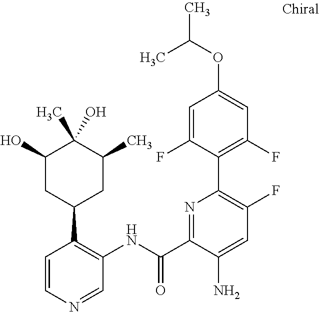 [SMILES: CC(C)Oc1cc(F)c(-c2nc(C(=O)Nc3cnccc3[C@H]3C[C@@H](O)[C@](C)(O)[C@@H](C)C3)c(N)cc2F)c(F)c1] | 545.2 | 0.8 | 3-amino-6-(2,6-difluoro-4- isopropoxyphenyl)-N-(4- ((1R,3R,4R,5S)-3,4- dihydroxy-4,5- dimethylcyclohexyl)pyridin- 3-yl)-5-fluoropicolinamide |
| 100 | 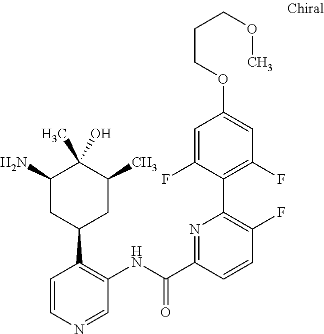 [SMILES: COCCCOc1cc(F)c(-c2nc(C(=O)Nc3cnccc3[C@H]3C[C@@H](N)[C@](C)(O)[C@@H](C)C3)ccc2F)c(F)c1] | 559.3 | 0.67 | N-(4-((1R,3R,4R,5S)-3- amino-4-hydroxy-4,5- dimethylcyclohexyl)pyridin- 3-yl)-6-(2,6-difluoro-4-(3- methoxypropoxy)phenyl)- 5-fluoropicolinamide |
| 101 | 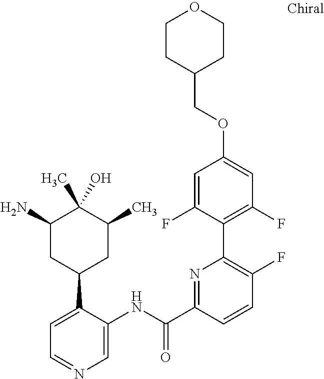 [SMILES: C[C@H]1C[C@@H](c2ccncc2NC(=O)c2ccc(F)c(-c3c(F)cc(OCC4CCOCC4)cc3F)n2)C[C@@H](N)[C@]1(C)O] | 585.3 | 0.72 | N-(4-((1R,3R,4R,5S)-3- amino-4-hydroxy-4,5- dimethylcyclohexyl)pyridin- 3-yl)-6-(2,6-difluoro-4- ((tetrahydro-2H-pyran-4- yl)methoxy)phenyl)-5- fluoropicolinamide |
| 102 | 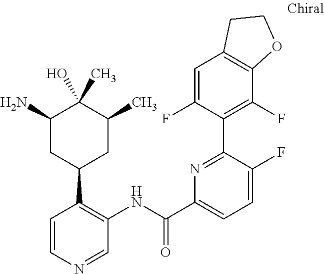 [SMILES: C[C@H]1C[C@@H](c2ccncc2NC(=O)c2ccc(F)c(-c3c(F)cc4c(c3F)OCC4)n2)C[C@@H](N)[C@]1(C)O] | 513.3 | 0.61 | N-(4-((1R,3R,4R,5S)-3- amino-4-hydroxy-4,5- dimethylcyclohexyl)pyridin- 3-yl)-6-(5,7-difluoro-2,3- dihydrobenzofuran-6-yl)- 5-fluoropicolinamide |
| 103 | 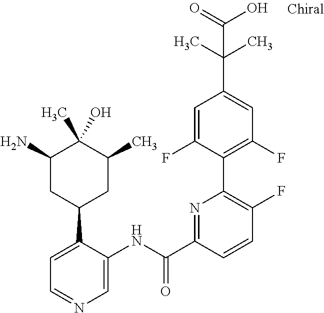 [SMILES: C[C@H]1C[C@@H](c2ccncc2NC(=O)c2ccc(F)c(-c3c(F)cc(C(C)(C)C(=O)O)cc3F)n2)C[C@@H](N)[C@]1(C)O] | 557.4 | 0.62 | 2-(4-(6-(4- ((1R,3R,4R,5S)-3-amino- 4-hydroxy-4,5- dimethylcyclohexyl)pyridin- 3-ylcarbamoyl)-3- fluoropyridin-2-yl)-3,5- difluorophenyl)-2- methylpropanoic acid |
| 104 | 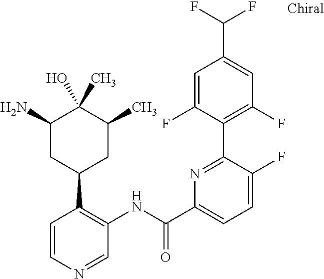 [SMILES: C[C@H]1C[C@@H](c2ccncc2NC(=O)c2ccc(F)c(-c3c(F)cc(C(F)F)cc3F)n2)C[C@@H](N)[C@]1(C)O] | 521.3 | 0.62 | N-(4-((1R,3R,4R,5S)-3- amino-4-hydroxy-4,5- dimethylcyclohexyl)pyridin- 3-yl)-6-(4- (difluoromethyl)-2,6- difluorophenyl)-5- fluoropicolinamide |
| 105 | 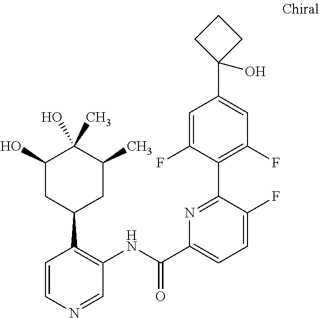 [SMILES: C[C@H]1C[C@@H](c2ccncc2NC(=O)c2ccc(F)c(-c3c(F)cc(C4(O)CCC4)cc3F)n2)C[C@@H](O)[C@]1(C)O] | 542.2 | 0.70 | 6-(2,6-difluoro-4-(1- hydroxycyclobutyl)phenyl)- N-(4-((1R,3R,4R,5S)-3,4- dihydroxy-4,5- dimethylcyclohexyl)pyridin- 3-yl)-5-fluoropicolinamide |
| 106 | 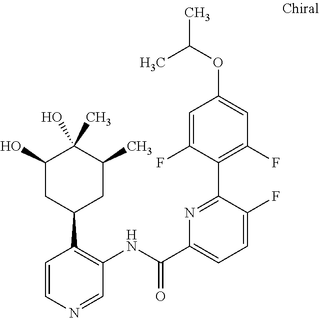 [SMILES: CC(C)Oc1cc(F)c(-c2nc(C(=O)Nc3cnccc3[C@H]3C[C@@H](O)[C@](C)(O)[C@@H](C)C3)ccc2F)c(F)c1] | 530.2 | 0.79 | 6-(2,6-difluoro-4- isopropoxyphenyl)-N-(4- ((1R,3R,4R,5S)-3,4- dihydroxy-4,5- dimethylcyclohexyl)pyridin- 3-yl)-5-fluoropicolinamide |
| 107 | 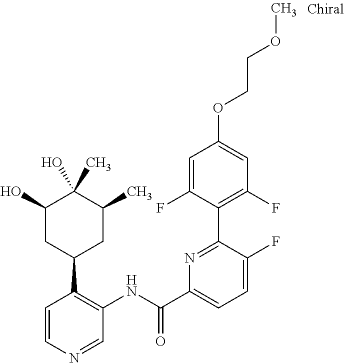 [SMILES: COCCOc1cc(F)c(-c2nc(C(=O)Nc3cnccc3[C@H]3C[C@@H](O)[C@](C)(O)[C@@H](C)C3)ccc2F)c(F)c1] | 546.3 | 0.70 | 6-(2,6-difluoro-4-(2- methoxyethoxy)phenyl)- N-(4-((1R,3R,4R,5S)-3,4- dihydroxy-4,5- dimethylcyclohexyl)pyridin- 3-yl)-5-fluoropicolinamide |
| 108 | 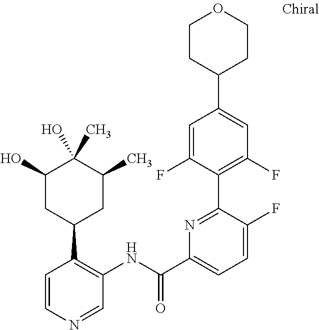 [SMILES: C[C@H]1C[C@@H](c2ccncc2NC(=O)c2ccc(F)c(-c3c(F)cc(C4CCOCC4)cc3F)n2)C[C@@H](O)[C@]1(C)O] | 556.3 | 0.73 | 6-(2,6-difluoro-4- (tetrahydro-2H-pyran-4- yl)phenyl)-N-(4- ((1R,3R,4R,5S)-3,4- dihydroxy-4,5- dimethylcyclohexyl)pyridin- 3-yl)-5-fluoropicolinamide |
| 109 | 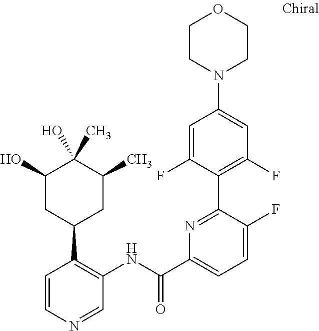 [SMILES: C[C@H]1C[C@@H](c2ccncc2NC(=O)c2ccc(F)c(-c3c(F)cc(N4CCOCC4)cc3F)n2)C[C@@H](O)[C@]1(C)O] | 557.4 | 0.71 | 6-(2,6-difluoro-4- morpholinophenyl)-N-(4- ((1R,3R,4R,5S)-3,4- dihydroxy-4,5- dimethylcyclohexyl)pyridin- 3-yl)-5-fluoropicolinamide |
| 110 | 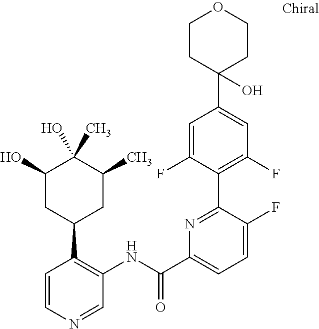 [SMILES: C[C@H]1C[C@@H](c2ccncc2NC(=O)c2ccc(F)c(-c3c(F)cc(C4(O)CCOCC4)cc3F)n2)C[C@@H](O)[C@]1(C)O] | 572.2 | 0.64 | 6-(2,6-difluoro-4-(4- hydroxytetrahydro-2H- pyran-4-yl)phenyl)-N-(4- ((1R,3R,4R,5S)-3,4- dihydroxy-4,5- dimethylcyclohexyl)pyridin- 3-yl)-5-fluoropicolinamide |
| 111 | 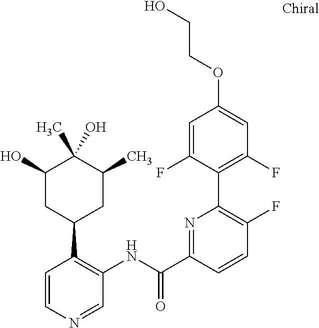 [SMILES: C[C@H]1C[C@@H](c2ccncc2NC(=O)c2ccc(F)c(-c3c(F)cc(OCCO)cc3F)n2)C[C@@H](O)[C@]1(C)O] | 532.2 | 0.62 | 6-(2,6-difluoro-4-(2- hydroxyethoxy)phenyl)-N- (4-((1R,3R,4R,5S)-3,4- dihydroxy-4,5- dimethylcyclohexyl)pyridin- 3-yl)-5-fluoropicolinamide |
| 112 | 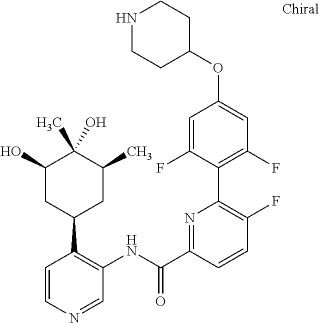 [SMILES: C[C@H]1C[C@@H](c2ccncc2NC(=O)c2ccc(F)c(-c3c(F)cc(OC4CCNCC4)cc3F)n2)C[C@@H](O)[C@]1(C)O] | 571.3 | 0.59 | 6-(2,6-difluoro-4- (piperidin-4-yloxy)phenyl)- N-(4-((1R,3R,4R,5S)-3,4- dihydroxy-4,5- dimethylcyclohexyl)pyridin- 3-yl)-5-fluoropicolinamide |
| 113 | 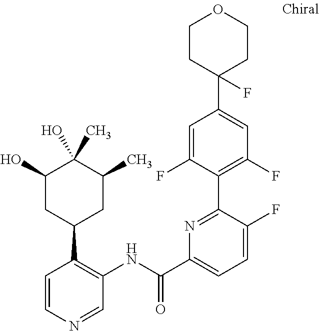 [SMILES: C[C@H]1C[C@@H](c2ccncc2NC(=O)c2ccc(F)c(-c3c(F)cc(C4(F)CCOCC4)cc3F)n2)C[C@@H](O)[C@]1(C)O] | 574.4 | 0.74 | 6-(2,6-difluoro-4-(4- fluorotetrahydro-2H- pyran-4-yl)phenyl)-N-(4- ((1R,3R,4R,5S)-3,4- dihydroxy-4,5- dimethylcyclohexyl)pyridin- 3-yl)-5-fluoropicolinamide |
| 114 | 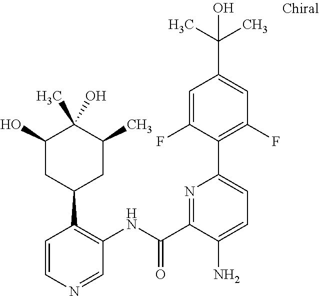 [SMILES: C[C@H]1C[C@@H](c2ccncc2NC(=O)c2nc(-c3c(F)cc(C(C)(C)O)cc3F)ccc2N)C[C@@H](O)[C@]1(C)O] | 527.2 | 0.68 | 3-amino-6-(2,6-difluoro-4- (2-hydroxypropan-2- yl)phenyl)-N-(4- ((1R,3R,4R,5S)-3,4- dihydroxy-4,5- dimethylcyclohexyl)pyridin- 3-yl)picolinamide |
| 115 | 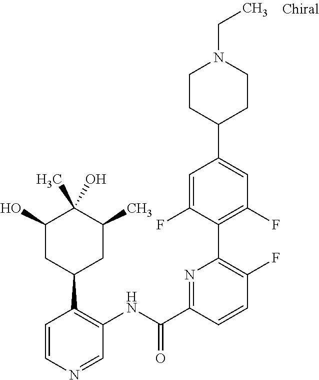 [SMILES: CCN1CCC(c2cc(F)c(-c3nc(C(=O)Nc4cnccc4[C@H]4C[C@@H](O)[C@](C)(O)[C@@H](C)C4)ccc3F)c(F)c2)CC1] | 583.3 | 0.64 | N-(4-((1R,3R,4R,5S)-3,4- dihydroxy-4,5- dimethylcyclohexyl)pyridin- 3-yl)-6-(4-(1- ethylpiperidin-4-yl)-2,6- difluorophenyl)-5- fluoropicolinamide |
| 116 | 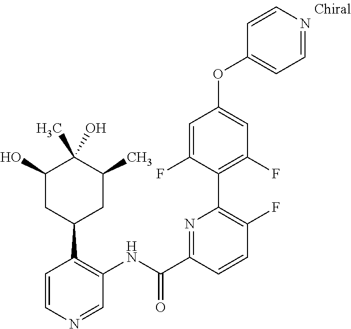 [SMILES: C[C@H]1C[C@@H](c2ccncc2NC(=O)c2ccc(F)c(-c3c(F)cc(Oc4ccncc4)cc3F)n2)C[C@@H](O)[C@]1(C)O] | 565.3 | 0.58 | 6-(2,6-difluoro-4-(pyridin- 4-yloxy)phenyl)-N-(4- ((1R,3R,4R,5S)-3,4- dihydroxy-4,5- dimethylcyclohexyl)pyridin- 3-yl)-5-fluoropicolinamide |
| 117 | 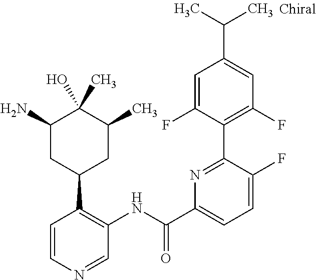 [SMILES: CC(C)c1cc(F)c(-c2nc(C(=O)Nc3cnccc3[C@H]3C[C@@H](N)[C@](C)(O)[C@@H](C)C3)ccc2F)c(F)c1] | 513.2 | 0.75 | N-(4-((1R,3R,4R,5S)-3- amino-4-hydroxy-4,5- dimethylcyclohexyl)pyridin- 3-yl)-6-(2,6-difluoro-4- isopropylphenyl)-5- fluoropicolinamide |
| 118 | 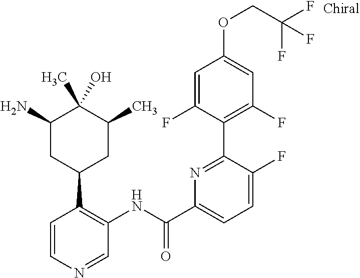 [SMILES: C[C@H]1C[C@@H](c2ccncc2NC(=O)c2ccc(F)c(-c3c(F)cc(OCC(F)(F)F)cc3F)n2)C[C@@H](N)[C@]1(C)O] | 569.2 | 0.74 | N-(4-((1R,3R,4R,5S)-3- amino-4-hydroxy-4,5- dimethylcyclohexyl)pyridin- 3-yl)-6-(2,6-difluoro-4- (2,2,2- trifluoroethoxy)phenyl)-5- fluoropicolinamide |
| 119 | 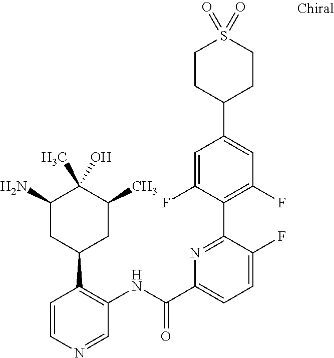 [SMILES: C[C@H]1C[C@@H](c2ccncc2NC(=O)c2ccc(F)c(-c3c(F)cc(C4CCS(=O)(=O)CC4)cc3F)n2)C[C@@H](N)[C@]1(C)O] | 603.2 | 0.60 | N-(4-((1R,3R,4R,5S)-3- amino-4-hydroxy-4,5- dimethylcyclohexyl)pyridin- 3-yl)-6-(4-(1,1- dioxidotetrahydro-2H- thiopyran-4-yl)-2,6- difluorophenyl)-5- fluoropicolinamide |
| 120 | 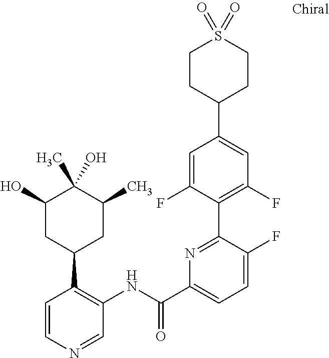 [SMILES: C[C@H]1C[C@@H](c2ccncc2NC(=O)c2ccc(F)c(-c3c(F)cc(C4CCS(=O)(=O)CC4)cc3F)n2)C[C@@H](O)[C@]1(C)O] | 604.2 | 0.65 | N-(4-((1R,3R,4S,5S)-3,4- dihydroxy-4,5- dimethylcyclohexyl)pyridin- 3-yl)-6-(4-(1,1- dioxidotetrahydro-2H- thiopyran-4-yl)-2,6- difluorophenyl)-5- fluoropicolinamide |

### Synthesis of (+/−)-6-(2,6-difluorophenyl)-N-(4-(1R,3R,4R,5S)-4-ethyl-3,4-dihydroxy-5-methylcyclohexyl)pyridin-3-yl)-5-fluoropicolinamide

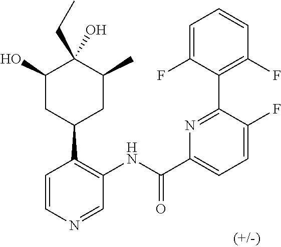 [SMILES: CC[C@]1(O)[C@H](O)C[C@H](c2ccncc2NC(=O)c2ccc(F)c(-c3c(F)cccc3F)n2)C[C@@H]1C]

To a solution of (+/−)-(1R,2R,4R,6S)-4-(3-aminopyridin-4-yl)-1-ethyl-6-methylcyclohexane-1,2-diol (1.0 equiv.) in DMF (0.5 M) was added 6-(2,6-difluorophenyl)-5-fluoropicolinic acid (1.1 equiv.), 1-hydroxy-7-azabenzotriazole (1.3 equiv.), and EDC (1.3 equiv.) sequentially. The reaction mixture was stirred for overnight. After quenched with NaHCO₃, the reaction mixture was extracted with EtOAc. The organic layer was washed with NaHCO3, water, and brine, dried over anhydrous Na2SO4, filtered, and concentrated in vacuo to yield (+/−)-6-(2,6-difluorophenyl)-N-(4-((1R,3R,4R,5S)-4-ethyl-3,4-dihydroxy-5-methylcyclohexyl)pyridin-3-yl)-5-fluoropicolinamide. LCMS (m/z): 486.2 (MH⁺), Rₜ=0.69 min.

### Synthesis of (+/−)-6-(2,6-difluorophenyl)-N-(4-((1R,3S,4R)-4-ethyl-4-hydroxy-3-methyl-5-oxocyclohexyl)pyridin-3-yl)-5-fluoropicolinamide

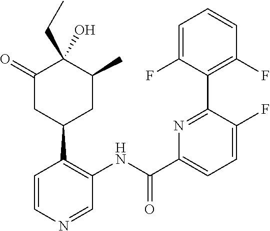 [SMILES: CC[C@]1(O)C(=O)C[C@H](c2ccncc2NC(=O)c2ccc(F)c(-c3c(F)cccc3F)n2)C[C@@H]1C]

To a solution of 6-(2,6-difluorophenyl)-N-(4-((1R,3R,4R,5S)-4-ethyl-3,4-dihydroxy-5-methylcyclohexyl)pyridin-3-yl)-5-fluoropicolinamide (1.0 equiv.) in DCM (0.5 M) and DMF (0.15 M) was added Dess-Martin periodinane (1.05 equiv.). The reaction mixture was stirred for 4 h. To the reaction mixture, sat NaHCO₃/sat Na₂S₂O₃ (8:1, 5 mL) and EtOAc (5 mL) were added and stirred vigorously for 3 h. EtOAc layer was separated and washed with brine and dried over anhydrous Na2SO4, filtered off and concentrated in vacuo. The crude (+/−)-6-(2,6-difluorophenyl)-N-(4-((1R,3S,4R)-4-ethyl-4-hydroxy-3-methyl-5-oxocyclohexyl)pyridin-3-yl)-5-fluoropicolinamide was used for next step. LCMS (m/z): 484.1 (MH⁺), Rₜ=0.76 min.

### Synthesis of (+/−)-N-(4-((1R,3R,4R,5S)-3-(benzylamino)-4-ethyl-4-hydroxy-5-methylcyclohexyl)pyridin-3-yl)-6-(2,6-difluorophenyl)-5-fluoropicolinamide

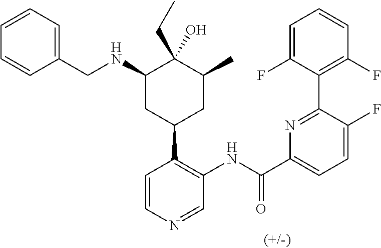 [SMILES: CC[C@@]1(O)[C@@H](C)C[C@@H](c2ccncc2NC(=O)c2ccc(F)c(-c3c(F)cccc3F)n2)C[C@H]1NCc1ccccc1]

To a solution of crude (+/−)-6-(2,6-difluorophenyl)-N-(4-((1R,3S,4R)-4-ethyl-4-hydroxy-3-methyl-5-oxocyclohexyl)pyridin-3-yl)-5-fluoropicolinamide (1 equiv.) in DCM (0.3 M) was added benzylamine (3 equiv.) followed by 4 Å molecular sevies. The solution was stirred for 2 days at room temperature and cooled to −78° C. and LiBH₄ (2 M in THF) (1.1 equiv.) was added dropwise. The mixture was allowed to warm up to rt over 3 h. The mixture was diluted with EtOAc and washed with sat. sodium bicarbonate (2×), brine, dried over sodium sulfate, filtered and concentrated. Two diastereomers were obtained in ˜1:1 ratio on HPLC. (+/−)-N-(4-((1R,3R,4R,5S)-3-(benzylamino)-4-ethyl-4-hydroxy-5-methylcyclohexyl)pyridin-3-yl)-6-(2,6-difluorophenyl)-5-fluoropicolinamide was purified by SiO2 chromatography. LCMS (m/z): 575.1 (MH⁺), Rₜ=0.71 min.

### Synthesis of N-(4-((1S,3S,4S,5R)-3-amino-4-ethyl-4-hydroxy-5-methylcyclohexyl)pyridin-3-yl)-6-(2,6-difluorophenyl)-5-fluoropicolinamide and N-(4-((1R,3R,4R,5S)-3-amino-4-ethyl-4-hydroxy-5-methylcyclohexyl)pyridin-3-yl)-6-(2,6-difluorophenyl)-5-fluoropicolinamide

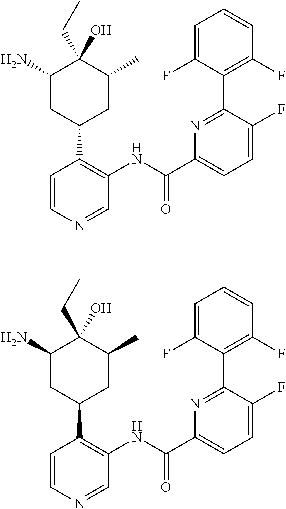 [SMILES: CC[C@]1(O)[C@H](C)C[C@H](c2ccncc2NC(=O)c2ccc(F)c(-c3c(F)cccc3F)n2)C[C@@H]1N ; CC[C@]1(O)[C@H](N)C[C@H](c2ccncc2NC(=O)c2ccc(F)c(-c3c(F)cccc3F)n2)C[C@@H]1C]

To a solution of (+/−)-N-(4-((1R,3R,4R,5S)-3-(benzylamino)-4-ethyl-4-hydroxy-5-methylcyclohexyl)pyridin-3-yl)-6-(2,6-difluorophenyl)-5-fluoropicolinamide (1.0 equiv.) in MeOH (0.1 M) was added Pd(OH)₂ (3.6 equiv.). The reaction mixture was degassed by N₂ stream for 15 min. After flushing with hydrogen gas, the reaction mixture with hydrogen balloon was stirred for 3.5 h. The crude product was purified by prep HPLC. The pure fraction was free-based and concentrated to yield (+/−)-N-(4-((1R,3R,4R,5S)-3-amino-4-ethyl-4-hydroxy-5-methylcyclohexyl)pyridin-3-yl)-6-(2,6-difluorophenyl)-5-fluoropicolinamide. LCMS (m/z): 485.1 (MH⁺), Rₜ=0.6 min. Upon chiral SFC separation, two enantiomers were obtained. N-(4-((1S,3S,4S,5R)-3-amino-4-ethyl-4-hydroxy-5-methylcyclohexyl)pyridin-3-yl)-6-(2,6-difluorophenyl)-5-fluoropicolinamide (>99% ee), Rₜ=1.28 min (IC column, Methanol+0.1% DEA=45%) and N-(4-((1R,3R,4R,5S)-3-amino-4-ethyl-4-hydroxy-5-methylcyclohexyl)pyridin-3-yl)-6-(2,6-difluorophenyl)-5-fluoropicolinamide (>99% ee), Rₜ=2.13 min (IC column, Methanol+0.1% DEA=45%). ¹H NMR (400 MHz, CDCl3) δ 9.94 (s, 1H), 9.36 (s, 1H), 8.47-8.34 (m, 2H), 7.78 (t, 1H), 7.52 (s, 1H), 7.23-7.03 (m, 3H), 3.05-2.91 (m, 1H), 2.85-2.73 (m, 1H), 2.73-2.61 (m, 1H), 2.22-2.13 (m, 1H), 1.94-1.80 (m, 1H), 1.78-1.62 (m, 3H), 1.49-1.36 (m, 2H), 1.34-1.19 (m, 2H), 1.03 (s, 3H), 0.93 (d, 3H).

### Synthesis of 3-amino-6-bromo-N-(4-((1R,3R,4R,5S)-3-(tert-butyldimethylsilyloxy)-4-hydroxy-4,5-dimethylcyclohexyl)pyridin-3-yl)picolinamide

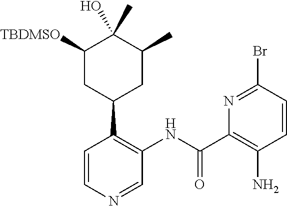 [SMILES: C[C@H]1C[C@@H](c2ccncc2NC(=O)c2nc(Br)ccc2N)C[C@@H](O[Si](C)(C)C(C)(C)C)[C@]1(C)O]

Following Method 6, (1R,2R,4R,6S)-4-(3-aminopyridin-4-yl)-2-(tert-butyldimethylsilyloxy)-1,6-dimethylcyclohexanol and 3-amino-6-bromopicolinic acid were coupled and following addition of EtOAc and washing with H₂O, NaCl_{(sat.)} and drying over MgSO₄, 3-amino-6-bromo-N-(4-((1R,3R,4R,5S)-3-(tert-butyldimethylsilyloxy)-4-hydroxy-4,5-dimethylcyclohexyl)pyridin-3-yl)picolinamide was obtained. LCMS (m/z): 549.1/551.1 (MH⁺), Rₜ=0.99 min.

### Synthesis of 3-amino-N-(4-((1R,3R,4R,5S)-3-(tert-butyldimethylsilyloxy)-4-hydroxy-4,5-dimethylcyclohexyl)pyridin-3-yl)-6-(4,4,5,5-tetramethyl-1,3,2-dioxaborolan-2-yl)picolinamide

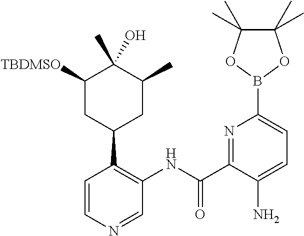 [SMILES: C[C@H]1C[C@@H](c2ccncc2NC(=O)c2nc(B3OC(C)(C)C(C)(C)O3)ccc2N)C[C@@H](O[Si](C)(C)C(C)(C)C)[C@]1(C)O]

To a microwave vessel was added 3-amino-6-bromo-N-(4-((1R,3R,4R,5S)-3-(tert-butyldimethylsilyloxy)-4-hydroxy-4,5-dimethylcyclohexyl)pyridin-3-yl)picolinamide (1 equiv.), 4,4,4′,4′,5,5,5′,5′-octamethyl-2,2′-bi(1,3,2-dioxaborolane) (1.2 equiv.), tricyclohexylphosphine (0.25 equiv), Pd₂(dba)₃ (0.125 equiv) and dioxane. The reaction was degassed for 5 min, and then potassium acetate ((3.0 equiv) was added. The reaction mixture was microwaved at 120° C. for 10 min. The reaction mixture was diluted with EtOAc, which was filtered though Celite pad. The volatile material was removed to yield the crude 3-amino-N-(4-((1R,3R,4R,5S)-3-(tert-butyldimethylsilyloxy)-4-hydroxy-4,5-dimethylcyclohexyl)pyridin-3-yl)-6-(4,4,5,5-tetramethyl-1,3,2-dioxaborolan-2-yl)picolinamide. LCMS (m/z): 515.2 (MH⁺ for boronic acid), Rₜ=0.76 min.

### Synthesis of 3-amino-6-bromo-N-(4-((1R,3R,4R,5S)-3-(tert-butyldimethylsilyloxy)-4-hydroxy-4,5-dimethylcyclohexyl)pyridin-3-yl)-5-fluoropicolinamide

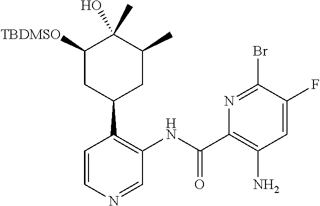 [SMILES: C[C@H]1C[C@@H](c2ccncc2NC(=O)c2nc(Br)c(F)cc2N)C[C@@H](O[Si](C)(C)C(C)(C)C)[C@]1(C)O]

Following Method 6, (1R,2R,4R,6S)-4-(3-aminopyridin-4-yl)-2-(tert-butyldimethylsilyloxy)-1,6-dimethylcyclohexanol and 3-amino-6-bromo-5-fluoropicolinic acid were coupled and following addition of EtOAc and washing with H₂O, NaCl_{(sat.)} and drying over MgSO₄, 3-amino-6-bromo-N-(4-((1R,3R,4R,5S)-3-(tert-butyldimethylsilyloxy)-4-hydroxy-4,5-dimethylcyclohexyl)pyridin-3-yl)-5-fluoropicolinamide was obtained. LCMS (m/z): 567.1/569.1 (MH⁺), Rₜ=1.01 min.

### Synthesis of 3-amino-6-bromo-N-(4-((1R,3R,4R,5S)-3-(tert-butyldimethylsilyloxy)-4-ethyl-4-hydroxy-5-methylcyclohexyl)pyridin-3-yl)picolinamide

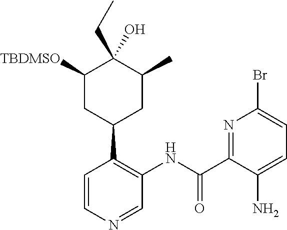 [SMILES: CC[C@@]1(O)[C@@H](C)C[C@@H](c2ccncc2NC(=O)c2nc(Br)ccc2N)C[C@H]1O[Si](C)(C)C(C)(C)C]

Following Method 6, (1R,2R,4R,6S)-4-(3-aminopyridin-4-yl)-2-(tert-butyldimethylsilyloxy)-1,6-dimethylcyclohexanol and 3-amino-6-bromopicolinic acid were coupled and following addition of EtOAc and washing with H₂O, NaCl_{(sat.)} and drying over MgSO₄, 3-amino-6-bromo-N-(4-((1R,3R,4R,5S)-3-(tert-butyldimethylsilyloxy)-4-ethyl-4-hydroxy-5-methylcyclohexyl)pyridin-3-yl)picolinamide was obtained. LCMS (m/z): 563.1/565.1 (MH⁺), Rₜ=1.06 min.

### Synthesis of 3-amino-N-(4-((1R,3R,4R,5S)-3-(tert-butyldimethylsilyloxy)-4-ethyl-4-hydroxy-5-methylcyclohexyl)pyridin-3-yl)-6-(4,4,5,5-tetramethyl-1,3,2-dioxaborolan-2-yl)picolinamide

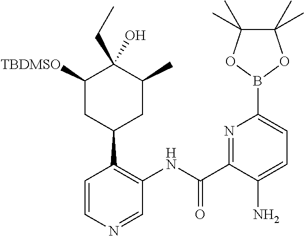 [SMILES: CC[C@@]1(O)[C@@H](C)C[C@@H](c2ccncc2NC(=O)c2nc(B3OC(C)(C)C(C)(C)O3)ccc2N)C[C@H]1O[Si](C)(C)C(C)(C)C]

To a microwave vessel was added 3-amino-6-bromo-N-(4-((1R,3R,4R,5S)-3-(tert-butyldimethylsilyloxy)-4-ethyl-4-hydroxy-5-methylcyclohexyl)pyridin-3-yl)picolinamide (1 equiv.), 4,4,4′,4′,5,5,5′,5′-octamethyl-2,2′-bi(1,3,2-dioxaborolane) (1.2 equiv.), tricyclohexylphosphine (0.25 equiv), Pd₂(dba)₃ (0.125 equiv) and dioxane. The reaction was degassed for 5 min, and then potassium acetate ((3.0 equiv) was added. The reaction mixture was microwaved at 120° C. for 10 min. The reaction mixture was diluted with EtOAc, which was filtered though Celite pad. The volatile material was removed to yield the crude 3-amino-N-(4-((1R,3R,4R,5S)-3-(tert-butyldimethylsilyloxy)-4-ethyl-4-hydroxy-5-methylcyclohexyl)pyridin-3-yl)-6-(4,4,5,5-tetramethyl-1,3,2-dioxaborolan-2-yl)picolinamide. LCMS (m/z): 529.2 (MH⁺ for boronic acid), Rₜ=0.86 min.

### Synthesis of (+/−)-3-amino-6-bromo-N-(4-((1R,3R,4R,5S)-4-(fluoromethyl)-3,4-dihydroxy-5-methylcyclohexyl)pyridin-3-yl)picolinamide

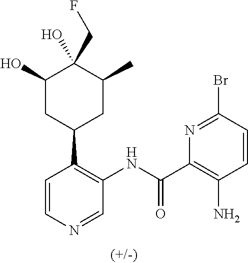 [SMILES: C[C@H]1C[C@@H](c2ccncc2NC(=O)c2nc(Br)ccc2N)C[C@@H](O)[C@@]1(O)CF]

Following Method 6, (+/−)-(1R,2R,4R,6S)-4-(3-aminopyridin-4-yl)-1-(fluoromethyl)-6-methylcyclohexane-1,2-diol and 3-amino-6-bromopicolinic acid were coupled and following addition of EtOAc and washing with H₂O, NaCl_{(sat.)} and drying over MgSO₄, (+/−)-3-amino-6-bromo-N-(4-((1R,3R,4R,5S)-4-(fluoromethyl)-3,4-dihydroxy-5-methylcyclohexyl)pyridin-3-yl)picolinamide was obtained. LCMS (m/z): 453/455 (MH⁺), Rₜ=0.55 min.

### Synthesis of (+/−)-3-amino-N-(4-((1R,3R,4R,5S)-4-(fluoromethyl)-3,4-dihydroxy-5-methylcyclohexyl)pyridin-3-yl)-6-(4,4,5,5-tetramethyl-1,3,2-dioxaborolan-2-yl)picolinamide

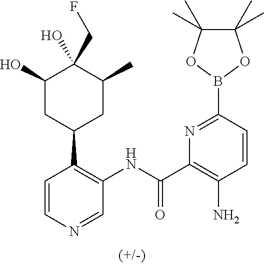 [SMILES: C[C@H]1C[C@@H](c2ccncc2NC(=O)c2nc(B3OC(C)(C)C(C)(C)O3)ccc2N)C[C@@H](O)[C@@]1(O)CF]

To a microwave vessel was added (+/−)-3-amino-6-bromo-N-(4-((1R,3R,4R,5S)-4-(fluoromethyl)-3,4-dihydroxy-5-methylcyclohexyl)pyridin-3-yl)picolinamide (1 equiv.), 4,4,4′,4′,5,5,5′,5′-octamethyl-2,2′-bi(1,3,2-dioxaborolane) (1.2 equiv.), tricyclohexylphosphine (0.25 equiv), Pd₂(dba)₃ (0.125 equiv) and dioxane. The reaction was degassed for 5 min, and then potassium acetate ((3.0 equiv) was added. The reaction mixture was microwaved at 120° C. for 10 min. The reaction mixture was diluted with EtOAc, which was filtered though Celite pad. The volatile material was removed to yield the crude (+/−)-3-amino-N-(4-((1R,3R,4R,5S)-4-(fluoromethyl)-3,4-dihydroxy-5-methylcyclohexyl)pyridin-3-yl)-6-(4,4,5,5-tetramethyl-1,3,2-dioxaborolan-2-yl)picolinamide. LCMS (m/z): 419.0 (MH⁺ for boronic acid), Rₜ=0.41 min.

### Synthesis of 2-(benzyloxy)-3,6-difluoropyridine

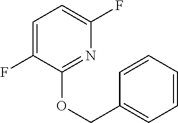 [SMILES: Fc1ccc(F)c(OCc2ccccc2)n1]

To a sealed tube, 2,3,6-trifluoropyridine (2 equiv.), benzyl alcohol (1 equiv.), potassium carbonate (15 quiv.), and NMP (0.5 M) were added. The reaction mixture was heated at 100° C. for overnight. After cooled down, the reaction mixture was extracted with EtOAc and the organic layer was washed with water and brine. The crude product was purified by ISCO (gradient EtOAc in Heptane) to yield 2-(benzyloxy)-3,6-difluoropyridine. LCMS (m/z): 222.0 (MH⁺), Rₜ=1.04 min. ¹H-NMR (400 MHz, CDCl₃) δ ppm 8.45-7.30 (m, 5H), 6.44 (m, 1H), 5.42 (s, 2H).

### Synthesis of 3,6-difluoropyridin-2-ol

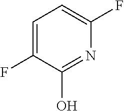

To a solution of 2-(benzyloxy)-3,6-difluoropyridine (1 equiv.) in MeOH (5 ml) was added Pd—C (0.1 equiv.). After degassed with N₂ stream, the reaction mixture was flushed with hydrogen gas, equipped with hydrogen balloon, and stirred for overnight at room temperature. The reaction mixture was filtered through Celite pad and washed with EtOAc. The volatile material was evaporated in vacuo. The crude 3,6-difluoropyridin-2-ol was obtained in 88% yield, which was used for the next step without purification. LCMS (m/z): 132.0 (MH⁺), Rₜ=0.36 min.

### Synthesis of 3,6-difluoropyridin-2-yl trifluoromethanesulfonate

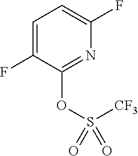 [SMILES: O=S(=O)(Oc1nc(F)ccc1F)C(F)(F)F]

To a solution of 3,6-difluoropyridin-2-ol (1 equiv.) in DCM (0.3 M) was added pyridine (1.5 equiv.) and trifluoromethanesulfonic anhydride (1.05 equiv at 0° C. The reaction mixture was warmed up to room temperature for 1 h. After quenched with NaHCO₃ solution, the reaction mixture was extracted with EtOAc. The organic layer was washed with saturated NaHCO₃ solution and brine, dried over anhydrous Na₂SO₄, filtered, and dried in vacuo. The crude 3,6-difluoropyridin-2-yl trifluoromethanesulfonate was immediately used for the next reaction.

### Synthesis of 2-chloro-6-(difluoromethoxy)pyridine

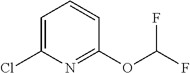 [SMILES: FC(F)Oc1cccc(Cl)n1]

A solution of 6-chloropyridin-2-ol (1.0 equiv.), sodium 2-chloro-2,2-difluoroacetate (2.0 equiv.) and sodium hydroxide (1.1 equiv.) in DMF (0.77 M) was heated at 55° C. for 18 hrs, the reaction mixture was then partitioned between EtOAc and sat. NaHCO₃ solution, the aqueous was extracted by EtOAc for 3 more times, combined organic was washed by water and brine, dried over anhydrous sodium sulfate, concentrated in vacuo. The crude product was purified via silica gel to yield 2-chloro-6-(difluoromethoxy)pyridine in 53% yield: LCMS (m/z): 180.0 (MH⁺), Rₜ=0.87 min. ¹H NMR (400 M Hz, CHLOROFORM-d) δ ppm 7.69 (t, J=8.0 Hz, 1H), 7.44 (t, J=72 Hz, 1H), 7.14 (d, J=7.8 Hz, 1H), 6.83 (d, J=7.8 Hz, 1H).

### Synthesis of 2-bromo-6-(2,2,2-trifluoroethoxy)pyridine

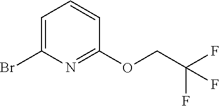 [SMILES: FC(F)(F)COc1cccc(Br)n1]

To a solution of 2,6-dibromopyridine (1.0 equiv.) in DMF (2.0 M) was added sodium hydride (60% in mineral oil, 1.1 equiv.) at 0° C., the mixture was stirred at 0° C. for 10 mins, 2,2,2-trifluoroethanol (1.2 equiv.) was added, the reaction mixture was then heated to 60° C. for 1.5 hr. the reaction mixture was then partitioned between EtOAc and water, the aqueous was extracted by EtOAc for 3 more times, combined organic was washed by water and brine, dried over anhydrous sodium sulfate, concentrated in vacuo. The crude product was purified via silica gel (5% DCM/heptane) to yield 2-bromo-6-(2,2,2-trifluoroethoxy)pyridine in 40% yield: LCMS (m/z): 180.0 (MH⁺), Rₜ=0.87 min. ¹H NMR (400 M Hz, CHLOROFORM-d) δ ppm 7.50 (t, J=7.8 Hz, 1H), 7.17 (d, J=7.0 Hz, 1H), 6.83 (dd, J=8.2, 0.8 Hz, 1H), 4.74 (q, J=8.3 Hz, 2H).

### Method 7

### Synthesis of 5-amino-N-(4-((1R,3R,4R,5S)-3,4-dihydroxy-4,5-dimethylcyclohexyl)pyridin-3-yl)-3′-fluoro-2,2′-bipyridine-6-carboxamide

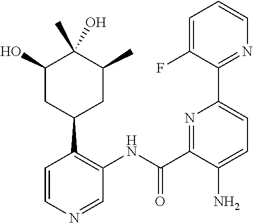 [SMILES: C[C@H]1C[C@@H](c2ccncc2NC(=O)c2nc(-c3ncccc3F)ccc2N)C[C@@H](O)[C@]1(C)O]

To a microwave vial (5 mL), 3-amino-N-(4-((1R,3R,4R,5S)-3-(tert-butyldimethylsilyloxy)-4-hydroxy-4,5-dimethylcyclohexyl)pyridin-3-yl)-6-(4,4,5,5-tetramethyl-1,3,2-dioxaborolan-2-yl)picolinamide (40 mg, 0.067 mmol), 2-bromo-3-fluoropyridine (17.70 mg, 0.101 mmol), PdCl₂(dppf) (7.36 mg, 10.06 μmol), DME (0.503 ml) and 2M Na₂CO₃ solution (0.168 ml) were added. The reaction mixture was degassed by N₂ stream for 10 min. The reaction mixture was heated in a microwave at 120° C. for 10 min. To the reaction mixture, anhydrous sodium sulfate was added to remove water and diluted with EtOAc. The mixture was filtered and concentrated in vacuo to yield 5-amino-N-(4-((1R,3R,4R,5S)-3-(tert-butyldimethylsilyloxy)-4-hydroxy-4,5-dimethylcyclohexyl)pyridin-3-yl)-3′-fluoro-2,2′-bipyridine-6-carboxamide. LCMS (m/z): 566.2 (MH⁺), Rₜ=0.95 min. The crude product was dissolved in MeOH and THF (1:1, 1 mL) followed by 0.5 mL of 3N HCl solution. After 1 h, the mixture was basified with Na₂CO₃ solution and worked up with EtOAc. The concentrated crude product was purified via prep HPLC. The pure fractions were lyophilized to yield 5-amino-N-(4-((1R,3R,4R,5S)-3,4-dihydroxy-4,5-dimethylcyclohexyl)pyridin-3-yl)-3′-fluoro-2,2′-bipyridine-6-carboxamide as the TFA salt (3.9 mg). LCMS (m/z): 452.1 (MH⁺), Rₜ=0.47 min. ¹H-NMR (DMSO, 400 MHz)-δ 10.44 (s, 1H), 9.28 (s, 1H), 8.53 (d, J=4 Hz, 1H), 8.45 (d, J=4 Hz, 1H), 8.12 (d, J=8 Hz, 1H), 7.78 (m, 1H), 7.72 (m, 1H), 7.52 (m, 2H), 7.43 (d, J=8 Hz, 1H), 7.29 (bs, 2H), 3.12 (m, 1H), 2.49 (m, 1H), 1.78 (m, 1H), 1.61 (m, 2H), 1.53 (m, 1H), 1.31 (m, 1H), 0.92 (s, 3H), 0.77 (d, J=8 Hz, 3H).

Alternatively, the above Suzuki conditions were employed using 3-amino-6-bromo-N-(4-((1R,3R,4R,5S)-3-(tert-butyldimethylsilyloxy)-4-hydroxy-4,5-dimethylcyclohexyl)pyridin-3-yl)picolinamide and boronic acids to yield after deprotection, compounds in Table 2.

The following compounds were prepared using Method 7 for Suzuki reaction and Method 6 for deprotection:

TABLE 2

| | | LC/MS | LC/MS | |
|---|---|---|---|---|
| Ex. | | (M + H on | (Rf on | Chemical |
| No. | Structure | UPLC) | UPLC) | Name |
| 121 | 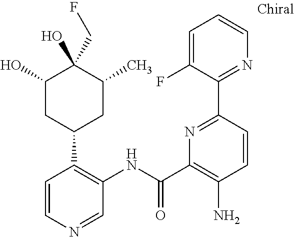 [SMILES: C[C@@H]1C[C@H](c2ccncc2NC(=O)c2nc(-c3ncccc3F)ccc2N)C[C@H](O)[C@]1(O)CF] | 470.1 | 0.52 | 5-amino-3′-fluoro-N- (4-((1S,3S,4S,5R)- 4-(fluoromethyl )- 3,4-dihydroxy-5- methylcyclohexyl) pyridin-3-yl)-2,2′- bipyridine-6- carboxamide |
| 122 | 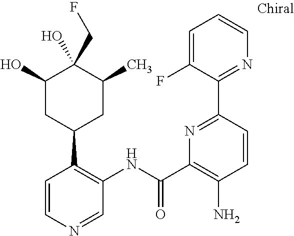 [SMILES: C[C@H]1C[C@@H](c2ccncc2NC(=O)c2nc(-c3ncccc3F)ccc2N)C[C@@H](O)[C@@]1(O)CF] | 470.1 | 0.52 | 5-amino-3′-fluoro-N- (4-((1R,3R,4R,5S)- 4-(fluoromethyl )- 3,4-dihydroxy-5- methylcyclohexyl) pyridin-3-yl)-2,2′- bipyridine-6- carboxamide |
| 123 | 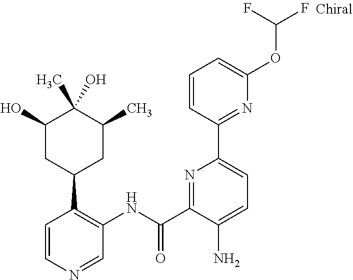 [SMILES: C[C@H]1C[C@@H](c2ccncc2NC(=O)c2nc(-c3cccc(OC(F)F)n3)ccc2N)C[C@@H](O)[C@]1(C)O] | 500.1 | 0.70 | 5-amino-6′- (difluoromethoxy)- N-(4- ((1R,3R,4R,5S)-3,4- dihydroxy-4,5- dimethylcyclohexyl) pyridin-3-yl)-2,2′- bipyridine-6- carboxamide |
| 124 | 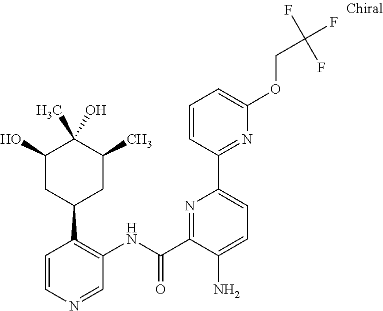 [SMILES: C[C@H]1C[C@@H](c2ccncc2NC(=O)c2nc(-c3cccc(OCC(F)(F)F)n3)ccc2N)C[C@@H](O)[C@]1(C)O] | 532.1 | 0.76 | 5-amino-N-(4- ((1R,3R,4R,5S)-3,4- dihydroxy-4,5- dimethylcyclohexyl) pyridin-3-yl)-6′- (2,2,2- trifluoroethoxy)-2,2′- bipyridine-6- carboxamide |
| 125 | 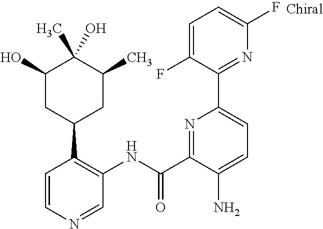 [SMILES: C[C@H]1C[C@@H](c2ccncc2NC(=O)c2nc(-c3nc(F)ccc3F)ccc2N)C[C@@H](O)[C@]1(C)O] | 470.1 | 0.64 | 5-amino-N-(4- ((1R,3R,4R,5S)-3,4- dihydroxy-4,5- dimethylcyclohexyl) pyridin-3-yl)-3′,6′- difluoro-2,2′- bipyridine-6- carboxamide |
| 126 | 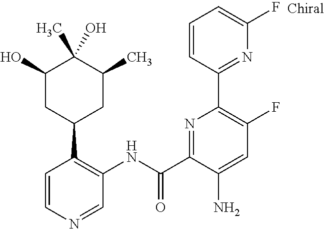 [SMILES: C[C@H]1C[C@@H](c2ccncc2NC(=O)c2nc(-c3cccc(F)n3)c(F)cc2N)C[C@@H](O)[C@]1(C)O] | 470.1 | 0.55 | 5-amino-N-(4- ((1R,3R,4R,5S)-3,4- dihydroxy-4,5- dimethylcyclohexyl) pyridin-3-yl)-3,6′- difluoro-2,2′- bipyridine-6- carboxamide |
| 127 | 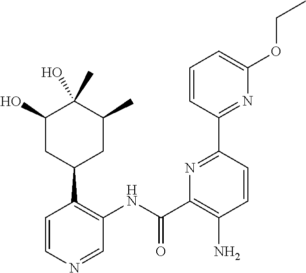 [SMILES: CCOc1cccc(-c2ccc(N)c(C(=O)Nc3cnccc3[C@H]3C[C@@H](O)[C@](C)(O)[C@@H](C)C3)n2)n1] | 478.2 | 0.63 | 5-amino-N-(4- ((1R,3R,4R,5S)-3,4- dihydroxy-4,5- dimethylcyclohexyl) pyridin-3-yl)-6′- ethoxy-2,2′- bipyridine-6- carboxamide |
| 128 | 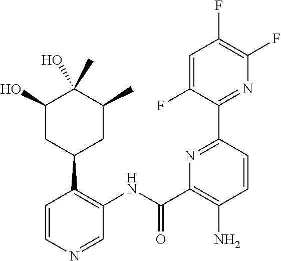 [SMILES: C[C@H]1C[C@@H](c2ccncc2NC(=O)c2nc(-c3nc(F)c(F)cc3F)ccc2N)C[C@@H](O)[C@]1(C)O] | 488.1 | 0.64 | 5-amino-N-(4- ((1R,3R,4R,5S)-3,4- dihydroxy-4,5- dimethylcyclohexyl) pyridin-3-yl)-3′,5′,6′- trifluoro-2,2′- bipyridine-6- carboxamide |
| 129 | 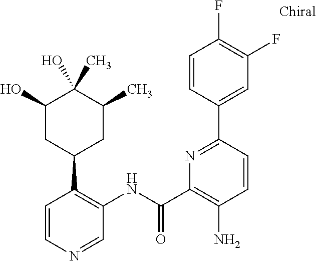 [SMILES: C[C@H]1C[C@@H](c2ccncc2NC(=O)c2nc(-c3ccc(F)c(F)c3)ccc2N)C[C@@H](O)[C@]1(C)O] | 469.1 | 0.66 | 3-amino-6-(3,4- difluorophenyl)-N (4-((1R,3R,4R,5S)- 3,4-dihydroxy-4,5- dimethylcyclohexyl) pyridin-3- yl)picolinamide |
| 130 | 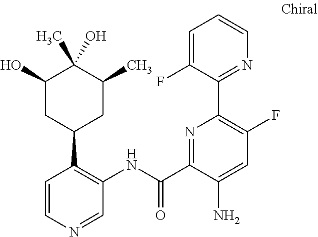 [SMILES: C[C@H]1C[C@@H](c2ccncc2NC(=O)c2nc(-c3ncccc3F)c(F)cc2N)C[C@@H](O)[C@]1(C)O] | 470.1 | 0.52 | 5-amino-N-(4- ((1R,3R,4R,5S)-3,4- dihydroxy-4,5- dimethylcyclohexyl) pyridin-3-yl)-3,3′- difluoro-2,2′- bipyridine-6- carboxamide |
| 131 | 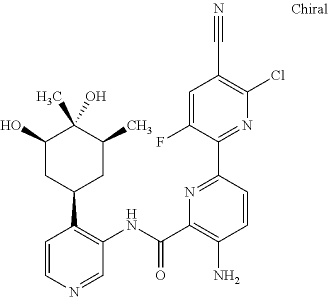 [SMILES: C[C@H]1C[C@@H](c2ccncc2NC(=O)c2nc(-c3nc(Cl)c(C#N)cc3F)ccc2N)C[C@@H](O)[C@]1(C)O] | 511.1 | 0.66 | 5-amino-6′-chloro- 5′-cyano-N-(4- ((1R,3R,4R,5S)-3,4- dihydroxy-4,5- dimethylcyclohexyl) pyridin-3-yl)-3′- fluoro-2,2′- bipyridine-6- carboxamide |
| 132 | 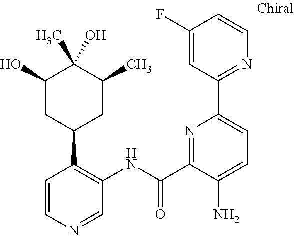 [SMILES: C[C@H]1C[C@@H](c2ccncc2NC(=O)c2nc(-c3cc(F)ccn3)ccc2N)C[C@@H](O)[C@]1(C)O] | 452.1 | 0.39 | 5-amino-N-(4- ((1R,3R,4R,5S)-3,4- dihydroxy-4,5- dimethylcyclohexyl) pyridin-3-yl)-4′- fluoro-2,2′- bipyridine-6- carboxamide |
| 133 | 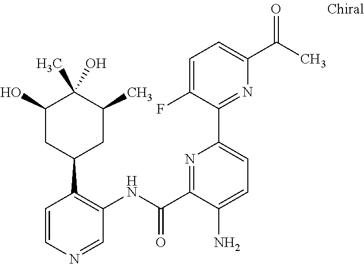 [SMILES: CC(=O)c1ccc(F)c(-c2ccc(N)c(C(=O)Nc3cnccc3[C@H]3C[C@@H](O)[C@](C)(O)[C@@H](C)C3)n2)n1] | 494.2 | 0.60 | 6′-acetyl-5-amino-N- (4-((1R,3R,4R,5S)- 3,4-dihydroxy-4,5- dimethylcyclohexyl) pyridin-3-yl)-3′- fluoro-2,2′- bipyridine-6- carboxamide |
| 134 | 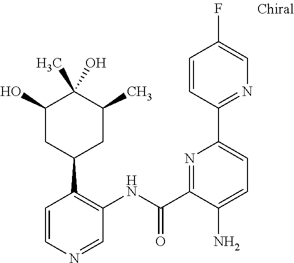 [SMILES: C[C@H]1C[C@@H](c2ccncc2NC(=O)c2nc(-c3ccc(F)cn3)ccc2N)C[C@@H](O)[C@]1(C)O] | 452.1 | 0.53 | 5-amino-N-(4- ((1R,3R,4R,5S)-3,4- dihydroxy-4,5- dimethylcyclohexyl) pyridin-3-yl)-5′- fluoro-2,2′- bipyridine-6- carboxamide |
| 135 | 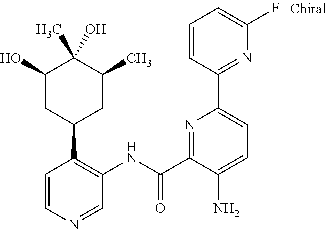 [SMILES: C[C@H]1C[C@@H](c2ccncc2NC(=O)c2nc(-c3cccc(F)n3)ccc2N)C[C@@H](O)[C@]1(C)O] | 452.1 | 0.57 | 5-amino-N-(4- ((1R,3R,4R,5S)-3,4- dihydroxy-4,5- dimethylcyclohexyl) pyridin-3-yl)-6′- fluoro-2,2′- bipyridine-6- carboxamide |
| 136 | 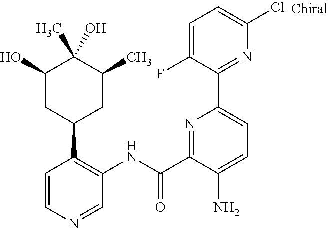 [SMILES: C[C@H]1C[C@@H](c2ccncc2NC(=O)c2nc(-c3nc(Cl)ccc3F)ccc2N)C[C@@H](O)[C@]1(C)O] | 486.1 | 0.61 | 5-amino-6′-chloro- N-(4- ((1R,3R,4R,5S)-3,4- dihydroxy-4,5- dimethylcyclohexyl) pyridin-3-yl)-3′- fluoro-2,2′- bipyridine-6- carboxamide |
| 137 | 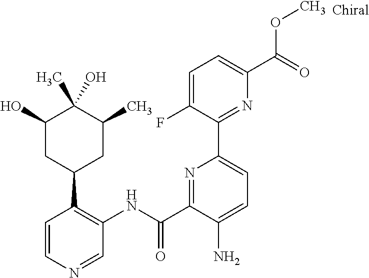 [SMILES: COC(=O)c1ccc(F)c(-c2ccc(N)c(C(=O)Nc3cnccc3[C@H]3C[C@@H](O)[C@](C)(O)[C@@H](C)C3)n2)n1] | 510.2 | 0.55 | methyl 5′-amino-6′- (4-((1R,3R,4R,5S)- 3,4-dihydroxy-4,5- dimethylcyclohexyl) pyridin-3- ylcarbamoyl)-3- fluoro-2,2′- bipyridine-6- carboxylate |
| 138 | 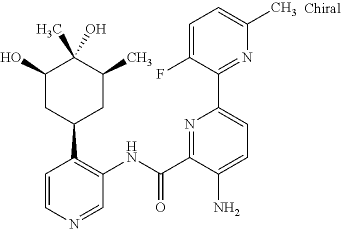 [SMILES: Cc1ccc(F)c(-c2ccc(N)c(C(=O)Nc3cnccc3[C@H]3C[C@@H](O)[C@](C)(O)[C@@H](C)C3)n2)n1] | 466.1 | 0.47 | 5-amino-N-(4- ((1R,3R,4R,5S)-3,4- dihydroxy-4,5- dimethylcyclohexyl) pyridin-3-yl)-3′- fluoro-6′-methyl- 2,2′-bipyridine-6- carboxamide |
| 139 | 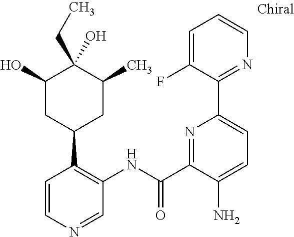 [SMILES: CC[C@]1(O)[C@H](O)C[C@H](c2ccncc2NC(=O)c2nc(-c3ncccc3F)ccc2N)C[C@@H]1C] | 466.1 | 0.52 | 5-amino-N-(4- ((1R,3R,4R,5S)-4- ethyl-3,4-dihydroxy- 5- methylcyclohexyl) pyridin-3-yl)-3′-fluoro- 2,2′-bipyridine-6- carboxamide |
| 140 | 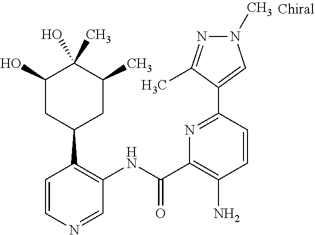 [SMILES: Cc1nn(C)cc1-c1ccc(N)c(C(=O)Nc2cnccc2[C@H]2C[C@@H](O)[C@](C)(O)[C@@H](C)C2)n1] | 451.2 | 0.46 | 3-amino-N-(4- ((1R,3R,4R,5S)-3,4- dihydroxy-4,5- dimethylcyclohexyl) pyridin-3-yl)-6-(1,3- dimethyl-1H- pyrazol-4- yl)picolinamide |
| 141 | 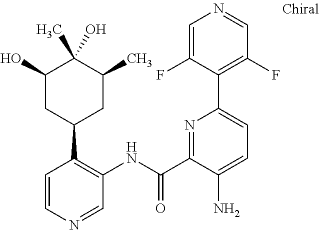 [SMILES: C[C@H]1C[C@@H](c2ccncc2NC(=O)c2nc(-c3c(F)cncc3F)ccc2N)C[C@@H](O)[C@]1(C)O] | 470.1 | 0.52 | 5-amino-N-(4- ((1R,3R,4R,5S)-3,4- dihydroxy-4,5- dimethylcyclohexyl) pyridin-3-yl)-3′,5′- difluoro-2,4′- bipyridine-6- carboxamide |
| 142 | 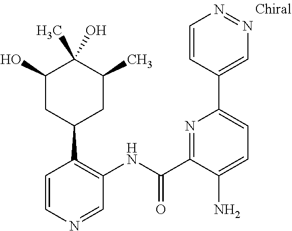 [SMILES: C[C@H]1C[C@@H](c2ccncc2NC(=O)c2nc(-c3ccnnc3)ccc2N)C[C@@H](O)[C@]1(C)O] | 435.2 | 0.38 | 3-amino-N-(4- ((1R,3R,4R,5S)-3,4- dihydroxy-4,5- dimethylcyclohexyl) pyridin-3-yl)-6- (pyridazin-4- yl)picolinamide |
| 143 | 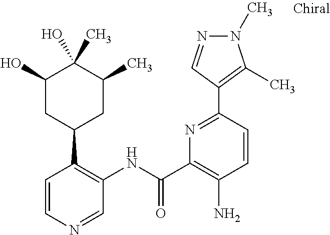 [SMILES: Cc1c(-c2ccc(N)c(C(=O)Nc3cnccc3[C@H]3C[C@@H](O)[C@](C)(O)[C@@H](C)C3)n2)cnn1C] | 451.2 | 0.48 | 3-amino-N-(4- ((1R,3R,4R,5S)-3,4- dihydroxy-4,5- dimethylcyclohexyl) pyridin-3-yl)-6-(1,5- dimethyl-1H- pyrazol-4- yl)picolinamide |
| 144 | 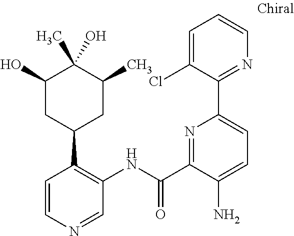 [SMILES: C[C@H]1C[C@@H](c2ccncc2NC(=O)c2nc(-c3ncccc3Cl)ccc2N)C[C@@H](O)[C@]1(C)O] | 468.1 | 0.52 | 5-amino-3′-chloro- N-(4- ((1R,3R,4R,5S)-3,4- dihydroxy-4,5- dimethylcyclohexyl) pyridin-3-yl)-2,2′- bipyridine-6- carboxamide |
| 145 | 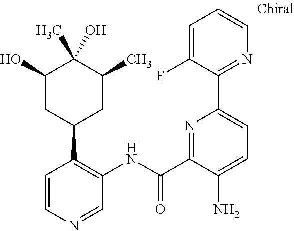 [SMILES: C[C@H]1C[C@@H](c2ccncc2NC(=O)c2nc(-c3ncccc3F)ccc2N)C[C@@H](O)[C@]1(C)O] | 452.1 | 0.47 | 5-amino-N-(4- ((1R,3R,4R,5S)-3,4- dihydroxy-4,5- dimethylcyclohexyl) pyridin-3-yl)-3′- fluoro-2,2′- bipyridine-6- carboxamide |
| 146 | 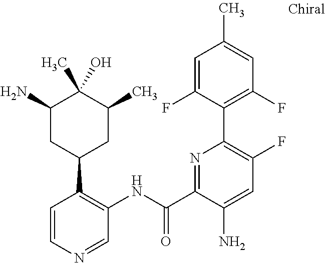 [SMILES: Cc1cc(F)c(-c2nc(C(=O)Nc3cnccc3[C@H]3C[C@@H](N)[C@](C)(O)[C@@H](C)C3)c(N)cc2F)c(F)c1] | 500.1 | 0.64 | 3-amino-N-(4- ((1R,3R,4R,5S)-3- amino-4-hydroxy- 4,5- dimethylcyclohexyl) pyridin-3-yl)-6-(2,6- difluoro-4- methylphenyl)-5- fluoropicolinamide |
| 147 | 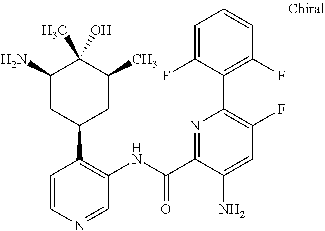 [SMILES: C[C@H]1C[C@@H](c2ccncc2NC(=O)c2nc(-c3c(F)cccc3F)c(F)cc2N)C[C@@H](N)[C@]1(C)O] | 486.0 | 0.60 | 3-amino-N-(4- ((1R,3R,4R,5S)-3- amino-4-hydroxy- 4,5- dimethylcyclohexyl) pyridin-3-yl)-6-(2,6- difluorophenyl)-5- fluoropicolinamide |

In addition to LC/MS and LC characterization, representative compounds were analyzed by ¹H-NMR. The following are typical spectra of the compounds of the invention.

TABLE 3

| Ex. No. | ¹H-NMR data |
|---|---|
| 2 | (400 MHz, CD₃OD) δ ppm ppm 9.02 (s, 1 H), 8.50 (d, 1 H), 8.40 (dd, 1 |
| | H), 7.98-8.05 (m, 1 H), 7.69 (d, 1 H), 7.09 (d, 1 H), 3.17-3.28 (m, 1 H), |
| | 3.09 (dd, 1 H), 2.78 (q, 2 H), 2.08 (dd, 1 H), 1.86 (q, 2 H), 1.67-1.75 (m, |
| | 1 H), 1.47 (q, 1 H), 1.31 (t, 3 H), 1.16 (s, 3 H), 0.96 (d, 3 H) |
| 4 | (400 MHz, CD₃OD) δ ppm 0.97 (d, 3 H) 1.16 (s, 3 H) 1.47 (q, 1 H) 1.64- |
| | 1.75 (m, 1 H) 1.85 (q, 2 H) 1.99-2.15 (m, 1 H) 3.08 (dd, 1 H) 3.17-3.28 |
| | (m, 1 H) 3.46 (s, 3 H) 4.57 (s, 2 H) 7.20 (d, 2 H) 7.69 (d, 1 H) 8.03 (t, 1 |
| | H) 8.41 (dd, 1 H) 8.51 (d, 1 H) 8.99 (s, 1 H) |
| 5 | (400 MHz, CD₃OD) δ ppm 9.19 (s, 1 H) 8.55 (d, 1 H) 8.40 (dd, 1 H) 8.01 |
| | (t, 1 H) 7.80 (d, 1 H) 6.87 (d, 2 H) 4.12-4.22 (m, 2 H) 3.87-3.98 (m, 2 |
| | H) 3.22-3.30 (m, 1 H) 3.12 (dd, 1 H) 2.05-2.17 (m, 1 H) 1.82-1.97 |
| | (m, 2 H) 1.69-1.81 (m, 1 H) 1.50 (q, 1 H) 1.18 (s, 3 H) 0.98 (d, 3 H) |
| 6 | (400 MHz, CD₃OD) δ 9.07 (s, 1 H) 8.52 (d, 1 H) 8.44 (dd, 1 H) 8.05 (t, 1 |
| | H) 7.71 (d, 1 H) 7.37 (td, 1 H) 7.14 (td, 1 H) 4.21-4.34 (m, 2 H) 3.75- |
| | 3.88 (m, 2 H) 3.46 (s, 3 H) 3.18-3.30 (m, 1 H) 3.12 (dd, 1 H) 2.05-2.18 |
| | (m, 1 H) 1.78-1.94 (m, 2 H) 1.65-1.78 (m, 1 H) 1.52 (q, 1 H) 1.18 (s, 3 |
| | H) 0.99 (d, 3 H) |
| 7 | (400 MHz, CD₃OD) δ 9.16 (s, 1 H) 8.54 (d, 1 H) 8.40 (dd, 1 H) 8.01 (t, 1 |
| | H) 7.78 (d, 1 H) 6.86 (d, 2 H) 4.20 - 4.27 (m, 2 H) 3.80 (m, 2 H) 3.45 (s, |
| | 3 H) 3.21-3.30 (m, 1 H) 3.12 (dd, 1 H) 2.06-2.16 (m, 1 H) 1.82-1.97 |
| | (m, 2 H) 1.69-1.81 (m, 1 H) 1.50 (q, 1 H) 1.18 (s, 3 H) 0.99 (d, 3 H) |
| 8 | (400 MHz, CD₃OD) δ 9.09 (s, 1 H) 8.53 (d, 1 H) 8.41 (dd, 1 H) 8.03 (t, |
| | J = 8.61 Hz, 1 H) 7.73 (d, 1 H) 7.13 (d, 2 H) 3.19-3.30 (m, 1 H) 3.12 (dd, |
| | 1 H) 2.60 (s, 3 H) 2.04-2.16 (m, 1 H) 1.81-1.95 (m, 2 H) 1.70-1.80 |
| | (m, 1 H) 1.49 (q, 1 H) 1.18 (s, 3 H) 0.98 (d, 3 H) |
| 9 | (400 MHz, CD₃OD) δ 0.91 (d, 3 H) 1.09 (s, 3 H) 1.23-1.42 (m, 1 H) 1.51- |
| | 1.75 (m, 3 H) 1.94 (d, 1 H) 2.96-3.14 (m, 1 H) 3.29 (s, 3 H) 3.48 (dd, 1 |
| | H) 7.46 (d, 1 H) 7.88 (d, 2 H) 8.08 (t, 1 H) 8.37 (d, 1 H) 8.47 (dd, 1 H) |
| | 8.92 (s, 1 H) |
| 10 | (400 MHz, CD₃OD) δ 0.91 (d, 3 H) 1.09 (s, 3 H) 1.30-1.43 (m, 1 H) 1.51- |
| | 1.76 (m, 3 H) 1.88-1.98 (m, 1 H) 2.98-3.12 (m, 1 H) 3.29 (s, 3 H) |
| | 3.48 (dd, 1 H) 7.46 (d, 1 H) 7.88 (d, 2 H) 8.08 (t, 1 H) 8.37 (d, 1 H) 8.46 |
| | (dd, 1 H) 8.92 (s, 1 H) |
| 16 | (400 MHz, CDCl₃) δ10.00 (s, 1H), 9.41 (s, 1H), 8.45-8.31 (m, 2 H), 7.74 |
| | (t, 1H), 7.17 (d, 1H), 6.66 (d, 2H), 3.00 (br. s., 1 H) 3.88 (s, 3H), 2.67 (dd, |
| | 1H), 1.82-1.62 (m, 3H), 1.36 (d, 1H), 1.26 (bs, 1H), 1.03 (s, 2H), 0.94 (d, |
| | 3H), 0.88 (s, 1H). |
| 27 | (400 MHz, CDCl₃) δ 9.93 (s, 1H), 9.37 (s, 1H), 8.35-8.50 (m, 2H), 7.78 |
| | (t, 1H), 7.42-7.63 (m, 1H), 7.03-7.23 (m, 3H), 3.07-2.90 (m, 1H), |
| | 2.71-2.51 (m, 1H), 1.92 (dd, 1H), 1.79-1.59 (m, 3H), 1.43-1.29 (m, 1H), |
| | 1.26 (bs, 1H), 1.03 (s, 3H), 0.98-0.84 (m, 3H). |
| 145 | (400 MHz, DMSO-d₆) δ 10.2 (s, 1H), 9.08 (s, 1H), 8.52 (m, 1H), 8.31 (d, |
| | 1H), 8.10 (d, 1H), 7.77 (m, 1H), 7.49 (m, 1H), 7.41 (m, 2H), 7.26. (bs, |
| | 2H), 4.48 (d, 1H), 4.07 (s, 1H), 3.35 (m, 1H), 3.22 (m, 1H), 1.74 (m, 1H), |
| | 1.57 (m, 2H), 1.3 (2H, m), 0.94 (s, 3H), 0.84 (d, 3H). |
| 47 | (400 MHz, CDCl₃) δ10.08 (s, 1 H) 9.30 (s, 1 H) 8.57 (s, 1 H) 8.42 (d, 1 |
| | H) 7.37-7.44 (m, 1 H) 7.18-7.23 (m, 1 H) 7.05 (t, 1 H) 6.11 (br. s., 2 H) |
| | 3.58-3.66 (m, 1 H) 3.02 (m, 1 H) 1.99-2.10 (m, 2 H) 1.75 (dd, 1 H) |
| | 1.67-1.71 (m, 1 H) 1.29-1.40 (m, 1 H) 1.14 (s, 3 H) 0.93 (d, 3 H) |
| 59 | (400 MHz, DMSO-d₆) δ 10.34 (bs, 1H), 8.62 (s, 1H), 8.32-8.37 (m, 2H), |
| | 8.18 (m, 1H), 7.68 (m, 1H), 7.33 (m, 3H), 4.87 (d, 1H), 4.54 (m, 1H), |
| | 4.11 (s, 1H), 3.71 (m, 1H), 3.61 (m, 1H), 3.39 (m, 1H), 2.53 (m, 1H), |
| | 1.80 (m, 2H), 1.51 (m, 3H), 0.85 (d, 3H). |
| 61 | (400 MHz, CDCl₃) δ 9.90 (bs, 1H), 9.34 (s, 1H), 8.43 (d, 1H), 8.41 (m, |
| | 1H), 7.78 (t, 1H), 7.51 (m, 1H), 7.19 (d, 1H), 7.1 (dd, 1H), 3.57 (m, 1H), |
| | 3.0 (m, 1H), 2.0 (m, 1H), 1.76-1.69 (m, 2H), 1.66-1.59 (m, 2H), 1.35- |
| | 1.26 (m, 2H), 1.12 (s, 3H), 0.9 (d, 3H). |
| 147 | (400 MHz, METHANOL-d4) δ ppm 7.94 (s, 1 H) 7.78 (d, J = 5.09 Hz, 1 |
| | H) 7.08 (d, J = 5.09 Hz, 1 H) 3.67 (m, 1 H) 2.84-3.04 (m, 1 H) 1.69-1.95 |
| | (m, 2 H) 1.69-1.79 (m, 1 H) 1.41-1.57 (m, 10 H) 1.29-1.41 (m, 1 H) |
| | 1.08 (s, 3 H) 1.03 (d, J = 6.65 Hz, 3 H) |
| 65 | (400 MHz, METHANOL-d4) δ ppm 0.81-0.97 (m, 3 H) 1.04 (s, 3 H) |
| | 1.20-1.44 (m, 2 H) 1.48-1.68 (m, 2 H) 1.69-1.82 (m, 2 H) 1.87 (d, |
| | J = 10.96 Hz, 1 H) 2.08 (dd, J = 9.39, 3.91 Hz, 2 H) 2.72 (dd, J = 12.13, |
| | 3.52 Hz, 1 H) 3.00-3.16 (m, 1 H) 3.52-3.71 (m, 2 H) 3.86-4.04 (m, 2 |
| | H) 4.69 (dt, J = 7.92, 4.06 Hz, 1 H) 6.83 (d, J = 10.17 Hz, 2 H) 7.44 (d, |
| | J = 5.48 Hz, 1 H) 7.94 (t, J = 8.80 Hz, 1 H) 8.25-8.42 (m, 2 H) 8.93 (s, 1 |
| | H) |
| 64 | (400 MHz, <CDCl3>) δ ppm 9.95 (s, 1H), 9.34 (s, 1H), 8.35-8.40 (m, |
| | 2H), 7.73 (dd, J = 8.0, 8.0, 1H), 7.17 (d, J = 4.8, 1H), 6.61 (d, J = 10.0, 2H), |
| | 4.59 (septet, J = 6.0, 1H), 2.96-3.04 (m, 1H), 2.70 (dd, J = 12.0, 3.6, 1H), |
| | 1.90-1.96 (m, 1H), 1.80-1.87 (m, 4H), 1.70-1.76 (m, 1H), 1.62-1.69 (m, |
| | 1H), 1.43-1.52 (m, 1H), 1.39 (d, J = 6.0, 6H), 1.30-1.39 (m, 1H), 1.04 (s, |
| | 3H), 0.94 (d, J = 6.8, 3H). |
| 71 | (400 MHz, <CDCl₃>) δ ppm 9.96 (s, 1H), 9.38 (s, 1H), 8.39-8.53 (m, |
| | 2H), 7.76 (dd, J = 8.8, 8.8, 1H), 7.17 (d, J = 4.8, 1H), 6.98 (d, J = 9.6, 2H), |
| | 4.09-4.13 (m, 2H), 3.51-3.58 (m, 2H), 2.95-3.02 (m, 1H), 2.82-2.87 (m, |
| | 1H), 2.67 (dd, J = 12.0, 4.0, 1H), 1.88-1.96 (m, 1H), 1.80-1.87 (m, 4H), |
| | 1.70-1.76 (m, 1H), 1.62-1.69 (m, 1H), 1.43-1.52 (m, 1H), 1.30-1.39 (m, |
| | 1H), 1.03 (s, 3H), 0.91 (d, J = 6.8, 3H). |
| 73 | (400 MHz, <cd3od>) d ppm 0.89 (d, J = 6.65 Hz, 3 H) 1.07 (s, 3 H) 1.17- |
| | 1.45 (m, 2 H) 1.56 (s, 6 H) 1.58-1.74 (m, 2 H) 1.84-1.96 (m, 1 H) 2.96- |
| | 3.10 (m, 1 H) 3.49 (dd, J = 11.74, 4.30 Hz, 1 H) 7.30 (d, J = 9.39 Hz, 2 H) |
| | 7.44 (d, J = 5.09 Hz, 1 H) 7.97 (t, J = 8.80 Hz, 1 H) 8.29-8.43 (m, 2 H) |
| | 8.91 (s, 1 H) |
| 74 | (400 MHz, <cd3od>) d ppm 0.89 (d, J = 6.65 Hz, 3 H) 1.07 (s, 3 H) 1.20- |
| | 1.44 (m, 2 H) 1.56 (s, 6 H) 1.58-1.74 (m, 2 H) 1.86-1.98 (m, 1 H) 2.98- |
| | 3.10 (m, 1 H) 3.49 (dd, J = 11.74, 4.30 Hz, 1 H) 7.30 (d, J = 9.39 Hz, 2 H) |
| | 7.44 (d, J = 5.09 Hz, 1 H) 7.97 (t, J = 8.80 Hz, 1 H) 8.28-8.44 (m, 2 H) |
| | 8.91 (s, 1 H) |
| 75 | (400 MHz, <cd3od>) d ppm 0.90 (d, J = 6.65 Hz, 3 H) 1.09 (s, 3 H) 1.25- |
| | 1.41 (m, 2 H) 1.50-1.76 (m, 2 H) 1.88-1.98 (m, 1 H) 2.99-3.11 (m, 1 |
| | H) 3.43-3.57 (m, 1 H) 4.83 (m, 2 H) 4.94 (d, J = 6.65 Hz, 2 H) 7.46 (d, |
| | J = 5.48 Hz, 1 H) 7.53 (d, J = 9.00 Hz, 2 H) 8.02 (t, J = 8.80 Hz, 1 H) 8.31- |
| | 8.47 (m, 2 H) 8.99 (s, 1 H) |
| 76 | (400 MHz, <cd3od>) d ppm 0.88 (d, J = 7.04 Hz, 3 H) 1.07 (s, 3 H) 1.23- |
| | 1.39 (m, 2 H) 1.49-1.77 (m, 2 H) 1.86-1.99 (m, 1 H) 2.91-3.10 (m, 1 |
| | H) 3.48 (dd, J = 11.74, 4.30 Hz, 1 H) 4.76-4.84 (m, 2 H) 4.91 (d, J = 6.65 |
| | Hz, 2 H) 7.43 (d, J = 5.09 Hz, 1 H) 7.51 (d, J = 9.00 Hz, 2 H) 8.00 (t, |
| | J = 8.61 Hz, 1 H) 8.30-8.44 (m, 2 H) 8.92 (s, 1 H) |
| 77 | (400 MHz, <cd3od>) d ppm 0.89 (d, J = 6.65 Hz, 3 H) 1.07 (s, 3 H) 1.22- |
| | 1.41 (m, 2 H) 1.49-1.73 (m, 2 H) 1.81-1.96 (m, 1 H) 2.02-2.15 (m, 2 |
| | H) 2.47 (t, J = 8.22 Hz, 2 H) 2.89-3.10 (m, 1 H) 3.37-3.54 (m, 3 H) 4.54 |
| | (s, 2 H) 7.12 (d, J = 8.22 Hz, 2 H) 7.44 (d, J = 5.48 Hz, 1 H) 7.99 (t, J = 8.61 |
| | Hz, 1 H) 8.24-8.44 (m, 2 H) 8.92 (s, 1 H) |
| 78 | (400 MHz, <cd3od>) d ppm 0.89 (d, J = 6.65 Hz, 3 H) 1.07 (s, 3 H) 1.21- |
| | 1.44 (m, 2 H) 1.50-1.74 (m, 2 H) 1.91 (d, J = 9.78 Hz, 1 H) 2.09 (quin, |
| | J = 7.63 Hz, 2 H) 2.47 (t, J = 8.02 Hz, 2 H) 2.97-3.8 (m, 1 H) 3.40-3.54 |
| | (m, 3 H) 4.54 (s, 2 H) 7.12 (d, J = 8.22 Hz, 2 H) 7.44 (d, J = 5.09 Hz, 1 H) |
| | 7.99 (t, J = 8.80 Hz, 1 H) 8.26-8.46 (m, 2 H) 8.92 (s, 1 H) |
| 79 | (400 MHz, <cd3od>) d ppm 0.88 (d, J = 6.65 Hz, 3 H) 1.07 (s, 3H) 1.21- |
| | 1.43 (m, 1 H) 1.51-1.71 (m, 3H) 1.80-2.14 (m, 9 H) 3.04 (t, J = 12.52 |
| | Hz, 1 H) 3.40-3.56 (m, 1 H) 7.31 (d, J = 9.39 Hz, 2 H) 7.44 (d, J = 5.09 |
| | Hz, 1 H) 7.97 (t, J = 8.80 Hz, 1 H) 8.29-8.48 (m, 2 H) 8.93 (s, 1 H) |
| 80 | (400 MHz, <cd3od>) d ppm 0.88 (d, J = 6.65 Hz, 3 H) 1.07 (s, 3 H) 1.33 |
| | (q, J = 12.52 Hz, 1 H) 1.49-1.74 (m, 3 H) 1.80-2.13 (m, 9 H) 2.94-3.11 |
| | (m, 1 H) 3.49 (dd, J = 11.74, 4.30 Hz, 1 H) 7.31 (d, J = 9.39 Hz, 2 H) 7.44 |
| | (d, J = 5.48 Hz, 1 H) 7.97 (t, J = 8.80 Hz, 1 H) 8.26-8.52 (m, 2 H) 8.93 (s, |
| | 1 H) |
| 148 | (400 MHz, <cd3od>) d ppm 0.95 (d, J = 6.65 Hz, 3 H) 1.18 (s, 3 H) 1.49 |
| | (q, J = 12.52 Hz, 1 H) 1.56-1.68 (m, 1 H) 1.69-1.81 (m, 1 H) 1.83-2.03 |
| | (m, 4 H) 2.04-2.21 (m, 2 H) 3.12 (dd, J = 12.52, 3.91 Hz, 1 H) 3.31- |
| | 3.41 (m, 1 H) 3.63-3.76 (m, 3 H) 3.88 (dd, J = 11.74, 1.96 Hz, 1 H) 4.50 |
| | (tt, J = 5.97, 3.23 Hz, 1 H) 6.84 (d, J = 10.17 Hz, 2 H) 8.01 (t, J = 8.80 Hz, 1 |
| | H) 8.09 (d, J = 5.87 Hz, 1 H) 8.35-8.47 (m, 1 H) 8.64 (d, J = 5.87 Hz, 1 H) |
| | 9.44 (s, 1 H) |

### KinaseGlo Pim1 ATP Depletion Assay

The activity of PIM1 is measured using a luciferase-luciferin based ATP detection reagent to quantify ATP depletion resulting from kinase-catalyzed phosphoryl transfer to a peptide substrate. Compounds to be tested are dissolved in 100% DMSO and directly distributed into white 384-well plates at 0.5 μl per well. To start the reaction, 10 μl of 5 nM Pim1 kinase and 80 μM BAD peptide (RSRHSSYPAGT-OH) in assay buffer (50 mM HEPES pH 7.5, 5 mM MgCl₂, 1 mM DTT, 0.05% BSA) is added into each well. After 15 minutes, 10 μl of 40 μM ATP in assay buffer is added. Final assay concentrations are 2.5 nM PIM1, 20 μM ATP, 40 μM BAD peptide and 2.5% DMSO. The reaction is performed until approximately 50% of the ATP is depleted, then stopped with the addition of 20 μl KinaseGlo Plus (Promega Corporation) solution. The stopped reaction is incubated for 10 minutes and the remaining ATP detected via luminescence on the Victor2 (Perkin Elmer). Indicated compounds of the foregoing examples were tested by the Pim1 ATP depletion assay and found to exhibit an IC₅₀ values as shown in Table 4, below. IC₅₀, the half maximal inhibitory concentration, represents the concentration of a test compound that is required for 50% inhibition of its target in vitro.

### KinaseGlo Pim2 ATP Depletion Assay

The activity of PIM2 is measured using a luciferase-luciferin based ATP detection reagent to quantify ATP depletion resulting from kinase-catalyzed phosphoryl transfer to a peptide substrate. Compounds to be tested are dissolved in 100% DMSO and directly distributed into white 384-well plates at 0.5 μl per well. To start the reaction, 10 μl of 10 nM Pim2 kinase and 20 μM BAD peptide (RSRHSSYPAGT-OH) in assay buffer (50 mM HEPES pH 7.5, 5 mM MgCl₂, 1 mM DTT, 0.05% BSA) is added into each well. After 15 minutes, 10 μl of 8 μM ATP in assay buffer is added. Final assay concentrations are 5 nM PIM2, 4 μM ATP, 10 μM BAD peptide and 2.5% DMSO. The reaction is performed until approximately 50% of the ATP is depleted, then stopped with the addition of 20 μl KinaseGlo Plus (Promega Corporation) solution. The stopped reaction is incubated for 10 minutes and the remaining ATP detected via luminescence on the Victor2 (Perkin Elmer). Indicated compounds of the foregoing examples were tested by the Pim2 ATP depletion assay and found to exhibit an IC₅₀ values as shown in Table 4, below.

### KinaseGlo Pim3 ATP Depletion Assay

The activity of PIM3 is measured using a luciferase-luciferin based ATP detection reagent to quantify ATP depletion resulting from kinase-catalyzed phosphoryl transfer to a peptide substrate. Compounds to be tested are dissolved in 100% DMSO and directly distributed into white 384-well plates at 0.5 μl per well. To start the reaction, 10 μl of 10 nM Pim3 kinase and 200 μM BAD peptide (RSRHSSYPAGT-OH) in assay buffer (50 mM HEPES pH 7.5, 5 mM MgCl₂, 1 mM DTT, 0.05% BSA) is added into each well. After 15 minutes, 10 μl of 80 μM ATP in assay buffer is added. Final assay concentrations are 5 nM PIM1, 40 μM ATP, 100 μM BAD peptide and 2.5% DMSO. The reaction is performed until approximately 50% of the ATP is depleted, then stopped by the addition of 20 μl KinaseGlo Plus (Promega Corporation) solution. The stopped reaction is incubated for 10 minutes and the remaining ATP detected via luminescence on the Victor2 (Perkin Elmer). Indicated compounds of the foregoing examples were tested by the Pim3 ATP depletion assay and found to exhibit an IC₅₀ values as shown in Table 4, below.

### Cell Proliferation Assay

KMS11 (human myeloma cell line), were cultured in IMDM supplemented with 10% FBS, sodium pyruvate and antibiotics. Cells were plated in the same medium at a density of 2000 cells per well into 96 well tissue culture plates, with outside wells vacant, on the day of assay.

Test compounds supplied in DMSO were diluted into DMSO at 500 times the desired final concentrations before dilution into culture media to 2 times final concentrations. Equal volumes of 2× compounds were added to the cells in 96 well plates and incubated at 37° C. for 3 days.

After 3 days plates were equilibrated to room temperature and equal volume of CellTiter-Glow Reagent (Promega) was added to the culture wells. The plates were agitated briefly and luminescent signal was measured with luminometer. The percent inhibition of the signal seen in cells treated with DMSO alone vs. cells treated with control compound was calculated and used to determine EC₅₀ values (i.e., the concentration of a test compound that is required to obtain 50% of the maximum effect in the cells) for tested compounds, as shown in Tables 4 and 5.

Using the procedures of the Kinase Glo Pim1, 2 and 3 ATP depletion assays the IC₅₀ concentration of indicated compounds of the previous examples were determined as shown in the following table 4.

Using the procedures of Cell Proliferation Assay, the EC₅₀ concentrations of compounds of the examples were determined in KMS11 cells as shown in Table 4.

TABLE 4

| Example | Pim1 | Pim2 | Pim3 | KMS11-luc |
|---|---|---|---|---|
| No. | IC50 μM | IC50 μM | IC50 μM | EC50 μM |
| 25 | | 2.0093 | | |
| 26 | | 0.0208 | | |
| 27 | | 0.0038 | | 0.14 |
| 28 | | 0.2294 | | |
| 29 | | 0.0149 | | 3.27 |
| 30 | | 0.0022 | | 0.13 |
| 31 | | 0.0934 | | 10.00 |
| 32 | 0.0004 | 0.0010 | 0.0014 | 0.88 |
| 33 | 0.0005 | 0.0011 | 0.0018 | 0.58 |
| 34 | 0.0006 | 0.0013 | 0.0017 | 0.61 |
| 35 | 0.0495 | 0.6765 | 0.1503 | 10.00 |
| 36 | 0.5247 | 5.4123 | 0.3074 | |
| 37 | 0.0013 | 0.0049 | 0.0025 | |
| 38 | 0.0036 | 0.0243 | 0.0093 | 10.00 |
| 39 | 0.0047 | 0.0255 | 0.0045 | 10.00 |
| 40 | 0.2091 | 2.8822 | 0.3853 | 10.00 |
| 41 | 0.0009 | 0.0023 | 0.0029 | |
| 42 | 0.0446 | 0.8464 | 0.4343 | 10.00 |
| 43 | 0.0004 | 0.0020 | 0.0020 | 1.54 |
| 44 | 0.0086 | 0.0442 | 0.0293 | 10.00 |
| 45 | 0.0004 | 0.0012 | 0.0014 | 0.45 |
| 46 | 0.0014 | 0.0109 | 0.0035 | 10.00 |
| 47 | 0.0010 | 0.0025 | 0.0019 | 1.31 |
| 48 | 0.0004 | 0.0013 | 0.0014 | 0.87 |
| 49 | 0.0715 | 1.6426 | 0.2164 | 10.00 |
| 50 | 0.0006 | 0.0035 | 0.0015 | 4.78 |
| 51 | 0.0053 | 0.0254 | 0.0042 | 10.00 |
| 52 | 0.0020 | 0.0135 | 0.0036 | 9.74 |
| 53 | 0.0010 | 0.0114 | 0.0022 | 10.00 |
| 54 | 0.0033 | 0.0679 | 0.0121 | 10.00 |
| 55 | 0.0127 | 0.7402 | 0.2201 | 10.00 |
| 56 | 0.0004 | 0.0053 | 0.0029 | 7.96 |
| 57 | 0.0151 | 0.2268 | 0.1018 | 6.62 |
| 58 | 0.0004 | 0.0034 | 0.0026 | 6.06 |
| 59 | 0.0003 | 0.0015 | 0.0013 | 1.07 |
| 60 | 0.0193 | 1.7127 | 0.3288 | 10.00 |
| 61 | 0.0003 | 0.0017 | 0.0014 | 0.32 |
| 62 | 0.0395 | 1.7262 | 0.3963 | 10.00 |
| 125 | | 0.0059 | | |
| 126 | | 0.0431 | | 8.03 |
| 127 | | 0.0200 | | 1.39 |
| 128 | | 0.0097 | | 10.00 |
| 129 | | 0.0201 | | 3.30 |
| 130 | | 0.0088 | | 8.96 |
| 131 | | 0.0516 | | 10.00 |
| 132 | | 0.1623 | | 7.14 |
| 133 | | 0.0054 | | 5.37 |
| 134 | | 0.0507 | | 4.15 |
| 135 | | 0.0142 | | 10.00 |
| 136 | 0.0010 | 0.0046 | 0.0019 | 5.75 |
| 137 | 0.0015 | 0.0054 | 0.0023 | 8.58 |
| 138 | 0.0017 | 0.0129 | 0.0026 | 10.00 |
| 139 | 0.0008 | 0.0040 | 0.0022 | 1.71 |
| 140 | 0.0075 | 0.1304 | 0.0152 | 10.00 |
| 141 | 0.0011 | 0.0021 | 0.0022 | 4.87 |
| 142 | 0.1516 | 0.7689 | 0.2453 | 10.00 |
| 143 | 0.0088 | 0.1205 | 0.0087 | 6.69 |
| 144 | 0.0007 | 0.0077 | 0.0023 | 4.22 |
| 145 | 0.0015 | 0.0092 | 0.0040 | 3.24 |

### Pim1, Pim2, Pim3 AlphaScreen Assay

Pim 1, Pim 2 & Pim 3 AlphaScreen assays using high ATP (11-125×ATP Km) were used to determine the biochemical activity of the inhibitors. The activity of Pim 1, Pim 2, & Pim 3 is measured using a homogeneous bead based system quantifying the amount of phosphorylated peptide substrate resulting from kinase-catalyzed phosphoryl transfer to a peptide substrate. Compounds to be tested are dissolved in 100% DMSO and directly distributed to a white 384-well plate at 0.25 μl per well. To start the reaction, 5 μl of 100 nM Bad peptide (Biotin-AGAGRSRHSSYPAGT-OH) and ATP (concentrations described below) in assay buffer (50 mM Hepes, pH=7.5, 5 mM MgCl₂, 0.05% BSA, 0.01% Tween-20, 1 mM DTT) is added to each well. This is followed by the addition of 5 μl/well of Pim 1, Pim 2 or Pim 3 kinase in assay buffer (concentrations described below). Final assay concentrations (described below) are in 2.5% DMSO. The reactions are performed for ˜2 hours, then stopped by the addition of 10 μl of 0.75 μg/ml anti-phospho Ser/Thr antibody (Cell Signaling), 10 μg/ml Protein A AlphaScreen beads (Perkin Elmer), and 10 μg/ml streptavidin coated AlphaScreen beads in stop/detection buffer (50 mM EDTA, 95 mM Tris, pH=7.5, 0.01% Tween-20). The stopped reactions are incubated overnight in the dark. The phosphorylated peptide is detected via an oxygen anion initiated chemiluminescence/fluorescence cascade using the Envision plate reader (Perkin Elmer).

AlphaScreen Assay Conditions

| | | b-BAD | | ATP Km |
|---|---|---|---|---|
| Enzyme | Enzyme conc. | peptide conc. | ATP conc. | (app) |
| source | (nM) | (nM) | (uM) | (uM) |
| Pim 1 (INV) | 0.0025 | 50 | 2800 | 246 |
| Pim 2 (INV) | 0.01 | 50 | 500 | 4 |
| Pim 3 (NVS) | 0.005 | 50 | 2500 | 50 |

Indicated compounds of the foregoing examples were tested by the Pim 1, Pim 2 & Pim 3 AlphaScreen assays and found to exhibit an IC₅₀ values as shown in Table 5, below. IC₅₀, the half maximal inhibitory concentration, represents the concentration of a test compound that is required for 50% inhibition of its target in vitro under the described assay conditions.

Using the procedures of Cell Proliferation Assay, the EC₅₀ concentration of indicated compounds of the examples in were determined in KMS11 cells as shown in Table 5.

TABLE 5

| Example | Pim1 | Pim2 | Pim3 | KMS11-luc |
|---|---|---|---|---|
| No. | IC50 μM | IC50 μM | IC50 μM | EC50 μM |
| 1 | 0.00007 | 0.00320 | 0.00145 | 0.020 |
| 2 | 0.00005 | 0.00268 | 0.00133 | 0.059 |
| 3 | 0.00010 | 0.01058 | 0.00194 | 0.126 |
| 4 | 0.00009 | 0.00433 | 0.00243 | 0.034 |
| 6 | 0.00014 | 0.02488 | 0.00240 | 0.202 |
| 7 | 0.00008 | 0.00631 | 0.00209 | 0.057 |
| 8 | 0.00008 | 0.00698 | 0.00204 | 0.234 |
| 9 | 0.00854 | 1.01074 | 1.61812 | 4.699 |
| 10 | 0.01461 | 7.11414 | 4.15023 | >10 |
| 11 | 0.00013 | 0.00810 | 0.00143 | |
| 12 | 0.02623 | 1.43942 | 0.30270 | |
| 13 | 0.00007 | 0.00433 | 0.00116 | 0.042 |
| 14 | 0.01372 | 0.77568 | 0.23064 | 5.042 |
| 15 | 0.00008 | 0.00600 | 0.00104 | |
| 16 | 0.00425 | 0.78040 | 0.07295 | |
| 17 | 0.02843 | 4.64607 | 0.64608 | |
| 18 | 0.00067 | 0.04697 | 0.01017 | 0.714 |
| 19 | 0.00610 | 0.11135 | 0.06574 | 0.247 |
| 20 | 0.23477 | 15.85922 | 3.52924 | |
| 21 | 0.00095 | 0.10666 | 0.00896 | 1.056 |
| 22 | 0.00085 | 0.20070 | 0.03946 | 0.382 |
| 23 | 0.00058 | 0.02865 | 0.01008 | |
| 24 | 0.02208 | 4.94378 | 0.78607 | |
| 25 | 0.11119 | 8.12929 | 0.99220 | |
| 26 | 0.00190 | 0.10944 | 0.01724 | |
| 27 | 0.00011 | 0.00573 | 0.00099 | 0.138 |
| 28 | 0.01835 | 0.47593 | 0.13834 | |
| 29 | 0.00670 | 0.42825 | 0.20065 | 3.269 |
| 30 | 0.00071 | 0.05011 | 0.02283 | 0.133 |
| 31 | 0.21524 | 22.06609 | 5.47883 | >10 |
| 61 | 0.00196 | 0.16391 | 0.07362 | 0.317 |
| 63 | | 0.00573 | | 0.084 |
| 64 | 0.00002 | 0.00154 | 0.00054 | 0.165 |
| 65 | 0.00003 | 0.00214 | 0.00073 | 0.038 |
| 66 | 0.00003 | 0.00292 | 0.00164 | 0.013 |
| 67 | 0.00001 | 0.00250 | 0.00096 | 0.127 |
| 68 | | 0.00302 | | 0.024 |
| 69 | 0.00009 | 0.00642 | 0.00420 | 0.423 |
| 70 | 0.00004 | 0.00221 | 0.00173 | 0.136 |
| 71 | 0.00002 | 0.00147 | 0.00072 | 0.081 |
| 72 | | 0.00400 | | 0.098 |
| 73 | 0.00042 | 0.15616 | 0.06306 | 0.189 |
| 74 | 0.01884 | 6.33767 | 2.86626 | 3.468 |
| 75 | 0.03914 | 8.64389 | 5.89821 | 5.781 |
| 76 | 0.00082 | 0.26377 | 0.11726 | 1.145 |
| 77 | 0.00097 | 0.09369 | 0.08327 | 0.136 |
| 78 | 0.01873 | 1.56944 | 0.96851 | >10 |
| 79 | 0.00673 | 0.61871 | 0.47010 | >10 |
| 80 | 0.00015 | 0.12638 | 0.05496 | 0.169 |
| 81 | 0.00003 | 0.00442 | 0.00094 | 0.409 |
| 82 | 0.00002 | 0.00268 | 0.00118 | 0.043 |
| 83 | 0.00003 | 0.00371 | 0.00071 | 9.299 |
| 84 | 0.00001 | 0.00280 | 0.00048 | 0.343 |
| 85 | 0.00001 | 0.00199 | 0.00032 | 0.706 |
| 86 | 0.00003 | 0.00274 | 0.00246 | 0.066 |
| 87 | 0.00001 | 0.00191 | 0.00041 | 0.057 |
| 88 | 0.00003 | 0.00580 | 0.00559 | 0.101 |
| 148 | 0.00002 | 0.00145 | 0.00065 | 0.085 |
| 89 | 0.00002 | 0.00147 | 0.00035 | 0.022 |
| 90 | 0.00047 | 0.21472 | 0.06444 | 0.557 |
| 91 | 0.00007 | 0.00240 | 0.00162 | 0.102 |
| 92 | 0.00009 | 0.00389 | 0.00220 | 0.091 |
| 93 | 0.00009 | 0.00844 | 0.00123 | 1.397 |
| 94 | 0.00030 | 0.11407 | 0.04776 | 3.231 |
| 95 | 0.00004 | 0.00385 | 0.00237 | 0.174 |
| 96 | 0.01866 | 8.72322 | 2.62291 | >10 |
| 97 | 0.00024 | 0.02890 | 0.00824 | 0.405 |
| 98 | 0.00083 | 0.13332 | 0.04022 | 0.427 |
| 99 | 0.00096 | 0.10701 | 0.04549 | 1.149 |
| 100 | 0.00004 | 0.00215 | 0.00069 | 0.340 |
| 101 | 0.00004 | 0.00221 | 0.00085 | 0.334 |
| 102 | 0.00002 | 0.00150 | 0.00061 | 0.175 |
| 103 | 0.00143 | 0.01407 | 0.05889 | >10 |
| 104 | 0.00007 | 0.00756 | 0.00437 | 0.823 |
| 105 | 0.00055 | 0.11987 | 0.07829 | 2.984 |
| 106 | 0.00042 | 0.18262 | 0.06924 | 1.536 |
| 107 | 0.00089 | 0.25269 | 0.07416 | 3.182 |
| 108 | 0.00020 | 0.06619 | 0.03068 | 0.206 |
| 109 | 0.00023 | 0.13843 | 0.04128 | 0.781 |
| 110 | 0.00054 | 0.16560 | 0.10637 | 0.322 |
| 111 | 0.00083 | 0.17276 | 0.03874 | 0.538 |
| 112 | 0.00011 | 0.05648 | 0.01826 | 4.153 |
| 113 | 0.00055 | 0.12818 | 0.12659 | 0.248 |
| 114 | 0.00034 | 0.02174 | 0.00693 | 0.924 |
| 115 | 0.00018 | 0.10376 | 0.03279 | 0.310 |
| 116 | 0.00265 | 0.67215 | 0.18883 | >10 |
| 117 | 0.00002 | 0.00139 | 0.00095 | 0.256 |
| 118 | 0.00007 | 0.00357 | 0.00161 | 0.539 |
| 119 | | 0.00153 | | 0.578 |
| 120 | 0.00026 | 0.04870 | 0.03145 | 0.723 |
| 121 | 0.10124 | 25.00000 | 2.32171 | |
| 122 | 0.00953 | 1.34848 | 0.28637 | 1.745 |
| 123 | 0.03516 | 20.21699 | 0.45406 | |
| 124 | 0.02167 | >25 | 0.73556 | |
| 125 | 0.02306 | 2.13802 | 0.60138 | |
| 126 | 0.04011 | 14.16042 | 3.52315 | 8.034 |
| 127 | 0.01193 | 9.18427 | 0.61942 | 1.393 |
| 128 | 0.02904 | 2.31488 | 1.06995 | >10 |
| 129 | 0.05770 | 8.16479 | 1.71476 | 3.297 |
| 130 | 0.00982 | 2.14137 | 0.38306 | 8.965 |
| 131 | 0.14583 | 17.00252 | 5.08818 | >10 |
| 132 | 2.53010 | >25 | >25 | 7.137 |
| 133 | 0.00719 | 2.62705 | 0.18206 | 5.372 |
| 134 | 0.07872 | 14.27704 | 3.38681 | 4.150 |
| 135 | 0.03350 | 5.68761 | 1.13285 | >10 |
| 145 | 0.02525 | | 0.95254 | 3.243 |
| 146 | 0.00002 | 0.00166 | 0.00053 | 0.049 |
| 147 | 0.00004 | 0.00237 | 0.00053 | 0.032 |

1. A compound of Formula (I): 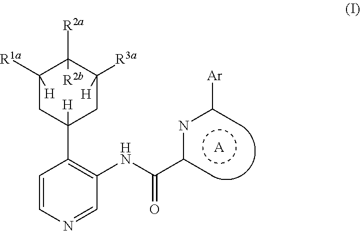
wherein:
groups attached to the cyclohexyl ring that are depicted inside the ring are all syn to each other, and all groups attached to the cyclohexyl ring that are depicted outside the cyclohexyl ring are syn to one another;
R^{1a} and R^{3a} are selected from hydroxyl, C1-C4 alkyl, —(CH₂)₁₋₃Z, C1-C4 haloalkyl, C1-C4 alkoxy, C1-C4 haloalkoxy, C1-C4 hydroxyalkyl, and amino,
R^{2a} is selected from C1-C4 alkyl, —(CH₂)₁₋₃Z, C1-C4 haloalkyl, and C1-C4 hydroxyalkyl,
wherein Z is —OH, NH₂, —NHC(O)Q, or —OC(O)Q, where Q is H or C1-C4 alkyl optionally substituted with one or more halo, OH, NH₂, OMe, or CN;
R^{2b} is OH;
ring A is a 5 or 6 membered aromatic ring selected from pyridinyl, pyrimidinyl, pyrazinyl, and thiazolyl and having N positioned as shown in Formula (I);
Ring A is optionally substituted with 1 or 2 groups selected from halo, CN, NH₂, hydroxy, C1-C4 alkyl, C1-C4 haloalkyl, C1-C4 alkoxy, and C1-C4 haloalkoxy;
Ar is an aromatic ring selected from phenyl, pyridinyl, pyrimidinyl, pyrazinyl, pyridazinyl, thiazolyl, and pyrazolyl, or a 3-6 membered cycloalkyl or cycloalkenyl, each of which is optionally fused to an additional C₅₋₆ cycloalkyl, C₅₋₆ heterocyclyl, C₅₋₆ heteroaryl or phenyl; and
Ar is optionally substituted with up to three groups independently selected from halo, CN, NH₂, hydroxy, C1-C4 haloalkyl, —S(O)ₚ-Q², C1-C4 haloalkoxy, —(CH₂)₀₋₃—OQ², —O—(CH₂)₁₋₃—OQ², COOQ², C(O)Q², —(CR′₂)₁₋₃—OR′ or —(CR′₂)₁₋₃—OR′ where each R′ is independently H or Me or C₂₋₄ alkyl, and an optionally substituted member selected from the group consisting of C₁₋₆ alkyl, C₁₋₆ alkoxy, C₁₋₆ alkylthio, C₁₋₆ alkylsulfonyl, C₃₋₇ cycloalkyl, C₅₋₇ cycloalkenyl, C₃₋₇ heterocycloalkyl, C₄₋₆ cyclic ether, C₅₋₁₀ heteroaryl, and C₆₋₁₀ aryl, each of which is optionally substituted with up to two groups selected from halo, CN, NH₂, hydroxy, oxo, C₁₋₄haloalkyl, C₁₋₄ alkoxy, and Q²;
where Q² is H or a 4-7 membered cyclic ether, phenyl, C₅₋₆ heteroaryl, or C₁₋₆ alkyl, each of which is optionally substituted with one or more halo, oxo, OH, NH₂, COOH, COOMe, COOEt, COONH₂, COONHMe, COONMe₂, OMe, OEt, or CN,
and p is 0-2;
or a pharmaceutically acceptable salt thereof.

2. The compound of claim 1, wherein R^{1a} and R^{3a} are different.

3. The compound of claim 1, wherein R^{1a} is OH.

4. The compound of claim 1, wherein R^{1a} is OH and R^{3a} is Me.

5. The compound of claim 1, wherein R^{1a} is NH₂ and R^{3a} is Me.

6. The compound of claim 1, wherein Ar is substituted with one to three groups selected from F, Cl, NH₂, Me, Et, OMe, OEt, OCF₃, OCHF₂, OCH₂CF₃, CN, CF₃, SMe, SOMe, SO₂Me, —COOMe, —C(O)Me, —C(Me)₂-OH, MeOCH₂—, HOCH₂—, hydroxyethyl, hydroxyethoxy, methoxyethyl, methoxyethoxy, oxetanyl (e.g., 3-oxetanyl), isopropoxy, tetrahydropyranyloxy (e.g., 4-tetrahydropyranyloxy), cyclopropyl, and CN.

7. The compound of claim 1, wherein Ar is substituted on at least one position adjacent to the ring atom of Ar that is attached to ring A.

8. The compound of claim 1, wherein Ar is phenyl or 2-pyridinyl, and is substituted with up to three groups selected from F, Cl, NH₂, Me, Et, OMe, OEt, OCF₃, OCHF₂, OCH₂CF₃, CN, CF₃, SMe, SOMe, SO₂Me, —COOMe, —C(O)Me, —C(Me)₂-OH, MeOCH₂—, HOCH₂—, hydroxyethyl, hydroxyethoxy, methoxyethyl, methoxyethoxy, oxetanyl (e.g., 3-oxetanyl), isopropoxy, tetrahydropyranyloxy (e.g., 4-tetrahydropyranyloxy), cyclopropyl, and CN.

9. The compound of claim 8, wherein ring A is substituted with at least one halo or NH₂.

10. The compound of claim 1, wherein Ring A is pyridinyl.

11. The compound of claim 10, wherein exactly one of R^{1a} and R^{3a} is the same as R^{2a}.

12. The compound of claim 11, wherein one of R^{1a} and R^{3a} is Me, and the other one is OH or NH₂.

13. The compound of claim 1, wherein R^{2a} is selected from CH₂F, —CH₂OH, —CH₂OAc, Et and Me.

14. The compound of claim 1, wherein at least one of R^{1a} and R^{3a} is Me.

15. The compound of claim 1, which is optically active and has a lower IC-50 than its opposite enantiomer on Pim kinase.

16. The compound of claim 1, which is an optically active compound of Formula IIa or IIb: 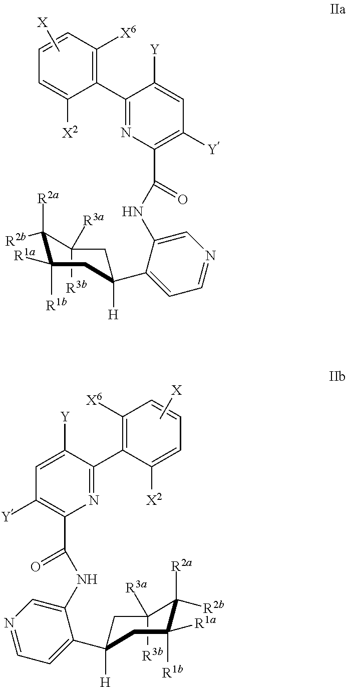
wherein, X, X² and X⁶ are independently selected from H, halo, CN, Me, OMe, OEt, OCHF₂, OCH₂CF₃, MeOCH₂—, HOCH₂—, hydroxyethyl, hydroxyethoxy, methoxyethyl, methoxyethoxy, F, Cl, NH₂, Me, Et, OCF₃, CF₃, SMe, SOMe, SO₂Me, —COOMe, —C(O)Me, —C(Me)₂-OH, MeOCH₂—, HOCH₂—, hydroxyethyl, hydroxyethoxy, methoxyethyl, methoxyethoxy, oxetanyl (e.g., 3-oxetanyl), isopropoxy, tetrahydropyranyloxy (e.g., 4-tetrahydropyranyloxy), cyclopropyl, and CN;
R^{1b} and R^{3b} are both H;
Y and Y′ are independently selected from H, halo, and NH₂;
or a pharmaceutically acceptable salt thereof.

17. The compound of claim 16, wherein X² and X⁶ are each F.

18. The compound of claim 16, wherein Y is F and Y′ is H or NH₂.

19. The compound of claim 16, wherein X is H, F, Cl, Me, Et, OMe, OEt, OCF₃, OCHF₂, OCH₂CF₃, CN, CF₃, SMe, SOMe, SO₂Me, —COOMe, —C(O)Me, —C(Me)₂-OH, MeOCH₂—, HOCH₂—, hydroxyethyl, hydroxyethoxy, methoxyethyl, methoxyethoxy, 3-oxetanyl, 4-tetrahydropyranyloxy, cyclopropyl, or CN.

20. The compound of claim 16, wherein one of R^{1a} and R^{3a} is NH₂ or OH, and the other one is Me.

21. The compound of claim 16, wherein R^{2b} is OH.

22. The compound of claim 16, wherein R^{2a} is Me, —CH₂OH, —CH₂F, or Et.

23. The compound of claim 16, which is a compound of Formula IIa.

24. The compound of claim 16, which is a compound of Formula IIb.

25. A compound selected from the group consisting of the compounds in Tables 1 and 2, and the pharmaceutically acceptable salts thereof.

26. A pharmaceutical composition comprising a compound of claim 1, admixed with at least one pharmaceutically acceptable excipient.

27. The pharmaceutical composition of claim 26, which comprises at least two pharmaceutically acceptable excipients.

28. The pharmaceutical composition of claim 26, which further comprises an additional agent for treatment of cancer.

29. The pharmaceutical composition of claim 28, wherein the additional therapeutic agent is selected from irinotecan, topotecan, gemcitabine, 5-fluorouracil, cytarabine, daunorubicin, PI3 Kinase inhibitors, mTOR inhibitors, DNA synthesis inhibitors, leucovorin, carboplatin, cisplatin, taxanes, tezacitabine, cyclophosphamide, vinca alkaloids, imatinib, anthracyclines, rituximab, and trastuzumab.

30. A method of treating a disease or condition mediated by PIM kinase, comprising administering to a subject in need thereof a therapeutically effective amount of a compound according to claim 1, or a pharmaceutically acceptable salt thereof.

31. The method of claim 30, wherein the disease is selected from carcinoma of the lungs, pancreas, thyroid, ovaries, bladder, breast, prostate or colon, melanoma, myeloid leukemia, multiple myeloma, erythro leukemia, villous colon adenoma, and osteosarcoma; or the disease is an autoimmune disorder.

32. The method of claim 31, wherein the disease is an autoimmune disorder.

33. The method of claim 32, wherein the autoimmune disorder is selected from Crohn's disease, inflammatory bowel disease, rheumatoid arthritis, and chronic inflammatory diseases.
